(19) United States
(12) Patent Application Publication
(10) Pub. No.: US 2026/0365664 A1
(43) Pub. Date: Nov. 19, 2026

(54) HARRINGTONINE DERIVATIVE, PHARMACEUTICAL COMPOSITION THEREOF, PREPARATION METHOD THEREFOR, AND USE THEREOF

(71) Applicant: NANKAI UNIVERSITY, Tianjin (CN)

(72) Inventors: Li CHEN, Tianjin (CN); Yuanqiang GUO, Tianjin (CN); Ling QIAO, Tianjin (CN); Mingli QI, Tianjin (CN); Yue ZHANG, Tianjin (CN); Ying ZHAO, Tianjin (CN); Jiantong HOU, Tianjin (CN)

(21) Appl. No.: 19/114,508

(22) PCT Filed: Sep. 22, 2023
(86) PCT No.: PCT/CN2023/120776

§ 371 Date: Mar. 24, 2025

(30) Foreign Application Priority Data
Sep. 22, 2022 (CN) 202211161020.8

(51) Int. Cl. C07D 491/20; A61K 31/55; A61P 35/00; A61P 35/02

(52) U.S. Cl. CPC C07D 491/20; A61K 31/55; A61P 35/00; A61P 35/02

(57) ABSTRACT

A harringtonine derivative, pharmaceutical composition thereof, preparation method therefor, and use thereof are provided. The harringtonine derivative and pharmaceutical composition thereof provided herein have excellent anti-tumor activity.

This application claims the priority of the prior invention patent application submitted by the applicant on Sep. 22, 2022, to China National Intellectual Property Administration, with the application No. 202211161020.8, titled “HARRINGTONINE DERIVATIVE, PHARMACEUTICAL COMPOSITION THEREOF, PREPARATION METHOD THEREFOR, AND USE THEREOF”, which is incorporated herein by reference in its entirety.

### TECHNICAL FIELD

The disclosure relates to harringtonine derivative, pharmaceutical composition thereof, preparation method therefor, and use thereof, and belongs to the field of pharmaceutical compounds thereof.

### BACKGROUND

Cephalotaxus ester alkaloids, such as harringtonine 1 (HT), homoharringtonine 2 (HHT), deoxyharringtonine 3 (DHT) and isoharringtonine 4 (IHT) are broad-spectrum anticancer drugs. Among them, homoharringtonine 2 was introduced into the Pharmacopoeia of Chinese in 1990 for the clinical treatment of acute non-lymphocytic leukemia, and has been clinically applied so far.

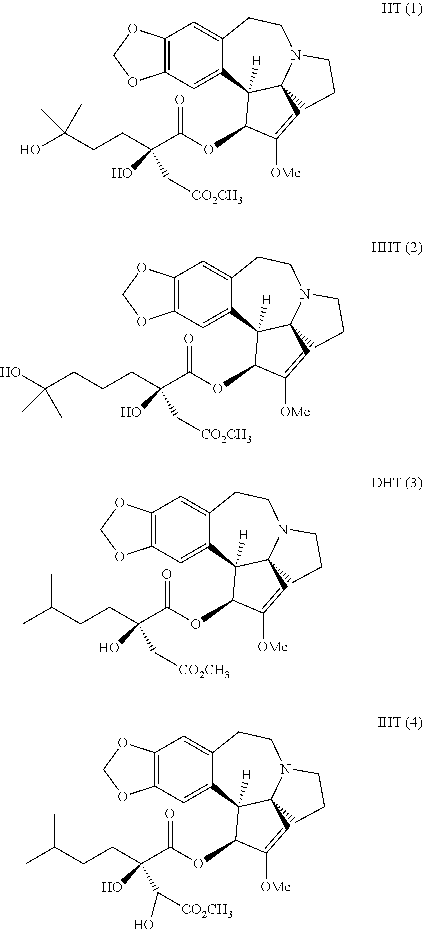

At present, the alkaloids of Cephalotaxus esters are isolated from Cephalotaxus plants. Natural Cephalotaxus ester alkaloids account for only a small part of the total alkaloids, and mainly exist in the non-renewable parts of plants. The Cephalotaxus ester alkaloids are not only present in natural plant materials at low concentrations, but also are mixed with many similarly similar congeners, so that it is very difficult to isolate high pure Cephalotaxus ester alkaloids from natural products. At the same time, the abundance of plants in the genus Cephalotaxus has plummeted and it has become a rare protected plant. Therefore, the separation of Cephalotaxus ester alkaloids from plants has been far from meeting the needs of clinical applications. The inventors have previously been granted Chinese Patent ZL201510925650.1, which disclosed a class of cephalotaxine alkaloid compounds, their synthetic methods, and applications. Some of the exemplified compounds in the patent demonstrated certain inhibitory activity against HL-60 human leukemia cells. Currently, there is a need for further research on the derivatives of such compounds to develop drug candidates with improved anticancer activity.

### SUMMARY

To address the aforementioned technical issues, the present disclosure provides a compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof:

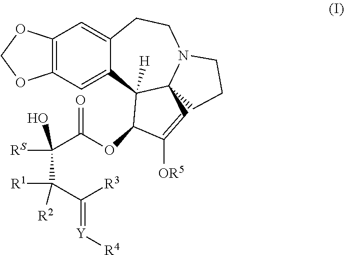

wherein R^{S} is optionally selected from (R^{N})m-G-(CH₂)n- and (R^{Q})m-G-(CH₂)ₙ—;
R^{N} represents a nitrogen-containing substituent, such as a nitro group, or the following groups which are unsubstituted or optionally substituted with one, two, or more R^{a}: —NR⁶R⁷, —NR⁶—C(═Y—R⁴)—NR⁶R⁷;
each of R⁶, R^{6′}, and R⁷ is the same or different and is independently selected from H, or the following groups which are unsubstituted or optionally substituted with one, two, or more R^{b}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹⁰C(═O)—, R¹⁰C(═O)O—, R¹⁰C(═O)NH—, R¹⁰OC(═O)—, R¹⁰NHC(═O)—, R¹⁰S(O)₂—, R¹⁰S(O)₂O—, R¹⁰OS(O)₂—, R¹⁰S(O)—, tri(C₁₋₂₀ alkyloxy)silyl, C₁₋₂₀ alkyl, tri(C₁₋₂₀ alkyl)silyl;
R^{Q} represents H, halogen, —OH, —CN, or the following groups which are unsubstituted or optionally substituted with one, two, or more R^{e}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹⁰C(═O)—, R¹⁰C(═O)O—, R¹⁰OC(═O)—, R¹⁰S(O)₂—, R¹⁰S(O)₂O—, R¹⁰OS(O)₂—, R¹⁰S(O)—, tri(C₁₋₂₀ alkyloxy)silyl C₁₋₂₀ alkyl, tri(C₁₋₂₀ alkyl)silyl;
G represents a chemical bond, C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, or 5-20 membered heteroaryl group. When G is not a chemical bond, the different substituents attached to G can be spaced apart by 1-10 atoms on G, for example, by 1, 2, 3, 4, 5, 6, 7, 8, 9, or 10 atoms. For instance, when G is a phenyl group, the different substituents attached to G can be positioned 1, 2, 3, 4, or 5 carbon atoms apart, meaning that the substituents on G can be in the ortho, meta, and/or para positions, preferably in the meta and/or para positions;
each m is the same or different and is independently selected from integers greater than 1;
each n is the same or different and is independently selected from integers greater than 1;
each Y is the same or different and is independently selected from O, S, or N, provided that when Y is selected from O or S, R⁴ does not exist;
R¹ and R² are the same or different and are independently selected from H, C₁₋₄ alkyl, or halogen.
R³ is selected from R⁸, R⁸—O—, R⁸—S—;
R⁴ does not exist, or is selected from H, or the following groups which are unsubstituted or optionally substituted with one, two, or more R^{d}: R⁸—O—, R⁸—S—, R⁹C(═O)O—, R¹⁰OC(═O)O—, R¹⁰NHC(═O)—, R¹⁰NHC(═O)O—, R¹⁰C(═O)NH—;
R⁵ is selected from H or C₁₋₄ alkyl;
each of R^{a}, R^{b}, R^{c}, and R^{d} is the same or different and is independently selected from halogen, —OH, —CN, —NO₂, oxo (═O), or the following groups which are unsubstituted or optionally substituted with one, two, or more R^{e}: C₁₋₂₀ alkyl, C₂₋₂₀ alkenyl, C₃₀ cycloalkyl, C₃₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹¹C(═O)—, R¹¹C(═O)O—, R¹¹C(═O)NH—, R¹¹OC(═O)—, R¹¹NHC(═O)—, R¹¹S(O)₂—, R¹¹S(O)₂O—, R¹¹OS(O)₂—, R¹¹S(O)—, —NR¹²R¹³, —NH—C(═Y)—NR¹²R¹³, tri(C₁₋₂₀ alkyloxy)silyl C₁₋₂₀ alkyl, tri(C₁₋₂₀ alkyl)silyl;
each of R⁸, R⁹, R¹⁰, and R¹¹ is the same or different and is independently selected from H, C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, R^{11′}C(═O)—, R^{11′}C(═O)O—, R^{11′}C(═O)NH—, R^{11′}OC(═O)—, R^{11′}NHC(═O)—, R^{11′}S(O)₂—, R^{11′}S(O)₂O—, R^{11′}OS(O)₂—, R^{11′}S(O)—, —NR¹²R¹³, —NH—C(═Y)—NR¹²R¹³, tri(C₁₋₂₀ alkyloxy)silyl C₁₋₂₀ alkyl, tri(C₁₋₂₀ alkyl)silyl;
each of R^{11′}, R¹², and R¹³ is the same or different and is independently selected from H, or the following groups which are unsubstituted or optionally substituted with one, two, or more R^{f}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₁₀ aryl, 5-20 membered heteroaryl;
each of R^{e} and R^{f} is the same or different and is independently selected from halogen, —OH, —CN, —NO₂, —NH₂, oxo (═O), or the following groups which are unsubstituted or optionally substituted with one, two, or more R^{g}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R⁴C(═O)—, R⁴C(═O)O—, R⁴OC(═O)—, R⁴S(O)₂—, R⁴S(O)₂O—, R⁴OS(O)₂—, R⁴S(O)—;
each R¹⁴ is the same or different and is independently selected from C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, or 5-20 membered heteroaryl;
each R^{g} is the same or different and is independently selected from halogen, —OH, —CN, —NO₂, —NH₂, oxo (═O), C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, or 5-20 membered heteroarylthio;
optionally, one, two, or more carbon atoms of the C₃₋₂₀ cycloalkyl or 3-20 membered heterocyclyl may be optionally substituted with oxygen to form a carbonyl group.

According to the embodiments of the present disclosure, G represents a chemical bond, C₁₋₈ alkyl, C₂₋₈ alkenyl, C₃₋₈ cycloalkyl, C₃₋₈ cycloalkenyl, 3-15 membered heterocyclyl, C₆₋₁₅ aryl, or 5-15 membered heteroaryl, such as a chemical bond, C₁₋₈ alkyl, C₂₋₈ alkenyl, phenyl, or naphthyl.

According to the embodiments of the present disclosure, m represents an integer from 1 to 20, preferably an integer from 1 to 10, such as 1, 2, 3, 4, 5, 6, 7, 8, 9, or 10.

According to the embodiments of the present disclosure, n represents an integer from 1 to 20, preferably an integer from 1 to 10, such as 1, 2, 3, 4, 5, 6, 7, 8, 9, or 10.

According to the embodiments of the present disclosure, halogen represents F, Cl, Br, or I.

According to the embodiments of the present disclosure, Y is selected from O, N, or S.

According to the embodiments of the present disclosure, R³ is selected from H, C₁₋₈ alkyl, C₂₋₈ alkenyl, C₃₋₁₅ cycloalkyl, C₃₋₈ cycloalkenyl, 3-15 membered heterocyclyl, C₆₋₁₅ aryl, 5-15 membered heteroaryl, C₁₋₈ alkyl-O—, C₂₋₈ alkenyl —O—, C₃₋₁₅ cycloalkyl —O—, C₃₋₁₅ cycloalkenyl —O—, 3-15 membered heterocyclyl —O—, C₆₋₁₅ aryl —O—, 5-15 membered heteroaryl —O—, C₁₋₈ alkyl —S—, C₂₋₈ alkenyl —S—, C₃₋₁₅ cycloalkyl —S—, C₃₋₁₅ cycloalkenyl —S—, 3-15 membered heterocyclyl —S—, C₆₋₁₅ aryl —S—, or 5-15 membered heteroaryl —S—.

According to the embodiments of the present disclosure, R⁴ does not exist or is selected from H.

According to the embodiments of the present disclosure, R represents a nitrogen-containing substituent, such as a nitro group, or the following groups which are unsubstituted or optionally substituted with one, two, or more R^{a}: —NR⁶R⁷, —NH—C(═Y—R⁴)—NR⁶R⁷.

According to the embodiments of the present disclosure, R^{N} represents a nitro group, or the following groups which are unsubstituted or optionally substituted with one, two, or more R^{a}: —NR⁶R⁷, —NH—C(═Y—R⁴)—NR⁶R⁷, where Y is selected from O, N, or S, and R⁴ does not exist or is selected from H.

According to the embodiments of the present disclosure, R^{N} is selected from —NR⁶R⁷, —NH—C(═O)—NR⁶R⁷, —NH—C(═S)—NR⁶R⁷, or —NH—C(═NH)—NR⁶R⁷.

Alternatively, according to the embodiments of the present disclosure, R^{N} is selected from a nitro group, an amino group, or a phenyl group substituted at the ortho, meta, and/or para positions with a nitro or amino group, where the amino group is unsubstituted or substituted with one, two, or more substituents selected from C₁₋₈ alkylcarbonyl, C₁₋₈ alkoxycarbonyl, C₃₋₈ cycloalkyl carbonyl, C₃₋₈ cycloalkoxycarbonyl, C₆₋₁₀ arylcarbonyl, C₆₋₁₀ aryloxycarbonyl, or C₂₋₈ alkenyl.

According to the embodiments of the present disclosure, R⁶ is selected from H, or the following groups which are unsubstituted or optionally substituted with one, two, or more R^{b}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, R¹⁰C(═O)—, R¹⁰C(═O)O—, R¹⁰OC(═O)—, R¹⁰S(O)₂—, R¹⁰S(O)₂O—, R¹⁰OS(O)—, R¹⁰S(O)—, tri(C₁₋₂₀ alkyloxy)silyl C₁₋₂₀ alkyl, or tri(C₁₋₂₀ alkyl)silyl.

According to the embodiments of the present disclosure, R⁷ is selected from H, or the following groups which are unsubstituted or optionally substituted with one, two, or more R^{b}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, R¹⁰C(═O)—, R¹⁰C(═O)O—, R¹⁰OC(═O)—, R¹⁰S(O)₂—, R¹⁰S(O)₂O—, R¹⁰OS(O)—, R¹⁰S(O)—, tri(C₁₋₂₀ alkyloxy)silyl C₁₋₂₀ alkyl, or tri(C₁₋₂₀ alkyl)silyl.

According to the embodiments of the present disclosure, R⁷ is selected from H, or the following groups which are unsubstituted or optionally substituted with one, two, or more R^{b}: C₁₋₈ alkyl, C₂₋₈ alkenyl, C₃₋₁₅ cycloalkyl, C₃₋₈ cycloalkenyl, 3-15 membered heterocyclyl, C₆₋₁₅ aryl, 5-15 membered heteroaryl, R¹⁰C(═O)—, R¹⁰C(═O)O—, R¹⁰OC(═O)—, R¹⁰S(O)₂—, R¹⁰S(O)₂O—, R¹⁰OOS(O)₂—, R¹⁰S(O)—, tri(C₁₋₈ alkyloxy)silyl C₁₋₈ alkyl, or tri(C₁₋₈ alkyl)silyl.

According to the embodiments of the present disclosure, R¹⁰ is selected from C₁₋₈ alkyl, C₂₋₈ alkenyl, C₃₋₁₀ cycloalkyl, C₃₋₁₀ cycloalkenyl, 3-8 membered heterocyclyl, C₆₋₁₀ aryl, 5-10 membered heteroaryl, or NH₂.

According to the embodiments of the present disclosure, each R^{a} and R^{b} is the same or different and is independently selected from halogen, —OH, —CN, —NO₂, or the following groups which are unsubstituted or optionally substituted with one, two, or more R^{d}: C₁₋₈ alkyl, C₂₋₈ alkenyl, C₃₋₈ cycloalkyl, C₃₋₈ cycloalkenyl, 3-15 membered heterocyclyl, C₆₋₁₅ aryl, 5-15 membered heteroaryl, C₁₋₈ alkyloxy, C₂₋₈ alkenyl alkyloxy, C₃₋₈ is cycloalkyloxy, C₃₋₈ cycloalkenyloxy, 3-15 membered heteroaryl cycloyloxy, C₆₋₁₅ aryloxy, 5-15 membered heteroaryloxy, C₁₋₈ alkylthio, C₂₋₈ alkenylthio, C₃₋₈ cycloalkylthio, C₃₋₈ cycloalkenylthio, 3-15 membered heterocyclylthio, C₆₋₁₅ arylthio, 5-15 membered heteroarylthio, R¹¹C(═O)—, R¹¹C(═O)O—, R¹¹OC(═O)—, R¹¹S(O)₂—, R¹¹S(O)₂O—, R¹¹OS(O)₂—, R¹¹S(O)—, —NR¹²R¹³, —NH—C(═Y)—NR¹²R¹³, or tri(C1-20 alkyloxy)silyl C₁₋₂₀ alkyl.

According to exemplary embodiments of the present disclosure, R⁷ is selected from, for example, H, or the following groups which are unsubstituted or optionally substituted with one, two, or more R^{e}: C₁₋₈ alkyl, C₃₋₈ cycloalkyl, C₂₋₈ alkenyl, tri(C₁₋₈ alkyloxy)silyl C₁₋₈ alkyl, phenyl, biphenyl, or naphthyl.

For example, R⁷ is selected from H, or the following groups which are unsubstituted or optionally substituted with one, two, or more R^{e}: methyl, ethyl, n-propyl, isopropyl, n-butyl, isobutyl, tert-butyl, n-pentyl, n-hexyl, n-heptyl, cyclopropyl, cyclobutyl, cyclopentyl, cyclohexyl, cycloheptyl, allyl, phenyl,

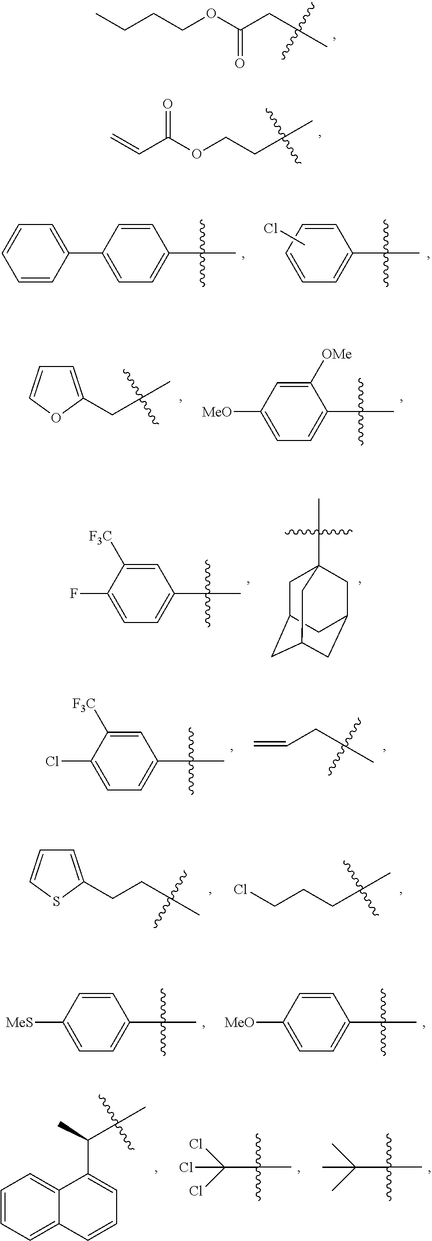

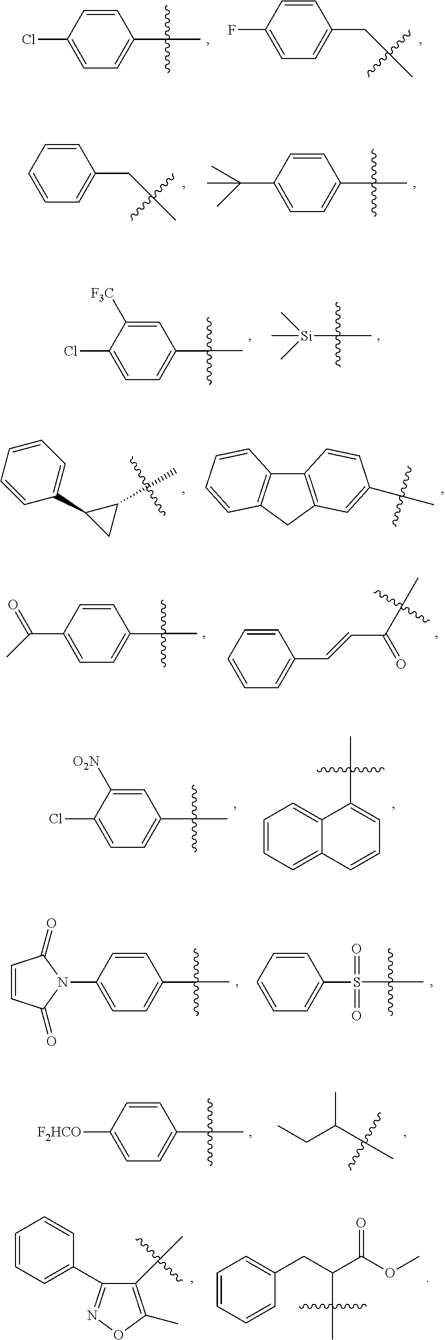

According to an embodiment of the present disclosure, the compound represented by formula (I) may be selected from the following compounds represented by formula (I-1):

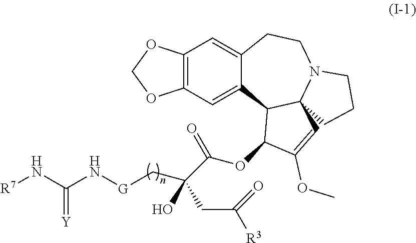

Wherein R⁷, G, n and R³ are independently selected from the above definitions, and Y represents O, S or NH.

According to the embodiment of the disclosure, R^{Q} is selected from the following groups: H, halogen, —OH, —CN, unsubstituted or optionally substituted by one, two or more R^{e}: C₁₋₈ alkyl, C₂₋₈ alkenyl, C₃₋₁₅ cycloalkyl, C₃₋₈ cycloalkenyl, 3-15 membered heterocyclyl, C₆₋₁₅ aryl, 5-15 membered heteroaryl, C₁₋₈ alkyloxy, C₂₋₈ alkenyl alkyloxy, C₃₋₈ cycloalkyloxy, C₃₋₈ cycloalkenyloxy, 3-15 membered heteroaryl cycloyloxy, C₆₋₁₅ aryloxy, 5-15 membered heteroaryloxy, C₁₋₈ alkylthio, C₂₋₈ alkenylthio, C₃₋₈ cycloalkylthio, C₃₋₁₅ cycloalkenylthio, 3-15 membered heterocyclylthio, C₆₋₁₅ arylthio, 5-15 membered heteroarylthio.

According to an embodiment of the present disclosure, R^{Q} is selected from the following groups: H, halogen, —OH, —CN, unsubstituted or optionally substituted by one, two or more R^{e}: C₁₋₄ alkyl, C₂₋₄ alkenyl, C₃₋₁₀ cycloalkyl, C₃₋₁₀ cycloalkenyl, 3-10 membered heterocyclyl, C₆₋₁₀ aryl, 5-10 membered heteroaryl.

According to an exemplary embodiment of the present disclosure, R^{Q} is selected from halogen or the above groups substituted by halogen. According to the embodiment of the disclosure, R³ is selected from H, C₁₋₈ alkyl, C₂₋₈ alkenyl, C₃₋₈ cycloalkyl, C₃₋₈ cycloalkenyl, 3-15 membered heterocyclyl, C₆₋₁₅ aryl, 5-15 membered heteroaryl, C₁₋₈ alkyl —O—, C₂₋₈ alkenyl —O—, C₃₋₁₅ cycloalkyl —O—. C₁₋₈ alkyl —S—, C₂₋₈ alkenyl —S—, C₃₋₁₅ cycloalkyl —S—, C₃₋₁₅ cycloalkenyl —S—, 3-15 membered heterocyclyl —S—, C₆₋₁₅ aryl —S—, 5-15 membered heteroaryl —S—. According to an exemplary embodiment of the present disclosure, R³ is selected from H, methyl, methoxy and methylthio. According to the embodiment of the present disclosure, the compound represented by formula (I) can be selected from the following compounds represented by formula (I-2), (I-4) or (I-5):

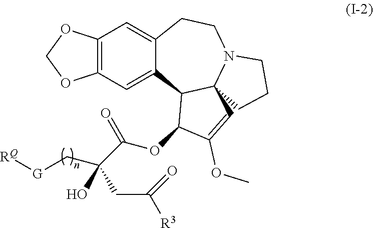

wherein R^{Q}, G, n and R³ are independently selected from the above definitions.

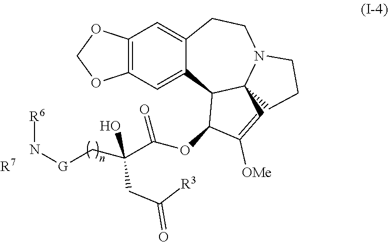

wherein R⁶, R⁷, G, n and R³ are independently selected from the above definitions.

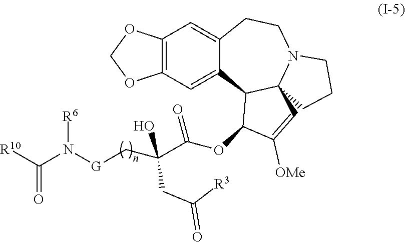

wherein R⁶, R¹⁰, G, n and R³ are independently selected from the above definitions.

According to embodiments of the present disclosure, Y is selected from N.

According to exemplary embodiments of the present disclosure, R^{Q} is selected from H, unsubstituted or optionally substituted with one, two or more R^{e}: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl.

According to exemplary embodiments of the present disclosure, R^{Q} is selected from the following groups unsubstituted or optionally substituted with one, two or more R^{e}: C₁₋₄ alkyl, C₂₋₄ alkenyl, C₃₋₁₀ cycloalkyl, C₃₋₁₀ cycloalkenyl, 3-10 membered heterocyclyl, C₆₋₁₀ aryl, 5-10 membered heteroaryl, for example unsubstituted or optionally substituted with one, two or more R^{e}: Methyl, ethyl, propyl, isopropyl, vinyl, propenyl, allyl, thienyl, dihydrobenzofuranyl.

According to embodiments of the present disclosure, R³ is selected from H, C₁₋₄ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl.

According to embodiments of the present disclosure, R⁴ is selected from the following groups unsubstituted or optionally substituted with one, two or more R^{d}: R⁸—O—, R⁸—S—, R⁹C(═O)O—.

According to embodiments of the present disclosure, each R⁸ and R⁹ are the same or different, independently of each other, selected from H, C₁₋₈ alkyl, C₂₋₈ alkenyl, C₃₋₈ is cycloalkyl, C₃₋₈ is cycloalkenyl, 3-15 membered heterocyclyl, C₆₋₁₅ aryl, 5-15 membered heteroaryl, —NR¹²R¹³.

According to embodiments of the present disclosure, each R¹² and R³ are the same or different, independently of each other, selected from H, unsubstituted or optionally substituted with one, two or more R^{f} of the following groups: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl.

According to embodiments of the present disclosure, R⁸ is selected from H, —OH, acryloyl, isobutyryl, isopentyl,

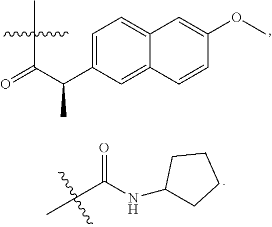 [SMILES: *C(=O)[C@H](C)c1ccc2cc(OC)ccc2c1 ; *C(=O)NC1CCCC1]

According to embodiments of the present disclosure, R⁹ is selected from C₁₋₈ alkyl, C₂₋₈ alkenyl, NH₂.

According to embodiments of the present disclosure, each R^{d} is the same or different, independently of each other, selected from halogen, —OH, —CN, —NO₂, unsubstituted or optionally substituted with one, two or more R^{e}: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, For example, selected from the following groups unsubstituted or optionally substituted with one, two or more R^{e}: phenyl, naphthyl.

According to embodiments of the present disclosure, each R^{e} is the same or different, independently of each other, selected from halogen, —OH, —CN, —NO₂, the following groups unsubstituted or optionally substituted by one, two or more R^{e}: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, C₁₋₈ alkyloxy, C₂₋₈ alkenyl alkyloxy, C₃₋₁₅ cycloalkyloxy, C₃₋₁₅ cycloalkenyloxy, 3-15 membered heteroaryl cycloyloxy, C₆₋₁₅ aryloxy, 5-15 membered heteroaryloxy, C₁₋₈ alkylthio, C₂₋₈ alkenylthio, C₃₋₁₅ cycloalkylthio, C₃₋₈ cycloalkenylthio, 3-15 membered heterocyclylthio, C₆₋₁₅ arylthio, 5-15 membered heteroarylthio.

According to embodiments of the present disclosure, each R^{f} is the same or different, independently of each other, selected from halogen, —OH, —CN, —NO₂, C₁₋₈ alkyl, C₂₋₈ alkenyl, C₃₋₁₅ cycloalkyl, C₃₋₈ cycloalkenyl, 3-15 membered heterocyclyl, C₆₋₁₅ aryl, 5-15 membered heteroaryl, C₁₋₈ alkyloxy, C₂₋₈ alkenyl alkyloxy, C₃₋₁₅ cycloalkyloxy, C₃₋₈ cycloalkenyloxy, 3-15 membered heteroaryl cycloyloxy, C₆₋₁₅ aryloxy, 5-15 membered heteroaryloxy, C₁₋₈ alkylthio, C₂₋₈ alkenylthio, C₃₋₈ cycloalkylthio, C₃₋₈ cycloalkenylthio, 3-15 membered heterocyclylthio, C₆₋₁₅ arylthio, 5-15 membered heteroarylthio.

According to embodiments of the present disclosure, the compound shown in formula (I) may be selected from the compounds shown in formula (I-3) below:

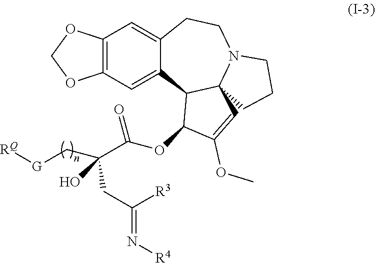

wherein R^{Q}, G, n, R³ and R⁴ are independently selected from the above definitions.

According to exemplary embodiments of the present disclosure, the compound shown in formula (I) described above may be selected from the following compounds:

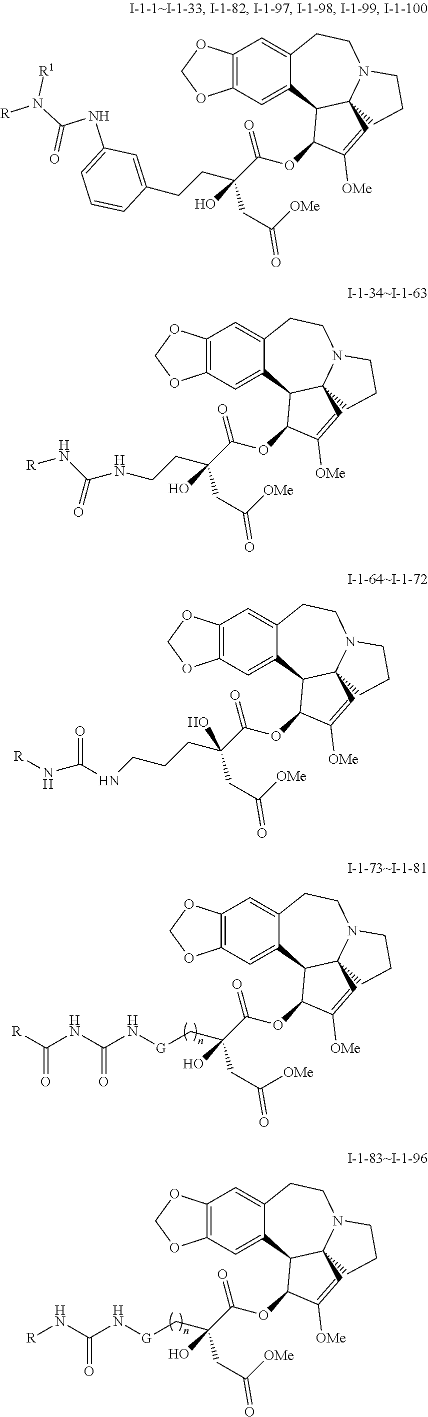

wherein the groups R, G, R⁴ and n have the definitions described in the following table:

| No. | Compds | R | R¹ | No. | Compds | R | No. | Compds | R | G | n | R⁴ |
|---|---|---|---|---|---|---|---|---|---|---|---|---|
| 1 | I-1-1 | 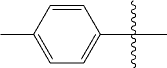 [SMILES: *c1ccc(C)cc1] | H | 39 | I-1-34 | H | 69 | I-1-64 | H | | | |
| 2 | I-1-2 | cyclopentyl | H | 40 | I-1-35 | n-propyl | 70 | I-1-65 | n-penty1 | | | |
| 3 | I-1-3 | H | H | 41 | I-1-36 | n-pentyl | 71 | I-1-66 | cyclopentyl | | | |
| 4 | I-1-4 | n-propyl | H | 42 | I-1-37 | n-hexyl | 72 | I-1-67 | 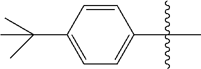 [SMILES: *c1ccc(C(C)(C)C)cc1] | | | |
| 5 | I-1-5 | n-pentyl | H | 43 | I-1-38 | cyclopropyl | 73 | I-1-68 | 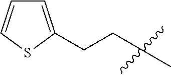 [SMILES: *CCc1cccs1] | | | |
| 6 | I-1-6 | n-hexyl | H | 44 | I-1-39 | cyclobutyl | 74 | I-1-69 | 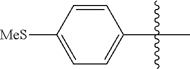 [SMILES: *c1ccc(SC)cc1] | | | |
| 7 | I-1-7 | cyclopropyl | H | 45 | I-1-40 | cyclohexyl | 75 | I-1-70 | 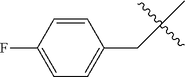 [SMILES: *Cc1ccc(F)cc1] | | | |
| 8 | I-1-8 | cyclobutyl | H | 46 | I-1-41 | cycloheptyl | 76 | I-1-71 | 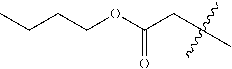 [SMILES: *CC(=O)OCCCC] | | | |
| 9 | I-1-9 | cyclohexyl | H | 47 | I-1-42 | allyl | 77 | I-1-72 | 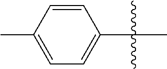 [SMILES: *c1ccc(C)cc1] | | | |
| 10 | I-1-10 | cycloheptyl | H | 48 | I-1-43 | t-butyl | 78 | I-1-73 | 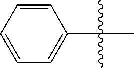 [SMILES: *c1ccccc1] | 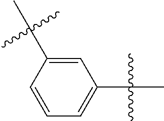 [SMILES: *c1cccc(*)c1] | 2 | |
| 11 | I-1-11 | allyl | H | 49 | I-1-44 | CCl₃ | 79 | I-1-74 | n-pentyl | 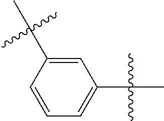 [SMILES: *c1cccc(*)c1] | 2 | |
| 12 | I-1-12 | t-butyl | H | 50 | I-1-45 | TMS | 80 | I-1-75 | cyclopentyl | 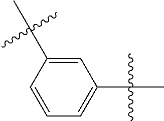 [SMILES: *c1cccc(*)c1] | 2 | |
| 13 | I-1-13 | CCl₃ | H | 51 | I-1-46 | 3-chloropropyl | 81 | I-1-76 | 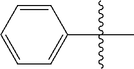 [SMILES: *c1ccccc1] | — | 2 | |
| 14 | I-1-14 | TMS | H | 52 | I-1-47 | 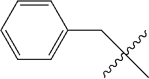 [SMILES: *Cc1ccccc1] | 82 | I-1-77 | n-pentyl | — | 2 | |
| 15 | I-1-15 | 3-chloropropyl | H | 53 | I-1-48 | 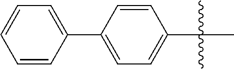 [SMILES: *c1ccc(-c2ccccc2)cc1] | 83 | I-1-78 | cyclopentyl | — | 2 | |
| 16 | I-1-16 | 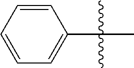 [SMILES: *c1ccccc1] | H | 54 | I-1-49 | 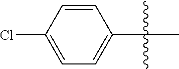 [SMILES: *c1ccc(Cl)cc1] | 84 | I-1-79 | 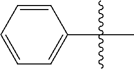 [SMILES: *c1ccccc1] | — | 3 | |
| 17 | I-1-17 | 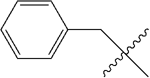 [SMILES: *Cc1ccccc1] | H | 55 | I-1-50 | 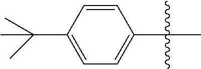 [SMILES: *c1ccc(C(C)(C)C)cc1] | 85 | I-1-80 | n-pentyl | — | 3 | |
| 18 | I-1-18 | 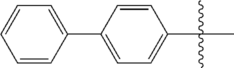 [SMILES: *c1ccc(-c2ccccc2)cc1] | H | 56 | I-1-51 | 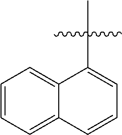 [SMILES: *c1cccc2ccccc12] | 86 | I-1-81 | cyclopentyl | | 3 | |
| 19 | I-1-19 | 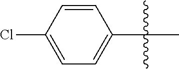 [SMILES: *c1ccc(Cl)cc1] | H | 57 | I-1-52 | 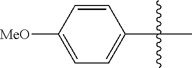 [SMILES: *c1ccc(OC)cc1] | 87 | I-1-83 | n-pentyl | 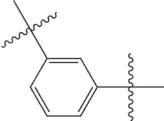 [SMILES: *c1cccc(*)c1] | 2 | H |
| 20 | I-1-20 | 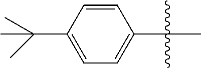 [SMILES: *c1ccc(C(C)(C)C)cc1] | H | 58 | I-1-53 | 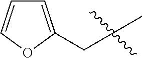 [SMILES: *Cc1ccco1] | 88 | I-1-84 | cyclopentyl | 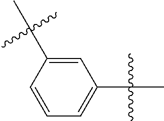 [SMILES: *c1cccc(*)c1] | 2 | H |
| 21 | I-1-21 | 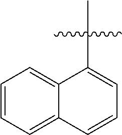 [SMILES: *c1cccc2ccccc12] | H | 59 | I-1-54 | 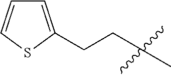 [SMILES: *CCc1cccs1] | 89 | I-1-85 | 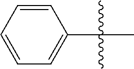 [SMILES: *c1ccccc1] | 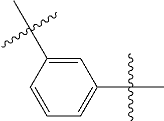 [SMILES: *c1cccc(*)c1] | 2 | H |
| 22 | I-1-22 | 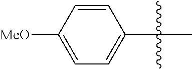 [SMILES: *c1ccc(OC)cc1] | H | 60 | I-1-55 | 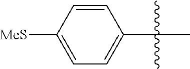 [SMILES: *c1ccc(SC)cc1] | 90 | I-1-86 | i-Pr | 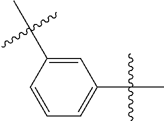 [SMILES: *c1cccc(*)c1] | 2 | i-Pr |
| 23 | I-1-23 | 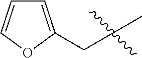 [SMILES: *Cc1ccco1] | H | 61 | I-1-56 | 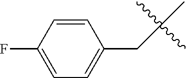 [SMILES: *Cc1ccc(F)cc1] | 91 | I-1-87 | i-Pr | — | 2 | i-Pr |
| 24 | I-1-24 | 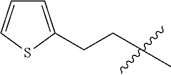 [SMILES: *CCc1cccs1] | H | 62 | I-1-57 | 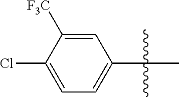 [SMILES: *c1ccc(Cl)c(C(F)(F)F)c1] | 92 | I-1-88 | 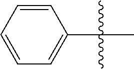 [SMILES: *c1ccccc1] | — | 2 | H |
| 25 | I-1-25 | 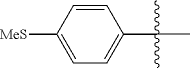 [SMILES: *c1ccc(SC)cc1] | H | 63 | I-1-58 | 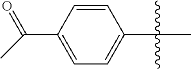 [SMILES: *c1ccc(C(C)=O)cc1] | 93 | I-1-89 | n-pentyl | — | 2 | H |
| 26 | I-1-26 | 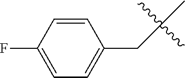 [SMILES: *Cc1ccc(F)cc1] | H | 64 | I-1-59 | 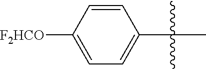 [SMILES: *c1ccc(OC(F)F)cc1] | 94 | I-1-90 | cyclopentyl | — | 2 | H |
| 27 | I-1-27 | 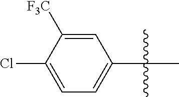 [SMILES: *c1ccc(Cl)c(C(F)(F)F)c1] | H | 65 | I-1-60 | 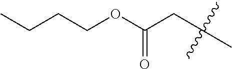 [SMILES: *CC(=O)OCCCC] | 95 | I-1-91 | 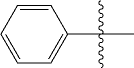 [SMILES: *c1ccccc1] | — | 3 | H |
| 28 | I-1-28 | 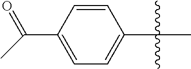 [SMILES: *c1ccc(C(C)=O)cc1] | H | 66 | I-1-61 | 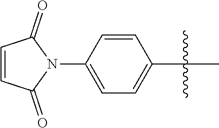 [SMILES: *c1ccc(N2C(=O)C=CC2=O)cc1] | 96 | I-1-92 | n-penty1 | — | 3 | H |
| 29 | I-1-29 | 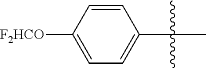 [SMILES: *c1ccc(OC(F)F)cc1] | H | 67 | I-1-62 | 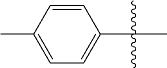 [SMILES: *c1ccc(C)cc1] | 97 | I-1-93 | cyclopentyl | | 3 | H |
| 30 | I-1-30 | 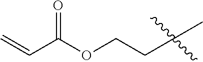 [SMILES: *CCOC(=O)C=C] | H | 68 | I-1-63 | cyclopentyl | 98 | I-1-94 | n-pentanoyl | 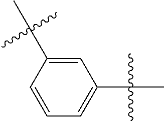 [SMILES: *c1cccc(*)c1] | 2 | H |
| 31 | I-1-31 | 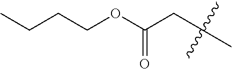 [SMILES: *CC(=O)OCCCC] | H | | | | 99 | I-1-95 | cyclopentyloxy | 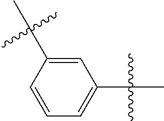 [SMILES: *c1cccc(*)c1] | 2 | H |
| 32 | I-1-32 | 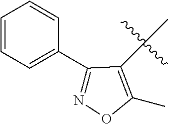 [SMILES: *c1c(-c2ccccc2)noc1C] | H | | | | 100 | I-1-96 | benzoyl | 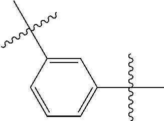 [SMILES: *c1cccc(*)c1] | 2 | H |
| 33 | I-1-33 | 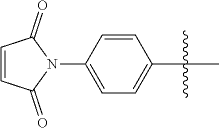 [SMILES: *c1ccc(N2C(=O)C=CC2=O)cc1] | H | | | | | | | | | |
| 34 | I-1-82 | 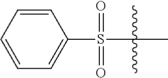 [SMILES: *S(=O)(=O)c1ccccc1] | H | | | | | | | | | |
| 35 | I-1-97 | 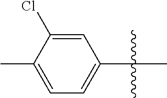 [SMILES: *c1ccc(C)c(Cl)c1] | H | | | | | | | | | |
| 36 | I-1-98 | 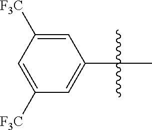 [SMILES: *c1cc(C(F)(F)F)cc(C(F)(F)F)c1] | H | | | | | | | | | |
| 37 | I-1-99 | 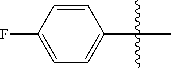 [SMILES: *c1ccc(F)cc1] | H | | | | | | | | | |
| 38 | I-1-100 | CH₃ | CH₃ | | | | | | | | | |

According to exemplary embodiments of the present disclosure, the compound shown in formula (I) described above may be selected from the following compounds:

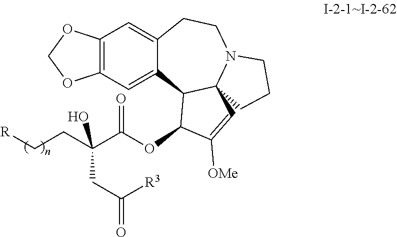

wherein the groups R, R³ and n have the definitions described in the following table:

| No. | Compds | R | R³ | n |
|---|---|---|---|---|
| 101 | I-2-1 | 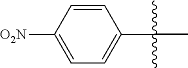 [SMILES: *c1ccc([N+](=O)[O-])cc1] | OMe | 1 |
| 102 | I-2-2 | 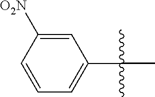 [SMILES: *c1cccc([N+](=O)[O-])c1] | OMe | 1 |
| 103 | I-2-3 | 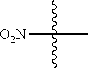 [SMILES: *[N+](=O)[O-]] | OMe | 1 |
| 104 | I-2-4 | 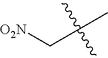 [SMILES: *C[N+](=O)[O-]] | OMe | 1 |
| 105 | I-2-5 | 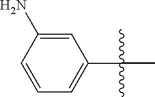 [SMILES: *c1cccc(N)c1] | OMe | 1 |
| 106 | I-2-6 | 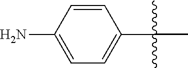 [SMILES: *c1ccc(N)cc1] | OMe | 1 |
| 107 | I-2-7 | 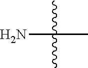 [SMILES: *N] | OMe | 1 |
| 108 | I-2-8 | 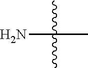 [SMILES: *N] | OMe | 2 |
| 109 | I-2-9 | 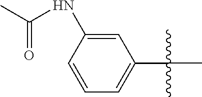 [SMILES: *c1cccc(NC(C)=O)c1] | OMe | 1 |
| 110 | I-2-10 | 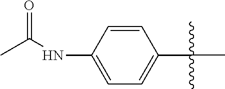 [SMILES: *c1ccc(NC(C)=O)cc1] | OMe | 1 |
| 111 | I-2-11 | 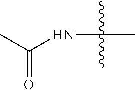 [SMILES: *NC(C)=O] | OMe | 1 |
| 112 | I-2-12 | 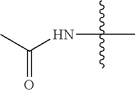 [SMILES: *NC(C)=O] | OMe | 2 |
| 113 | I-2-13 | 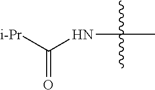 [SMILES: *NC(=O)C(C)C] | OMe | 1 |
| 114 | I-2-14 | 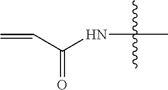 [SMILES: *NC(=O)C=C] | OMe | 1 |
| 115 | I-2-15 | 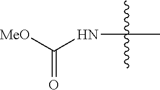 [SMILES: *NC(=O)OC] | OMe | 1 |
| 116 | I-2-16 | 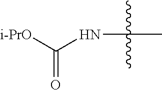 | OMe | 2 |
| 117 | I-2-17 | 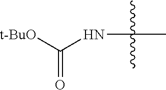 [SMILES: *NC(=O)OC(C)(C)C] | OMe | 1 |
| 118 | I-2-18 | 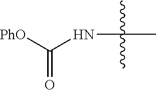 [SMILES: *NC(=O)Oc1ccccc1] | OMe | 1 |
| 119 | I-2-19 | 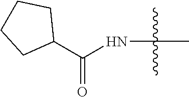 [SMILES: *NC(=O)C1CCCC1] | OMe | 2 |
| 120 | I-2-20 | 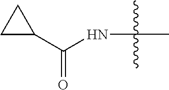 [SMILES: *NC(=O)C1CC1] | OMe | 2 |
| 121 | I-2-21 | 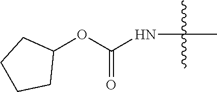 [SMILES: *NC(=O)OC1CCCC1] | OMe | 2 |
| 122 | I-2-22 | 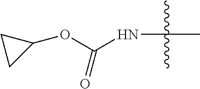 [SMILES: *NC(=O)OC1CC1] | OMe | 2 |
| 123 | I-2-23 | 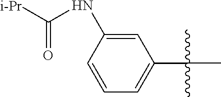 [SMILES: *c1cccc(NC(=O)C(C)C)c1] | OMe | 1 |
| 124 | I-2-24 | 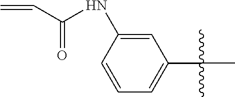 [SMILES: *c1cccc(NC(=O)C=C)c1] | OMe | 1 |
| 125 | I-2-25 | 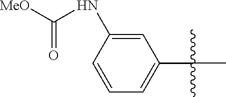 [SMILES: *c1cccc(NC(=O)OC)c1] | OMe | 1 |
| 126 | I-2-26 | 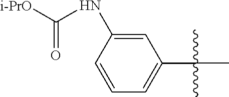 | OMe | 1 |
| 127 | I-2-27 | 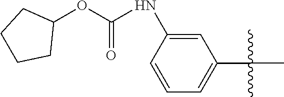 [SMILES: *c1cccc(NC(=O)OC2CCCC2)c1] | OMe | 1 |
| 128 | I-2-28 | 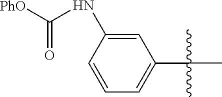 [SMILES: *c1cccc(NC(=O)Oc2ccccc2)c1] | OMe | 1 |
| 129 | I-2-29 | 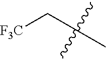 [SMILES: *CC(F)(F)F] | OMe | 1 |
| 130 | I-2-30 | 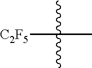 | OMe | 1 |
| 131 | I-2-31 | 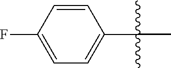 [SMILES: *c1ccc(F)cc1] | OMe | 1 |
| 132 | I-2-32 | 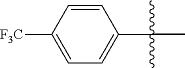 [SMILES: *c1ccc(C(F)(F)F)cc1] | OMe | 1 |
| 133 | I-2-33 | 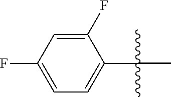 [SMILES: *c1ccc(F)cc1F] | OMe | 1 |
| 134 | I-2-34 | 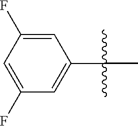 [SMILES: *c1cc(F)cc(F)c1] | OMe | 1 |
| 135 | I-2-35 | 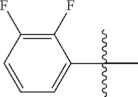 [SMILES: *c1cccc(F)c1F] | OMe | 1 |
| 136 | I-2-36 | 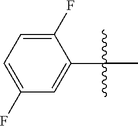 [SMILES: *c1cc(F)ccc1F] | OMe | 1 |
| 137 | I-2-37 | 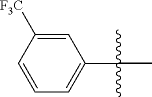 [SMILES: *c1cccc(C(F)(F)F)c1] | OMe | 1 |
| 138 | I-2-38 | 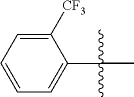 [SMILES: *c1ccccc1C(F)(F)F] | OMe | 1 |
| 139 | I-2-39 | 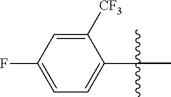 [SMILES: *c1ccc(F)cc1C(F)(F)F] | OMe | 1 |
| 140 | I-2-40 | 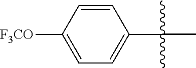 [SMILES: *c1ccc(OC(F)(F)F)cc1] | OMe | 1 |
| 141 | I-2-41 | 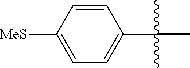 [SMILES: *c1ccc(SC)cc1] | OMe | 1 |
| 142 | I-2-42 | 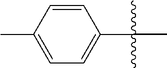 [SMILES: *c1ccc(C)cc1] | OMe | 1 |
| 143 | I-2-43 | 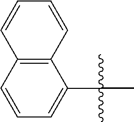 [SMILES: *c1cccc2ccccc12] | OMe | 1 |
| 144 | I-2-44 | 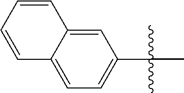 [SMILES: *c1ccc2ccccc2c1] | OMe | 1 |
| 145 | I-2-45 | 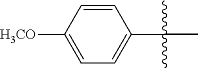 [SMILES: *c1ccc(OC)cc1] | OMe | 1 |
| 146 | I-2-46 | 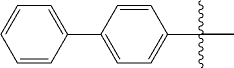 [SMILES: *c1ccc(-c2ccccc2)cc1] | OMe | 1 |
| 147 | I-2-47 | 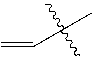 [SMILES: *C=C] | Me | 1 |
| 148 | I-2-48 | 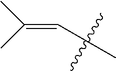 [SMILES: *C=C(C)C] | Me | 1 |
| 149 | I-2-49 | 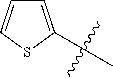 [SMILES: *c1cccs1] | Me | 1 |
| 150 | I-2-50 | 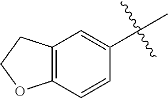 [SMILES: *c1ccc2c(c1)CCO2] | Me | 1 |
| 151 | I-2-51 | i-Pr | Me | 1 |
| 152 | I-2-52 | i-Pr | SMe | 1 |
| 153 | I-2-53 | 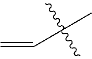 [SMILES: *C=C] | SMe | 1 |
| 154 | I-2-54 | 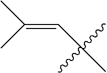 [SMILES: *C=C(C)C] | SMe | 1 |
| 155 | I-2-55 | 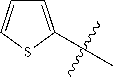 [SMILES: *c1cccs1] | SMe | 1 |
| 156 | I-2-56 | 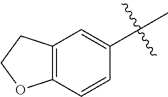 [SMILES: *c1ccc2c(c1)CCO2] | SMe | 1 |
| 157 | I-2-57 | C₂H₅ | H | 1 |
| 158 | I-2-58 | 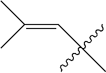 [SMILES: *C=C(C)C] | H | 1 |
| 159 | I-2-59 | 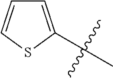 [SMILES: *c1cccs1] | H | 1 |
| 160 | I-2-60 | 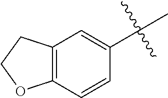 [SMILES: *c1ccc2c(c1)CCO2] | H | 1 |
| 161 | I-2-61 | i-Pr | H | 1 |
| 162 | I-2-62 | 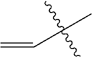 [SMILES: *C=C] | H | 1 |

According to exemplary embodiments of the present disclosure, the compound shown in formula (I) described above may be selected from the following compounds:

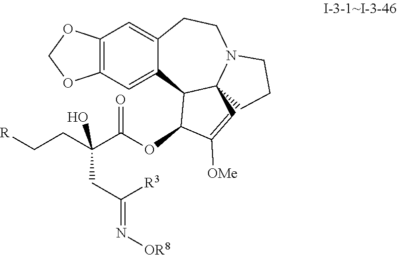

wherein the groups R, R³ and R⁸ have the definitions described in the following table:

| No. | Compds | R | R³ | R⁸ | Z/E |
|---|---|---|---|---|---|
| 163 | I-3-1 | 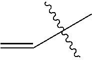 [SMILES: *C=C] | Me | H | E |
| 164 | I-3-2 | 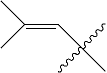 [SMILES: *C=C(C)C] | Me | H | E |
| 165 | I-3-3 | 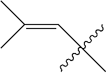 [SMILES: *C=C(C)C] | Me | H | Z |
| 166 | I-3-4 | 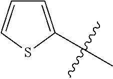 [SMILES: *c1cccs1] | Me | H | E |
| 167 | I-3-5 | 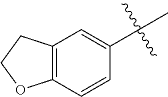 [SMILES: *c1ccc2c(c1)CCO2] | Me | H | Z |
| 168 | I-3-6 | 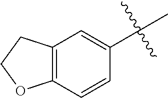 [SMILES: *c1ccc2c(c1)CCO2] | Me | H | E |
| 169 | I-3-7 | i-Pr | H | H | Z |
| 170 | I-3-8 | i-Pr | H | H | E |
| 171 | I-3-9 | 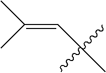 [SMILES: *C=C(C)C] | H | H | Z |
| 172 | I-3-10 | 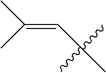 [SMILES: *C=C(C)C] | H | H | E |
| 173 | I-3-11 | Et | H | H | Z |
| 174 | I-3-12 | Et | H | H | E |
| 175 | I-3-13 | i-Pr | H | Ac | Z |
| 176 | I-3-14 | 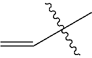 [SMILES: *C=C] | H | Ac | Z |
| 177 | I-3-15 | 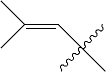 [SMILES: *C=C(C)C] | H | Ac | E |
| 178 | I-3-16 | i-Pr | Me | acrylamide | E |
| 179 | I-3-17 | i-Pr | Me | isobutyryl | E |
| 180 | I-3-18 | 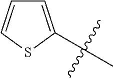 [SMILES: *c1cccs1] | Me | isobutyryl | E |
| 181 | I-3-19 | 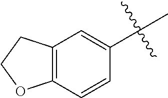 [SMILES: *c1ccc2c(c1)CCO2] | Me | acrylamide | E |
| 182 | I-3-20 | 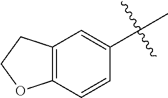 [SMILES: *c1ccc2c(c1)CCO2] | Me | isobutyryl | E |
| 183 | I-3-21 | 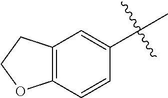 [SMILES: *c1ccc2c(c1)CCO2] | Me | isobutyryl | Z |
| 184 | I-3-22 | i-Pr | H | acrylamide | E |
| 185 | I-3-23 | i-Pr | H | isobutyryl | E |
| 186 | I-3-24 | Et | H | isobutyryl | Z |
| 187 | I-3-25 | 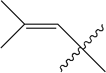 [SMILES: *C=C(C)C] | H | isobutyryl | Z |
| 188 | I-3-26 | i-Pr | Me | 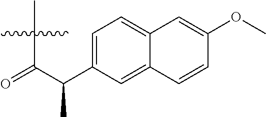 [SMILES: *C(=O)[C@H](C)c1ccc2cc(OC)ccc2c1] | E |
| 189 | 1-3-27 | 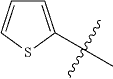 [SMILES: *c1cccs1] | Me | 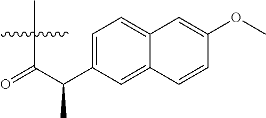 [SMILES: *C(=O)[C@H](C)c1ccc2cc(OC)ccc2c1] | E |
| 190 | 1-3-28 | 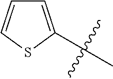 [SMILES: *c1cccs1] | Me | 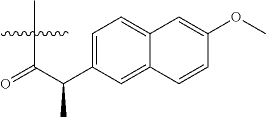 [SMILES: *C(=O)[C@H](C)c1ccc2cc(OC)ccc2c1] | Z |
| 191 | 1-3-29 | 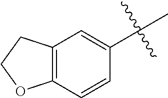 [SMILES: *c1ccc2c(c1)CCO2] | Me | 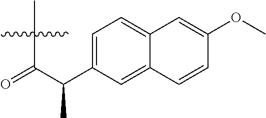 [SMILES: *C(=O)[C@H](C)c1ccc2cc(OC)ccc2c1] | E |
| 192 | 1-3-30 | 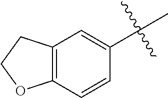 [SMILES: *c1ccc2c(c1)CCO2] | Me | 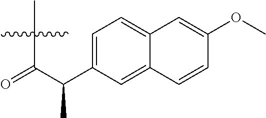 [SMILES: *C(=O)[C@H](C)c1ccc2cc(OC)ccc2c1] | Z |
| 193 | I-3-31 | i-Pr | H | 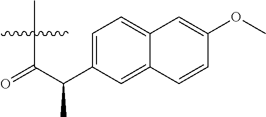 [SMILES: *C(=O)[C@H](C)c1ccc2cc(OC)ccc2c1] | E |
| 194 | I-3-32 | Et | H | 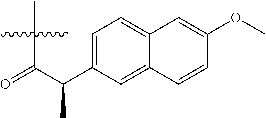 [SMILES: *C(=O)[C@H](C)c1ccc2cc(OC)ccc2c1] | O |
| 195 | 1-3-33 | 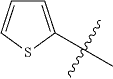 [SMILES: *c1cccs1] | H | 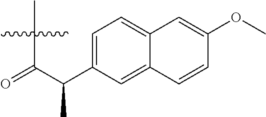 [SMILES: *C(=O)[C@H](C)c1ccc2cc(OC)ccc2c1] | E |
| 196 | I-3-34 | 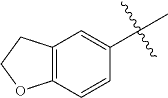 [SMILES: *c1ccc2c(c1)CCO2] | H | 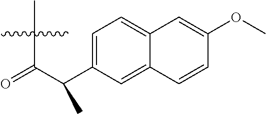 [SMILES: *C(=O)[C@H](C)c1ccc2cc(OC)ccc2c1] | E |
| 197 | 1-3-35 | i-Pr | Me | isopentyl | E |
| 198 | I-3-36 | i-Pr | H | isopentyl | E |
| 199 | I-3-37 | Et | H | isopentyl | E |
| 200 | 1-3-38 | 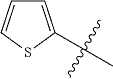 [SMILES: *c1cccs1] | H | isopentyl | E |
| 201 | I-3-39 | i-Pr | Me | 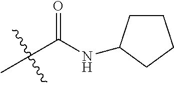 [SMILES: *C(=O)NC1CCCC1] | E |
| 202 | 1-3-40 | i-Pr | Me | 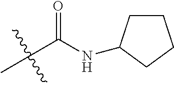 [SMILES: *C(=O)NC1CCCC1] | Z |
| 203 | I-3-41 | 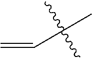 [SMILES: *C=C] | Me | 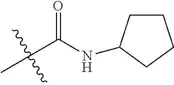 [SMILES: *C(=O)NC1CCCC1] | E |
| 204 | I-3-42 | 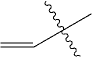 [SMILES: *C=C] | Me | 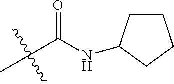 [SMILES: *C(=O)NC1CCCC1] | Z |
| 205 | I-3-43 | 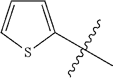 [SMILES: *c1cccs1] | Me | 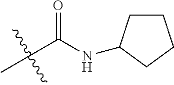 [SMILES: *C(=O)NC1CCCC1] | E |
| 206 | 1-3-44 | 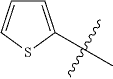 [SMILES: *c1cccs1] | Me | 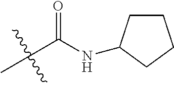 [SMILES: *C(=O)NC1CCCC1] | Z |
| 207 | 1-3-45 | 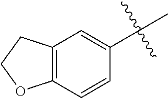 [SMILES: *c1ccc2c(c1)CCO2] | Me | 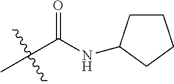 [SMILES: *C(=O)NC1CCCC1] | E |
| 208 | I-3-46 | i-Pr | H | 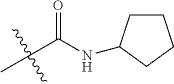 [SMILES: *C(=O)NC1CCCC1] | E |

Unless otherwise indicated, while the definitions of each of the above moieties are used to describe the structure of specific compounds covered by the compounds of the general formula, it should be appreciated by those skilled in the art that the present specification likewise documents an independent definition of each of the above moieties, which independent definition may be used, either alone or in conjunction with the other moieties, to further delimit one, two, or more corresponding substituents in the compounds of the general formula, and thereby further delimit the compound of the general formula.

The present disclosure also provides a method for the preparation of the compounds shown in formula (I), their racemates, stereoisomers, reciprocal isomers, isotopic markers, N-oxides, hydrates, solvates, polycrystallines, metabolites, pharmaceutically acceptable salts, pharmaceutically acceptable esters, or pre-drug compounds thereof, wherein the method of preparation may comprise one of the methods selected from among the following:

### Method (1)

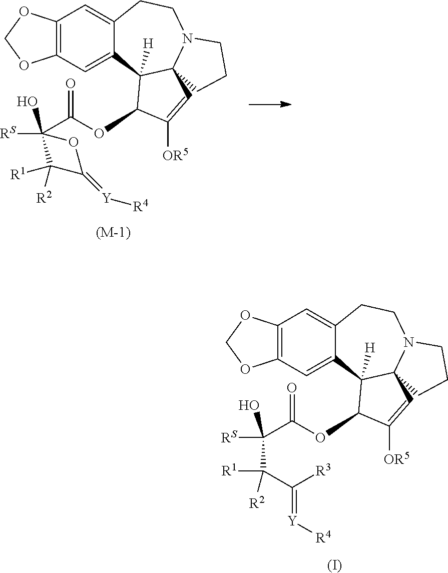

wherein R¹, R², R⁴, R³, Y have the definitions described above.
R³ is selected from R⁸—O—.
the step (1) is carried out in the presence of a base and R⁸—OH to prepare a compound of formula (I).

### Method (2)

when the substituent R^{S} of the compound of formula (I) contains an amino group, the compound of formula (I) is prepared by reduction using the corresponding nitro compound as substrate.

Preferably, the reduction is carried out in the presence of Zn and AcOH.

### Method (3)

when the substituent R^{S} of a compound of formula (I) contains an acylamino group, the compound of formula (I) is prepared by acylation reaction using the corresponding amino compound as substrate.

### Method (4)

when the substituent group R^{S} of the compound of formula (I) contains a urea group, the compound of formula (I) is prepared by reacting with an isocyanate compound using the corresponding amino compound as substrate.

### Method (5)

when the substituent group R^{S} of the compound of formula (I) contains a guanidine group, the compound of formula (I) is prepared by reacting with nitrile amines, acyl nitrile amines, or differently substituted carbodiimide analogs using the corresponding amino compound as substrate.

### Method (6)

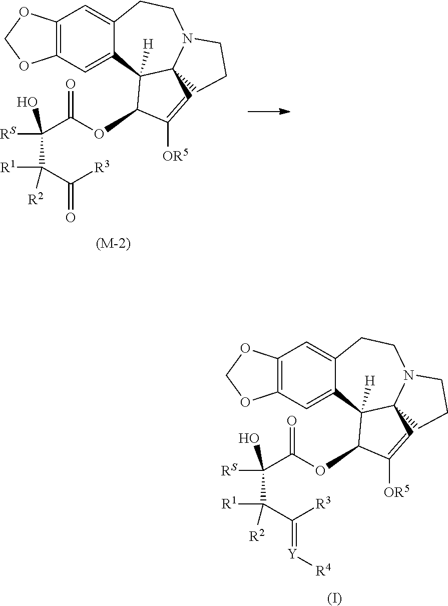

wherein R¹, R², R³, R⁵ have the definitions described above.
R⁴ is selected from hydroxyl.
Y is selected from N.
the step (1) is carried out in the presence of base and hydroxylamine hydrochloride to prepare the compound of formula (I).

### Method (7)

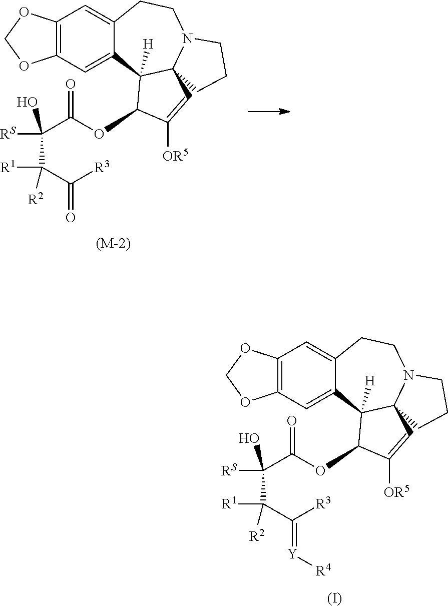

wherein R¹, R², R³, R⁵ have the definitions described above.
R⁴ is selected from R⁹C(═O)O—.
Y is selected from N.
the step (1) is carried out in the presence of a base and C₁₋₂₀ alkyl-C(O)—O—C(O)—C₁₋₂₀ alkyl to prepare the compound of formula (I).

### Method (8)

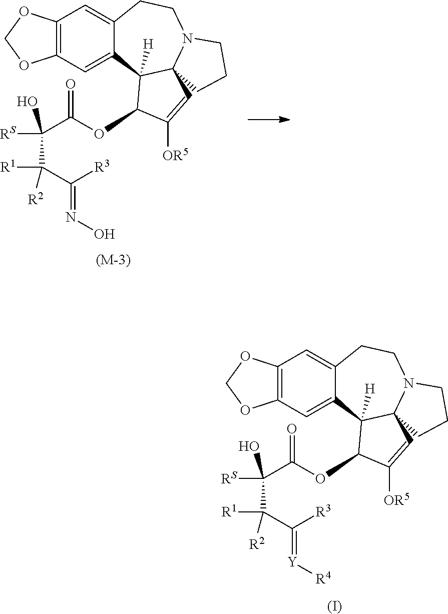

wherein R¹, R², R³, R⁵ have the definitions described above.
R⁴ is selected from R⁹C(═O)O—.
Y is selected from N.
the step (1) is reacted in the presence of a base and R⁹C(═O)Cl or R⁹C(═O)OH to prepare a compound of formula (I).

### Method (9)

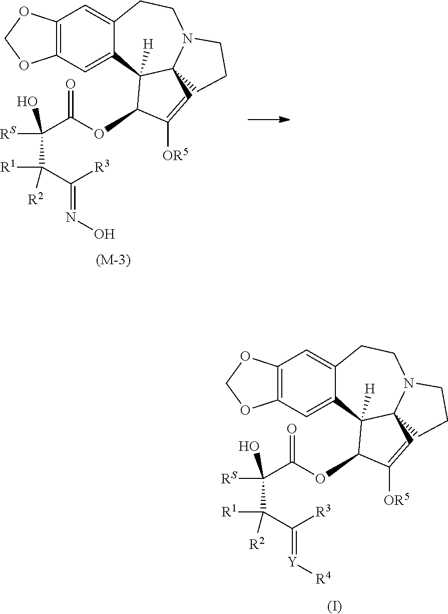

wherein R¹, R², R³, R⁵ have the definitions described above.
R⁴ is selected from R⁸—O—.
R⁸ is selected from C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl.
Y is selected from N.
the step (1) is performed in the presence of a base and R⁸-L to prepare a compound of formula (I).
L is a leaving group, e.g. halogen, preferably Br or I.

### Method (10)

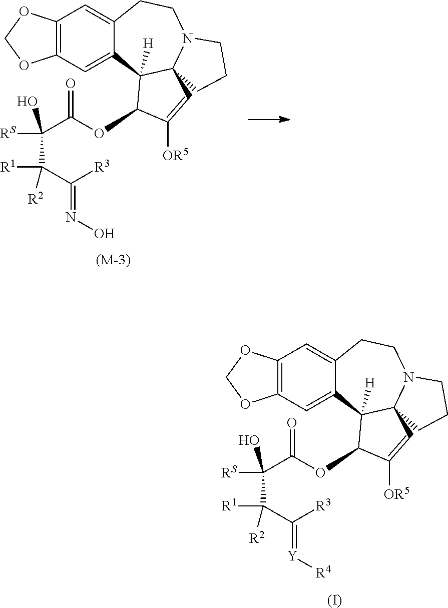

wherein R¹, R², R³, R⁵ have the definitions described above.
R⁴ is selected from R¹⁰NHC(═O)O—.
Y is selected from N.
the step (1) is reacted in the presence of R¹⁰—N═C═O to prepare a compound of formula (I).

Optionally, the preparation method further comprises the step of reacting the prepared compound of formula (I) with a pharmaceutically acceptable acid or base to form a salt.

The present disclosure also provides a pharmaceutical composition comprising at least one of the compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof.

The present disclosure also provides a pharmaceutical composition comprising a therapeutically effective amount of at least one of the compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof.

According to embodiments of the present disclosure, the pharmaceutical composition further comprises one or more pharmaceutically acceptable excipients.

According to embodiments of the present disclosure, the pharmaceutical composition may further comprise one or more additional therapeutic agents.

The present disclosure also provides the use of at least one of the compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof, in manufacturing a drug.

According to embodiments of the present disclosure, the compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof, the drug or the pharmaceutical composition is used for the prevention and/or treatment of cells growing by uncontrolled proliferation and/or survival, an inappropriate cellular immune response, or an inappropriate cellular inflammatory response, or a disease accompanied by uncontrolled cell growth, proliferation and/or survival, an inappropriate cellular immune response, or an inappropriate cellular inflammatory response, and in particular, the disease is, for example, hematological neoplasms, solid tumors and/or metastases thereof, such as leukemia and myelodysplastic syndromes, malignant lymphomas, head and neck tumors including brain tumors and brain metastases, thoracic tumors including non-small cell lung tumors and small cell lung tumors, gastrointestinal tumors, endocrine tumors, breast tumors and other gynecological tumors, tumors of the urinary system including renal tumors, cystinomas, and prostate tumors, skin tumors and sarcomas, and/or metastases thereof.

The present disclosure also provides the use of the compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof, the drug or pharmaceutical composition, for the inhibition of a tumor cell, wherein said tumor cell is selected from a hematologic tumor and/or tumor cells of solid tumors, such as breast cancer cells, hepatocellular carcinoma cells, lung adenocarcinoma cells, leukemia cells, adriamycin-resistant strains of leukemia cells, glioblastoma cells, cervical cancer cells, colon cancer cells, and/or brain microvascular endothelial cells.

The present disclosure also provides the use of the compound as shown in formula (I), and racemate, stereoisomer, reciprocal isomer, isotopic marker, N-oxide, hydrate, solvate, polymorph, metabolite, pharmaceutically acceptable salt, pharmaceutically acceptable ester, or prodrug thereof in manufacturing a drug, wherein the drug can be used in the treatment of a mammalian hyperproliferative conditions, or for inhibiting, blocking, reducing, minimizing, etc. cell proliferation and/or cell division and/or causing apoptosis. Hyperproliferative conditions include, but are not limited to, psoriasis, keloids and other hyperplasias affecting the skin, benign prostatic hyperplasia (BpH), solid tumors such as breast cancer, respiratory tract cancer, lung cancer, brain cancer, reproductive organ cancer, gastrointestinal tract cancer, urinary tract cancer, eye cancer, liver cancer, skin cancer, head and neck cancer, thyroid cancer, parathyroid cancer, and distal metastases thereof, the disease also include lymphoma, sarcoma and leukemia.

The drug may also be used to inhibit tumor cells, wherein the tumor cells are selected from tumor cells of hematological tumors and/or solid tumors, such as breast cancer cells, hepatocellular carcinoma cells, lung adenocarcinoma cells, leukemia cells, adriamycin-resistant strains of leukemia cells, glioblastoma cells, cervical cancer cells, colon cancer cells, and/or brain microvascular endothelial cells.

According to embodiments of the present disclosure, examples of the tumor cells may be selected from human breast cancer cells MCF-7, hepatocellular carcinoma cells HepG2, lung adenocarcinoma cells A549, human leukemia cells HL-60, human leukemia cells adriamycin-resistant strain HL-60/ADR, human glioblastoma cells U-87MG, human cervical cancer cells HeLa, human colon cancer cells HCT-116, at least one of human colon cancer cell Sw620, human colon cancer cell LoVo, and human brain microvascular endothelial cell hCMEC/D3.

When used as a drug, the compound of the present disclosure may be administered in the form of a pharmaceutical composition. The composition may be prepared in a manner well known in the pharmaceutical field, and they may be administered by a variety of routes, depending on whether local or systemic treatment is desired and the area to be treated. They may be administered topically (e.g., transdermally, dermally, ocularly, and mucosally including intranasal, vaginal, and rectal delivery), pulmonary (e.g., by inhalation or blowing in a powder or aerosol, including through a nebulizer, intratracheal, intranasal), orally, or parenterally. Parenteral administration includes intravenous, intra-arterial, subcutaneous, intraperitoneal or intramuscular injection or infusion, or intracranial e.g., intrathecal or intracerebroventricular administration. Parenteral administration may be in the form of a single large dose, or may be administered by, for example, a continuous infusion pump. Pharmaceutical compositions and formulations for topical administration may include transdermal patches, ointments, lotions, creams, gels, drops, suppositories, sprays, liquids, powder formulations, and dispersions. Conventional drug carriers, water, powder or oil-based matrices, thickeners, and the like may be necessary or desired.

In preparing the compositions of the present disclosure, the active ingredient is usually mixed with an excipient, diluted by means of an excipient or packed into such a carrier in the form of, for example, a capsule, a small medicine capsule, a paper or other container. When the excipient is used as a diluent, it may be a solid, semi-solid or liquid substance used as a solvent, carrier or medium for the active ingredient.

Thus, the compositions may be in the following forms: tablets, pills, dispersions, ingots, small capsules, flat capsules, elixirs, suspensions, emulsions, solutions, syrups, aerosols (solid or dissolved in a liquid solvent), ointments containing, for example, up to 10% by weight of the active compound, soft and hard gelatine capsules, suppositories, sterile injectable solutions, and aseptically packaged powders.

Some examples of suitable excipients include lactose, glucose, sucrose, sorbitol, mannitol, starch, gum arabic, calcium phosphate, alginate, yarrow gum, gelatin, calcium silicate, microcrystalline cellulose, polyvinylpyrrolidone, cellulose, water, syrup, and methyl cellulose.

### Advantageous Effects

The tritiated derivatives and pharmaceutical compositions thereof provided by the present disclosure have excellent anti-tumor activity. Moreover, the preparation method of the derivatives also has the advantages of cheap and easy availability of raw materials and easy operation.

### DEFINITION AND DESCRIPTION OF TERMS

Unless otherwise indicated, the definitions of groups and terms recorded in the specification and claims of the present application, including their definitions as examples, exemplary definitions, preferred definitions, definitions recorded in tables, definitions of specific compounds in the embodiments, and the like, may be arbitrarily combined and combined with each other. The definitions of the moieties and the structures of the compounds after such combinations and combinations should be understood to be within the scope documented in the specification and/or claims of this application.

Unless otherwise indicated, the numerical ranges documented in this specification and claims correspond to at least one specific integer value documented for each of them. For example, the numerical range “1-10” is equivalent to documenting each of the integer values in the numerical range “1-10”, i.e., 1, 2, 3, 4, 5, 6, 7, 8, 9, 10. In addition, when some numerical ranges are defined as“number”, it should be understood that the two endpoints of the range, each integer in the range, and each decimal in the range are recorded. For example, the term “number from 0 to 10” should be understood as documenting not only each of the integers 0, 1, 2, 3, 4, 5, 6, 7, 8, 9, and 10, but also at least the sum of each of those integers with 0.1, 0.2, 0.3, 0.4, 0.5, 0.6, 0.7, 0.8, and 0.9, respectively.

It is to be understood that in describing one, two or more herein, “more” shall mean an integer greater than 2, e.g., greater than or equal to 3, e.g., 3, 4, 5, 6, 7, 8, 9 or 10.

The term “halogen” refers to fluorine, chlorine, bromine and iodine.

The term “C₁₋₂₀ alkyl” as used in this invention refers to a straight or branched saturated monovalent hydrocarbon group having 1 to 20 carbon atoms. For example, “C₁₋₁₀ alkyl” refers to straight and branched alkyl groups having 1, 2, 3, 4, 5, 6, 7, 8, 9 or 10 carbon atoms, and “C₁₋₈ alkyl” refers to straight and branched alkyl groups having 1, 2, 3, 4, 5, 6, 7 or 8 carbon atoms. “C₁₋₈ alkyl” refers to straight and branched alkyl groups with 1, 2, 3, 4, 5, 6, 7 or 8 carbon atoms, the alkyl groups are, for example, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl butyl, 3,3-dimethylbutyl, 2,2-dimethylbutyl, 1,1-dimethylbutyl, 2,3-dimethylbutyl, 1,3-dimethylbutyl, 1,2-dimethylbutyl, or isomers thereof.

The term “C₂₋₂₀ alkenyl” as used in this invention preferably refers to a straight or branched monovalent hydrocarbon group comprising one or more double bonds and having from 2 to 20 carbon atoms, preferably as “C₂₋₈ alkenyl”. “C₂₋₈ alkenyl” should be understood as preferably denoting a straight or branched monovalent hydrocarbon group comprising one or more double bonds and having 2, 3, 4, 5, 6, 7 or 8 carbon atoms, in particular 2 or 3 carbon atoms (“C₂₋₃ alkenyl”), it should be understood that, in case the alkenyl group comprises more than one double bond, the double bonds may be separated from each other or conjugated. The alkenyl group is, for example, vinyl, allyl, (E)-2-methylvinyl, (Z)-2-methylvinyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2 alkenyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 2-methylprop-2-enyl, 2-methylprop-2-enyl, 2-methylpropan-2-enyl, 2-methylpropan-2-enyl, 2-methylpropen-2-enyl, 2-methylpropen-2-enyl, 2-methylpropen-2-enyl, 3-methyllene, 2-methanolenyl, 2-methyldien-2-enyl prop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl alkenyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethyl (Z)-1-methylbut-1-enyl, 1,1-dimethylbut-1-enyl, 1,1-dimethylprop-2-enyl, 1-ethylprop-1-enyl, 1-propylvinyl, 1-isopropylvinyl.

The term “C₃₋₂₀ cycloalkyl” as used in this invention preferably refers to a saturated monovalent monocyclic, bicyclic (e.g., bridged, spiro) hydrocarbon ring or tricyclic alkane having 3, 4, 5, 6, 7, 8, 9 or 10 carbon atoms, the C₃₋₁₀ cycloalkyl group may be a monocyclic hydrocarbon group such as cyclopropyl, cyclobutyl, cyclopentyl, cyclohexyl, cycloheptyl, cyclooctyl, cyclononyl, or cyclodecyl or a bicyclic hydrocarbon group such as lobelinyl, indolyl, hexahydropyridyl, tetrahydronaphthalenyl, decahydronaphthalenyl, dicyclo[2.1.1]hexyl, dicyclo[2.2.1]heptyl, dicyclo[2.2.1]heptenyl, 6,6-dimethyldicyclo[3.1.1]heptyl, 2,6,6-trimethylbicyclo[3.1.1]heptyl, bicyclo[2.2.2]octyl, 2,7-diazaspiro[3,5]nonanyl, 2,6-diazaspiro[3,4]octanyl, or tricyclic hydrocarbon groups such as adamantyl.

The term “C₅₋₂₀ cycloalkenyl” as used in this invention preferably refers to a conjugated or non-conjugated monocyclic or bicyclic monovalent hydrocarbon ring with 5 to 20 carbon atoms, preferably “C₅₋₁₀ cycloalkenyl”, with an unsaturation degree of 1, 2 or 3. The term “C₅₋₁₀ cycloalkenyl” preferably refers to an unsaturated monocyclic or bicyclic hydrocarbon ring having 5, 6, 7, 8, 9 or 10 carbon atoms, the C₅₋₁₀ cycloalkenyl group may be a monocyclic hydrocarbon group such as 3-cyclopentenyl, 1-cyclohexenyl, 2-cyclohexenyl, 2,4-cyclopentadienyl, 2,5-cyclohexadienyl or 1,3,5-cycloheptatrienyl or 1,3,6-cycloheptatrienyl or a bicyclic hydrocarbon group such as hexahydroxynaphthalene ring, octahydroxynaphthalene ring. Unless otherwise specified, the term “C₅₋₂₀ cycloheptenyl” includes all possible isomeric forms thereof, such as their positional or conformational isomers.

Unless otherwise defined, the term “3-20-membered heterocyclyl refers to a saturated or unsaturated non-aromatic ring or ring system, e.g., a 4-, 5-, 6-, or 7-membered monocyclic ring, a 7-, 8-, 9-, or 5-membered dicyclic ring (e.g., a thick ring, a bridged ring, a spirocycle), or a 10-membered tricyclic ring system, and comprises at least one of, e.g., 1, 2, 3, 4, 5, 6, 8, 9, 9, 10-membered tricyclic ring. 1, 2, 3, 4, 5 or more heteroatoms selected from O, S and N, wherein N and S may also optionally be oxidized to various oxidation states to form a nitrogen oxide, —S(O)— or —S(O)₂— state. Preferably, the heterocyclic group may be selected from “3-10 element heterocyclic groups”, the heterocyclic group may be connected to the rest of the molecule via any of the carbon atoms or a nitrogen atom, if present, the heterocyclic group may contain a ring of thickened or bridged rings as well as a ring of spiro rings. In particular, the heterocyclic groups may include, but are not limited to: a 4-membered ring such as azetidinyl, oxetidinyl, a 5-membered ring such as tetrahydrofuranyl, dioxetidinyl, pyrrolidinyl, imidazolidinyl, pyrazolidinyl, pyrrolidinyl, or a 6-membered ring such as tetrahydropyranyl, piperidinyl, morpholino, dithiane, thiomorpholino, piperazinyl, or trithiane, or a 7-membered ring such as diazepane. Optionally, the heterocyclic group may be benzoic thickened, the heterocyclic group may be bicyclic, such as, but not limited to, a 5,5-membered ring such as a hexahydrocyclopenta[c]pyrrolo-2(1H)-yl ring or a 5,6-membered bicyclic ring such as a hexahydropyrrolo[1,2-a]pyrazin-2(1H)-yl ring. The heterocyclic group may be partially unsaturated, i.e., it may contain one or more double bonds, such as, but not limited to, dihydrofuranyl, dihydropyranyl, 2,5-dihydro-1H-pyrrolyl, 4H-[1,3,4]thiadiazinyl, 4,5-dihydrooxazolyl, or 4H-[1,4]thiazinyl, or, alternatively, it may be benzoic and densely packed, for example, but not limited to, dihydroisoquinolinyl. When the 3-10-membered heterocyclic group is attached to other groups to constitute a compound of the present disclosure, it can be a carbon atom on the 3-10-membered heterocyclic group attached to other groups, or it can be a heterocyclic atom on the 3-10-membered heterocyclic group ring attached to other groups. For example, when the 3-10-membered heterocyclic group is selected from a piperazinyl group, it can be a nitrogen atom on the piperazinyl group attached to other groups. Or when the 3-10-membered heterocyclic group is selected from piperidinyl, it may be that the nitrogen atom on the ring of the piperidinyl group and the carbon atom in the opposite position thereof are connected to other groups.

The term “C₆₋₂₀ aryl” as used in this invention preferably refers to a monovalent aromatic or partially aromatic monocyclic or bicyclic (e.g., thickened, bridged, spiro) hydrocarbon ring having 6 to 20 carbon atoms, which may be a monoaromatic ring or a polyaromatic ring held together, in particular a ring having 6 carbon atoms (“C₆-aryl”), such as phenyl, or biphenyl, or a ring having 9 carbon atoms (“C₉-aryl”), such as indanyl or indenyl, or a ring having 10 carbon atoms (“C₁₀-aryl”), such as naphthyl. When the C₆₋₁₀ aryl group is substituted, it may be mono or polysubstituted. Moreover, there is no restriction on its substitution site, e.g. it can be an ortho-, para- or meta-substitution.

The term “5-20-membered heteroaryl” as used in this invention preferably refers to including a monovalent monocyclic, bicyclic (e.g., thickened, bridged, spiro) or tricyclic aromatic ring system having 5 to 10 ring atoms and comprising 1-5 heteroatoms independently selected from N, O, and S, and preferably 1-3 heteroatoms independently selected from N, O, and S, and, in addition, in each case may be benzene-conjugated. In each case, it may be benzo[aryl]thickened. “Heteraryl” also means a group in which a heteraryl ring is fused with one or more aryl, alicyclic or heterocyclic rings, where the base or point of the connection is on the heteraryl ring. Non-limiting examples include 1-, 2-, 3-, 5-, 6-, 7- or 8-indolizinyl, 1-, 3-, 4-, 5-, 6- or 7-isoindolyl, 2-, 3-, 4-, 5-, 6- or 7-indolyl, 2-, 3-, 4-, 5-, 6-, or 7-indazolyl, 2-, 4-, 5-, 6-, 7-, or 8-purinyl, 1-, 2-, 3-, 4-, 6-quinazinyl, 2-, 3-, 4-, 5-, 6-, 7- or 8-quinolinyl, 1-, 3-, 4-, 5-, 6-, 7- or 8-isoquinolinyl, 1-, 4-, 5-, 6-, 7- or 8-phthalazinyl, 2-, 3-, 4-, 5- or 6-naphthyridinyl, 2-, 3-, 5-, 6-, 7- or 8-quinazolinyl, 1-, 4-, 5-, 6-, 7-, 7- or 8-quinazolyl, 2-, 4-, 5-, 6-, 7- or 8-quinazolyl, 2-, 4-, 5-, 6-, 7- or 7-quinazolinyl, 1-, 2-, 3-, 5-, 5-, 6-, 7- or 8-quinazolinyl or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH-carbazolinyl, 1-, 2-, 3-, 4-, 5-, 6-, 7-, or 8-carbazolylcarbazolinyl, 1-, 3-, 4-, 5-, 6-, 7-, 8-, or 9-carbazolinyl 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-acridinyl, 2-, 3-, 4-, 5-, 6-, 8-, 9-, or 10-phenanthridinyl, 1-, 2-, 3-, 4-, 6-, 7-, 8-, or 10-carbazolylcarbazolyl, 1-, 2-, 3-, 4-, 6-, 7-, 8-, or 10-carbazolinyl-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenazinyl, 2-, 3-, 4-, 5-, 6-, or 1-, 3-, 4-, 5-, 6-, 7-, 8-, 9-, or 10-benzoisoquinolinyl, 2-, 3-4- or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrazino[2,3-d]-o-cyclozine radical, 1-, 3-, or 5-1H-pyrazolo[4,3-d]-azolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10-, or 11-4H-pyrido[2,3-c]carbazolyl, 2-, 3-, 6-, or 7-imidazo[1,2-b][1,2,4]triazinyl, 7-benzo[b]thiophene, 2-, 4-, 5-, 6-, or 7-benzazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzazinyl, 1-, 2-, 3-, 5-, 6-, 7-, 8-, 9-, 10-, or 11-1H-pyrrolo[1,2-b][2]benzazepinyl (benzazapinyl). Exemplary thickened heteroaryl groups include, but are not limited to, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benz[b]thiophenyl, 2-, 4-, 5-, 6-, or 7-benzazolyl, 2-, 4-, 5-, 6- or 7-benzimidazolyl and 2-, 4-, 5-, 6- or 7-benzothiazolyl. When the 5-10-membered heteroaryl group is attached to other groups to constitute a compound of the present disclosure, it can be a carbon atom on the 5-10-membered heteroaryl ring attached to other groups or a heteroatom on the 5-10-membered heteroaryl ring attached to other groups. When the 5-10-membered heteroaryl group is substituted, it can be monosubstituted or polysubstituted. Moreover, there is no restriction on the substitution site, which can be, for example, the substitution of a hydrogen attached to a carbon atom on the heteroaryl ring, or the substitution of a hydrogen attached to a heteroatom on the heteroaryl ring.

The term “spiro ring” refers to a ring system in which two rings share one ring-forming atom.

The term “thick ring” refers to a ring system in which two rings share two ring-forming atoms.

The term “bridged ring” means a ring system in which two rings share more than three ring-forming atoms. Unless otherwise indicated, a heterocyclic, heteroaryl or subheteroaryl group includes all possible isomeric forms thereof, e.g., its positional isomers. Thus, for some illustrative, non-limiting examples, forms which are substituted or bonded to other groups at one, two or more of their 1-, 2-, 3-, 4-, 5-, 6-, 7-, 8-, 9-, 10-positions, etc., if present, may be included, including pyridin-2-yl, pyridin-2-yl, pyridin-3-yl, pyridin-3-yl, pyridin-4-yl and sub pyridin-4-yl, thienyl or thienylidene including thiophen-2-yl, thiophen-2-yl, thiophen-3-yl, and thiophen-3-yl, pyrazol-1-yl, pyrazol-3-yl, pyrazol-4-yl, and pyrazol-5-yl.

The term “oxo” refers to an oxy substitution (═O) formed by oxidation of a carbon, nitrogen or sulfur atom in a substituent.

Unless otherwise indicated, the definition of a term herein applies equally to the group comprising the term, e.g., the definition of C₁₋₂₀ alkyl applies also to the term C₁₋₂₀ alkyloxy.

In the term “tris(C₁₋₂₀ alkyloxy)silyl” and the term “tris(C₁₋₂₀ alkyl)silyl”, each C₁₋₂₀ alkyl group may be the same as or different from each other, independently of each other, as selected from the definitions described above.

Group abbreviations used herein have meanings familiar in the art, e.g., Me for methyl, Et for ethyl, Ph for phenyl, TMS for trimethylsilyl, i-Pr for isopropyl, Ac for acetyl.

It will be appreciated by those skilled in the art that the compounds shown in formula (I) may be present in the form of various pharmaceutically acceptable salts. These compounds can form acid addition salts if they have a basic center, base addition salts if they have an acidic center, and internal salts if they contain both an acidic center (e.g., carboxyl) and a basic center (e.g., amino).

The compounds of the disclosure can exist in the form of solvent compounds (e.g., hydrates), wherein the compounds of the disclosure comprise a polar solvent as a structural element of the lattice of the compounds, in particular, e.g., water, methanol or ethanol. The amount of polar solvent, in particular water, may be present in a stoichiometric or non-stoichiometric ratio.

The compounds of the disclosure can exist in the form of stereoisomers, the stereoisomers can be enantiomers and cis-trans-isomers, or enantiomers and diastereomers.

Depending on their molecular structure, the compounds of the disclosure may be chiral and thus may exist in various enantiomeric forms. These compounds may thus exist in the racemic form or in an optically active form. The compounds of the present disclosure cover isomers or mixtures thereof, racemates, in which the respective chiral carbon is in the R or S configuration. The compounds of the present disclosure or intermediates thereof can be isolated as enantiomeric compounds by chemical or physical methods well known to those skilled in the art, or used in this form for synthesis. In the case of racemic amines, diastereoisomers are made from mixtures by reaction with optically active splitting reagents. Examples of suitable splitting reagents are optically active acids, such as the R and S forms of tartaric acid, diacetyltartaric acid, dibenzoyltartaric acid, mandelic acid, malic acid, lactic acid, suitable N-protected amino acids (e.g., N-benzoylproline or N-phenylsulfonylproline), or various optically active camphorsulfonic acids. Chromatographic enantiomeric splitting can also be advantageously carried out with the aid of optically active splitting reagents (e.g. dinitrobenzoylphenylglycine immobilized on silica gel, cellulose triacetate or other carbohydrate derivatives or chirally derivatized isobutenate polymers). Suitable eluents for this purpose are water-containing or alcohol-containing solvent mixtures, e.g., hexane/isopropanol/acetonitrile.

The term “cis-trans isomer” refers to an isomer in which the substituent atoms associated with the carbon-carbon double bond are in different orientations. The substituent atoms (other than H) on either side of the carbon-carbon double bond may be in the E or Z configuration. In the “E” (opposite) or “chair” configuration, the substituent is on the opposite side of the carbon-carbon double bond. In the “Z” (same-side) or “boat” configuration, the substituent is oriented on the same side of the carbon-carbon double bond. Compounds having a mixture of “cis” and “trans” species are designated as “cis/trans”.

The symbols “R”, “S”, “E”, “Z” are as described herein, “cis”, “trans”, etc. refer to atomic configurations relative to the core molecule and as described in IUPAC Recommendations for Fundamental Stereochemistry (Section E)), Pure Appl. Chem. 1976, 45: 13-30.

Individual isomers of the compounds of the disclosure can be prepared by either isomer-specific synthesis or by separating from a mixture of isomers. Conventional separating techniques include the separating of isomeric mixtures of either starting materials or final products by employing optically active salts to form free bases of the respective isomers of the isomeric pairs (followed by hierarchical crystallization and regeneration of the free bases), by forming esters or amides of the respective isomers of the isomeric pairs (followed by chromatographic separation and removal of chiral coagents), or by employing preparative TLC (thin layer chromatography) or chiral HPLC columns.

The term “therapeutic effective dose” means the amount of an active compound or drug that a researcher, veterinarian, physician, or other clinician is looking for in a tissue, system, animal, individual, or person to cause a biological or medical response, and it includes one or more of the following: (1) to prevent disease: For example, prevention of diseases, disorders or conditions in individuals who are susceptible to them but have not yet experienced or developed the pathology or symptoms of the disease. (2) Suppression of disease: for example, suppression of a disease, disorder or condition in an individual who is experiencing or presenting with the pathology or symptoms of a disease, disorder or condition (i.e. preventing the further development of the pathology and/or symptoms). (3) Remission of disease: for example, remission of disease, disorder or condition (i.e. reversal of pathology and/or symptom) in an individual who is experiencing or presenting with the pathology or symptom of a disease, disorder or condition. The therapeutic effective dose can be estimated initially from cell culture assays, or the initial dose can be estimated from in vivo data. Using these preliminary guidelines, an effective dose for humans can be determined by a person of ordinary skill in the art. In addition, the toxicity and therapeutic efficacy of the compounds described herein can also be determined by standard drug procedures in cell cultures or laboratory animals, such as by the determination of LD₅₀ and ED₅₀.

“Excipients” in this invention described herein are given in “Handbook of Pharmaceutical Excipients, 2nd Edition, 1994, edited by A Wade and PJ Weller”.

The “carriers” or “diluents” described in the present disclosure are well known in the pharmaceutical field and are described in, for example, Remington's Pharmaceutical Sciences, Mack Publishing Co. A.R. Gennaro, ed. 1985).

The choice of drug carrier, excipient or diluent may be based on the intended route of administration and standard pharmacy practice. The pharmaceutical compositions may include or in addition contain any suitable adhesive, lubricant, suspension agent, coating agent, solubilizer, buffer, flavoring agent, surfactant, thickener, preservative (including antioxidant), etc. as a carrier, excipient or diluent, as well as substances that are included to make the formulation isotonic with the blood of the recipient.

Pharmaceutical formulations in which the carrier is solid suitable for oral administration are most preferably in the form of unit dose formulations, such as pills, capsules or tablets each containing a predetermined amount of the active compound. The tablets can be prepared by pressing or molding, optionally with more than one auxiliary ingredient. Pressed tablets can be prepared by pressing the active compound in free-flowing form (such as powder or granules), optionally mixed with a binder, lubricant, inert diluent, lubricating substance, surfactant, or dispersant, in a suitable machine. The molded tablet can be prepared by molding the active compound and the inert liquid diluent. The tablets can be optionally coated, or optionally printed with symbols if not coated. Capsules can be prepared by filling the capsule shell with the active compound alone or in combination with more than one auxiliary ingredient and then sealing in a conventional manner. Flat capsules are similar to capsules in which the active compound is sealed in a rice paper film along with any auxiliary ingredients. The active compound can also be formulated as dispersible granules, which can be suspended in water, for example, or sprinkled on food prior to administration. The granules can be packaged in, for example, a sachet. Wherein the carrier may be a liquid formulation suitable for oral administration, it may be presented as a solution or suspension in the form of an aqueous or non-aqueous liquid, or as an oil-in-water liquid emulsion.

The term “pharmaceutically acceptable salt” includes a suitable acid addition salt or base salt thereof. For suitable pharmaceutical salts see J Pharm Sci, 66, 199, 1977, Berge et al.

The term “pharmaceutically acceptable ester” refers to the use of an organic acid or alcohol/hydroxide to form an ester with a functional group in the structure of a compound of the disclosure that can be esterified.

The term “isotopic marker” means that at least one atom in the compound of the disclosure is replaced by an isotope. Examples of the isotopes include isotopes of hydrogen, carbon, nitrogen, oxygen, phosphorus, sulfur, fluorine, and chlorine, such as the corresponding ²H, ³H, ¹³C, ¹⁴C, ¹⁵N, ¹⁷O, ¹⁸O, ³¹P, ³²P, ³⁵S, ¹⁸F, and ³⁶Cl. Replacement with an isotope such as deuterium (i.e., ²H) may provide certain therapeutic advantages resulting from greater metabolic stability, such as an increased in vivo half-life or a reduced dose requirement. Certain therapeutic advantages may therefore be preferred in certain embodiments. For example, the present disclosure includes compounds of general formula (I) in which any hydrogen atom is replaced by a deuterium atom.

The term “prodrug compound” refers to a covalently bonded compound which releases in vivo the active parent drug according to general formula (I). Such a prodrug is typically a compound of the disclosure in which more than one of the appropriate groups has been modified such that the modification may be reversed after administration to a human or mammalian subject. Reversal is usually accomplished by enzymes naturally present in such subjects, although a second agent may be administered with such a prodrug to allow reversal in vivo. Examples of such modifications include the pharmaceutically acceptable esters described above, wherein such reversal may be performed by esterases and the like.

The term “polymorphs” refers to compounds of the disclosure in various crystalline, polycrystalline and hydrated forms. It is well established in the pharmaceutical industry that chemical compounds in any of these forms can be isolated by means of purification and/or separation of the solvents used in the synthetic preparation of such compounds.

The term “administration” indicates that the pharmaceutical compositions of the disclosure may be suitable for rectal, intranasal, endobronchial, topical (including oral and sublingual), vaginal or parenteral (including subcutaneous, intramuscular, intravenous, intra-arterial and intradermal), intraperitoneal or intrathecal administration. Preferably, the formulation is a formulation for oral administration. The formulation may conveniently be presented in a unit dosage form, i.e. in the form of a discrete portion comprising a unit dose or a plurality of units or sub-units of a unit dose. As examples, the formulations may be in the form of tablets and extended-release capsules and may be prepared by any method well known in the field of pharmacy.

The formulations of the present disclosure for oral administration may be presented as: discrete units such as capsules, gels, drops, flat capsules, pills or tablets each containing a predetermined amount of active agent, as powders or granules, as solutions, emulsions or suspensions of active agents in aqueous liquids or non-aqueous liquids, or as oil-in-water liquid emulsions or water-in-oil liquid emulsions, or as regimentally injected formulations and the like. Preferably, each dose of these compositions contains from 1 to 250 mg and more preferably 10-100 mg of the active ingredient.

For compositions for oral administration (e.g., tablets and capsules), they also include solvents and/or commonly used excipients, such as binders, e.g., sugar syrups, gum arabic, gelatin, sorbitol, astragali gum, polyvinylpyrrolidone (polyvinyl ketone), methylcellulose, ethylcellulose, sodium carboxymethylcellulose, hydroxypropylmethylcellulose, sucrose and starch, fillers and carriers, e.g., cornstarch, gelatin, lactose, sucrose, microcrystalline cellulose, kaolin, mannitol, dicalcium phosphate, sodium chloride, and alginate, and lubricants such as magnesium stearate, sodium stearate, and other metal stearates, glyceryl stearate, stearic acid, silicone fluids, talc, oil, and colloidal silica. Flavoring agents such as peppermint, wintergreen oil, and cherry spice may also be used. Colorants may need to be added to make the dosage form easily identifiable. Tablets may also be coated by methods well known in the art.

Other formulations suitable for oral administration include lozenges comprising a flavored matrix, typically sucrose and an active agent in gum arabic or gum astragali, soft lozenges comprising an inert matrix such as gelatin and glycerin, or sucrose and an active agent in gum arabic, and mouthwashes comprising an active agent in a suitable liquid carrier.

Other forms of administration include solutions or emulsions that are administered intravenously, intra-arterially, intrathecally, subcutaneously, intradermally, intraperitoneally or intramuscularly and are prepared from sterile or sterilizable solutions. The injectable forms typically contain between 10-1000 mg, preferably between 10-250 mg of active ingredient per dose.
the form of administration may also be co-administration, i.e. the administration of more than one compound of the disclosure in combination with more than one other active agent. In this case, the compound of the present disclosure can be administered continuously, simultaneously or sequentially with more than one other active agent.

### DETAILED DESCRIPTION

The technical embodiments of the present disclosure will be described in further detail below in connection with specific examples. It should be understood that the following examples are provided solely for the purpose of illustrating and explaining the present disclosure and should not be construed as a limitation of its scope of protection. Any technology realized based on the above contents of the present disclosure falls within the intended scope of protection of the present disclosure.

Unless otherwise indicated, the raw materials and reagents used in the following examples are commercially available or can be prepared by known methods.

### ANALYTICAL METHODS

¹H nuclear magnetic resonance (NMR) spectroscopy was performed at room temperature in the solvents using a Bruker AV 400 spectrometer, unless otherwise indicated. In all cases, the NMR data were consistent with the proposed structures. The characteristic chemical shifts (8) are given in parts per million using the conventional abbreviations used to designate the main peaks: e.g., s, single peak, d, dichotomous peak, t, triple peak, q, quadruple peak, dd, double dichotomous peak, broad. Mass spectra were recorded using an Agilent 1290 Infinity/6460 triple quad LCMS. When thin layer chromatography (TLC) is used, it refers to silica gel TLC.

### Example 1: Synthesis of Tritiated Alkaloid Analogs Containing a Nitro-Substituted Benzene Ring

### Step 1.1

### Preparation of Compound 5

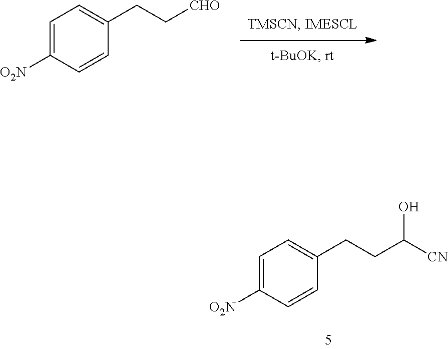 [SMILES: O=CCCc1ccc([N+](=O)[O-])cc1 ; N#CC(O)CCc1ccc([N+](=O)[O-])cc1]

Under argon protection, p-nitrophenyl propionaldehyde (0.18 g, 1 mmol), NC-TMS (0.12 g, 1.2 mmol), IMESCL (0.03 g, 0.1 mmol) were dissolved in THF (5 mL), and t-BuOK (0.01 g, 0.1 mmol) was added with stirring at room temperature for 5 min. The reaction was monitored by TLC plate. After complete, concentrated, quenched by adding EtOAc (10 mL), 2 M HCl (5 mL), partitioned, extracted with EtOAc (2×10 mL), combined organic phases, washed with sodium chloride solution, dried with anhydrous sodium sulfate, filtered and concentrated. Column separation PE:EA=4:1.

Compound 5: light yellow liquid, 1.90 g, yield 94%. ¹H NMR (400 MHz, CDCl₃) δ 8.17 (d, J=8.3 Hz, 2H), 7.38 (d, J=8.3 Hz, 2H), 4.49 (t, J=6.5 Hz, 1H), 3.26 (s, 1H), 2.97 (t, J=7.8 Hz, 2H), 2.20 (ddd, J=11.4, 8.9, 5.6 Hz, 2H) ppm.

Same as step 1.1, the difference from Example 1 is that m-nitropropionaldehyde and 3-nitropropionaldehyde were used instead of p-nitropropionaldehyde to obtain the following compounds 6 and 7, respectively:

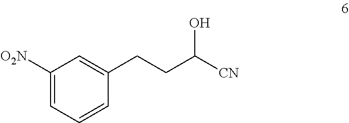 [SMILES: N#CC(O)CCc1cccc([N+](=O)[O-])c1]

Compound 6: light yellow liquid, 8.10 g, yield 94%. ¹H NMR (400 MHz, CDCl₃) δ 8.11 (dd, J=7.6, 1.4 Hz, 2H), 7.56 (dt, J=7.7, 1.5 Hz, 1H), 7.50 (td, J=7.5, 1.1 Hz, 1H), 4.51 (t, J=6.5 Hz, 1H), 2.98 (t, J=7.8 Hz, 2H), 2.30-2.15 (m, 2H) ppm.

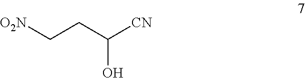

Compound 7: colorless liquid, 7.2 g, yield 79%. ¹H NMR (400 MHz, CDCl₃) δ 4.75-4.61 (m, 2H), 4.61-4.51 (m, 1H), 2.64-2.54 (m, 1H), 2.53-2.42 (m, 1H) ppm.

### Step 1.2

### Preparation of Compound 8

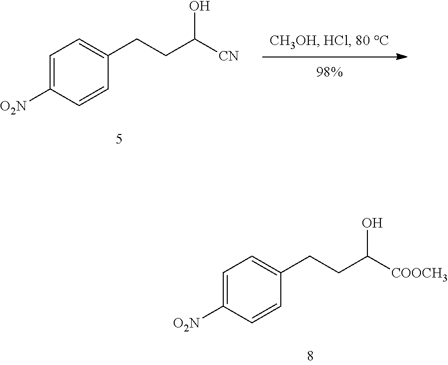 [SMILES: N#CC(O)CCc1ccc([N+](=O)[O-])cc1 ; COC(=O)C(O)CCc1ccc([N+](=O)[O-])cc1]

General operation: under argon protection, compound 5 (1.90 g, 9.21 mmol) was dissolved in CH₃OH (30 mL) solution, added concentrated HCl (6.5 mL), the mixture was heated to 80 °C and refluxed about overnight, after the reaction was completely monitored by TLC plate, it was cooled down to room temperature, concentrated, and then quenched by the addition of EtOAc (20 mL), H₂O, partitioned and extracted by EtOAc (2×20 mL). The organic phases were combined, washed with sodium chloride solution, dried over anhydrous sodium sulfate, filtered and concentrated.

Compound 8: light yellow liquid, 2.15 g, 98% yield. ¹H NMR (400 MHz, CDCl₃) δ 8.15 (d, J=8.7 Hz, 2H), 7.37 (d, J=8.8 Hz, 2H), 4.19 (dd, J=8.1, 3.8 Hz, 1H), 3.77 (s, 3H), 2.97-2.76 (m, 2H), 2.24-2.08 (m, 2H) ppm.

Same as step 1.2, the difference is replacing ingredient 5 with 6 and 7, compounds 9 and 10 can be prepared, respectively:

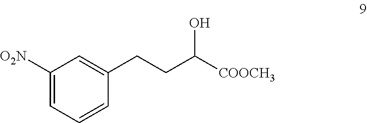 [SMILES: COC(=O)C(O)CCc1cccc([N+](=O)[O-])c1]

Compound 9: light yellow liquid, 9.38 g, 98% yield. H NMR (400 MHz, CDCl₃) δ 8.11-8.02 (m, 2H), 7.55 (d, J=7.5 Hz, 1H), 7.45 (t, J=7.8 Hz, 1H), 4.19 (dd, J=8.0, 3.9 Hz, 1H), 3.77 (s, 3H), 2.97-2.79 (m, 2H), 2.16 (dddd, J=13.7, 9.7, 7.2, 3.9 Hz, 1H), 2.06-1.92 (m, 1H) ppm.

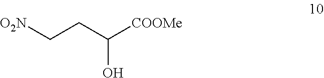 [SMILES: COC(=O)C(O)CC[N+](=O)[O-]]

Compound 10: yellowish liquid, 6.7 g, 60% yield. ¹H NMR (400 MHz, CDCl₃) δ 4.63-4.47 (m, 2H), 4.31 (dd, J=8.7, 4.1 Hz, 1H), 3.82 (s, 3H), 2.58 (ddd, J=14.6, 11.5, 7.2 Hz, 1H), 2.27 (dt, J=14.8, 7.6 Hz, 1H) ppm.

### Step 1.3

### Preparation of Compound 11

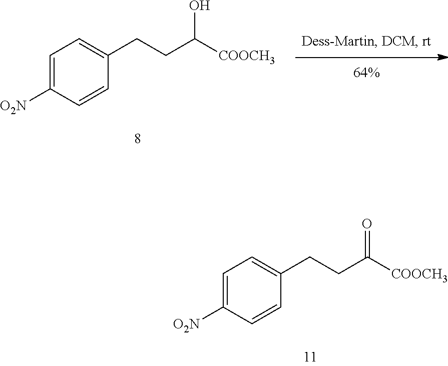 [SMILES: COC(=O)C(O)CCc1ccc([N+](=O)[O-])cc1 ; COC(=O)C(=O)CCc1ccc([N+](=O)[O-])cc1]

Compound 8 (2.15 g, 9.00 mmol) was dissolved in CH₂Cl₂ (80 mL) solution, Dess-Martin (11.45 g, 27.0 mmol) was added, and the reaction was carried out overnight at room temperature. After the reaction was monitored for completeness by TLC plate, the reaction was quenched by the addition of saturated sodium bicarbonate solution and saturated sodium thiosulphate solution (1:1), partitioned, and the organic phases were extracted by CH₂Cl₂ (2×40 mL) was extracted, the organic phases were combined, washed with sodium bicarbonate solution, washed with sodium chloride, dried with anhydrous sodium sulfate, filtered, and concentrated. Column separation PE:EA=4:1.

Compound 11: light yellow liquid, 1.37 g, 64% yield. ¹H NMR (400 MHz, CDCl₃) δ 8.15 (d, J=8.7 Hz, 2H), 7.38 (d, J=8.7 Hz, 2H), 3.87 (s, 3H), 3.24 (t, J=7.1 Hz, 2H), 3.07 (t, J=7.3 Hz, 2H) ppm.

Same as step 1.3, the difference is that compounds 12 and 13 were prepared by replacing raw material 8 with 9 and 10, respectively.

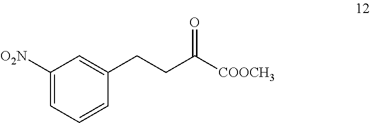 [SMILES: COC(=O)C(=O)CCc1cccc([N+](=O)[O-])c1]

Compound 12: light yellow liquid, 6.82 g, 73% yield. ¹H NMR (400 MHz, CDCl₃) δ 8.08 (dd, J=7.5, 1.4 Hz, 2H), 7.56 (d, J=7.6 Hz, 1H), 7.47 (t, J=8.1 Hz, 1H), 3.87 (s, 3H), 3.26 (t, J=7.3 Hz, 2H), 3.08 (t, J=7.3 Hz, 2H) ppm.

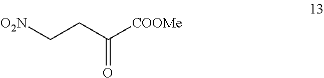 [SMILES: COC(=O)C(=O)CC[N+](=O)[O-]]

Compound 13: colorless liquid, 4.5 g, 68% yield. ¹H NMR (400 MHz, CDCl₃) δ 4.73 (t, J=5.9 Hz, 2H), 3.93 (s, 3H), 3.53 (t, J=5.9 Hz, 2H) ppm.

### Step 1.4

### Preparation of Compound 14

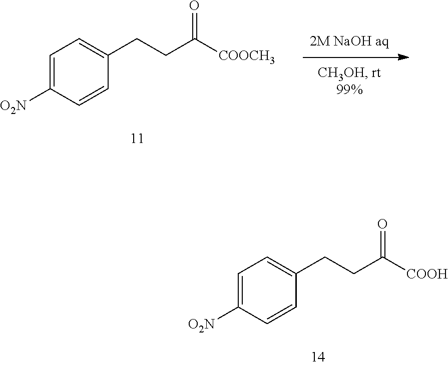 [SMILES: COC(=O)C(=O)CCc1ccc([N+](=O)[O-])cc1 ; O=C(O)C(=O)CCc1ccc([N+](=O)[O-])cc1]

General operation: a solution of CH₃OH (2 mL) of p-nitrophenyl cyclic side chain ketone ester 11 (2.00 g, 8.40 mmol) was added dropwise to an aqueous solution of 2 M NaOH (0.67 g, 16.80 mmol) at room temperature, and the reaction was carried out at room temperature (approx. 2 h). After the reaction was monitored for completeness on a TLC plate, the reaction was concentrated (methanol removed), Et₂O (10 mL) was added, and the liquid was partitioned. The aqueous phase was quenched with 2 M HCl (10 mL), adjusted to pH<1, extracted with Et₂O (3×10 mL), the organic phases were combined, washed with sodium chloride solution, dried over anhydrous sodium sulfate, filtered and concentrated.

Compound 14^{[36]}: pale yellow liquid, 1.00 g, 99% yield. ¹H NMR (400 MHz, CDCl₃) δ 8.15 (d, J=8.7 Hz, 2H), 7.39 (d, J=8.8 Hz, 2H), 3.31 (t, J=7.3 Hz, 2H), 3.09 (t, J=7.2 Hz, 2H) ppm.

Same as step 1.4, the difference is replacing ingredient 11 with 12 and 13, compounds 15 and 16 can be prepared, respectively:

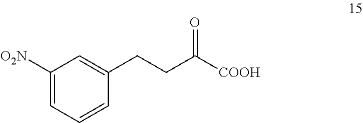 [SMILES: O=C(O)C(=O)CCc1cccc([N+](=O)[O-])c1]

Compound 15: light yellow liquid, 1.00 g, 99% yield. ¹H NMR (400 MHz, CDCl₃) δ 8.13-8.07 (m, 2H), 7.56 (d, J=7.6 Hz, 1H), 7.51-7.46 (m, 1H), 3.36 (t, J=7.3 Hz, 2H), 3.11 (t, J=7.3 Hz, 2H) ppm.

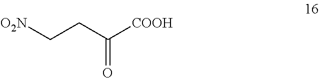

Compound 16: colorless liquid 4.3 g, 96% yield. ¹H NMR (400 MHz, CDCl₃) δ 4.66 (t, J=5.9 Hz, 2H), 3.48 (t, J=5.9 Hz, 2H) ppm.

### Step 1.5

### Preparation of Compound 17

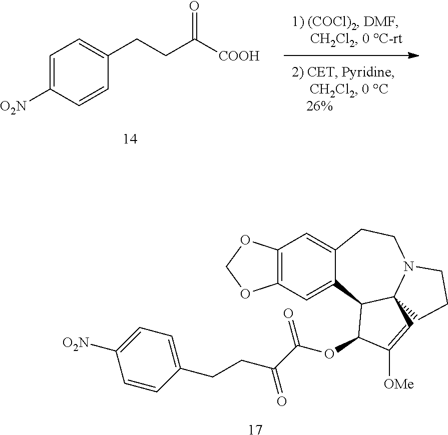

General operation: DMF (0.01 g, 0.02 mmol), oxalyl chloride (0.25 g, 2.0 mmol) were added dropwise to a solution of p-nitrophenyl side-chain α-keto acid 14 (0.23 g, 1 mmol) in CH₂Cl₂ (10 mL) under argon protection at 0 °C. The reaction was carried out overnight. The reaction was transferred to room temperature overnight and concentrated after the reaction was monitored by TLC plate. The crude product was obtained, which was used directly in the next step without further purification.

CET (0.16 g, 0.5 mmol) was dissolved in pyridine (1 mL) and CH₂Cl₂ (5 mL) solution under argon protection, and the above generated p-nitrophenyl side chain ketoyl chloride (0.48 g, 2.00 mmol) was added dropwise to the CH₂Cl₂ (5 mL) solution at 0 °C, and the reaction was carried out at 0 °C overnight. Warmed to room temperature, washed with plenty of water, partitioned, extracted with CH₂Cl₂ (2×10 mL), the organic phases were combined, and the organic phases were washed with sodium bicarbonate solution and sodium chloride solution, respectively, and dried with anhydrous sodium sulfate. Filtering, concentration, gradient column separation (CH₂Cl₂:Et₂O=200:1~100:1).

Compound 17: light yellow amorphous solid, 552 mg, yield 26%. ¹H NMR (400 MHz, CDCl₃) δ 8.12 (d, J=8.5 Hz, 2H), 7.24 (d, J=8.7 Hz, 2H), 6.56 (s, 1H), 6.45 (s, 1H), 5.88 (d, J=9.3 Hz, 1H), 5.80 (s, 1H), 5.75 (s, 1H), 5.11 (s, 1H), 3.82 (d, J=9.4 Hz, 1H), 3.70 (s, 3H), 3.21-3.09 (m, 1H), 3.10-3.02 (m, 2H), 2.94-2.85 (m, 1H), 2.81 (t, J=7.3 Hz, 2H), 2.65 (dt, J=7.2, 3.1 Hz, 1H), 2.61-2.54 (m, 2H), 2.30 (dd, J=14.6, 7.0 Hz, 1H), 2.00 (dd, J=8.4, 3.5 Hz, 1H), 1.94-1.86 (m, 1H), 1.78-1.71 (m, 2H) ppm. HRMS (ESI) m/z: Calcd for C₂₈H₂₉N₂O₈, [M+H]⁺ 521.1918, found 521.1945.

Same as step 1.5, the difference is that raw material 14 was replaced by 15 and 16 to prepare compounds 18 and 19, respectively:

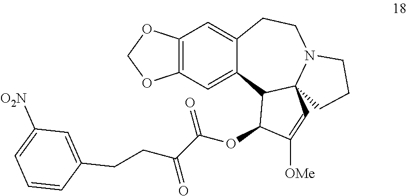

Compound 18: light yellow amorphous solid, 3.916 g, 52% yield. ¹H NMR (400 MHz, CDCl₃) δ 7.89 (dt, J=6.6, 2.5 Hz, 1H), 7.80 (s, 1H), 7.27 (d, J=6.5 Hz, 2H), 6.38 (s, 1H), 6.24 (s, 1H), 5.71 (d, J=9.2 Hz, 1H), 5.60 (d, J=1.4 Hz, 1H), 5.55 (d, J=1.4 Hz, 1H), 4.93 (s, 1H), 3.65 (d, J=9.4 Hz, 1H), 3.53 (s, 3H), 3.03-2.94 (m, 1H), 2.92-2.87 (m, 1H), 2.75-2.70 (m, 1H), 2.64 (t, J=7.3 Hz, 2H), 2.48-2.38 (m, 4H), 2.14 (dd, J=14.5, 7.0 Hz, 1H), 1.88-1.81 (m, 1H), 1.72 (ddd, J=12.2, 7.6, 4.6 Hz, 1H), 1.57 (dd, J=11.1, 5.7 Hz, 2H) ppm. HRMS (ESI) m/z: Calcd for C₂₈H₂₉N₂O₈, [M+H]⁺ 521.1918, found 521.1919.

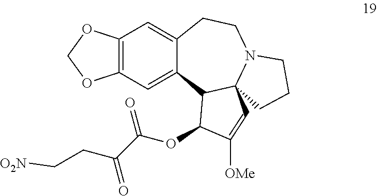

Compound 19: pale yellow amorphous solid, 2.84 g, 67% yield. HRMS (ESI) m/z: Calcd for C₂₂H₂₅N₂O₈, [M+H]⁺ 445.1605, found 445.1610.

### Step 1.6

### Preparation of Compound 20

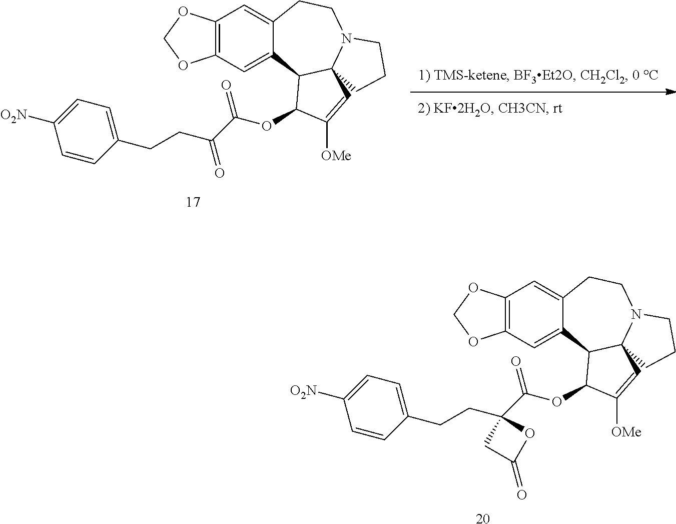

General operation: Under argon protection, boron trifluoride ethyl ether (0.57 g, 4 mmol) was added dropwise to a solution of p-nitrophenyltriacontane α-ketone ester base 17 (0.52 g, 1 mmol) in CH₂Cl₂ (10 mL) at 0 °C for 20 min, followed by the addition of TMS-ketene (0.25 g, 0.5 mmol) in CH₂Cl₂ (10 mL) dropwise, and the reaction was carried out overnight at 0 °C. The reaction was monitored by TLC plate for completeness, quenched by the addition of sodium bicarbonate solution, adjusted to pH=9, and extracted with CH₂Cl₂ (3×10 mL). The reaction was carried out overnight at 0 °C. After the reaction was monitored for completeness on a TLC plate, the reaction was quenched by the addition of sodium bicarbonate solution, adjusted to pH=9, and extracted with CH₂Cl₂ (3×10 mL). The organic phases were combined, washed with sodium chloride solution and concentrated to remove the solvent to give the intermediate product. At room temperature, the intermediate was dissolved in CH₃CN (10 mL) solution, KF·2H₂O (0.47 g, 5 mmol) was added, and the reaction was carried out for about 4 h. After the reaction was completely monitored by TLC plate, it was filtered, and the filtrate was dried with anhydrous sodium sulfate. The filtrate was extracted and concentrated. Column separation PE:EA=4:1, 0.5% TEA.

Compound 20: pale yellow amorphous solid, 40 mg, 22% yield. ¹′H NMR (400 MHz, CDCl₃) δ 8.15 (d, J=8.6 Hz, 2H), 7.28 (d, J=8.6 Hz, 2H), 6.61 (s, 1H), 6.58 (s, 1H), 5.92 (dd, J=9.5, 0.9 Hz, 1H), 5.77 (d, J=1.5 Hz, 1H), 5.60 (d, J=1.5 Hz, 1H), 5.13 (s, 1H), 3.84 (d, J=9.5 Hz, 1H), 3.73 (s, 3H), 3.15-3.00 (m, 3H), 2.98-2.84 (m, 2H), 2.65-2.51 (m, 4H), 2.43-2.20 (m, 3H), 2.08-1.97 (m, 1H), 1.96-1.88 (m, 1H), 1.82-1.71 (m, 2H) ppm. HRMS (ESI) m/z: Calcd for C₃₀H₃₁N₂O₉, [M+H]⁺ 563.2024, found 563.2026.

Same as step 1.6, the difference is replacing ingredient 17 with 18 and 19, compounds 21 and 22 can be prepared, respectively:

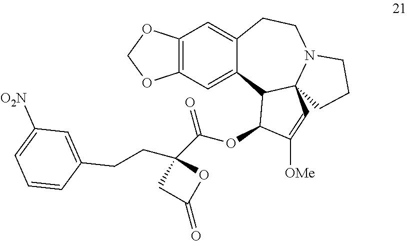

Compound 21: light yellow amorphous solid, 708 mg, 16% yield. ¹H NMR (400 MHz, CDCl₃) δ 8.09 (dt, J=5.9, 2.3 Hz, 1H), 8.01 (s, 1H), 7.47 (d, J=5.2 Hz, 2H), 6.63 (s, 1H), 6.57 (s, 1H), 5.94 (dd, J=9.4, 0.9 Hz, 1H), 5.79 (d, J=1.5 Hz, 1H), 5.65 (d, J=1.5 Hz, 1H), 5.15 (s, 1H), 3.86 (d, J=9.5 Hz, 1H), 3.76 (s, 3H), 3.16-3.05 (m, 2H), 3.01 (d, J 16.5 Hz, 1H), 2.93 (td, J=11.5, 6.9 Hz, 1H), 2.80 (d, J=16.5 Hz, 1H), 2.72 (td, J=12.9, 4.5 Hz, 1H), 2.64-2.56 (m, 3H), 2.46 (dd, J=14.2, 6.8 Hz, 1H), 2.36-2.25 (m, 1H), 2.15-2.06 ((m, 1H), 1.97-1.88 (m, 2H), 1.79-1.73 (m, 2H) ppm. HRMS (ESI)m/z: Calcd for C₃₀H₃₁N₂O₉, [M+H]⁺ 563.2024, found 563.2024.

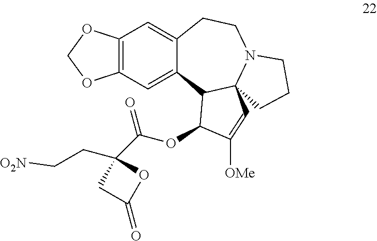

Compound 22: light yellow amorphous solid, 1.20 g, 76% yield. HRMS (ESI) m/z: Calcd for C₂₄H₂₇N₂O₉, [M+H]⁺ 487.1711, found 487.1712.

### Step 1.7

### Preparation of Compound I-2-1

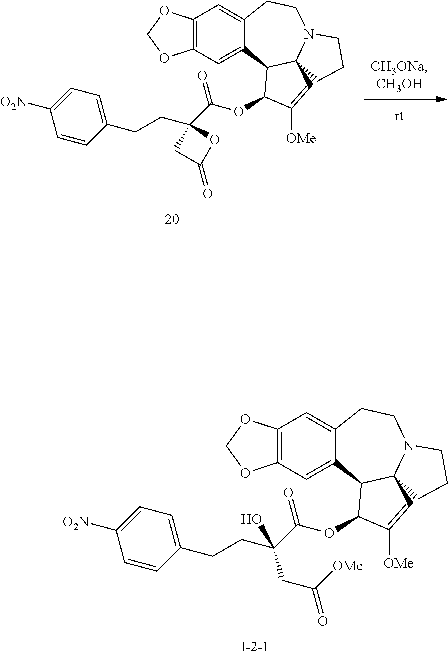 [SMILES: COC1=C[C@]23CCCN2CCc2cc4c(cc2[C@@H]3[C@@H]1OC(=O)[C@@]1(CCc2ccc([N+](=O)[O-])cc2)CC(=O)O1)OCO4 ; "20" ; COC(=O)C[C@](O)(CCc1ccc([N+](=O)[O-])cc1)C(=O)O[C@@H]1C(OC)=C[C@]23CCCN2CCc2cc4c(cc2[C@H]13)OCO4]

General operation: A CH₃OH solution of ready-made CH₃ONa (0.05 g, 1 mmol) was added slowly and dropwise to a CH₃OH (10 mL) solution of p-nitrophenyl trichostatin 20 (0.56 g, 1 mmol) under argon protection at room temperature (approx. 20 min). The reaction was monitored for completeness by TLC plate and then quenched by addition of a saturated ammonium chloride solution (10 mL) was quenched. Concentrate (to remove methanol), add 10 mL of water and 10 mL of CH₂Cl₂, stir and partition, the aqueous phase is extracted with CH₂Cl₂ (3×10 mL), the organic phases are combined and concentrated to remove some of the solvent. Wash with buffer solution (2×10 mL) with pH=6.8 until tritonine was completely removed, wash with sodium bicarbonate solution, sodium chloride solution and dry with anhydrous sodium sulfate. Filter was drawn and concentrated. Column separation PE:EA=4:1, 0.5% TEA.

Compound I-2-1: light yellow amorphous solid, 25 mg, yield 52%. ¹H NMR (400 MHz, CDCl₃) δ 8.13 (d, J=8.6 Hz, 2H), 7.25 (d, J=8.4 Hz, 2H), 6.58 (s, 1H), 6.56 (s, 1H), 6.04 (d, J=9.7 Hz, 1H), 5.83 (d, J=1.6 Hz, 1H), 5.74 (d, J=1.6 Hz, 1H), 5.11 (s, 1H), 3.81 (d, J=9.8 Hz, 1H), 3.69 (s, 3H), 3.65 (s, 1H), 3.59 (s, 3H), 3.19-3.05 (m, 2H), 2.94 (td, J=11.6, 6.9 Hz, 1H), 2.75 (td, J=12.7, 5.3 Hz, 1H), 2.66-2.54 (m, 2H), 2.49-2.32 (m, 2H), 2.29 (d, J=16.4 Hz, 1H), 2.11-1.96 (m, 2H), 1.92 (ddd, J=12.2, 7.6, 4.6 Hz, 1H), 1.82-1.69 (m, 4H) ppm. HRMS (ESI)m/z: Calcd for C₃₁H₃₅N₂O₁₀, [M+H]⁺ 595.2286, found 595.2288.

Same as step 1.7, the difference is that raw material 20 was replaced with 21 and 22 to prepare compounds I-2-2~I-2-4, respectively:

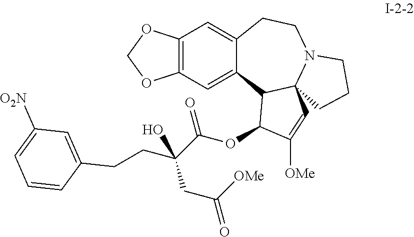

Compound I-2-2: light yellow amorphous solid, 516 mg, 69% yield. ¹H NMR (400 MHz, CDCl₃) δ 8.05 (d, J=7.0 Hz, 1H), 7.96 (s, 1H), 7.47-7.41 (m, 2H), 6.57 (s, 1H), 6.56 (s, 1H), 6.06 (d, J=9.7 Hz, 1H), 5.83 (s, 1H), 5.76 (s, 1H), 5.14 (s, 1H), 3.83 (d, J=9.8 Hz, 1H), 3.73 (s, 3H), 3.68 (s, 1H), 3.59 (s, 3H), 3.21-3.07 (m, 2H), 2.98 (td, J=11.8, 10.8, 6.3 Hz, 1H), 2.78 (td, J=13.0, 5.1 Hz, 1H), 2.70-2.59 (m, 2H), 2.46 (td, J=13.1, 4.4 Hz, 1H), 2.38 (dd, J=14.0, 6.8 Hz, 1H), 2.28 (d, J=16.6 Hz, 1H), 2.12-2.04 (m, 1H), 2.00-1.90 (m, 3H), 1.82-1.72 (m, 3H) ppm. HRMS (ESI)m/z: Calcd for C₃₁H₃₅N₂O₁₀, [M+H]⁺ 595.2286, found 595.2284.

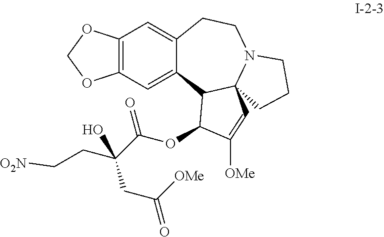

Compound I-2-3: Light yellow amorphous solid, 720 mg, 75% yield. HRMS (ESI) m/z: Calcd for C₂₅H₃₁N₂O₁₀, [M+H]⁺ 519.1973, found 519.1970.

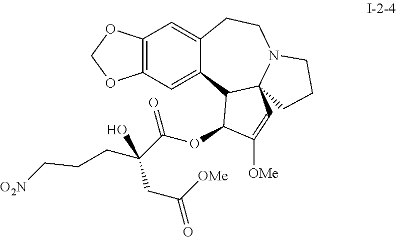

Compound I-2-4: light yellow amorphous solid, 680 mg, 70% yield. HRMS (ESI) m/z: Calcd for C₂₆H₃₃N₂O₁₀, [M+H]⁺ 533.2130, found 533.2135.

### Example 2 Preparation of Tricuspidate Base Analog I-2-5

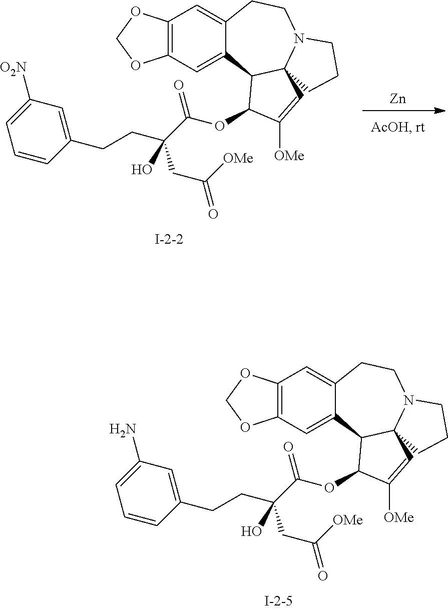

I-2-2 (1 mmol) was dissolved in 1 mL AcOH, Zn powder (20 mmol) was added, the reaction was carried out at room temperature, and the complete reaction was monitored by TLC plate, the mixture was filtered through diatomaceous earth, and the diatomaceous earth was washed by CH₂Cl₂, and the filtrate was obtained, to the filtrate was added CH₂Cl₂ and sodium bicarbonate solution, and it was stirred for 30 min, partitioned, and the aqueous phase was extracted with CH₂Cl₂, and the organic phases were combined, and the mixture was dried. Drying, filtration, concentration. Column chromatography gave a light yellow solid product of compounds I-2-5 of this title (93% yield). ¹H NMR (400 MHz, CDCl₃) δ 7.05 (t, J=7.7 Hz, 1H), 6.59 (s, 1H), 6.55 (s, 1H), 6.53-6.48 (m, 2H), 6.44 (t, J=2.0 Hz, 1H), 6.05 (d, J=9.8 Hz, 1H), 5.84 (d, J=1.6 Hz, 1H), 5.75 (d, J=1.6 Hz, 1H), 5.08 (s, 1H), 3.80 (d, J=9.8 Hz, 1H), 3.70 (s, 3H), 3.58 (s, 4H), 3.20-3.09 (m, 2H), 3.02-2.93 (m, 1H), 2.64-2.50 (m, 3H), 2.38 (dd, J=14.2, 6.8 Hz, 1H), 2.33-2.23 (m, 2H), 2.07-1.92 (m, 3H), 1.82-1.74 (m, 2H), 1.70 (dt, J=11.9, 5.7 Hz, 2H) ppm. HRMS (ESI) m/z: Calcd for C₃₁H₃₇N₂O₈, [M+H]⁺ 565.2544, found 565.2541.

### Example 3 Preparation of Compounds I-2-6~I-2-8

Referring to Example 2, the difference was that raw material I-2-2 was replaced with 1-2-1, 1-2-3 and I-2-4 to prepare compounds I-2-6~I-2-8, respectively:

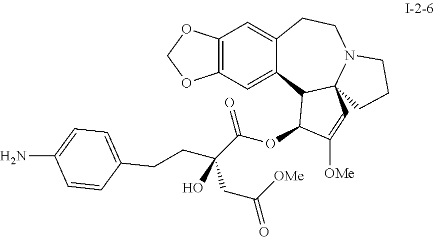

Compound I-2-6: light yellow amorphous product in 90% yield. HRMS (ESI) m/z: Calcd for C₃₁H₃₇N₂O₈, [M+H]⁺ 565.2544, found 565.2540.

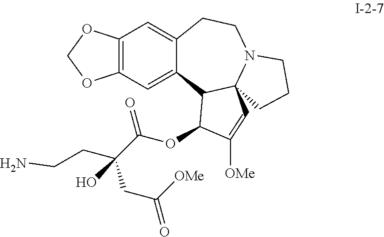

Light yellow amorphous solid product of Compound I-2-7 in 88% yield. HRMS (ESI) m/z: Calcd for C₂₅H₃₃N₂O₈, [M+H]⁺ 489.2231, found 489.2230.

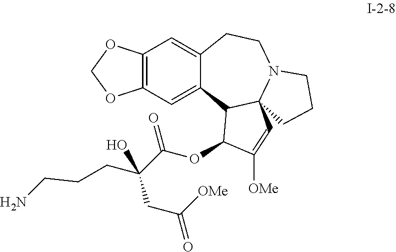

Light yellow amorphous solid product of compound I-2-8 in 92% yield. HRMS (ESI) m/z: Calcd for C₂₆H₃₅N₂O₈, [M+H]⁺ 503.2388, found 503.2392.

### Example 4 Preparation of Tritiated Alkaloid Analogs I-2-9

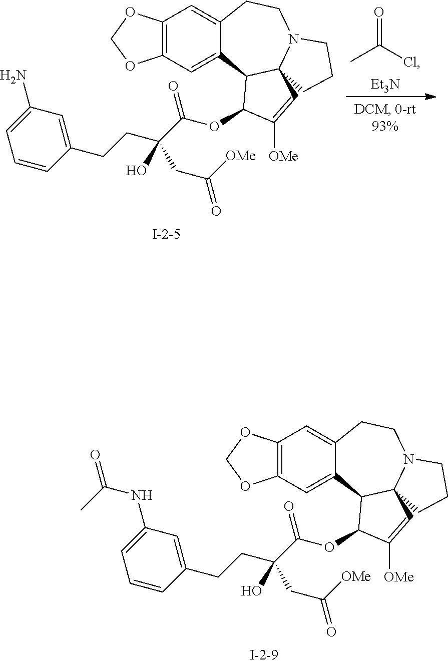

Under the protection of argon, TEA (3 mmol) and acetyl chloride (1.1 mmol) were added dropwise to CH₂Cl₂ (5 mL) of I-2-5 (1 mmol) at 0 °C, and after completion of the dropwise addition, the reaction was transferred to room temperature overnight. After the reaction was completely monitored on a TLC plate, the reaction was quenched by adding sodium bicarbonate solution, adjusted to pH=8, and extracted with CH₂Cl₂, and the organic phases were combined, dried, filtered, and concentrated. Column chromatography afforded a pale yellow solid product (93% yield) of the compound of this title (I-2-9). ¹H NMR (400 MHz, CDCl₃) δ 7.32 (d, J=7.9 Hz, 1H), 7.25 (s, 2H), 7.20 (t, J=7.8 Hz, 1H), 6.84 (d, J=7.5 Hz, 1H), 6.59 (s, 1H), 6.55 (s, 1H), 6.04 (d, J=9.7 Hz, 1H), 5.83 (d, J=1.4 Hz, 1H), 5.72 (d, J=1.5 Hz, 1H), 5.09 (s, 1H), 3.80 (d, J=9.8 Hz, 1H), 3.70 (s, 3H), 3.58 (s, 4H), 3.20-3.06 (m, 2H), 3.01-2.89 (m, 1H), 2.66-2.56 (m, 3H), 2.38 (dd, J=14.2, 6.9 Hz, 1H), 2.33-2.27 (m, 2H), 2.16 (s, 3H), 2.06-1.97 (m, 3H), 1.96-1.87 (m, 2H), 1.72 (dt, J=11.9, 5.7 Hz, 2H) ppm. HRMS (ESI) m/z: Calcd for C₃₃H₃₉N₂O₉, [M+H]⁺ 607.2650, found 607.2651.

### Example 5 Preparation of Compounds I-2-10 and I-2-12

Referring to Example 4, the difference was that raw material I-2-5 was replaced with I-2-6~I-2-8 to prepare compounds I-2-10~I-2-12, respectively:

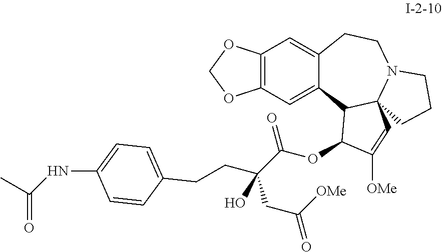

Compound I-2-10: light yellow solid product in 90% yield. HRMS (ESI) m/z: Calcd for C₃₃H₃₉N₂O₉, [M+H]⁺ 607.2650, found 607.2651.

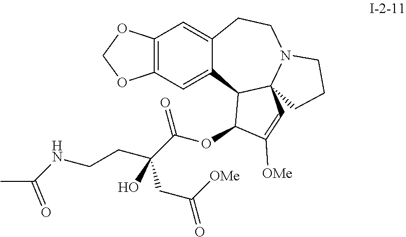 [SMILES: COC(=O)C[C@](O)(CCNC(C)=O)C(=O)O[C@@H]1C(OC)=C[C@]23CCCN2CCc2cc4c(cc2[C@H]13)OCO4]

Compound I-2-11: light yellow solid product, 88% yield. HRMS (ESI) m/z: Calcd for C₂₇H₃₅N₂O₉, [M+H]⁺ 531.2337, found 531.2339.

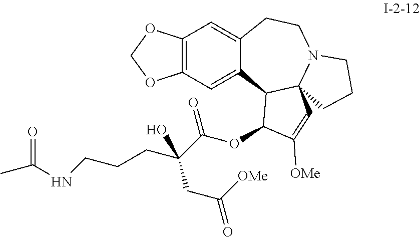 [SMILES: COC(=O)C[C@@](O)(CCCNC(C)=O)C(=O)O[C@@H]1C(OC)=C[C@]23CCCN2CCc2cc4c(cc2[C@H]13)OCO4]

Compound I-2-12: light yellow solid product, 88% yield. HRMS (ESI) m/z: Calcd for C₂₈H₃₇N₂O₉, [M+H]⁺ 545.2494, found 545.2490.

### Example 6 Preparation of Compounds I-2-13~I-2-22

Referring to the preparation of Compound I-2-9 of Example 5, the difference was that different chloroyl chloride instead of acetyl chloride was reacted with Compound I-2-7 or I-2-8 to prepare Compound I-2-13~I-2-22, respectively:

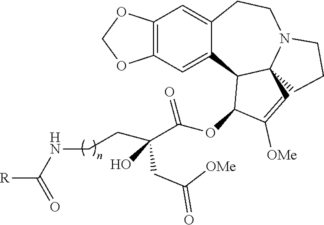

I-2-13~I-2-22

| | | | | | | [M + H]⁺ | [M + H]⁺ |
|---|---|---|---|---|---|---|---|
| number | compound | R | n | yield(%) | formula | Cal. | Found |
| 1 | I-2-13 | i-Pr | 1 | 82 | C₂₉H₃₈N₂O₉ | 559.2625 | 559.2620 |
| 2 | I-2-14 | vinyl | 1 | 85 | C₂₈H₃₄N₂O₉ | 543.2337 | 543.2235 |
| 3 | I-2-15 | MeO | 1 | 90 | C₂₇H₃₄N₂O₁₀ | 547.2286 | 547.2288 |
| 4 | I-2-16 | i-PrO | 1 | 87 | C₂₉H₃₈N₂O₁₀ | 575.2599 | 575.2597 |
| 5 | I-2-17 | t-BuO | 1 | 83 | C₃₀H₄₀N₂O₁₀ | 589.2756 | 589.2758 |
| 6 | I-2-18 | PhO | 1 | 86 | C₃₂H₃₆N₂O₁₀ | 609.2443 | 609.2440 |
| 7 | I-2-19 | cyclopentyl | 2 | 88 | C₃₂H₄₂N₂O₉ | 599.2963 | 599.2960 |
| 8 | I-2-20 | cyclopropyl | 2 | 89 | C₃₀H₃₈N₂O₉ | 571.2650 | 571.2654 |
| 9 | I-2-21 | cyclopentyloxyl | 2 | 86 | C₃₂H₄₂N₂O₁₀ | 615.2912 | 615.2908 |
| 10 | I-2-22 | cyclopropyloxyl | 2 | 87 | C₃₀H₃₈N₂O₁₀ | 587.2599 | 587.2593 |

### Example 7 Preparation of Compounds I-2-23~I-2-28 and I-1-100

Referring to Example 4, the difference is that instead of acetyl chloride, a different chloride was reacted with compound I-2-5 to prepare compounds I-2-23~I-2-28 and I-1-100, respectively:

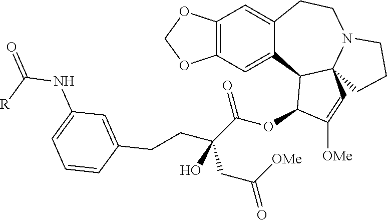

I-2-23~I-2-28

| | | | | | [M + H]⁺ | [M + H]⁺ |
|---|---|---|---|---|---|---|
| number | compound | R | yield(%) | formula | Cal. | Found |
| 1 | I-2-23 | i-Pr | 81 | C₃₅H₄₂N₂O₉ | 635.2963 | 635.2960 |
| 2 | I-2-24 | vinyl | 83 | C₃₄H₃₈N₂O₉ | 619.2650 | 619.2652 |
| 3 | I-2-25 | MeO | 89 | C₃₃H₃₈N₂O₁₀ | 623.2599 | 623.2600 |
| 4 | I-2-26 | i-PrO | 90 | C₃₅H₄₂N₂O₁₀ | 651.2912 | 651.2915 |
| 5 | I-2-27 | cyclopentyloxyl | 82 | C₃₇H₄₄N₂O₁₀ | 677.3069 | 677.3073 |
| 6 | I-2-28 | PhO | 88 | C₃₈H₄₀N₂O₁₀ | 685.2756 | 685.2750 |
| 7 | I-1-100 | Me2N | 84 | C₃₄H₄₁N₃O₉ | 636.2916 | 636.2922 |

### Example 8: Preparation of Tritiated Alkaloid Analog I-1-1

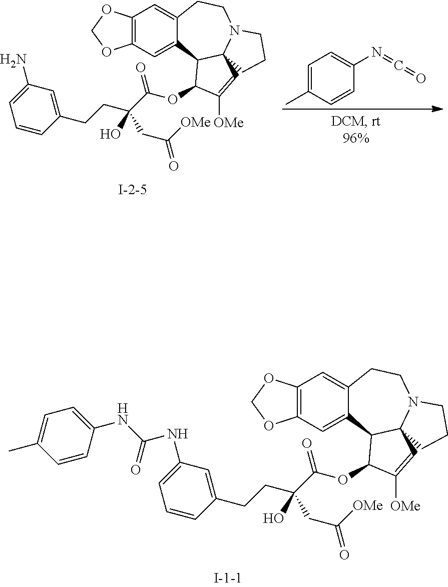

To CH₂Cl₂ (5 mL) of I-2-5 (1 mmol), p-toluene isocyanate (1 mmol) was added dropwise under argon protection and after completion of the dropwise addition, the reaction was carried out at room temperature overnight, after the reaction was monitored for completeness by TLC plate, it was concentrated. Column chromatography afforded a pale yellow solid product (96% yield) of compound I-1-1 of this title. ¹H NMR (400 MHz, CDCl₃) δ 7.23 (d, J=8.4 Hz, 3H), 7.19 (d, J=7.9 Hz, 1H), 7.13 (d, J=8.2 Hz, 2H), 7.04 (s, 1H), 6.83 (d, J=6.8 Hz, 1H), 6.70 (d, J=7.5 Hz, 1H), 6.56 (s, 1H), 6.54 (s, 1H), 6.02 (d, J=9.7 Hz, 1H), 5.82 (d, J=1.4 Hz, 1H), 5.71 (d, J=1.4 Hz, 1H), 5.07 (s, 1H), 3.79 (d, J=9.8 Hz, 1H), 3.68 (s, 3H), 3.58 (s, 3H), 3.20-3.03 (m, 2H), 3.00-2.85 (m, 1H), 2.68-2.52 (m, 3H), 2.36 (dd, J=14.2, 6.8 Hz, 2H), 2.32 (s, 3H), 2.28 (d, J=6.1 Hz, 1H), 2.08-1.97 (m, 3H), 1.97-1.85 (m, 2H), 1.72-1.65 (m, 2H) ppm. HRMS (ESI) m/z: Calcd for C₃₉H₄₄N₃O₉, [M+H]⁺ 698.3072, found 698.3072.

### Example 9: Preparation of Tritiated Alkaloid Analogs I-1-2, I-1-7, I-1-9, I-1-12, I-1-22, I-1-97, I-1-98 and I-1-99

Referring to Example 8, the difference is that compounds I-1-2 can be prepared by replacing p-tolyl isocyanate with cyclopentyl isocyanate, cyclopropyl isocyanate, cyclohexyl isocyanate, tert-butyl isocyanate, benzyl isocyanate, 4-methoxyphenyl isocyanate, 3-chloro-4-methylbenzoyl isocyanate, 3,5-di(trifluoromethyl)phenyl isocyanate, and 4-fluorophenyl isocyanate respectively, and compound I-1-2 can be prepared respectively, I-1-7, I-1-9, I-1-12, I-1-22, I-1-26, I-1-97, I-1-98 and I-1-99, respectively:

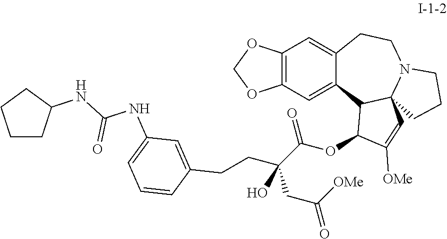

Compound I-1-2 of this title is a pale yellow solid product (69% yield). ¹H NMR (400 MHz, CDCl₃) δ 7.17 (d, J=4.9 Hz, 2H), 7.00 (s, 1H), 6.78 (d, 1H), 6.58 (s, 1H), 6.54 (s, 1H), 6.03 (d, J=9.7 Hz, 1H), 5.82 (d, J=1.4 Hz, 1H), 5.72 (d, J=1.3 Hz, 1H), 5.08 (s, 1H), 3.79 (d, J=9.8 Hz, 1H), 3.68 (s, 3H), 3.58 (s, 3H), 3.20-3.03 (m, 3H), 3.00-2.87 (m, 1H), 2.65-2.54 (m, 3H), 2.37 (dd, J=14.0, 6.8 Hz, 2H), 2.29 (d, J=16.5 Hz, 2H), 2.03 (d, J=8.7 Hz, 2H), 2.01-1.90 (m, 4H), 1.77-1.66 (m, 4H), 1.66-1.58 (m, 4H) ppm. HRMS (ESI) m/z: Calcd for C₃₇H₄₆N₃O₉, [M+H]⁺ 676.3229, found 676.3226.

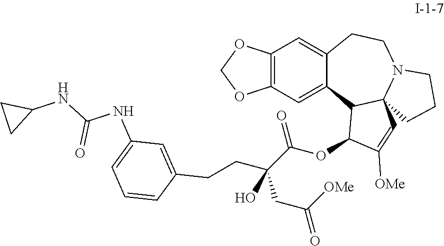

Compounds I-1-7 of this title are pale yellow solid products (92% yield). ¹H NMR (400 MHz, Chloroform-d) δ 7.24-7.20 (m, 1H), 7.11 (t, J=7.8 Hz, 1H), 7.04 (s, 2H), 6.71 (d, J=7.5 Hz, 1H), 6.52 (s, 1H), 6.48 (s, 1H), 5.97 (d, J=9.7 Hz, 1H), 5.76 (d, J=1.6 Hz, 1H), 5.65 (d, J=1.6 Hz, 1H), 5.20 (s, 1H), 5.02 (s, 1H), 3.73 (d, J=9.8 Hz, 1H), 3.63 (s, 3H), 3.58 (s, 1H), 3.51 (s, 3H), 3.15-2.99 (m, 2H), 2.88 (td, J=11.5, 6.8 Hz, 1H), 2.64-2.48 (m, 4H), 2.36-2.29 (m, 1H), 2.24 (dd, J=17.4, 6.2 Hz, 2H), 2.05-1.93 (m, 2H), 1.93-1.84 (m, 1H), 1.73 (d, J=8.0 Hz, 2H), 1.69-1.60 (m, 2H), 0.73 (q, J=4.7 Hz, 2H), 0.54 (q, J=5.1, 4.7 Hz, 2H) ppm. HRMS (ESI) m/z: Calcd for C₃₅H₄₂N₃O₉, [M+H]⁺ 648.2916, found 648.2915.

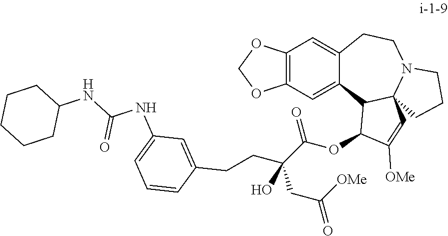

Compound I-1-9 of this title is a pale yellow solid product (89% yield). ¹H NMR (400 MHz, Chloroform-d) δ 7.20-7.13 (m, 2H), 7.01 (s, 1H), 6.84 (s, 1H), 6.76 (d, J=6.7 Hz, 1H), 6.58 (s, 1H), 6.54 (s, 1H), 6.02 (d, J=9.7 Hz, 1H), 5.82 (d, J=1.6 Hz, 1H), 5.72 (d, J=1.6 Hz, 1H), 5.08 (s, 1H), 3.79 (d, J=9.8 Hz, 1H), 3.67 (s, 3H), 3.64 (s, 1H), 3.59-3.63 (s, 1H), 3.58 (s, 3H), 3.19-3.05 (m, 2H), 2.92 (td, J=11.5, 6.9 Hz, 1H), 2.59 (ddt, J=17.6, 12.4, 6.6 Hz, 3H), 2.41-2.34 (m, 1H), 2.35-2.25 (m, 2H), 2.08-1.96 (m, 3H), 1.95-1.89 (m, 3H), 1.79-1.64 (m, 6H), 1.57 (dd, J=10.5, 6.4 Hz, 1H), 1.37-1.28 (m, 2H), 1.17-1.06 (m, 3H) ppm. HRMS (ESI) m/z: Calcd for C₃₈H₄₈N₃O₉, [M+H]⁺ 690.3385, found 690.3382.

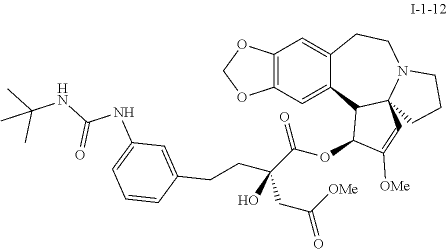

Compound I-1-12 of this title is a light yellow solid product (90% yield). ¹H NMR (400 MHz, Chloroform-d) δ 7.12-7.04 (m, 2H), 6.95 (s, 1H), 6.76 (d, J=12.6 Hz, 1H), 6.66 (d, J=6.8 Hz, 1H), 6.51 (s, 1H), 6.47 (s, 1H), 5.96 (d, J=9.7 Hz, 1H), 5.76 (d, J=1.5 Hz, 1H), 5.66 (d, J=1.6 Hz, 1H), 5.07-4.98 (m, 2H), 3.72 (d, J=9.8 Hz, 1H), 3.62-3.57 (m, 4H), 3.50 (s, 3H), 3.12-2.98 (m, 2H), 2.86 (td, J=11.6, 7.0 Hz, 1H), 2.60-2.47 (m, 3H), 2.31 (dd, J=14.4, 6.7 Hz, 1H), 2.23 (dd, J=14.7, 6.9 Hz, 2H), 2.02-1.92 (m, 2H), 1.90-1.82 (m, 1H), 1.70 (dd, J=12.1, 5.4 Hz, 2H), 1.67-1.61 (m, 2H), 1.28 (s, 9H) ppm. HRMS (ESI) m/z: Calcd for C₃₆H₄₆N₃O₉, [M+H]⁺ 664.3229, found 664.3226.

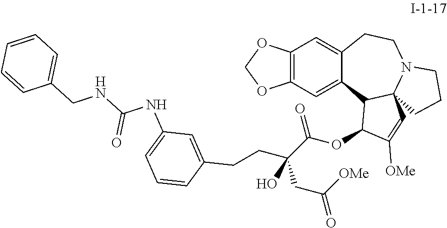

Compound I-1-17 of this title is a pale yellow solid product (93% yield). ¹H NMR (400 MHz, Chloroform-d) δ 7.24-7.17 (m, 5H), 7.09 (d, J=6.4 Hz, 2H), 6.95 (s, 1H), 6.82 (s, 1H), 6.71 (d, J=6.7 Hz, 1H), 6.47 (s, 1H), 6.43 (s, 1H), 5.93 (d, J=9.6 Hz, 1H), 5.71 (d, J=1.6 Hz, 1H), 5.59 (d, J=1.6 Hz, 1H), 5.48 (s, 1H), 5.00 (s, 1H), 4.40-4.28 (m, 2H), 3.69 (d, J=9.7 Hz, 1H), 3.60 (s, 3H), 3.54 (s, 1H), 3.50 (s, 3H), 3.12-2.99 (m, 2H), 2.86 (td, J=11.5, 6.7 Hz, 1H), 2.63-2.43 (m, 3H), 2.30 (dd, J=14.2, 6.6 Hz, 1H), 2.25-2.17 (m, 2H), 2.01-1.91 (m, 2H), 1.89-1.82 (m, 1H), 1.74-1.66 (m, J=7.7 Hz, 2H), 1.64-1.57 (m, 2H) ppm. HRMS (ESI) m/z: Calcd for C₃₉H₄₄N₃O₉, [M+H]⁺ 698.3072, found 698.3070.

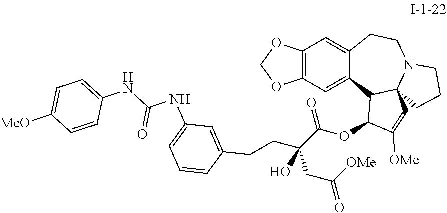

Compound I-1-22 of this title is a pale yellow solid product (90% yield). ¹H NMR (400 MHz, Chloroform-d) δ 7.42-7.38 (m, 2H), 7.23-7.15 (m, 3H), 7.10 (t, J=7.7 Hz, 1H), 7.03 (s, 1H), 6.80-6.75 (m, 2H), 6.72 (d, J=7.5 Hz, 1H), 6.50 (d, J=12.4 Hz, 2H), 5.99 (d, J=9.7 Hz, 1H), 5.80 (s, 1H), 5.69 (s, 1H), 5.04 (s, 1H), 3.76 (s, 1H), 3.72 (s, 3H), 3.69 (s, 1H), 3.64 (s, 3H), 3.54 (s, 3H), 3.16-3.04 (m, 2H), 2.88 (td, J=11.6, 6.9 Hz, 1H), 2.66-2.49 (m, 3H), 2.33 (td, J=6.7 Hz, J=12.0, 1H), 2.30-2.23 (m, 2H), 2.03-1.87 (m, 3H), 1.74 (m, 2H), 1.69-1.61 (m, 2H) ppm. HRMS (ESI) m/z: Calcd for C₃₉H₄₄N₃O₁₀, [M+H]⁺ 714.3021, found 714.3023.

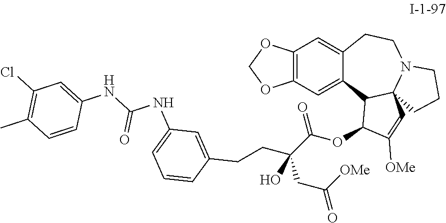

Compound I-1-97 of this title is a pale yellow solid product (88% yield). ¹H NMR (400 MHz, Chloroform-d) δ 7.70 (d, J=13.8 Hz, 2H), 7.37 (d, J=2.2 Hz, 1H), 7.23-7.16 (m, 1H), 7.15-7.08 (m, 2H), 7.07-6.98 (m, 2H), 6.73 (d, J=7.5 Hz, 1H), 6.57 (s, 1H), 6.52 (s, 1H), 6.02 (d, J=9.7 Hz, 1H), 5.82 (d, J=1.5 Hz, 1H), 5.73 (d, J=1.6 Hz, 1H), 5.07 (s, 1H), 3.80 (s, 1H), 3.77 (d, J=9.7 Hz, 1H), 3.65 (s, 3H), 3.55 (s, 3H), 3.18-3.03 (m, 2H), 2.95-2.85 (m, 1H), 2.65-2.50 (m, 3H), 2.39-2.29 (m, 2H), 2.25 (s, 3H), 2.18-2.12 (m, 1H), 2.07-1.87 (m, 3H), 1.80-1.73 (m, 2H), 1.73-1.66 (m, 2H). HRMS (ESI) m/z: Calcd for C₃₉H₄₃ClN₃O₉, [M+H]⁺ 732.2682, found 732.2678.

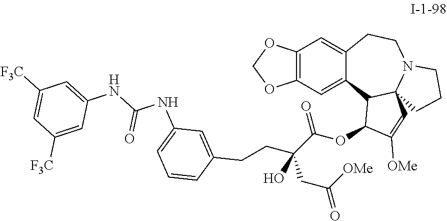

Compound I-1-98 of this title is a pale yellow solid product (84% yield). ¹H NMR (400 MHz, Chloroform-d) δ 8.36 (s, 1H), 8.00 (s, 2H), 7.95 (s, 1H), 7.46 (s, 1H), 7.42 (d, J=7.3 Hz 1H), 7.16 (t, J=7.8 Hz, 1H), 7.03 (s, 1H), 6.73 (d, J=7.6 Hz, 1H), 6.60 (s, 1H), 6.51 (s, 1H), 6.04 (d, J=9.7 Hz, 1H), 5.83 (d, J=1.5 Hz, 1H), 5.75 (d, J=1.5 Hz, 1H), 5.11 (s, 1H), 4.04 (s, 1H), 3.78 (d, J=9.7 Hz, 1H), 3.64 (s, 3H), 3.54 (s, 3H), 3.18-3.05 (m, 2H), 2.93 (td, J=11.4, 6.9 Hz, 1H), 2.63 (ddd, J=16.4, 13.4, 7.8 Hz, 3H), 2.42 (ddt, J=13.9, 10.5, 7.1 Hz, 2H), 2.30 (d, J=16.9 Hz, 1H), 2.10-1.97 (m, 2H), 1.97-1.88 (m, 1H), 1.82-1.70 (m, 4H). HRMS (ESI) m/z: Calcd for C₄₀H₄₀F₆N₃O₉, [M+H]⁺ 820.2663, found 820.2652.

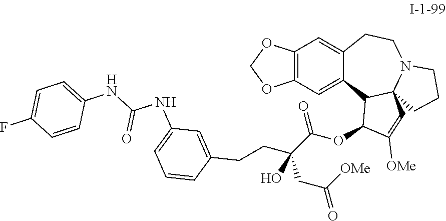

Compound I-1-99 of this title is a pale yellow solid product (84% yield). ¹H NMR (400 MHz, Chloroform-d) δ 7.85-7.62 (m, 2H), 7.32-7.22 (m, 2H), 7.19 (d, J=8.1 Hz, 1H), 7.12 (t, J=7.6 Hz, 1H), 7.05 (s, 1H), 6.90 (t, J=8.0 Hz, 2H), 6.74 (d, J=7.6 Hz, 1H), 6.55 (s, 1H), 6.50 (s, 1H), 6.01 (d, J=9.7 Hz, 1H), 5.82 (d, J=1.6 Hz, 1H), 5.72 (d, J=1.6 Hz, 1H), 5.06 (s, 1H), 3.77 (d, J=9.8 Hz, 1H), 3.66 (s, 3H), 3.59 (s, 1H), 3.55 (s, 3H), 3.19-3.05 (m, 2H), 2.90 (td, J=11.5, 6.8 Hz, 1H), 2.69-2.48 (m, 3H), 2.40-2.28 (m, 2H), 2.27 (d, J=16.7 Hz, 1H), 2.07-1.99 (m, 1H), 1.97 (d, J=16.9 Hz, 1H), 1.95-1.85 (m, 1H), 1.83-1.70 (m, 2H), 1.73-1.64 (m, 2H). HRMS (ESI) m/z: Calcd for C₃₈H₄₁FN₃O₉, [M+H]⁺ 702.2821, found 702.2829.

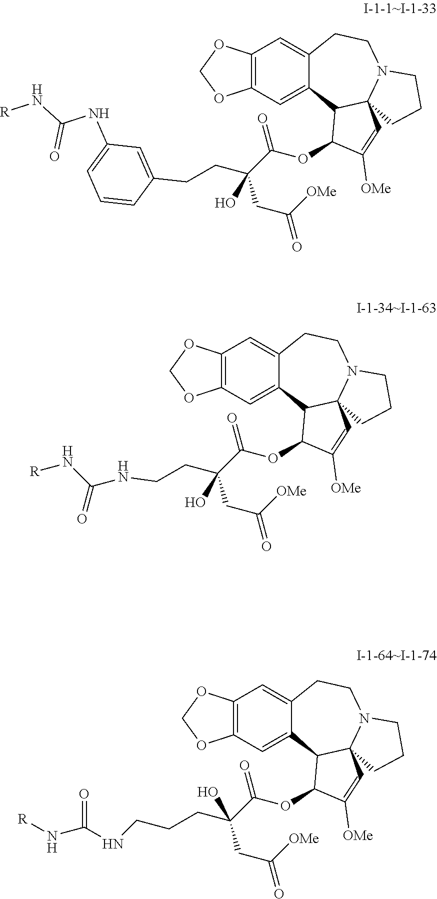

### Example 10: Preparation of Tritiated Base Analogs I-1-3~I-1-36 (Compounds Other than Example 9)

Referring to Example 8, the difference is that compounds I-1-3~I-1-33 were prepared with differently substituted isocyanates, respectively:

| | | | | | [M + H]⁺ | [M + H]⁺ |
|---|---|---|---|---|---|---|
| number | compound | R | yield(%) | formula | Cal. | Found |
| 1 | I-1-3 | H | 80 | C₃₂H₃₇N₃O₉ | 608.2603 | 608.2600 |
| 2 | I-1-4 | n-propyl | 82 | C₃₅H₄₃N₃O₉ | 650.3072 | 650.3075 |
| 3 | I-1-5 | n-pentyl | 89 | C₃₇H₄₇N₃O₉ | 678.3385 | 678.3383 |
| 4 | I-1-6 | n-hexyl | 91 | C₃₈H₄₉N₃O₉ | 692.3542 | 692.3544 |
| 5 | I-1-7 | cyclopropyl | 92 | C₃₅H₄₁N₃O₉ | 648.2916 | 648.2915 |
| 6 | I-1-8 | cyclobutyl | 88 | C₃₆H₄₃N₃O₉ | 662.3072 | 662.3070 |
| 7 | I-1-9 | cyclohexyl | 89 | C₃₈H₄₇N₃O₉ | 690.3385 | 690.3382 |
| 8 | I-1-10 | cycloheptyl | 88 | C₃₉H₄₉N₃O₉ | 704.3542 | 704.3540 |
| 9 | I-1-11 | allyl | 87 | C₃₄H₃₉N₃O₉ | 634.2759 | 634.2761 |
| 10 | I-1-12 | t-butyl | 90 | C₃₆H₄₅N₃O₉ | 664.3229 | 664.3226 |
| 11 | I-1-13 | CCl₃ | 88 | C₃₃H₃₆Cl₃N₃O₉ | 724.1590 | 724.1594 |
| 12 | I-1-14 | TMS | 88 | C₃₅H₄₅N₃O₉Si | 680.2998 | 680.2995 |
| 13 | I-1-15 | 3-chloropropyl | 88 | C₃₅H₄₂ClN₃O₉ | 684.2682 | 684.2684 |
| 14 | I-1-16 | 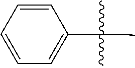 [SMILES: *c1ccccc1] | 90 | C₃₈H₄₁N₃O₉ | 684.2916 | 684.2913 |
| 15 | I-1-17 | 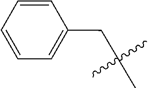 [SMILES: *Cc1ccccc1] | 93 | C₃₉H₄₃N₃O₉ | 698.3072 | 698.3070 |
| 16 | I-1-18 | 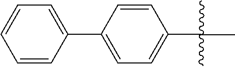 [SMILES: *c1ccc(-c2ccccc2)cc1] | 92 | C₄₄H₄₅N₃O₉ | 760.3229 | 760.3226 |
| 17 | I-1-19 | 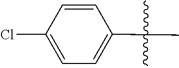 [SMILES: *c1ccc(Cl)cc1] | 94 | C₃₈H₄₀ClN₃O₉ | 718.2526 | 718.2523 |
| 18 | I-1-20 | 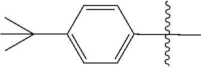 [SMILES: *c1ccc(C(C)(C)C)cc1] | 89 | C₄₂H₄₉N₃O₉ | 740.3542 | 740.3540 |
| 19 | I-1-21 | 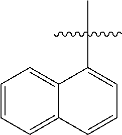 [SMILES: *c1cccc2ccccc12] | 92 | C₄₂H₄₃N₃O₉ | 734.3072 | 734.3072 |
| 20 | I-1-22 | 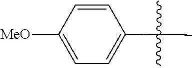 [SMILES: *c1ccc(OC)cc1] | 90 | C₃₉H₄₃N₃O₁₀ | 714.3021 | 714.3023 |
| 21 | I-1-23 | 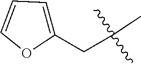 [SMILES: *Cc1ccco1] | 88 | C₃₇H₄₁N₃O₁₀ | 688.2625 | 688.2623 |
| 22 | I-1-24 | 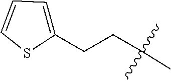 [SMILES: *CCc1cccs1] | 89 | C₃₈H₄₃N₃O₉S | 718.2793 | 718.2790 |
| 23 | I-1-25 | 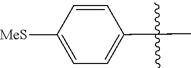 [SMILES: *c1ccc(SC)cc1] | 90 | C₃₉H₄₃N₃O₉S | 730.2793 | 730.2790 |
| 24 | I-1-26 | 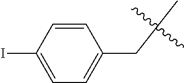 [SMILES: *Cc1ccc(I)cc1] | 91 | C₃₉H₄₂FN₃O₉ | 716.2978 | 716.2978 |
| 25 | I-1-27 | 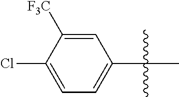 [SMILES: *c1ccc(Cl)c(C(F)(F)F)c1] | 90 | C₃₉H₃₉ClF₃N₃O₉ | 786.2400 | 786.2405 |
| 26 | I-1-28 | 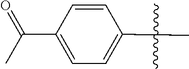 [SMILES: *c1ccc(C(C)=O)cc1] | 91 | C₄₀H₄₃N₃O₁₀ | 726.3021 | 726.3017 |
| 27 | I-1-29 | 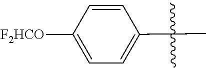 [SMILES: *c1ccc(OC(F)F)cc1] | 92 | C₃₉H₄₁F₂N₃O₁₀ | 750.2833 | 750.2830 |
| 28 | I-1-30 | 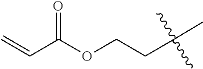 [SMILES: *CCOC(=O)C=C] | 90 | C₃₇H₄₃N₃O₁₁ | 706.2970 | 706.2973 |
| 29 | I-1-31 | 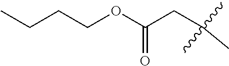 [SMILES: *CC(=O)OCCCC] | 93 | C₃₈H₄₇N₃O₁₁ | 722.3283 | 722.3288 |
| 30 | I-1-32 | 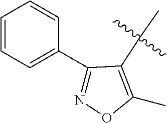 [SMILES: *c1c(-c2ccccc2)noc1C] | 92 | C₄₂H₄₄N₄O₁₀ | 765.3130 | 765.3136 |
| 31 | I-1-33 | 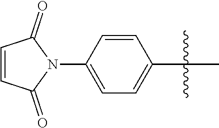 [SMILES: *c1ccc(N2C(=O)C=CC2=O)cc1] | 88 | C₄₂H₄₂N₄O₁₀ | 779.2923 | 779.2920 |

### Example 11: Preparation of Tritiated Base Analogs I-1-34~I-1-63

Referring to Example 8, the difference is that compounds I-1-34~I-1-63 were prepared by reacting different substituted isocyanates with compounds I-2-7, respectively:

| | | | | | [M + H]⁺ | [M + H]⁺ |
|---|---|---|---|---|---|---|
| number | compound | R | yield(%) | formula | Cal. | Found |
| 32 | I-1-34 | H | 85 | C₂₆H₃₃N₃O₉ | 532.2290 | 532.2294 |
| 33 | I-1-35 | n-propyl | 87 | C₂₉H₃₉N₃O₉ | 574.2759 | 574.2756 |
| 34 | I-1-36 | n-pentyl | 90 | C₃₁H₄₃N₃O₉ | 602.3072 | 602.3075 |
| 35 | I-1-37 | n-hexyl | 84 | C₃₂H₄₅N₃O₉ | 616.3229 | 616.3225 |
| 36 | I-1-38 | cyclopropyl | 89 | C₂₉H₃₇N₃O₉ | 572.2603 | 572.2600 |
| 37 | I-1-39 | cyclobutyl | 85 | C₃₀H₃₉N₃O₉ | 586.2725 | 586.2728 |
| 38 | I-1-40 | cyclohexyl | 84 | C₃₂H₄₃N₃O₉ | 614.3072 | 614.3077 |
| 39 | I-1-41 | cycloheptyl | 87 | C₃₃H₄₅N₃O₉ | 628.3229 | 628.3226 |
| 40 | I-1-42 | allyl | 86 | C₂₉H₃₇N₃O₉ | 572.2603 | 572.2609 |
| 41 | I-1-43 | t-butyl | 88 | C₃₀H₄₁N₃O₉ | 588.2916 | 588.2910 |
| 42 | I-1-44 | CCl₃ | 87 | C₂₇H₃₂Cl₃N₃O₉ | 648.1277 | 648.1280 |
| 43 | I-1-45 | TMS | 86 | C₂₉H₄₁N₃O₉Si | 604.2685 | 604.2680 |
| 44 | I-1-46 | 3-chloropropyl | 85 | C₂₉H₃₈ClN₃O₉ | 608.2369 | 608.2372 |
| 45 | I-1-47 | 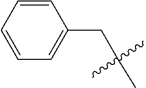 [SMILES: *Cc1ccccc1] | 92 | C₃₃H₃₉N₃O₉ | 622.2759 | 622.2755 |
| 46 | I-1-48 | 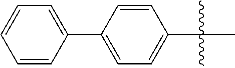 [SMILES: *c1ccc(-c2ccccc2)cc1] | 91 | C₃₈H₄₁N₃O₉ | 684.2916 | 684.2910 |
| 47 | I-1-49 | 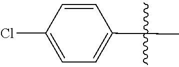 [SMILES: *c1ccc(Cl)cc1] | 93 | C₃₂H₃₆ClN₃O₉ | 642.2213 | 642.2210 |
| 48 | I-1-50 | 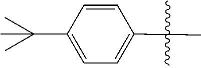 [SMILES: *c1ccc(C(C)(C)C)cc1] | 89 | C₃₆H₄₅N₃O₉ | 664.3229 | 664.3235 |
| 49 | I-1-51 | 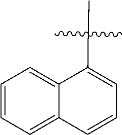 [SMILES: *c1cccc2ccccc12] | 91 | C₃₆H₃₉N₃O₉ | 658.2759 | 658.2756 |
| 50 | I-1-52 | 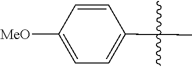 [SMILES: *c1ccc(OC)cc1] | 89 | C₃₃H₃₉N₃O₁₀ | 638.2708 | 638.2700 |
| 51 | I-1-53 | 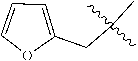 [SMILES: *Cc1ccco1] | 87 | C₃₁H₃₇N₃O₁₀ | 612.2552 | 612.2553 |
| 52 | I-1-54 | 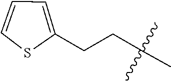 [SMILES: *CCc1cccs1] | 86 | C₃₂H₃₉N₃O₉S | 642.2480 | 642.2485 |
| 53 | I-1-55 | 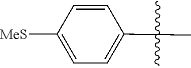 [SMILES: *c1ccc(SC)cc1] | 89 | C₃₃H₃₉N₃O₉S | 654.2480 | 654.2483 |
| 54 | I-1-56 | 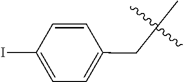 [SMILES: *Cc1ccc(I)cc1] | 91 | C₃₃H₃₉FN₃O₉ | 640.2665 | 640.2660 |
| 55 | I-1-57 | 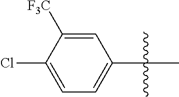 [SMILES: *c1ccc(Cl)c(C(F)(F)F)c1] | 90 | C₃₃H₃₅ClF₃N₃O₉ | 710.2087 | 710.2082 |
| 56 | I-1-58 | 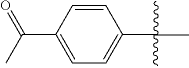 [SMILES: *c1ccc(C(C)=O)cc1] | 93 | C₃₄H₃₉N₃O₁₀ | 650.2708 | 650.2712 |
| 57 | I-1-59 | 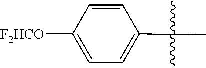 [SMILES: *c1ccc(OC(F)F)cc1] | 91 | C₃₃H₃₇F₂N₃O₁₀ | 674.2520 | 674.2524 |
| 58 | I-1-60 | 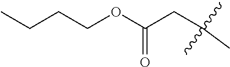 [SMILES: *CC(=O)OCCCC] | 92 | C₃₂H₄₃N₃O₁₁ | 646.2970 | 646.2978 |
| 59 | I-1-61 | 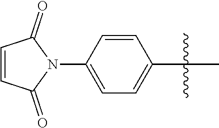 [SMILES: *c1ccc(N2C(=O)C=CC2=O)cc1] | 87 | C₃₆H₃₈N₄O₁₁ | 703.2610 | 703.2618 |
| 60 | I-1-62 | 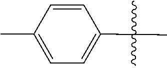 [SMILES: *c1ccc(C)cc1] | 86 | C₃₃H₃₉N₃O₉ | 622.2759 | 622.2750 |
| 61 | I-1-63 | cyclopentyl | 89 | C₃₁H₄₁N₃O₉ | 600.2916 | 600.2920 |

### Example 12: Preparation of Tritiated Base Analogs I-1-64~I-1-72

Referring to Example 8, the difference is that compounds I-1-64~I-1-72 were prepared by reacting different substituted isocyanates with compounds I-2-8, respectively:

| | | | | | [M + H]⁺ | [M + H]⁺ |
|---|---|---|---|---|---|---|
| number | compound | R | yield(%) | formula | Cal. | Found |
| 62 | I-1-64 | H | 85 | C₂₇H₃₅N₃O₉ | 546.2446 | 546.2442 |
| 63 | I-1-65 | n-pentyl | 90 | C₃₂H₄₅N₃O₉ | 616.3229 | 616.3223 |
| 64 | I-1-66 | cyclopentyl | 89 | C₃₂H₄₃N₃O₉ | 614.3072 | 614.3077 |
| 65 | I-1-67 | 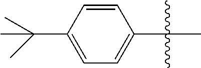 [SMILES: *c1ccc(C(C)(C)C)cc1] | 89 | C₃₇H₄₇N₃O₉ | 678.3385 | 678.3390 |
| 66 | I-1-68 | 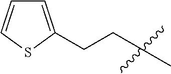 [SMILES: *CCc1cccs1] | 86 | C₃₃H₄₁N₃O₉S | 656.2636 | 656.2642 |
| 67 | I-1-69 | 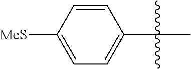 [SMILES: *c1ccc(SC)cc1] | 89 | C₃₄H₄₁N₃O₉S | 668.2636 | 668.2630 |
| 68 | I-1-70 | 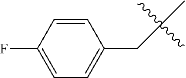 [SMILES: *Cc1ccc(F)cc1] | 91 | C₃₄H₄₀FN₃O₉ | 654.2821 | 654.2827 |
| 69 | I-1-71 | 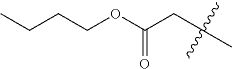 [SMILES: *CC(=O)OCCCC] | 92 | C₃₃H₄₅N₃O₁₁ | 660.3127 | 660.3120 |
| 70 | I-1-72 | 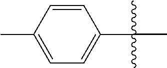 [SMILES: *c1ccc(C)cc1] | 86 | C₃₄H₄₁N₃O₉ | 636.2916 | 636.2924 |

### Example 13: Preparation of Tritiated Base Analog I-1-73

Referring to Example 8, the difference was that benzoyl isocyanate was used instead of p-tolyl isocyanate to react with Compound I-2-5 to prepare Compound I-1-73:

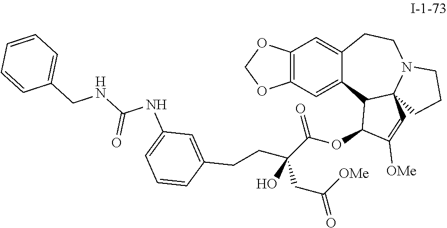

Pale yellow solid product (92% yield) of compound I-1-73 of this title. ¹H NMR (400 MHz, CDCl₃) δ 10.80 (s, 1H), 8.75 (s, 1H), 7.94 (s, 1H), 7.82 (d, J=8.6 Hz, 1H), 7.65 (d, J=7.4 Hz, 1H), 7.54 (t, J=7.6 Hz, 2H), 7.51-7.43 (m, 2H), 6.91 (s, 1H), 6.60 (s, 1H), 6.56 (s, 1H), 6.06 (d, J=9.9 Hz, 1H), 5.84 (d, J=1.4 Hz, 1H), 5.75 (d, J=1.4 Hz, 1H), 5.13 (s, 1H), 3.81 (d, J=9.8 Hz, 1H), 3.73 (s, 3H), 3.64 (s, 1H), 3.59 (s, 3H), 3.27-3.00 (m, 2H), 3.00-2.88 (m, 1H), 2.85-2.47 (m, 3H), 2.46-2.34 (m, 1H), 2.31 (d, J=16.6 Hz, 1H), 2.08-1.97 (m, 2H), 1.94 (d, J=10.2 Hz, 1H), 1.88-1.65 (m, 3H), 1.58 (s, 2H) ppm. HRMS (ESI) m/z: Calcd for C₃₉H₄₂N₃O₁₀, [M+H]⁺ 712.2865, found 712.2865.

### Example 14: Preparation of Tritiated Base Analogs I-1-74~I-1-81

Referring to Example 8, the difference is that different acyl isocyanates were used instead of p-tolyl isocyanate to react with compounds I-2-5, I-2-7, or I-2-8 to prepare compounds I-1-74~I-1-81, respectively:

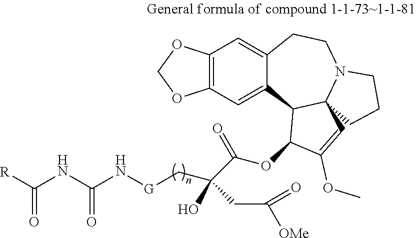

| | | | | | | | [M + H]⁺ | [M + H]⁺ |
|---|---|---|---|---|---|---|---|---|
| number | compound | R | G | | yield(%) | formula | Cal | Found |
| 71 | I-1-74 | n-pentyl | 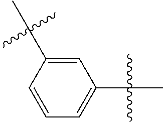 [SMILES: *c1cccc(*)c1] | 2 | 86 | C₃₈H₄₇N₃O₁₀ | 706.3334 | 706.3340 |
| 72 | I-1-75 | cyclopentyl | 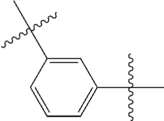 [SMILES: *c1cccc(*)c1] | 2 | 93 | C₃₈H₄₅N₃O₁₀ | 704.3178 | 704.3172 |
| 73 | I-1-76 | 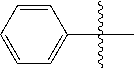 [SMILES: *c1ccccc1] | — | 2 | 92 | C₃₃H₃₇N₃O₁₀ | 636.2552 | 636.2560 |
| 74 | I-1-77 | n-pentyl | — | 2 | 93 | C₃₂H₄₃N₃O₁₀ | 630.3021 | 630.3026 |
| 75 | I-1-78 | cyclopentyl | — | 2 | 90 | C₃₂H₄₁N₃O₁₀ | 628.2865 | 628.2860 |
| 76 | I-1-79 | 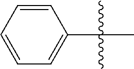 [SMILES: *c1ccccc1] | — | 3 | 93 | C₃₄H₃₉N₃O₁₀ | 650.2708 | 650.2700 |
| 77 | I-1-80 | n-pentyl | — | 3 | 91 | C₃₃H₄₅N₃O₁₀ | 644.3178 | 644.3170 |
| 78 | I-1-81 | cyclopentyl | | 3 | 92 | C₃₃H₄₃N₃O₁₀ | 642.3021 | 642.3026 |

### Example 15: Preparation of Tritiated Base Analog I-1-82

Referring to Example 8, the difference is that phenylsulfonyl isocyanate was used instead of p-tolyl isocyanate to prepare Compound I-1-82:

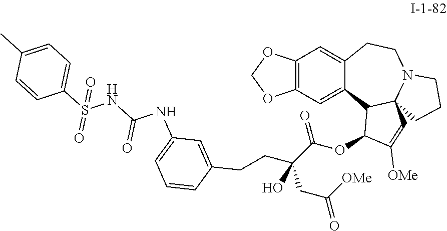

Pale yellow solid product (83% yield) of compound I-1-82 of this title. ¹H NMR (400 MHz, CDCl₃) δ 7.79 (d, J=7.1 Hz, 2H), 7.25-7.11 (m, 3H), 7.10-7.00 (m, 1H), 6.70 (d, J=5.8 Hz, 1H), 6.55 (s, 1H), 6.47 (s, 1H), 6.00 (d, J=9.4 Hz, 1H), 5.80 (d, J=1.4 Hz, 1H), 5.68 (d, J=1.4 Hz, 1H), 5.08 (s, 1H), 3.82 (d, J=9.1 Hz, 1H), 3.60 (s, 3H), 3.54 (s, 3H), 3.41-3.27 (m, 1H), 3.27-3.12 (m, 2H), 3.12-3.00 (m, 1H), 2.61-2.39 (m, 2H), 2.36 (s, 3H), 2.31-2.13 (m, 3H), 2.09-1.89 (m, 3H), 1.89-1.72 (m, 2H), 1.72-1.54 (m, 2H) ppm. HRMS (ESI) m/z: Calcd for C₃₉H₄₄N₃O₁₁S, [M+H]⁺ 762.2691, found 762.2691.

### Example 16: Preparation of Tritiated Base Analogs I-1-83~I-1-96

Referring to Example 8, the difference is that compounds I-1-83~I-1-% were prepared by reacting differently substituted nitrile amines, acyl nitrile amines or differently substituted carbodiimides with compounds I-2-5, I-2-7 or I-2-8, respectively:

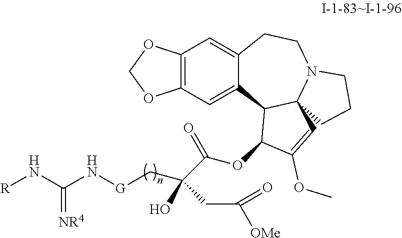

| | | | | | | | | [M + H]⁺ | [M + H]⁺ |
|---|---|---|---|---|---|---|---|---|---|
| number | compound | R | G | n | R⁴ | yield(%) | formula | Cal. | Found |
| 79 | I-1-83 | n-pentyl | 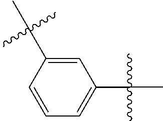 [SMILES: *c1cccc(*)c1] | 2 | H | 88 | C₃₇H₄₈N₄O₈ | 677.3545 | 677.3550 |
| 80 | I-1-84 | cyclopentyl | 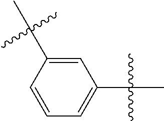 [SMILES: *c1cccc(*)c1] | 2 | H | 90 | C₃₇H₄₆N₄O₈ | 675.3388 | 675.3392 |
| 81 | I-1-85 | 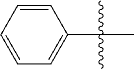 [SMILES: *c1ccccc1] | 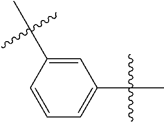 [SMILES: *c1cccc(*)c1] | 2 | H | 93 | C₃₈H₄₂N₄O₈ | 683.3075 | 683.3080 |
| 82 | I-1-86 | i-Pr | 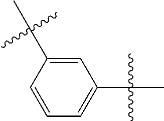 [SMILES: *c1cccc(*)c1] | 2 | i-Pr | 92 | C₃₈H₅₀N₄O₈ | 691.3701 | 691.3708 |
| 83 | I-1-87 | i-Pr | — | 2 | i-Pr | 91 | C₃₂H₄₆N₄O₈ | 615.3388 | 615.3384 |
| 84 | I-1-88 | 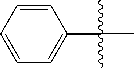 [SMILES: *c1ccccc1] | — | 2 | H | 90 | C₃₂H₃₈N₄O₈ | 607.2762 | 607.2768 |
| 85 | I-1-89 | n-pentyl | — | 2 | H | 89 | C₃₁H₄₄N₄O₈ | 601.3232 | 601.3238 |
| 86 | I-1-90 | cyclopentyl | — | 2 | H | 88 | C₃₁H₄₂N₄O₈ | 599.3075 | 599.3080 |
| 87 | I-1-91 | 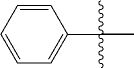 [SMILES: *c1ccccc1] | — | 3 | H | 90 | C₃₃H₄₀N₄O₈ | 621.2919 | 621.2927 |
| 88 | I-1-92 | n-penty1 | — | 3 | H | 87 | C₃₂H₄₆N₄O₈ | 615.3388 | 615.3382 |
| 89 | I-1-93 | cyclopentyl | | 3 | H | 87 | C₃₂H₄₄N₄O₈ | 613.3232 | 613.3236 |
| 90 | I-1-94 | n-pentanoyl | 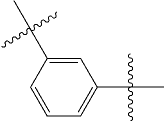 [SMILES: *c1cccc(*)c1] | 2 | H | 89 | C₃₈H₄₈N₄O₉ | 705.3494 | 705.3499 |
| 91 | I-1-95 | cyclopentyloxy | 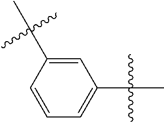 [SMILES: *c1cccc(*)c1] | 2 | H | 90 | C₃₈H₄₆N₄O₉ | 703.3338 | 703.3332 |
| 92 | I-1-96 | benzoyl | 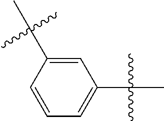 [SMILES: *c1cccc(*)c1] | 2 | H | 91 | C₃₉H₄₂N₄O₉ | 711.3025 | 711.3032 |

### Example 17: Preparation of β-cyclolactone tritiated alkaloids 23-40

Referring to Example 1 Step 1.6, the difference is that compounds 23-40 were prepared by reacting tricuspid alpha-keto ester bases with different substitutions in the side chain with trimethylsilyl enones, respectively:

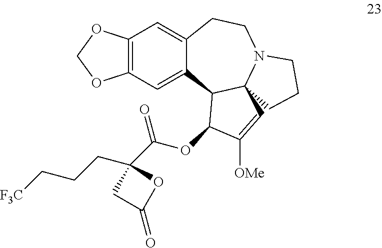 [SMILES: COC1=C[C@]23CCCN2CCc2cc4c(cc2[C@@H]3[C@@H]1OC(=O)[C@@]1(CCCC(F)(F)F)CC(=O)O1)OCO4]

Compound 23: light yellow solid, 23% yield. [α]_{D}²⁰=−97.9 (c 0.45, CHCl₃). ¹H NMR (400 MHz, CDCl₃) δ 6.60 (d, J=2.6 Hz, 2H), 5.92 (d, J=9.6 Hz, 1H), 5.86 (dd, J=7.3, 1.5 Hz, 2H), 5.09 (s, 1H), 3.82 (d, J=9.5 Hz, 1H), 3.69 (s, 3H), 3.13-3.03 (m, 2H), 3.00 (d, J=16.5 Hz, 1H), 2.95-2.89 (m, 1H), 2.74 (d, J=16.5 Hz, 1H), 2.62-2.57 (m, 2H), 2.35 (dd, J=14.2, 6.8 Hz, 1H), 2.09-2.00 (m, 3H), 1.91 (ddd, J=12.1, 7.6, 4.2 Hz, 2H), 1.80-1.71 (m, 3H), 1.56-1.47 (m, 2H). HRMS (ESI) m/z: Calcd for C₂₆H₂₉F₃NO₇, [M+H]⁺ 524.1891, found 524.1889.

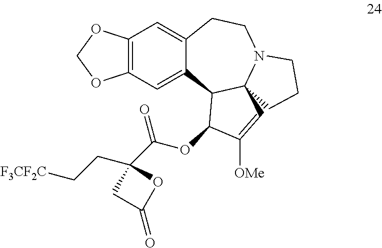

Compound 24: light yellow solid product (30% yield). [α]_{D}²⁰=−64.2 (c 0.7, CHCl₃). ¹H NMR (400 MHz, CDCl₃) δ 6.60 (d, J=4.2 Hz, 2H), 5.90 (d, J=9.5 Hz, 1H), 5.87 (dd, J=10.3, 1.5 Hz, 2H), 5.11 (s, 1H), 3.82 (d, J=9.4 Hz, 1H), 3.68 (s, 2H), 3.11-3.03 (m, 2H), 3.04 (d, J=16.4 Hz, 1H), 2.93 (td, J=11.9, 11.4, 6.8 Hz, 1H), 2.80 (d, J=16.4 Hz, 1H), 2.62-2.56 (m, 2H), 2.36 (dd, J=14.0, 6.5 Hz, 1H), 2.28-2.20 (m, 1H), 2.04 (ddd, J=12.6, 10.8, 7.0 Hz, 3H), 1.97-1.88 (m, 3H), 1.75-1.72 (m, 2H). HRMS (ESI) m/z: Calcd for C₂₆H₂₇F₅NO₇, [M+H]⁺ 560.1702, found 560.1700.

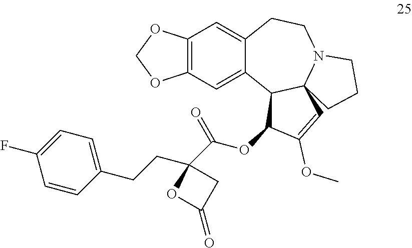 [SMILES: COC1=C[C@]23CCCN2CCc2cc4c(cc2[C@@H]3[C@@H]1OC(=O)[C@@]1(CCc2ccc(F)cc2)CC(=O)O1)OCO4]

Compound 25: light yellow amorphous solid in 50% yield. ¹H NMR (400 MHz, CDCl₃) δ 7.09 (dd, J=7.9, 5.7 Hz, 2H), 6.98 (t, J=8.6 Hz, 2H), 6.63 (s, 1H), 6.59 (s, 1H), 5.95 (d, J=9.5 Hz, 1H), 5.81 (s, 1H), 5.61 (s, 1H), 5.14 (s, 1H), 3.86 (d, J=9.5 Hz, 1H), 3.74 (s, 3H), 3.17-3.06 (m, 2H), 3.01 (d, J=16.5 Hz, 1H), 2.94 (td, J=11.7, 7.1 Hz, 1H), 2.83 (d, J=16.5 Hz, 1H), 2.64-2.56 (m, 2H), 2.48 (td, J=12.6, 4.7 Hz, 1H), 2.37 (dd, J=14.3, 6.9 Hz, 1H), 2.32-2.26 (m, 1H), 2.25-2.17 (m, 1H), 2.10-2.03 (m, 1H), 1.97-1.88 (m, 2H), 1.81-1.74 (m, 2H) ppm. HRMS (ESI) m/z: Calcd for C₃₀H₃₁FNO₇, [M+H]⁺ 536.2082, found 536.2085.

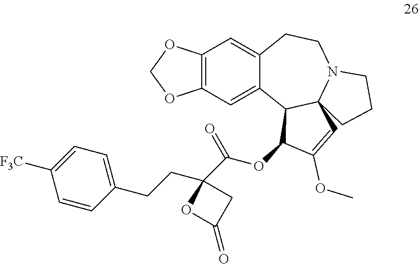 [SMILES: COC1=C[C@]23CCCN2CCc2cc4c(cc2[C@@H]3[C@@H]1OC(=O)[C@@]1(CCc2ccc(C(F)(F)F)cc2)CC(=O)O1)OCO4]

Compound 26: light yellow amorphous solid, 80% yield. ¹H NMR (400 MHz, CDCl₃) δ 7.54 (d, J=8.0 Hz, 2H), 7.24 (d, J=8.0 Hz, 2H), 6.62 (s, 1H), 6.58 (s, 1H), 5.94 (dd, J=9.5, 0.9 Hz, 1H), 5.78 (d, J=1.5 Hz, 1H), 5.57 (d, J=1.5 Hz, 1H), 5.13 (d, J=0.9 Hz, 1H), 3.85 (d, J=9.5 Hz, 1H), 3.73 (s, 3H), 3.10 (ddt, J=13.3, 8.9, 6.7 Hz, 2H), 3.02 (d, J=16.5 Hz, 1H), 2.93 (td, J=11.6, 6.9 Hz, 1H), 2.86 (d, J=16.5 Hz, 1H), 2.67-2.47 (m, 3H), 2.42-2.28 (m, 2H), 2.22 (ddd, J=14.1, 12.1, 4.7 Hz, 1H), 2.05 (dt, J=12.3, 9.6 Hz, 1H), 1.98-1.86 (m, 2H), 1.83-1.70 (m, 2H) ppm. HRMS (ESI) m/z: Calcd for C₃₁H₃₁F₃NO₇, [M+H]⁺ 586.2047, found 586.2053.

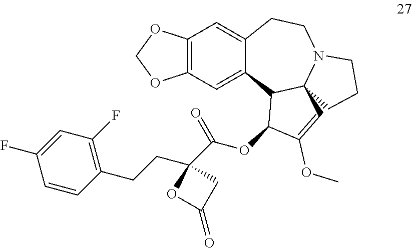

Compound 27: light yellow amorphous solid, 97% yield. ¹H NMR (400 MHz, CDCl₃) δ 7.07 (q, J=8.1 Hz, 1H), 6.84-6.72 (m, 2H), 6.61 (s, 1H), 6.56 (s, 1H), 5.93 (d, J=9.5 Hz, 1H), 5.80 (s, 1H), 5.64 (s, 1H), 5.11 (s, 1H), 3.83 (d, J=9.6 Hz, 1H), 3.71 (s, 3H), 3.16-3.04 (m, 2H), 3.01 (d, J=16.5 Hz, 1H), 2.92 (td, J=11.5, 7.0 Hz, 1H), 2.81 (d, J=16.6 Hz, 1H), 2.63-2.55 (m, 2H), 2.49 (dd, J=12.5, 4.7 Hz, 1H), 2.40-2.30 (m, 2H), 2.17 (td, J=13.1, 12.4, 4.9 Hz, 1H), 2.08-2.01 (m, 1H), 1.95-1.85 (m, 2H), 1.79-1.72 (m, 2H) ppm. HRMS (ESI) m/z: Calcd for C₃₀H₃₀F₂NO₇, [M+H]⁺ 554.1987, found 554.1990.

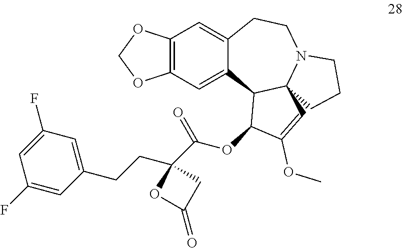 [SMILES: COC1=C[C@]23CCCN2CCc2cc4c(cc2[C@@H]3[C@@H]1OC(=O)[C@@]1(CCc2cc(F)cc(F)c2)CC(=O)O1)OCO4]

Compound 28: light yellow amorphous solid, 50% yield. ¹H NMR (400 MHz, CDCl₃) δ 6.71-6.56 (m, 5H), 5.93 (d, J=9.5 Hz, 1H), 5.81 (d, J=1.6 Hz, 1H), 5.69 (d, J=1.5 Hz, 1H), 5.13 (s, 1H), 3.85 (d, J=9.5 Hz, 1H), 3.74 (s, 3H), 3.12-3.05 (m, 2H), 2.99 (d, J=16.5 Hz, 1H), 2.92 (td, J=11.9, 7.2 Hz, 1H), 2.78 (d, J=16.5 Hz, 1H), 2.63-2.55 (m, 2H), 2.49 (td, J=12.8, 12.3, 5.0 Hz, 1H), 2.38-2.14 (m, 3H), 2.06-2.01 (m, 1H), 1.96-1.88 (m, 2H), 1.80-1.73 (m, 2H) ppm. HRMS (ESI) m/z: Calcd for C₃₀H₃₀F₂NO₇, [M+H]⁺ 554.1979, found 554.1990.

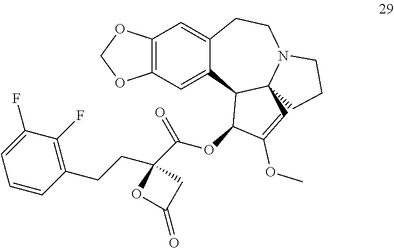 [SMILES: COC1=C[C@]23CCCN2CCc2cc4c(cc2[C@@H]3[C@@H]1OC(=O)[C@@]1(CCc2cccc(F)c2F)CC(=O)O1)OCO4]

Compound 29: light yellow amorphous solid, 95% yield. ¹H NMR (400 MHz, CDCl₃) δ 6.98-6.92 (m, 1H), 6.91-6.82 (m, 2H), 6.62 (s, 1H), 6.56 (s, 1H), 5.95 (d, J=9.5 Hz, 1H), 5.81 (d, J=1.5 Hz, 1H), 5.68 (d, J=1.5 Hz, 1H), 5.12 (s, 1H), 3.88-3.81 (m, 1H), 3.73 (s, 3H), 3.15-3.05 (m, 2H), 3.04-2.92 (m, 2H), 2.79 (d, J=16.5 Hz, 1H), 2.63-2.55 (m, 3H), 2.40-2.30 (m, 2H), 2.23-2.12 (m, 1H), 2.10-2.00 (m, 1H), 1.98-1.86 (m, 2H), 1.81-1.72 (m, 2H) ppm. HRMS (ESI) m/z: Calcd for C₃₀H₃₀F₂NO₇, [M+H]⁺ 554.1983, found 554.1990.

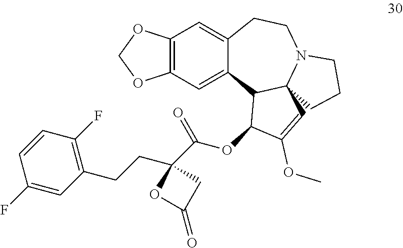

Compound 30: light yellow amorphous solid, 97% yield. ¹H NMR (400 MHz, CDCl₃) δ 7.05-6.96 (m, 2H), 6.87 (t, J=7.1 Hz, 1H), 6.61 (s, 1H), 6.56 (s, 1H), 5.93 (d, J=9.5 Hz, 1H), 5.80 (s, 1H), 5.66 (s, 1H), 5.11 (s, 1H), 3.84 (d, J=9.5 Hz, 1H), 3.72 (s, 3H), 3.15-3.06 (m, 2H), 3.01 (d, J=16.5 Hz, 1H), 2.92 (td, J=11.8, 7.0 Hz, 1H), 2.79 (d, J=16.5 Hz, 1H), 2.66-2.52 (m, 3H), 2.46 (dt, J=18.0, 9.1 Hz, 1H), 2.35 (dd, J=14.2, 6.8 Hz, 1H), 2.26-2.19 (m, 1H), 2.07-1.99 (m, 1H), 1.95-1.86 (m, 2H), 1.81-1.69 (m, 2H) ppm. HRMS (ESI) m/z: Calcd for C₃₀H₃₀F₂NO₇, [M+H]⁺ 554.1983, found 554.1990.

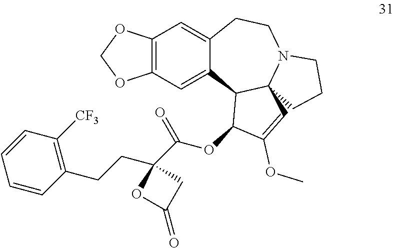 [SMILES: COC1=C[C@]23CCCN2CCc2cc4c(cc2[C@@H]3[C@@H]1OC(=O)[C@@]1(CCc2ccccc2C(F)(F)F)CC(=O)O1)OCO4]

Compound 31: light yellow amorphous solid, 89% yield. ¹H NMR (400 MHz, CDCl₃) δ 7.61 (dd, J=7.9, 1.4 Hz, 1H), 7.48 (dd, J=8.3, 6.9 Hz, 1H), 7.32 (t, J=7.7 Hz, 1H), 7.26 (d, J=7.6 Hz, 1H), 6.61 (s, 1H), 6.54 (s, 1H), 5.97 (dd, J=9.5, 1.0 Hz, 1H), 5.78 (d, J=1.5 Hz, 1H), 5.57 (d, J=1.5 Hz, 1H), 5.11 (d, J=1.0 Hz, 1H), 3.84 (d, J=9.6 Hz, 1H), 3.72 (s, 3H), 3.16-3.06 (m, 2H), 3.02 (d, J=16.5 Hz, 1H), 2.92 (td, J=11.7, 7.0 Hz, 1H), 2.80 (d, J=16.5 Hz, 1H), 2.70 (td, J=13.0, 4.6 Hz, 1H), 2.63-2.54 (m, 3H), 2.34 (dd, J=14.3, 6.9 Hz, 1H), 2.23-2.15 (m, 1H), 2.07-2.02 (m, 1H), 1.94-1.88 (m, 2H), 1.79-1.73 (m, 2H) ppm. HRMS (ESI) m/z: Calcd for C₃₁H₃₁F₃NO₇, [M+H]⁺ 586.2045, found 586.2053.

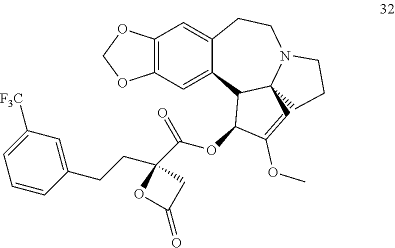

Compound 32: light yellow amorphous solid, 80% yield. ¹H NMR (400 MHz, CDCl₃) δ 7.48 (d, J=7.7 Hz, 1H), 7.45-7.36 (m, 2H), 7.31 (d, J=7.6 Hz, 1H), 6.62 (s, 1H), 6.57 (s, 1H), 5.96 (d, J=9.5 Hz, 1H), 5.79 (d, J=1.5 Hz, 1H), 5.61 (d, J=1.5 Hz, 1H), 5.13 (s, 1H), 3.85 (d, J=9.5 Hz, 1H), 3.73 (s, 3H), 3.18-3.04 (m, 2H), 2.98 (d, J=16.5 Hz, 1H), 2.93 (td, J=11.5, 6.9 Hz, 1H), 2.81 (d, J=16.5 Hz, 1H), 2.69-2.49 (m, 3H), 2.42-2.30 (m, 1H), 2.23 (td, J=12.9, 12.2, 4.7 Hz, 1H), 2.11-1.99 (m, 1H), 1.96-1.87 (m, 1H), 1.79-1.72 (m, 2H), 1.71-1.62 (m, 2H) ppm. HRMS (ESI) m/z: Calcd for C₃₁H₃₁F₃NO₇, [M+H]⁺ 586.2044, found 586.2053.

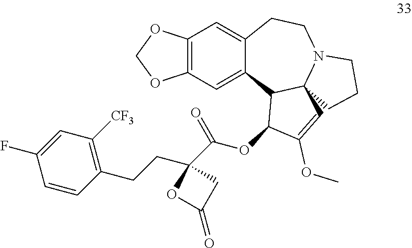

Compound 33: light yellow amorphous solid, 56% yield. ¹H NMR (400 MHz, CDCl₃) δ 7.32 (dd, J=9.1, 2.7 Hz, 1H), 7.26-7.22 (m, 1H), 7.18 (td, J=8.1, 2.6 Hz, 1H), 6.61 (s, 1H), 6.55 (s, 1H), 5.95 (d, J=9.5 Hz, 1H), 5.79 (d, J=1.5 Hz, 1H), 5.59 (d, J=1.6 Hz, 1H), 5.11 (s, 1H), 3.84 (d, J=9.5 Hz, 1H), 3.72 (s, 3H), 3.17-3.03 (m, 2H), 3.01 (d, J=16.6 Hz, 1H), 2.92 (td, J=11.6, 7.0 Hz, 1H), 2.80 (d, J=16.5 Hz, 1H), 2.72-2.44 (m, 4H), 2.34 (dd, J=14.3, 6.9 Hz, 1H), 2.18 (ddd, J=14.3, 12.1, 4.8 Hz, 1H), 2.08-1.98 (m, 1H), 1.97-1.79 (m, 2H), 1.82-1.70 (m, 2H) ppm. HRMS (ESI) m/z: Calcd for C₃₁H₃₀F₄NO₇, [M+H]⁺ 604.1952, found 604.1958.

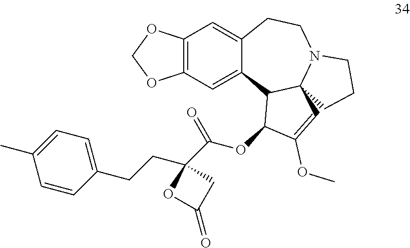

Compound 34: light yellow amorphous solid, 63% yield. ¹H NMR (400 MHz, CDCl₃) δ 7.54 (d, J=8.0 Hz, 2H), 7.24 (d, J=8.0 Hz, 2H), 6.62 (s, 1H), 6.58 (s, 1H), 5.94 (dd, J=9.5, 0.9 Hz, 1H), 5.78 (d, J=1.5 Hz, 1H), 5.57 (d, J=1.5 Hz, 1H), 5.13 (d, J=0.9 Hz, 1H), 3.85 (d, J=9.5 Hz, 1H), 3.73 (s, 3H), 3.10 (ddt, J=13.3, 8.9, 6.7 Hz, 2H), 3.02 (d, J=16.5 Hz, 1H), 2.93 (td, J=11.6, 6.9 Hz, 1H), 2.86 (d, J=16.5 Hz, 1H), 2.67-2.47 (m, 3H), 2.42-2.28 (m, 2H), 2.22 (ddd, J=14.1, 12.1, 4.7 Hz, 1H), 2.05 (dt, J=12.3, 9.6 Hz, 1H), 1.98-1.86 (m, 2H), 1.83-1.70 (m, 2H) ppm. HRMS (ESI) m/z: Calcd for C₃₁H₃₄NO₇, [M+H]⁺ 532.2331, found 532.2335.

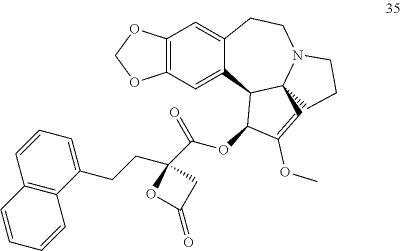 [SMILES: COC1=C[C@]23CCCN2CCc2cc4c(cc2[C@@H]3[C@@H]1OC(=O)[C@@]1(CCc2cccc3ccccc23)CC(=O)O1)OCO4]

Compound 35: light yellow amorphous solid, 89% yield. ¹H NMR (400 MHz, CDCl₃) δ 7.98-7.91 (m, 1H), 7.86 (dd, J=7.1, 2.2 Hz, 1H), 7.74 (d, J=8.2 Hz, 1H), 7.56-7.44 (m, 2H), 7.39 (dd, J=8.2, 7.0 Hz, 1H), 7.29-7.25 (m, 1H), 6.64 (s, 1H), 6.50 (s, 1H), 6.04 (dd, J=9.5, 0.9 Hz, 1H), 5.72 (d, J=1.5 Hz, 1H), 5.42 (d, J=1.5 Hz, 1H), 5.16 (s, 1H), 3.88 (d, J=9.6 Hz, 1H), 3.73 (s, 3H), 3.27-3.13 (m, 1H), 3.15-3.05 (m, 1H), 3.01-2.82 (m, 4H), 2.80 (d, J=16.5 Hz, 1H), 2.69-2.56 (m, 2H), 2.46-2.29 (m, 2H), 2.10-2.00 (m, 1H), 2.00-1.90 (m, 1H), 1.83-1.73 (m, 2H), 1.72-1.67 (m, 1H) ppm. HRMS (ESI) m/z: Calcd for C₃₄H₃₄NO₇, [M+H]⁺ 568.2326, found 568.2335.

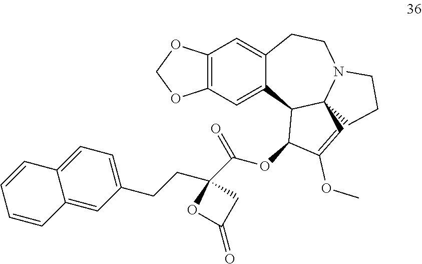 [SMILES: COC1=C[C@]23CCCN2CCc2cc4c(cc2[C@@H]3[C@@H]1OC(=O)[C@@]1(CCc2ccc3ccccc3c2)CC(=O)O1)OCO4]

Compound 36: light yellow amorphous solid, 85% yield. ¹H NMR (400 MHz, CDCl₃) δ 7.84-7.73 (m, 3H), 7.56 (s, 1H), 7.48-7.41 (m, 2H), 7.25 (d, J=8.5 Hz, 1H), 6.63 (s, 1H), 6.58 (s, 1H), 5.98 (d, J=9.5 Hz, 1H), 5.74 (s, 1H), 5.51 (s, 1H), 5.13 (s, 1H), 3.86 (d, J=9.5 Hz, 1H), 3.75 (s, 3H), 3.21-3.04 (m, 2H), 3.01 (d, J=16.5 Hz, 1H), 2.98-2.89 (m, 1H), 2.86 (d, J=16.5 Hz, 1H), 2.68-2.54 (m, 3H), 2.45 (dt, J=13.0, 6.4 Hz, 1H), 2.41-2.26 (m, 2H), 2.09-1.97 (m, 2H), 1.98-1.87 (m, 1H), 1.83-1.71 (m, 2H) ppm. HRMS (ESI) m/z: Calcd for C₃₄H₃₄NO₇, [M+H]⁺ 568.2321, found 568.2335.

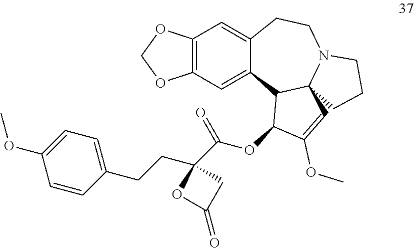

Compound 37: light yellow amorphous solid, 89% yield. ¹H NMR (400 MHz, CDCl₃) δ 7.03 (d, J=8.2 Hz, 2H), 6.82 (d, J=8.1 Hz, 2H), 6.61 (s, 1H), 6.58 (s, 1H), 5.94 (d, J=9.5 Hz, 1H), 5.80 (d, J=1.6 Hz, 1H), 5.61 (d, J=1.6 Hz, 1H), 5.12 (s, 1H), 3.84 (d, J=9.5 Hz, 1H), 3.78 (s, 3H), 3.73 (s, 3H), 3.18-3.03 (m, 2H), 2.96 (d, J=16.7 Hz, 1H), 2.97-2.87 (m, 1H), 2.78 (d, J=16.5 Hz, 1H), 2.66-2.54 (m, 2H), 2.45 (td, J=12.8, 4.8 Hz, 1H), 2.35 (dd, J=14.2, 6.9 Hz, 1H), 2.26 (td, J=13.1, 12.6, 4.6 Hz, 1H), 2.16 (ddd, J=16.5, 11.7, 4.7 Hz, 1H), 2.05 (dt, J=12.8, 9.6 Hz, 1H), 1.95-1.86 (m, 2H), 1.86-1.70 (m, 2H) ppm. HRMS (ESI) m/z: Calcd for C₃₁H₃₄NO₈, [M+H]⁺ 548.2276, found 548.2284.

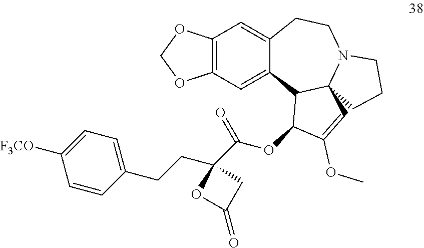

Compound 38: light yellow amorphous solid, 69% yield ¹H NMR (400 MHz, CDCl₃) ¹H NMR (400 MHz, CDCl3) δ 7.11 (s, 4H), 6.63 (s, 1H), 6.59 (s, 1H), 5.89 (dd, J=37.7, 9.1 Hz, 1H), 5.74 (d, J=10.2 Hz, 1H), 5.50 (d, 1H), 5.13 (d, J=14.4 Hz, 1H), 3.88 (d, J=9.4 Hz, 1H), 3.76 (s, 3H), 3.18 (ddt, J=13.5, 8.9, 6.5 Hz, 2H), 3.08 (d, J=16.5 Hz, 1H), 2.97 (td, J=11.4, 6.7 Hz, 1H), 2.72 (d, J=16.5 Hz, 1H), 2.50-2.27 (m, 3H), 2.24-2.16 (m, 2H), 2.00 (ddd, J=14.2, 12.2, 4.8 Hz, 1H), 1.98 (dt J=12.4, 9.7 Hz, 1H), 1.98-1.87 (m, 2H), 1.82-1.67 (m, 2H) ppm. HRMS (ESI) m/z: Calcd for C₃₁H₃₁F₃NO₈, [M+H]⁺ 602.2002, found 602.1993.

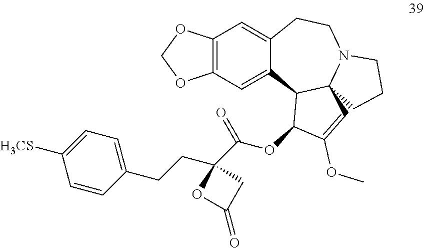

Compound 39: light yellow amorphous solid, 89% yield. ¹H NMR (400 MHz, CDCl₃) δ 7.18 (d, J=8.2 Hz, 2H), 7.04 (d, J=8.3 Hz, 2H), 6.61 (s, 1H), 6.58 (s, 1H), 5.94 (dd, J=9.5, 0.9 Hz, 1H), 5.80 (d, J=1.6 Hz, 1H), 5.59 (d, J=1.5 Hz, 1H), 5.11 (d, J=0.9 Hz, 1H), 3.84 (d, J=9.5 Hz, 1H), 3.72 (s, 3H), 3.17-3.05 (m, 2H), 2.98 (d, J=16.5 Hz, 1H), 2.92 (dt, J=12.0, 5.8 Hz, 1H), 2.83 (d, J=16.5 Hz, 1H), 2.63-2.55 (m, 2H), 2.49-2.40 (m, 4H), 2.35 (dd, J=14.2, 6.9 Hz, 1H), 2.30-2.13 (m, 2H), 2.09-2.01 (m, 1H), 1.95-1.85 (m, 2H), 1.80-1.70 (m, 2H) ppm. HRMS (ESI) m/z: Calcd for C₃₁H₃₄NO₇S, [M+H]⁺ 564.2056, found 564.2038.

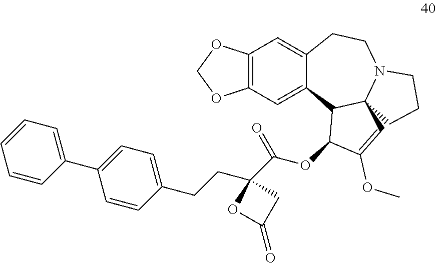

Compound 40: light yellow amorphous solid, 87% yield. ¹H NMR (400 MHz, CDCl₃) δ 7.58 (d, J=8.2 Hz, 2H), 7.52 (d, J=8.0 Hz, 2H), 7.44 (t, J=7.5 Hz, 2H), 7.39-7.30 (m, 1H), 7.20 (d, J=8.0 Hz, 2H), 6.63 (s, 1H), 6.59 (s, 1H), 5.97 (d, J=9.5 Hz, 1H), 5.80 (s, 1H), 5.60 (s, 1H), 5.12 (s, 1H), 3.86 (d, J=9.5 Hz, 1H), 3.74 (s, 3H), 3.18-3.06 (m, 2H), 3.02 (d, J=16.6 Hz, 1H), 2.94 (td, J=11.7, 7.9 Hz, 1H), 2.84 (d, J=16.5 Hz, 1H), 2.66-2.48 (m, 3H), 2.40-2.31 (m, 1H), 2.30-2.18 (m, 1H), 2.12-1.97 (m, 2H), 1.99-1.88 (m, 2H), 1.83-1.71 (m, 2H) ppm. HRMS (ESI) m/z: Calcd for C₃₆H₃₆NO₇, [M+H]⁺ 594.2478, found 594.2492.

### Example 18: Preparation of Tritiated Alkaloids I-2-29~I-2-46

Referring to Example 1 Step 1.7, the difference is that the ring opening methyl esterification reaction with Compounds 23-40 allows the preparation of Compounds I-2-29~I-2-46, respectively:

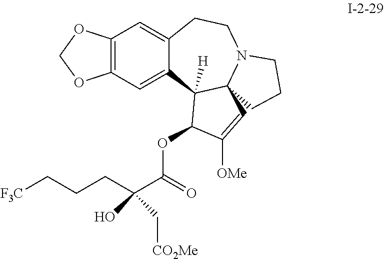

Compound I-2-29: light yellow solid product (67% yield). [α]_{D}²⁰=−72.1 (c 1, CHCl₃). ¹H NMR (400 MHz, CDCl₃) δ 6.62 (s, 1H), 6.53 (s, 1H), 6.01 (dd, J=9.8, 1.0 Hz, 1H), 5.86 (dd, J=5.9, 1.5 Hz, 2H), 5.05 (s, 1H), 3.77 (d, J=9.8 Hz, 1H), 3.72 (s, 1H), 3.66 (s, 3H), 3.57 (s, 3H), 3.14-3.04 (m, 2H), 2.94 (td, J=11.4, 6.8 Hz, 1H), 2.62-2.56 (m, 3H), 2.37 (dd, J=14.0, 6.7 Hz, 1H), 2.24 (d, J=16.5 Hz, 1H), 2.05-2.02 (m, 2H), 1.95-1.86 (m, 2H), 1.87 (d, J=16.5 Hz, 1H), 1.81-1.70 (m, 3H), 1.53-1.49 (m, 2H). HRMS (ESI) m/z: Calcd for C₂₇H₃₃F₃NO₈, [M+H]⁺ 556.2153, found 556.2145.

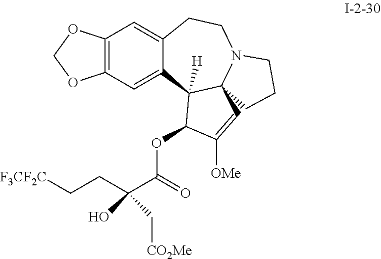

Compound I-2-30: light yellow solid product (85% yield). [α]_{D}²⁰=−80.2 (c 1.1, CHCl₃). ¹H NMR (400 MHz, CDCl₃) δ 6.64 (s, 1H), 6.53 (s, 1H), 6.03 (d, J=9.8 Hz, 1H), 5.87 (d, J=5.5 Hz, 2H), 5.07 (s, 1H), 3.78 (d, J=9.9 Hz, 1H), 3.70 (s, 1H), 3.63 (s, 3H), 3.58 (s, 3H), 3.13-3.03 (m, 2H), 2.95 (td, J=11.4, 6.6 Hz, 1H), 2.63-2.56 (m, 2H), 2.39 (dd, J=13.9, 6.5 Hz, 1H), 2.29 (d, J=16.6 Hz, 1H), 2.08-2.00 (m, 2H), 1.97-1.87 (m, 3H), 1.78-1.67 (m, 4H). HRMS (ESI) m/z: Calcd for C₂₇H₃₁F₅NO₈, [M+H]⁺ 592.1965, found 592.1962.

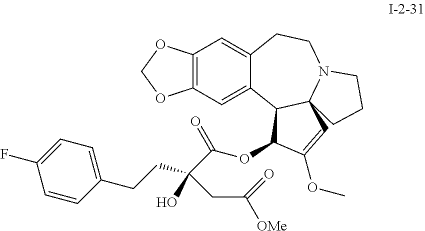 [SMILES: COC(=O)C[C@](O)(CCc1ccc(F)cc1)C(=O)O[C@@H]1C(OC)=C[C@]23CCCN2CCc2cc4c(cc2[C@H]13)OCO4]

Compound I-2-31: light yellow amorphous solid, 55% yield. ¹H NMR (400 MHz, CDCl₃) δ 7.05 (dd, J=8.5, 5.6 Hz, 2H), 6.95 (t, J=8.7 Hz, 2H), 6.58 (s, 1H), 6.55 (s, 1H), 6.05 (dd, J=9.8, 0.9 Hz, 1H), 5.84 (d, J=1.6 Hz, 1H), 5.73 (d, J=1.6 Hz, 1H), 5.09 (s, 1H), 3.81 (d, J=9.8 Hz, 1H), 3.69 (s, 3H), 3.61 (s, 1H), 3.59 (s, 3H), 3.21-3.06 (m, 2H), 2.97 (td, J=11.3, 6.7 Hz, 1H), 2.68-2.55 (m, 3H), 2.42-2.25 (m, 2H), 2.30 (d, J=16.4 Hz, 1H), 2.12-1.88 (m, 3H), 1.98 (d, J=16.4 Hz, 1H), 1.83-1.65 (m, 4H) ppm. HRMS (ESI) m/z: Calcd for C₃₁H₃₅FNO₈, [M+H]⁺ 568.2341, found 568.2347.

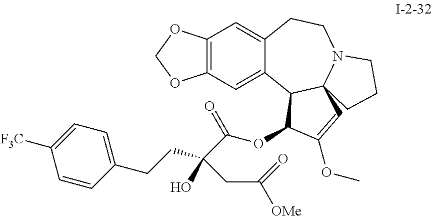 [SMILES: COC(=O)C[C@](O)(CCc1ccc(C(F)(F)F)cc1)C(=O)O[C@@H]1C(OC)=C[C@]23CCCN2CCc2cc4c(cc2[C@H]13)OCO4]

Compound I-2-32: light yellow amorphous solid, yield 50%. ¹H NMR (400 MHz, CDCl₃) δ 7.52 (d, J=7.9 Hz, 2H), 7.21 (d, J=7.9 Hz, 2H), 6.58 (s, 1H), 6.56 (s, 1H), 6.06 (d, J=9.8 Hz, 1H), 5.83 (d, J=1.5 Hz, 1H), 5.71 (d, J=1.5 Hz, 1H), 5.10 (s, 1H), 3.81 (d, J=9.8 Hz, 1H), 3.69 (s, 3H), 3.64 (s, 1H), 3.59 (s, 3H), 3.21-3.05 (m, 2H), 2.95 (td, J=11.6, 6.9 Hz, 1H), 2.71 (td, J=12.8, 5.2 Hz, 1H), 2.61 (dd, J=10.7, 7.7 Hz, 2H), 2.47-2.34 (m, 2H), 2.30 (d, J=16.5 Hz, 1H), 2.10-2.02 (m, 1H), 1.99 (d, J=16.4 Hz, 1H), 1.92 (ddd, J=12.2, 7.7, 4.5 Hz, 1H), 1.82-1.68 (m, 4H) ppm. HRMS (ESI) m/z: Calcd for C₂₉H₂₉F₃NO₆, [M+H]⁺ 618.2305, found 618.2315.

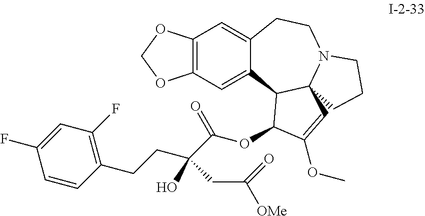 [SMILES: COC(=O)C[C@](O)(CCc1ccc(F)cc1F)C(=O)O[C@@H]1C(OC)=C[C@]23CCCN2CCc2cc4c(cc2[C@H]13)OCO4]

Compound I-2-33: light yellow amorphous solid, 82% yield. ¹H NMR (400 MHz, CDCl₃) δ 7.07 (q, J=8.1 Hz, 1H), 6.82-6.70 (m, 2H), 6.59 (s, 1H), 6.54 (s, 1H), 6.04 (d, J=9.8 Hz, 1H), 5.85 (d, J=1.6 Hz, 1H), 5.75 (d, J=1.6 Hz, 1H), 5.08 (s, 1H), 3.80 (d, J=9.8 Hz, 1H), 3.67 (s, 3H), 3.64 (s, 1H), 3.58 (s, 3H), 3.23-3.05 (m, 2H), 2.95 (td, J=11.5, 7.0 Hz, 1H), 2.66-2.54 (m, 3H), 2.46 (dd, J=12.0, 5.7 Hz, 1H), 2.36 (dd, J=14.2, 6.9 Hz, 1H), 2.28 (d, J=16.5 Hz, 1H), 2.09-1.99 (m, 1H), 1.96-1.87 (m, 2H), 1.80-1.72 (m, 2H), 1.73-1.65 (m, 2H) ppm. HRMS (ESI) m/z: Calcd for C₃₁H₃₄F₂NO₈, [M+H]⁺ 586.2248, found 586.2252.

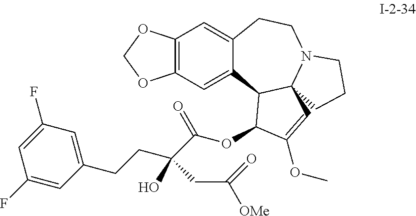 [SMILES: COC(=O)C[C@](O)(CCc1cc(F)cc(F)c1)C(=O)O[C@@H]1C(OC)=C[C@]23CCCN2CCc2cc4c(cc2[C@H]13)OCO4]

Compound I-2-34: light yellow amorphous solid, 70% yield. ¹H NMR (400 MHz, CDCl₃) δ 6.66-6.58 (m, 3H), 6.58 (s, 1H), 6.56 (s, 1H), 6.04 (d, J=9.5 Hz, 1H), 5.84 (m, 1H), 5.78 (s, 1H), 5.11 (s, 1H), 3.81 (d, J=9.6 Hz, 1H), 3.70 (s, 3H), 3.63 (s, 1H), 3.59 (s, 3H), 3.20-3.00 (m, 2H), 2.94 (td, J=11.6, 7.2 Hz, 1H), 2.66-2.49 (m, 3H), 2.41-2.24 (m, 3H), 2.09-1.86 (m, 3H), 1.82-1.68 (m, 4H) ppm. HRMS (ESI) m/z: Calcd for C₃₁H₃₄F₂NO₈, [M+H]⁺ 586.2246, found 586.2252.

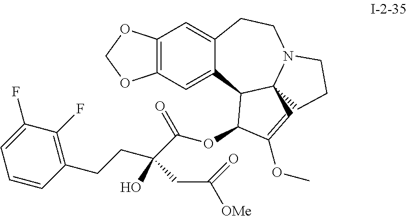 [SMILES: COC(=O)C[C@](O)(CCc1cccc(F)c1F)C(=O)O[C@@H]1C(OC)=C[C@]23CCCN2CCc2cc4c(cc2[C@H]13)OCO4]

Compound I-2-35: light yellow amorphous solid, 41% yield. ¹H NMR (400 MHz, CDCl₃) δ 6.93 (td, J=8.7, 4.4 Hz, 1H), 6.89-6.77 (m, 2H), 6.59 (s, 1H), 6.54 (d, J=5.7 Hz, 1H), 6.04 (d, J=9.8 Hz, 1H), 5.85 (d, J=1.6 Hz, 1H), 5.77 (d, J=1.6 Hz, 1H), 5.09 (s, 1H), 3.80 (d, J=9.8 Hz, 1H), 3.68 (s, 3H), 3.65 (s, 1H), 3.58 (s, 3H), 3.22-3.07 (m, 2H), 2.94 (td, J=11.6, 7.0 Hz, 1H), 2.66-2.54 (m, 3H), 2.48-2.32 (m, 2H), 2.28 (d, J=16.5 Hz, 1H), 2.09-2.00 (m, 1H), 1.96-1.88 (m, 2H), 1.82-1.72 (m, 2H), 1.70 (dd, J=9.8, 7.4 Hz, 2H) ppm. HRMS (ESI) m/z: Calcd for C₃₁H₃₄F₂NO₈, [M+H]⁺ 586.2243, found 586.2252.

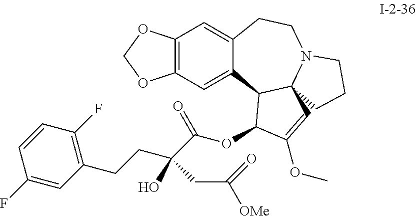 [SMILES: COC(=O)C[C@](O)(CCc1cc(F)ccc1F)C(=O)O[C@@H]1C(OC)=C[C@]23CCCN2CCc2cc4c(cc2[C@H]13)OCO4]

Compound I-2-36: light yellow amorphous solid, 47% yield. ¹H NMR (400 MHz, CDCl₃) δ 7.03-6.93 (m, 2H), 6.87 (dd, J=8.3, 5.3 Hz, 1H), 6.59 (s, 1H), 6.54 (s, 1H), 6.05 (d, J=9.7 Hz, 1H), 5.85 (d, J=1.5 Hz, 1H), 5.76 (d, J=1.6 Hz, 1H), 5.09 (s, 1H), 3.80 (d, J=9.8 Hz, 1H), 3.69 (s, 3H), 3.66 (s, 1H), 3.59 (s, 3H), 3.23-3.06 (m, 2H), 2.95 (td, J=11.6, 7.0 Hz, 1H), 2.71-2.48 (m, 4H), 2.37 (dd, J=14.2, 6.9 Hz, 1H), 2.28 (d, J=16.6 Hz, 1H), 2.05 (td, J=12.4, 9.5 Hz, 1H), 1.96-1.86 (m, 2H), 1.80-1.64 (m, 4H) ppm. HRMS (ESI) m/z: Calcd for C₃₁H₃₄F₂NO₈, [M+H]⁺ 586.2254, found 586.2252.

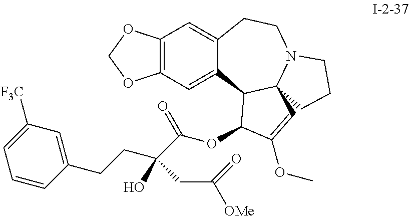 [SMILES: COC(=O)C[C@](O)(CCc1cccc(C(F)(F)F)c1)C(=O)O[C@@H]1C(OC)=C[C@]23CCCN2CCc2cc4c(cc2[C@H]13)OCO4]

Compound I-2-37: light yellow amorphous solid, 118 mg, 47% yield. ¹H NMR (400 MHz, CDCl₃) δ 7.45 (d, J=7.9 Hz, 1H), 7.38 (t, J=7.7 Hz, 1H), 7.34 (s, 1H), 7.30 (d, J=7.5 Hz, 1H), 6.58 (s, 1H), 6.56 (s, 1H), 6.07 (d, J=9.8 Hz, 1H), 5.84 (d, J=1.7 Hz, 1H), 5.75 (d, J=1.7 Hz, 1H), 5.12 (s, 1H), 3.82 (d, J=9.9 Hz, 1H), 3.69 (s, 3H), 3.66 (s, 1H), 3.59 (s, 3H), 3.21-3.07 (m, 2H), 3.00-2.90 (m, 1H), 2.73 (td, J=13.0, 5.0 Hz, 1H), 2.62 (t, J=9.2 Hz, 2H), 2.47-2.33 (m, 2H), 2.28 (d, J=16.5 Hz, 1H), 2.06 (q, J=10.1 Hz, 1H), 1.98-1.91 (m, 2H), 1.76 (dd, J=12.2, 7.5 Hz, 4H) ppm. HRMS (ESI)m/z: Calcd for C₂₉H₂₉F₃NO₆, [M+H]⁺ 1618.2307, found 618.2315.

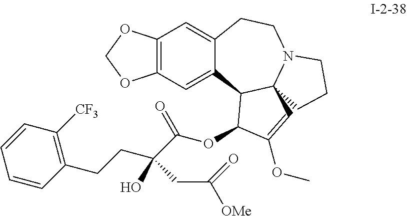 [SMILES: COC(=O)C[C@](O)(CCc1ccccc1C(F)(F)F)C(=O)O[C@@H]1C(OC)=C[C@]23CCCN2CCc2cc4c(cc2[C@H]13)OCO4]

Compound I-2-38: light yellow amorphous solid, 60% yield. ¹H NMR (400 MHz, CDCl₃) δ 7.59 (d, J=7.8 Hz, 1H), 7.45 (t, J=7.5 Hz, 1H), 7.27 (q, J=8.0 Hz, 2H), 6.56 (s, 1H), 6.53 (s, 1H), 6.03 (d, J=9.8 Hz, 1H), 5.84 (d, J=1.6 Hz, 1H), 5.71 (d, J=1.6 Hz, 1H), 5.08 (s, 1H), 3.79 (d, J=9.8 Hz, 1H), 3.69 (s, 3H), 3.68 (s, 1H), 3.60 (s, 3H), 3.17 (td, J=12.0, 11.3, 6.7 Hz, 1H), 3.15-3.05 (m, 1H), 2.94 (td, J=11.6, 7.0 Hz, 1H), 2.82 (td, J=13.0, 4.3 Hz, 1H), 2.66-2.52 (m, 3H), 2.35 (dd, J=14.2, 6.9 Hz, 1H), 2.28 (d, J=16.5 Hz, 1H), 2.08-1.98 (m, 1H), 1.92 (dt, J=9.0, 4.7 Hz, 1H), 1.86 (d, J=16.5 Hz, 1H), 1.75 (dt, J=13.8, 5.1 Hz, 2H), 1.68-1.59 (m, 2H) ppm. HRMS (ESI) m/z: Calcd for C₃₂H₃₅F₃NO₈, [M+H]⁺ 618.2312, found 618.2315.

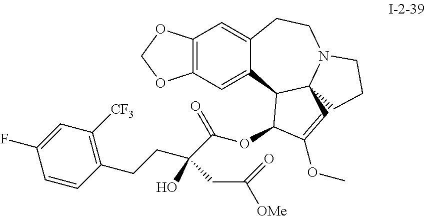 [SMILES: COC(=O)C[C@](O)(CCc1ccc(F)cc1C(F)(F)F)C(=O)O[C@@H]1C(OC)=C[C@]23CCCN2CCc2cc4c(cc2[C@H]13)OCO4]

Compound I-2-39: light yellow amorphous solid, 84% yield. ¹H NMR (400 MHz, CDCl₃) δ 7.30 (dd, J=9.4, 2.7 Hz, 1H), 7.26-7.18 (m, 1H), 7.20-7.11 (m, 1H), 6.57 (s, 1H), 6.53 (s, 1H), 6.02 (d, J=9.8 Hz, 1H), 5.84 (d, J=1.6 Hz, 1H), 5.73 (d, J=1.6 Hz, 1H), 5.09 (s, 1H), 3.79 (d, J=9.8 Hz, 1H), 3.69 (s, 3H), 3.68 (s, 1H), 3.60 (s, 3H), 3.22-3.05 (m, 2H), 2.94 (td, J=11.6, 7.0 Hz, 1H), 2.78 (td, J=13.3, 4.7 Hz, 1H), 2.64-2.45 (m, 3H), 2.35 (dd, J=14.2, 6.9 Hz, 1H), 2.27 (d, J=16.5 Hz, 1H), 2.08-1.96 (m, 1H), 1.95-1.89 (m, 1H), 1.86 (d, J=16.6 Hz, 1H), 1.81-1.63 (m, 4H) ppm. HRMS (ESI) m/z: Calcd for C₃₂H₃₄F₄NO₈, [M+H]⁺ 636.2212, found 636.2221.

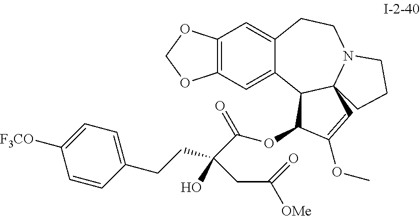 [SMILES: COC(=O)C[C@](O)(CCc1ccc(OC(F)(F)F)cc1)C(=O)O[C@@H]1C(OC)=C[C@]23CCCN2CCc2cc4c(cc2[C@H]13)OCO4]

Compound I-2-40: light yellow amorphous solid, 60% yield. ¹H NMR (400 MHz, CDCl₃) δ 7.11 (s, 4H), 6.58 (s, 1H), 6.56 (s, 1H), 6.05 (d, J=10.0 Hz, 1H), 5.83 (d, J=1.5 Hz, 1H), 5.71 (d, J=1.6 Hz, 1H), 5.10 (s, 1H), 3.81 (d, J=9.8 Hz, 1H), 3.69 (s, 3H), 3.59 (s, 3H), 3.56 (s, 1H), 3.20-3.03 (m, 2H), 2.96 (td, J=11.4, 6.9 Hz, 1H), 2.68-2.54 (m, 3H), 2.43-2.26 (m, 3H), 2.09-1.96 (m, 2H), 1.98-1.87 (m, 1H), 1.82-1.67 (m, 4H) ppm. HRMS (ESI) m/z: Calcd for C₃₂H₃₅F₃NO₉, [M+H]⁺ 634.2263, found 634.2264.

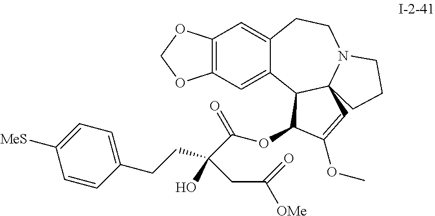 [SMILES: COC(=O)C[C@](O)(CCc1ccc(SC)cc1)C(=O)O[C@@H]1C(OC)=C[C@]23CCCN2CCc2cc4c(cc2[C@H]13)OCO4]

Compound I-2-41: light yellow amorphous solid, 56% yield. ¹′H NMR (400 MHz, CDCl₃) δ 7.18 (d, J=8.2 Hz, 2H), 7.03 (d, J=8.2 Hz, 2H), 6.59 (s, 1H), 6.55 (s, 1H), 6.06 (d, J=9.8 Hz, 1H), 5.84 (d, J=1.6 Hz, 1H), 5.73 (d, J=1.6 Hz, 1H), 5.09 (s, 1H), 3.80 (d, J=9.8 Hz, 1H), 3.69 (s, 3H), 3.61 (s, 1H), 3.58 (s, 3H), 3.20-3.06 (m, 2H), 2.95 (td, J=11.6, 7.0 Hz, 1H), 2.66-2.56 (m, 3H), 2.46 (s, 3H), 2.41-2.26 (m, 3H), 2.10-2.01 (m, 1H), 2.00-1.88 (m, 2H), 1.81-1.64 (m, 4H) ppm. HRMS (ESI) m/z: Calcd for C₃₂H₃₈NO₈S, [M+H]⁺ 596.2313, found 596.2318.

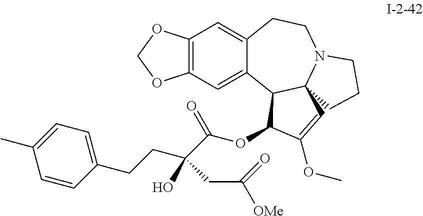 [SMILES: COC(=O)C[C@](O)(CCc1ccc(C)cc1)C(=O)O[C@@H]1C(OC)=C[C@]23CCCN2CCc2cc4c(cc2[C@H]13)OCO4]

Compound I-2-42: light yellow amorphous solid, 71% yield. ¹H NMR (400 MHz, CDCl₃) δ 7.10 (d, J=7.6 Hz, 2H), 7.02 (d, J=7.7 Hz, 2H), 6.60 (s, 1H), 6.57 (s, 1H), 6.08 (d, J=9.8 Hz, 1H), 5.84 (d, J=1.6 Hz, 1H), 5.74 (d, J=1.6 Hz, 1H), 5.11 (s, 1H), 3.82 (d, J=9.8 Hz, 1H), 3.71 (s, 4H), 3.59 (s, 3H), 3.26-3.06 (m, 2H), 2.99 (dt, J=12.6, 6.2 Hz, 1H), 2.76-2.54 (m, 3H), 2.44-2.27 (m, 6H), 2.15-2.02 (m, 1H), 2.01-1.89 (m, 2H), 1.84-1.68 (m, 4H) ppm. HRMS (ESI) m/z: Calcd for C₃₂H₃₈NO₈, [M+H]⁺ 564.2591, found 564.2597.

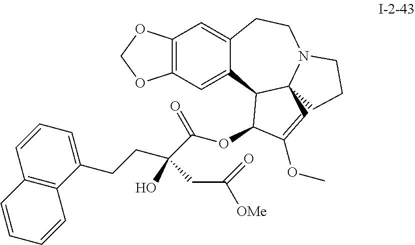 [SMILES: COC(=O)C[C@](O)(CCc1cccc2ccccc12)C(=O)O[C@@H]1C(OC)=C[C@]23CCCN2CCc2cc4c(cc2[C@H]13)OCO4]

Compound I-243: light yellow amorphous solid, 60% yield. ¹H NMR (400 MHz, CDCl₃) δ 8.09-7.85 (m, 2H), 7.75 (d, J=8.2 Hz, 1H), 7.51 (dd, J=6.3, 3.3 Hz, 2H), 7.42 (t, J=7.6 Hz, 1H), 7.36-7.26 (m, 1H), 6.59 (s, 1H), 6.55 (s, 1H), 6.20 (d, J=9.9 Hz, 1H), 5.91-5.82 (m, 1H), 5.71 (d, J=1.6 Hz, 1H), 5.21 (s, 1H), 3.88 (d, J=9.9 Hz, 1H), 3.82 (s, 1H), 3.71 (s, 3H), 3.64 (s, 3H), 3.33 (td, J=13.1, 7.7 Hz, 1H), 3.22-3.07 (m, 2H), 3.09-2.90 (m, 2H), 2.80-2.63 (m, 2H), 2.39 (dd, J=14.3, 6.9 Hz, 1H), 2.32 (d, J=16.6 Hz, 1H), 2.17-2.06 (m, 1H), 2.05-1.92 (m, 2H), 1.90 (d, J=16.5 Hz, 1H), 1.87-1.76 (m, 3H) ppm. HRMS (ESI) m/z: Calcd for C₃₈H₃₈NO₈, [M+H]⁺ 600.2592, found 600.2597.

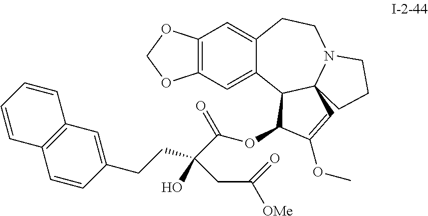 [SMILES: COC(=O)C[C@](O)(CCc1ccc2ccccc2c1)C(=O)O[C@@H]1C(OC)=C[C@]23CCCN2CCc2cc4c(cc2[C@H]13)OCO4]

Compound I-2-44: light yellow amorphous solid, 153 mg, 61% yield. ¹H NMR (400 MHz, CDCl₃) δ 7.83-7.70 (m, 3H), 7.54 (s, 1H), 7.50-7.37 (m, 2H), 7.25 (d, J=9.0 Hz, 1H), 6.59 (s, 1H), 6.57 (s, 1H), 6.09 (d, J=9.7 Hz, 1H), 5.81 (s, 1H), 5.69 (s, 1H), 5.11 (s, 1H), 3.83 (d, J=9.6 Hz, 1H), 3.71 (s, 3H), 3.67 (s, 1H), 3.60 (s, 3H), 3.20 (td, J=13.1, 7.9 Hz, 1H), 3.11 (q, J=8.0, 7.3 Hz, 1H), 2.96 (td, J=11.5, 7.0 Hz, 1H), 2.82 (td, J=12.8, 5.3 Hz, 1H), 2.68-2.47 (m, 3H), 2.44-2.30 (m, 2H), 2.05 (dt, J=25.3, 13.3 Hz, 2H), 1.97-1.73 (m, 5H) ppm. HRMS (ESI) m/z: Calcd for C₃₅H₃₈NO₈, [M+H]⁺ 600.2600, found 600.2597.

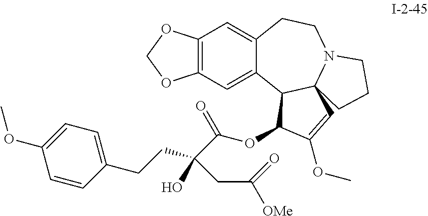 [SMILES: COC(=O)C[C@](O)(CCc1ccc(OC)cc1)C(=O)O[C@@H]1C(OC)=C[C@]23CCCN2CCc2cc4c(cc2[C@H]13)OCO4]

Compound I-245: light yellow amorphous solid, 56% yield ¹H NMR (400 MHz, CDCl₃) δ 7.02 (d, J=8.3 Hz, 2H), 6.82 (d, J=8.5 Hz, 2H), 6.59 (s, 1H), 6.55 (s, 1H), 6.06 (d, J=9.8 Hz, 1H), 5.83 (d, J=1.6 Hz, 1H), 5.73 (d, J=1.6 Hz, 1H), 5.09 (s, 1H), 3.81 (d, J=9.9 Hz, 1H), 3.78 (s, 4H), 3.70 (s, 3H), 3.58 (s, 3H), 3.21-3.07 (m, 2H), 2.97 (td, J=11.3, 6.5 Hz, 1H), 2.65-2.57 (m, 3H), 2.41-2.26 (m, 3H), 2.30 (d, J=16.4 Hz, 1H), 2.06 (dt, J=12.0, 9.6 Hz, 1H), 1.99-1.89 (m, 2H), 1.96 (d, J=16.4 Hz, 1H), 1.81-1.67 (m, 4H) ppm. HRMS (ESI) m/z: Calcd for C₃₂H₃₈NO₉, [M+H]⁺ 580.2544, found 580.2547.

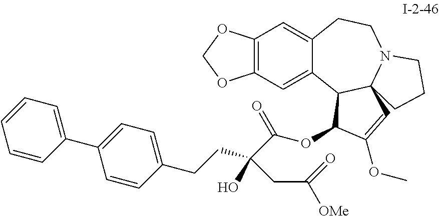 [SMILES: COC(=O)C[C@](O)(CCc1ccc(-c2ccccc2)cc1)C(=O)O[C@@H]1C(OC)=C[C@]23CCCN2CCc2cc4c(cc2[C@H]13)OCO4]

Compound I-2-46: light yellow amorphous solid, 50% yield. ¹H NMR (400 MHz, CDCl₃) δ 7.57 (d, J=7.0 Hz, 2H), 7.51 (d, J=7.9 Hz, 2H), 7.43 (t, J=7.5 Hz, 2H), 7.33 (t, J=6.8 Hz, 1H), 7.18 (d, J=7.8 Hz, 2H), 6.60 (s, 1H), 6.56 (s, 1H), 6.08 (d, J=9.8 Hz, 1H), 5.84 (d, J=1.6 Hz, 1H), 5.73 (d, J=1.7 Hz, 1H), 5.10 (s, 1H), 3.82 (d, J=9.8 Hz, 1H), 3.72 (s, 3H), 3.64 (s, 1H), 3.59 (s, 3H), 3.24-3.06 (m, 2H), 2.96 (td, J=11.6, 6.9 Hz, 1H), 2.76-2.55 (m, 3H), 2.47-2.28 (m, 3H), 2.10-2.02 (m, 1H), 1.99 (d, J=16.5 Hz, 1H), 1.93 (dd, J=9.6, 5.5 Hz, 1H), 1.83-1.72 (m, 4H) ppm. HRMS (ESI) m/z: Calcd for C₃₇H₄₀NO₈, [M+H]⁺ 626.2746, found 626.2754.

### Example 19: Preparation of Ketoxime Compounds

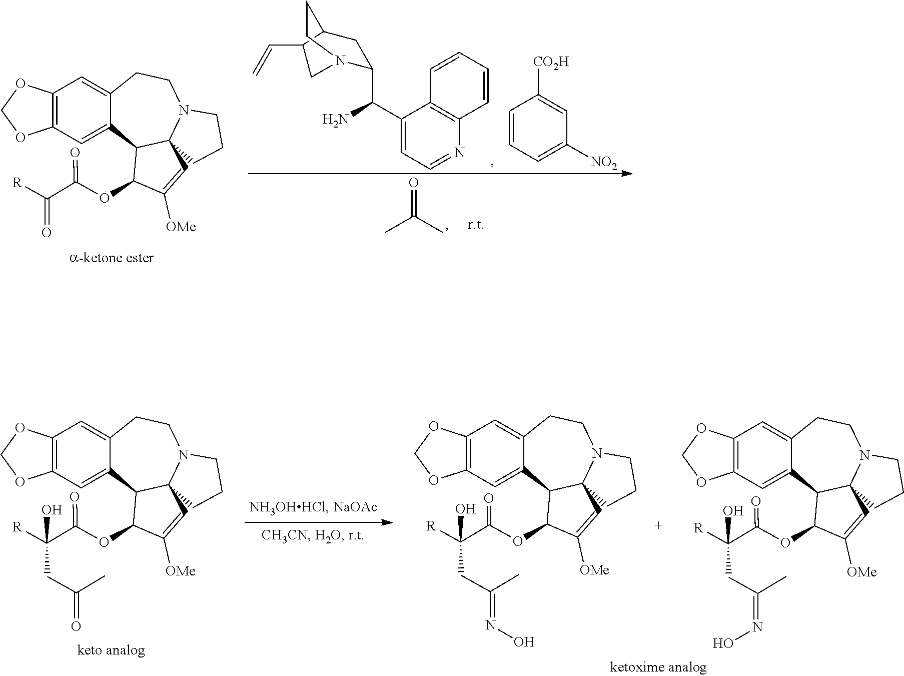

General operation: To a solution of α-keto ester (4 mmol) in acetone (5 mL) at room temperature under argon protection was added cinchonine catalyst (0.16 mmol), m-nitrobenzoic acid (0.8 mmol), and the reaction was carried out for 8 h. The ketone analogs were obtained by column chromatography separation after removal of most of the solvent.

At room temperature, the ketone compound (2 mmol) was dissolved in CH₃CN/H₂O (v/v=9: 1, 5 mL), and hydroxylamine hydrochloride (2 mmol) and sodium acetate (2.5 mmol) were added with stirring, and the reaction was quenched by adding saturated NaHCO₃ after TLC monitoring the reaction was complete and extracted by CH₂Cl₂, and the organic phases were combined and washed with saturated NaCl, and dried with anhydrous Na₂SO₄. The solvent was removed by spin evaporation to give the E/Z ketoxime analog.

Referring to the above method, the following compounds were obtained:

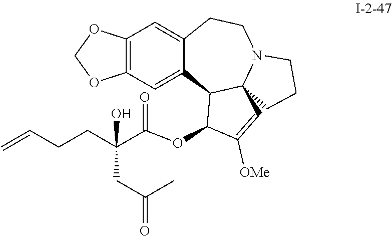

Compound I-2-47: Column chromatography PE:EA=7: 1, 0.5% TEA to give a yellow amorphous solid (87% yield). ¹H NMR (400 MHz, CDCl₃): δ 6.60 (s, 1H), 6.55 (s, 1H), 5.98 (d, J=9.8 Hz, 1H), 5.85 (dd, J=13.6, 1.5 Hz, 2H), 5.79-5.64 (m, 1H), 5.03 (s, 1H), 5.00-4.87 (m, 2H), 3.76 (d, J=9.8 Hz, 1H), 3.67 (s, 3H), 3.57 (s, 1H), 3.10 (ddd, J=23.8, 13.2, 7.6 Hz, 2H), 2.92 (td, J=11.6, 7.0 Hz, 1H), 2.63-2.52 (m, 2H), 2.46 (d, J=17.5 Hz, 1H), 2.36 (dd, J=14.1, 6.9 Hz, 1H), 2.22 (d, J=17.5 Hz, 1H), 2.11-1.95 (m, 2H), 1.89 (s, 3H), 1.87-1.67 (m, 4H), 1.55-1.38 (m, 2H) ppm, HRMS-ESI (m/z): Calcd for C₂₇H₃₄NO₇ [M+H]⁺ 484.2336. Found 484.2257.

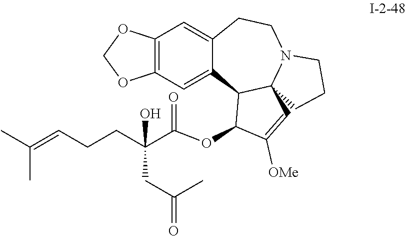

Compound I-2-48: Column chromatography PE:EA=7: 1, 0.5% TEA to give a yellow amorphous solid (96% yield). ¹H NMR (400 MHz, CDCl₃): δ 6.60 (s, 1H), 6.54 (s, 1H), 5.96 (d, J=9.8 Hz, 1H), 5.87-5.80 (m, 2H), 5.02 (s, 1H), 5.03-4.94 (m, 1H), 3.75 (d, J=9.8 Hz, 1H), 3.66 (s, 3H), 3.56 (s, 1H), 3.20-3.02 (m, 2H), 2.92 (td, J=11.5, 6.9 Hz, 1H), 2.62-2.51 (m, 2H), 2.42 (d, J=17.3 Hz, 1H), 2.36 (dd, J=14.2, 7.0 Hz, 1H), 2.13 (d, J=17.4 Hz, 1H), 2.07-1.93 (m, 3H), 1.91-1.85 (m, 1H), 1.88 (s, 3H), 1.75 (dt, J=13.8, 7.5 Hz, 4H), 1.65 (s, 3H), 1.56 (s, 3H) ppm. HRMS-ESI (m/z): Calcd for C₂₉H₃₈NO₇ [M+H]⁺ 512.2649. Found 512.2570.

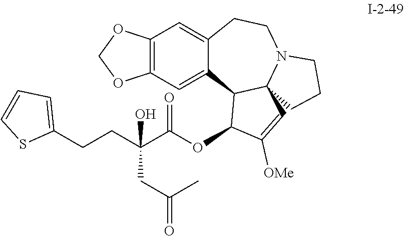

Compound I-249: Column chromatography PE:EA=5:1, 0.5% TEA to give a yellow amorphous solid (88% yield). ¹H NMR (400 MHz, CDCl₃): δ 7.10 (dd, J=5.1, 1.2 Hz, 1H), 6.90 (dd, J=5.2, 3.4 Hz, 1H), 6.73 (d, J=2.3 Hz, 1H), 6.60 (s, 1H), 6.56 (s, 1H), 6.04 (d, J=9.8 Hz, 1H), 5.80 (dd, J=11.2, 1.5 Hz, 2H), 5.05 (s, 1H), 3.78 (d, J=9.9 Hz, 1H), 3.70 (s, 1H), 3.67 (s, 3H), 3.22-3.04 (m, 2H), 3.00-2.82 (m, 2H), 2.65-2.53 (m, 3H), 2.49 (d, J=17.6 Hz, 1H), 2.38 (dd, J=14.2, 6.9 Hz, 1H), 2.20 (d, J=17.6 Hz, 1H), 2.10-1.97 (m, 1H), 1.94-1.88 (m, 1H), 1.89 (s, 3H), 1.84-1.69 (m, 4H) ppm. HRMS-ESI (m/z): Calcd for C₂₉H₃₄NO₇S [M+H]⁺ 540.2057. Found 540.1987.

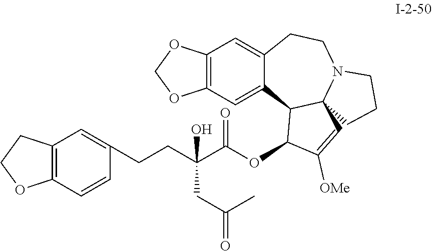

Compound I-2-50: Column chromatography PE:EA=5:1, 0.5% TEA to give a yellow amorphous solid (98% yield). ¹H NMR (400 MHz, CDCl₃): δ 6.94 (s, 1H), 6.82 (d, J=8.1 Hz, 1H), 6.68 (d, J=8.0 Hz, 1H), 6.57 (d, J=3.2 Hz, 2H), 6.03 (d, J=9.8 Hz, 1H), 5.77 (dd, J=26.9, 1.5 Hz, 2H), 5.07 (s, 1H), 4.53 (t, J=8.6 Hz, 2H), 3.79 (d, J=9.8 Hz, 1H), 3.69 (s, 3H), 3.66 (s, 1H), 3.16 (t, J=8.6 Hz, 2H), 3.14-3.04 (m, 2H), 2.93 (td, J=11.5, 7.0 Hz, 1H), 2.65-2.48 (m, 3H), 2.46 (d, J=17.5 Hz, 1H), 2.36 (dd, J=14.1, 6.9 Hz, 1H), 2.33-2.21 (m, 1H), 2.19 (d, J=17.5 Hz, 1H), 2.10-1.99 (m, 1H), 1.94-1.88 (m, 1H), 1.90 (s, 3H), 1.75 (p, J=9.2, 8.1 Hz, 2H), 1.72-1.59 (m, 2H) ppm. HRMS-ESI (m/z): Calcd for C₃₃H₃₈NO₈ [M+H]⁺ 576.2598. Found 576.2519.

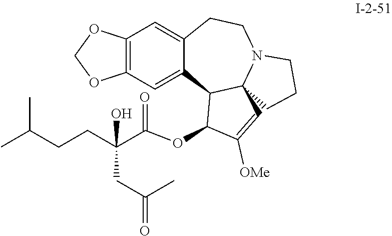

Compound I-2-51: Column chromatography PE:EA=5:1, 0.5% TEA to give a yellow amorphous solid (95% yield). HRMS-ESI (m/z): Calcd for C₂₈H₃₈NO₈ [M+H]⁺ 500.2643. Found 500.2648.

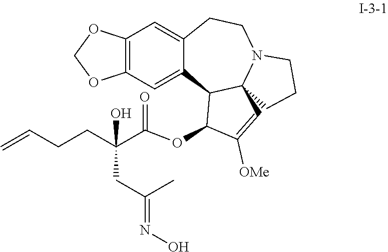

Compound I-3-1: Column chromatography PE:EA=5:1, 0.5% TEA to give a yellow amorphous solid (37% yield). ¹H NMR (400 MHz, CDCl₃): δ 8.06 (s, 1H), 6.58 (s, 1H), 6.55 (s, 1H), 5.85 (d, J=3.3 Hz, 2H), 5.79-5.66 (m, 1H), 5.04 (s, 1H), 5.00-4.88 (m, 2H), 3.77 (d, J=9.9 Hz, 1H), 3.67 (s, 3H), 3.21-3.02 (m, 2H), 2.99-2.86 (m, 1H), 2.65-2.53 (m, 2H), 2.38 (dd, J=14.1, 6.9 Hz, 1H), 2.10 (d, J=16.0 Hz, 1H), 2.05-1.98 (m, 2H), 1.95-1.89 (m, 1H), 1.78-1.71 (m, 1H), 1.63 (s, 3H), 1.59-1.47 (m, 1H), 0.91-0.80 (m, 6H) ppm. HRMS-ESI (m/z): Calcd for C₂₇H₃₅N₂O₇ [M+H]⁺ 499.2445. Found 499.2366.

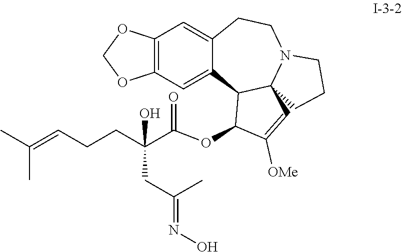

Compound I-3-2: Column chromatography PE:EA=5:1, 0.5% TEA to give a yellow amorphous solid (42% yield). ¹H NMR (400 MHz, CDCl₃): δ 6.60 (s, 1H), 6.58 (s, 1H), 5.93 (d, J=9.6 Hz, 1H), 5.83 (d, J=19.6 Hz, 2H), 5.06 (s, 1H), 5.05-4.96 (m, 2H), 3.81 (s, 1H), 3.67 (s, 3H), 3.35 (s, 1H), 3.20-3.05 (m, 2H), 2.94 (d, J=8.5 Hz, 1H), 2.90-2.75 (m, 1H), 2.63-2.48 (m, 3H), 2.39 (dd, J=14.3, 6.8 Hz, 1H), 2.18-2.00 (m, 3H), 1.89 (d, J=24.0 Hz, 4H), 1.72 (s, 3H), 1.66 (d, J=5.6 Hz, 3H), 1.57 (d, J=6.1 Hz, 3H) ppm. HRMS-ESI (m/z): Calcd for C₂₉H₃₉N₂O₇ [M+H]⁺ 527.2758. Found 527.2679.

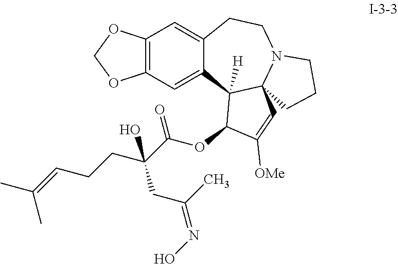

Compound I-3-3: Column chromatography PE:EA=5:1, 0.5% TEA to give a yellow amorphous solid (30% yield). ¹H NMR (400 MHz, CDCl₃): δ 7.60 (s, 1H), 6.58 (s, 1H), 6.55 (s, 1H), 5.93 (d, J=9.7 Hz, 1H), 5.84 (dd, J=8.9, 1.6 Hz, 2H), 5.04 (s, 1H), 5.04-4.95 (m, 1H), 3.77 (d, J=9.7 Hz, 1H), 3.67 (s, 3H), 3.63 (s, 1H), 3.22-3.03 (m, 2H), 2.98-2.86 (m, 1H), 2.63-2.52 (m, 2H), 2.37 (dd, J=14.2, 7.0 Hz, 1H), 2.07 (d, J=15.9 Hz, 1H), 2.04-1.83 (m, 4H), 1.82-1.69 (m, 3H), 1.65 (s, 6H), 1.56 (s, 3H), 1.53-1.36 (m, 2H) ppm. HRMS-ESI (m/z): Calcd for C₂₉H₃₉N₂O₇ [M+H]⁺ 527.2758. Found 527.2679.

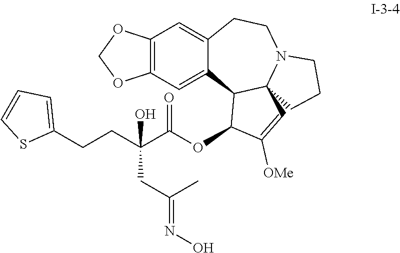

Compound I-3-4: Column chromatography PE:EA=4:1, 0.5% TEA to give a yellow amorphous solid (46% yield). ¹H NMR (400 MHz, CDCl₃): δ 7.09 (dd, J=5.1, 1.2 Hz, 1H), 6.89 (dd, J=5.1, 3.4 Hz, 1H), 6.73 (d, J=2.4 Hz, 1H), 6.58 (s, 1H), 6.56 (s, 1H), 5.99 (d, J=9.7 Hz, 1H), 5.77 (dd, J=34.4, 1.6 Hz, 2H), 5.06 (s, 1H), 3.87 (s, 1H), 3.78 (d, J=9.7 Hz, 1H), 3.67 (s, 3H), 3.24-3.02 (m, 2H), 2.99-2.83 (m, 2H), 2.65-2.53 (m, 2H), 2.55-2.42 (m, 1H), 2.39 (dd, J=14.5, 7.0 Hz, 1H), 2.13 (d, J=18.6 Hz, 1H), 2.10-1.95 (m, 3H), 1.95-1.79 (m, 3H), 1.82-1.68 (m, 4H), 1.63 (s, 3H) ppm. HRMS-ESI (m/z): Calcd for C₂₉H₃₅N₂O₇S [M+H]⁺ 555.2166. Found 555.2087.

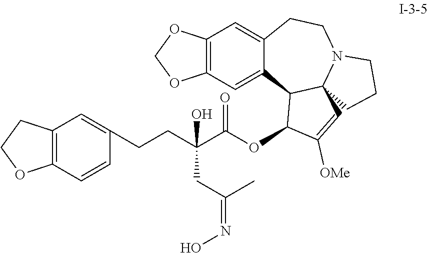

Compound I-3-5: Column chromatography PE:EA=4:1, 0.5% TEA to give a yellow amorphous solid (45% yield). ¹H NMR (400 MHz, CDCl₃): δ 6.95 (s, 1H), 6.84 (d, J=8.1 Hz, 1H), 6.67 (d, J=8.1 Hz, 1H), 6.57 (s, 1H), 6.56 (s, 1H), 5.99 (d, J=9.7 Hz, 1H), 5.73 (dd, J=54.7, 1.7 Hz, 2H), 5.08 (s, 1H), 4.53 (t, J=8.6 Hz, 2H), 3.80 (d, J=9.8 Hz, 1H), 3.70 (s, 3H), 3.23-3.14 (m, 1H), 3.16 (t, J=8.6, 7.7 Hz, 2H), 3.14-3.03 (m, 1H), 3.00-2.87 (m, 1H), 2.66-2.46 (m, 3H), 2.38 (dd, J=14.2, 7.0 Hz, 1H), 2.22-2.08 (m, 1H), 2.12 (d, J=17.4 Hz, 1H), 2.08-1.95 (m, 2H), 1.99 (d, J=17.4 Hz, 1H), 1.96-1.84 (m, 2H), 1.82-1.69 (m, 4H), 1.66 (s, 3H) ppm. HRMS-ESI (m/z): Calcd for C₃₃H₃₉N₂O₈S [M+H]⁺ 591.2707. Found 591.2628.

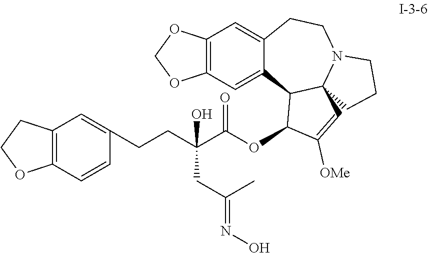

Compound I-3-6: Column chromatography PE:EA=4:1, 0.5% TEA to give a yellow amorphous solid (35% yield). ¹H NMR (400 MHz, CDCl3): δ 6.94 (s, 1H), 6.82 (d, J=8.1 Hz, 1H), 6.68 (d, J=8.1 Hz, 1H), 6.57 (s, 1H), 6.57 (s, 1H), 6.03 (d, J=9.8 Hz, 1H), 5.77 (dd, J=28.7, 1.5 Hz, 2H), 5.07 (s, 1H), 4.54 (t, J=8.7 Hz, 2H), 3.79 (d, J=9.9 Hz, 1H), 3.69 (s, 3H), 3.65 (s, 1H), 3.23-3.04 (m, 3H), 3.19 (t, J=8.6, 7.7 Hz, 2H), 3.00-2.86 (m, 1H), 2.65-2.48 (m, 4H), 2.46 (d, J=17.5 Hz, 1H), 2.36 (dd, J=14.1, 6.9 Hz, 1H), 2.28 (dd, J=25.2, 12.5 Hz, 1H), 2.19 (d, J=17.4 Hz, 1H), 1.90 (s, 4H), 1.82-1.69 (m, 2H), 1.69-1.60 (m, 2H) ppm. HRMS-ESI (m/z): Calcd for C₃₃H₃₉N₂O₈S [M+H]⁺ 591.2707. Found 591.2628.

### Example 20: Preparation of Aldoxime Compounds

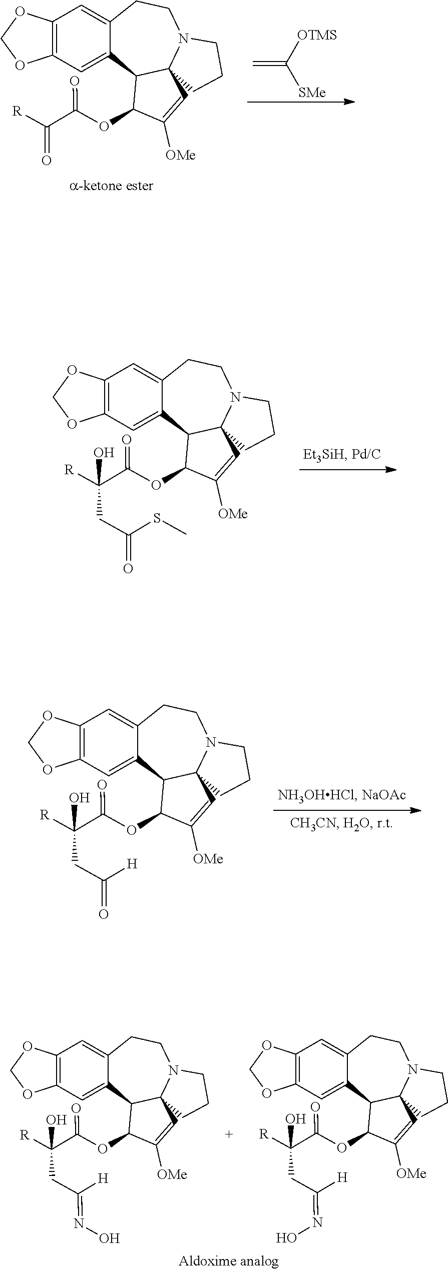

General operation: under argon protection, −60 °C, to α-keto ester (1 mmol) of CH₂Cl₂ (15 mL) solution dropwise to thioenol silicone ether (2 mmol), 10 min later, dropwise to BF₃—OEt₂ (3 mmol), TLC plate to monitor the reaction is complete, the addition of saturated NaHCO₃ quenching, CH₂Cl₂ extraction, the combined organic phase, saturated NaCl was washed and dried with anhydrous Na₂SO₄. The solvent was removed by spin evaporation to give the thioester intermediate.

To a solution of CH₂Cl₂ (5 mL) of the thioester at room temperature, Pd/C (0.1 mmol), Et₃SiH (2.5 mmol) were added and monitored until the reaction was complete. The reaction was quenched by the addition of saturated NaHCO₃, filtered through diatomaceous earth, extracted with CH₂Cl₂, the organic phases were combined, washed with saturated NaCl and dried over anhydrous Na₂SO₄. The solvent was removed by spin evaporation to give the aldehyde analog.

At room temperature, the intermediate aldehyde (2 mmol) was dissolved in CH₃CN/H₂O (v/v=9:1.5 mL), hydroxylamine hydrochloride (2 mmol), sodium acetate (2.5 mmol) were added with stirring, and the reaction was monitored by TLC plate until complete. Quenched by addition of saturated NaHCO₃, extracted with CH₂Cl₂, combined organic phases, washed with saturated NaCl and dried over anhydrous Na₂SO₄. The solvent was removed by spin distillation to give the E/Z aldoxime analog.

Referring to the above method, the following compounds were obtained:

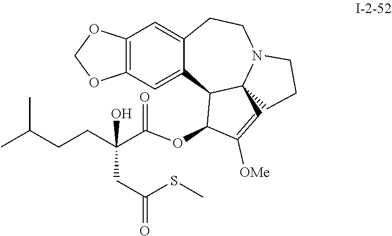

Compound I-2-52: obtained light pink colored amorphous solid (98% yield). ¹H NMR (400 MHz, CDCl₃): δ 6.61 (s, 1H), 6.52 (s, 1H), 6.00 (d, J=9.8 Hz, 1H), 5.86 (dd, J=6.5, 1.7 Hz, 2H), 5.04 (s, 1H), 3.78 (d, J=9.8 Hz, 1H), 3.67 (s, 3H), 3.39 (s, 1H), 3.15-3.03 (m, 2H), 2.98-2.89 (m, 1H), 2.61-2.55 (m, 2H), 2.52 (d, J=16.0 Hz, 1H), 2.36 (dd, J=14.1, 6.8 Hz, 1H), 2.22 (s, 3H), 2.00 (d, J=16.0 Hz, 1H), 1.94-1.86 (m, 1H), 1.81-1.69 (m, 2H), 1.51-1.34 (m, 3H), 1.33-1.21 (m, 2H), 0.99-0.92 (m, 1H), 0.82 (t, J=6.4 Hz, 6H) ppm. HRMS-ESI (m/z): Calcd for C₂₈H₃₈NO₇S [M+H]⁺ 532.2370. Found 532.2291.

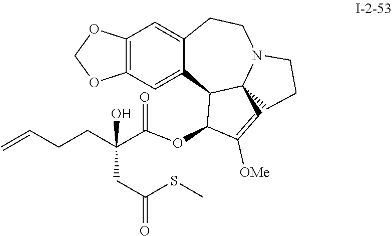

Compound I-2-53: obtained light pink colored amorphous solid (90% yield). ¹H NMR (400 MHz, CDCl₃): δ 6.58 (s, 1H), 6.50 (s, 1H), 5.98 (d, J=9.8 Hz, 1H), 5.82 (dd, J=6.5, 1.7 Hz, 2H), 5.69 (tt, J=13.1, 5.1 Hz, 1H), 5.03 (s, 1H), 4.98-4.85 (m, 2H), 3.76 (d, J=9.8 Hz, 1H), 3.65 (s, 3H), 3.41 (s, 1H), 3.12-3.00 (m, 2H), 2.97-2.83 (m, 1H), 2.61-2.52 (m, 2H), 2.50 (d, J=16.0 Hz, 1H), 2.34 (dd, J=14.1, 6.8 Hz, 1H), 2.19 (s, 3H), 2.11-2.02 (m, 1H), 2.07 (d, J=16.0 Hz, 1H), 1.93-1.75 (m, 3H), 1.72 (p, J=5.9, 4.8 Hz, 2H), 1.51 (tdd, J=25.4, 13.5, 4.8 Hz, 2H) ppm. HRMS-ESI (m/z): Calcd for C₂₄H₃₄NO₇ [M+H]⁺ 516.2057. Found 516.1978.

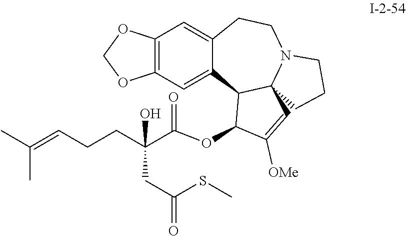

Compound I-2-54: obtained light pink colored amorphous solid (82% yield). ¹H NMR (400 MHz, CDCl₃): δ 6.59 (s, 1H), 6.50 (s, 1H), 5.99 (d, J=9.8 Hz, 1H), 5.83 (d, J=13.7 Hz, 2H), 5.03 (s, 1H), 5.02-4.96 (m, 1H), 3.77 (d, J=9.8 Hz, 1H), 3.65 (s, 3H), 3.39 (s, 1H), 3.16-3.01 (m, 2H), 2.98-2.85 (m, 1H), 2.62-2.52 (m, 2H), 2.48 (d, J=15.9 Hz, 1H), 2.35 (dd, J=14.2, 6.8 Hz, 1H), 2.21 (s, 3H), 2.01-1.94 (m, 1H), 1.98 (d, J=15.9 Hz, 1H), 1.92-1.85 (m, 1H), 1.74 (dd, J=13.7, 7.9 Hz, 3H), 1.64 (s, 3H), 1.54 (s, 3H), 1.52-1.35 (m, 3H) ppm. HRMS-ESI (m/z): Calcd for C₂₉H₃₈NO₇S [M+H]⁺ 544.2370. Found 544.2291.

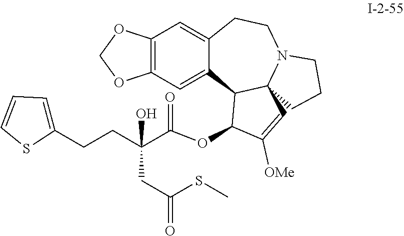

Compound I-2-55: obtained pink colored amorphous solid (82% yield). ¹H NMR (400 MHz, CDCl₃): δ 7.10 (dd, J=5.1, 1.3 Hz, 1H), 6.90 (dd, J=5.1, 3.4 Hz, 1H), 6.73 (d, J=2.4 Hz, 1H), 6.61 (s, 1H), 6.54 (s, 1H), 6.06 (d, J=9.8 Hz, 1H), 5.82 (dd, J=24.8, 1.6 Hz, 2H), 5.08 (s, 1H), 3.81 (d, J=9.8 Hz, 1H), 3.68 (s, 3H), 3.53 (s, 1H), 3.12 (ddd, J=21.9, 13.8, 7.7 Hz, 2H), 3.01-2.84 (m, 2H), 2.66-2.51 (m, 4H), 2.38 (dd, J=14.0, 6.7 Hz, 1H), 2.23 (s, 3H), 2.11 (d, J=16.0 Hz, 1H), 2.09-1.98 (m, 1H), 1.98-1.83 (m, 2H), 1.78 (ddt, J=17.6, 8.9, 4.7 Hz, 3H) ppm. HRMS-ESI (m/z): Calcd for C₂₉H₃₄NO₇S₂[M+H]⁺ 572.1777. Found 572.1698.

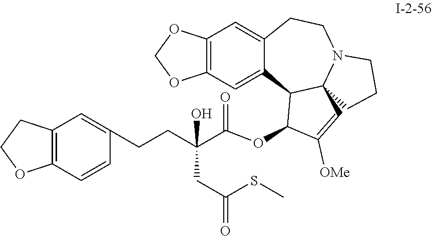

Compound I-2-56: obtained light pink amorphous solid (89% yield). ¹H NMR (400 MHz, CDCl₃): δ 6.94 (s, 1H), 6.82 (d, J=8.1 Hz, 1H), 6.68 (d, J=8.1 Hz, 1H), 6.58 (s, 1H), 6.55 (s, 1H), 6.06 (d, J=9.7 Hz, 1H), 5.80 (dd, J=33.6, 1.7 Hz, 2H), 5.09 (s, 1H), 4.53 (t, J=8.7 Hz, 2H), 3.81 (d, J=9.8 Hz, 1H), 3.70 (s, 3H), 3.49 (s, 1H), 3.16 (t, J=8.9 Hz, 2H), 3.11 (t, J=6.1 Hz, 2H), 3.01-2.86 (m, 1H), 2.66-2.54 (m, 3H), 2.52 (d, J=16.1 Hz, 1H), 2.36 (dd, J=14.1, 6.9 Hz, 1H), 2.32-2.26 (m, 1H), 2.23 (s, 3H), 2.09 (d, J=16.1 Hz, 1H), 2.07-1.98 (m, 1H), 1.92 (ddd, J=12.1, 7.6, 4.5 Hz, 1H), 1.83-1.61 (m, 4H) ppm. HRMS-ESI (m/z): Calcd for C₃₃H₃₈NO₈S [M+H]⁺ 608.2319. Found 607.2240.

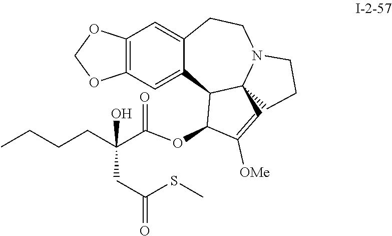

Compound I-2-57: column chromatography PE:EA=7: 1, 0.5% TEA to give yellow amorphous solid (49% yield). ¹H NMR (400 MHz, CDCl₃): δ 8.98 (d, J=2.9 Hz, 1H), 6.60 (s, 1H), 6.55 (s, 1H), 5.98 (d, J=8.8 Hz, 1H), 5.87 (dd, J=8.4, 5.6 Hz, 2H), 5.06 (s, 1H), 3.78 (d, J=9.8 Hz, 1H), 3.67 (s, 3H), 3.31 (s, 1H), 3.13-3.04 (m, 2H), 2.94 (ddd, J=12.4, 10.8, 6.7 Hz, 1H), 2.58 (td, J=9.3, 7.3 Hz, 2H), 2.36 (dd, J=13.9, 6.7 Hz, 1H), 2.29 (dt, J=17.0, 2.7 Hz, 1H), 2.06-2.01 (m, 1H), 1.99 (s, 1H), 1.90 (ddd, J=12.1, 7.8, 4.3 Hz, 1H), 1.75 (ddt, J=9.5, 8.1, 5.0 Hz, 3H), 1.67-1.62 (m, 1H), 1.52-1.44 (m, 2H), 1.12-1.01 (m, 1H), 0.90-0.82 (m, 4H) ppm. HRMS-ESI (m/z): Calcd for C₂₆H₃₄NO₇ [M+H]⁺ 472.2336. Found 471.2257.

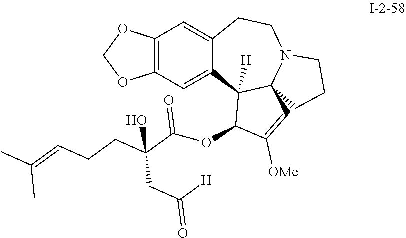

Compound I-2-58: column chromatography PE:EA=7: 1, 0.5% TEA to give a pale yellow amorphous solid (51% yield). ¹H NMR (400 MHz, CDCl₃): δ 8.98 (d, J=2.9 Hz, 1H), 6.59 (s, 1H), 6.55 (s, 1H), 5.98 (d, J=9.7 Hz, 1H), 5.86 (s, 2H), 5.06 (s, 1H), 5.00 (t, J=7.2 Hz, 1H), 3.79 (d, J=9.7 Hz, 1H), 3.67 (s, 3H), 3.37 (s, 1H), 3.15-3.01 (m, 2H), 2.97-2.89 (m, 1H), 2.64-2.52 (m, 2H), 2.36 (dd, J=14.0, 6.7 Hz, 1H), 2.27 (dd, J=17.1, 3.1 Hz, 1H), 2.10-1.95 (m, 3H), 1.95-1.84 (m, 2H), 1.84-1.70 (m, 4H), 1.65 (t, J=2.3 Hz, 3H), 1.56 (s, 3H) ppm. HRMS-ESI (m/z): Calcd for C₂₆H₃₆NO₇ [M+H]⁺ 498.2493. Found 498.2414.

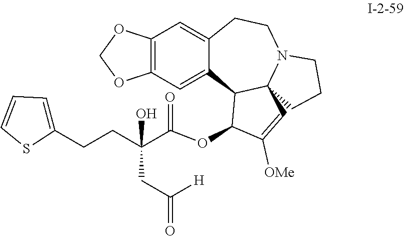

Compound I-2-59: Column chromatography PE:EA=5:1, 0.5% TEA to give yellow amorphous solid (54% yield). ¹H NMR (400 MHz, CDCl₃): δ 9.00 (d, J=2.5 Hz, 1H), 7.09 (d, J=5.2 Hz, 1H), 6.92-6.83 (m, 1H), 6.73 (d, J=3.4 Hz, 1H), 6.58 (s, 1H), 6.55 (s, 1H), 6.02 (d, J=9.7 Hz, 1H), 5.82 (d, J=9.8 Hz, 2H), 3.79 (d, J=9.8 Hz, 1H), 3.67 (s, 3H), 3.43 (s, 1H), 3.14-3.00 (m, 2H), 2.97-2.83 (m, 2H), 2.61-2.52 (m, 4H), 2.41-2.28 (m, 2H), 2.10-1.95 (m, 2H), 1.94-1.82 (m, 3H), 1.78-1.70 (m, 2H) ppm. HRMS-ESI (m/z): Calcd for C₂₈H₃₂NO₇S [M+H]⁺ 526.1900 Found 526.1821.

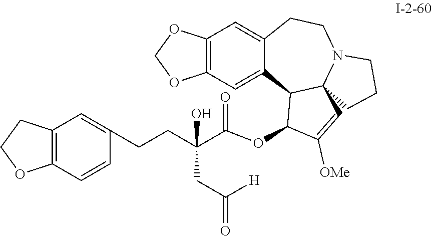

Compound I-2-60: column chromatography PE:EA=5:1, 0.5% TEA to give yellow amorphous solid (62% yield). ¹H NMR (400 MHz, CDCl₃): δ 9.01 (s, 1H), 6.96 (s, 1H), 6.85 (d, J=8.2 Hz, 1H), 6.68 (d, J=8.1 Hz, 1H), 6.58 (s, 2H), 6.05 (d, J=9.7 Hz, 1H), 5.82 (d, J=18.7 Hz, 2H), 5.11 (s, 1H), 4.54 (t, J=8.6 Hz, 2H), 3.82 (d, J=9.8 Hz, 1H), 3.72 (s, 3H), 3.43 (s, 1H), 3.24-3.02 (m, 4H), 3.01-2.87 (m, 1H), 2.65-2.55 (m, 3H), 2.41-2.25 (m, 2H), 2.08-1.98 (m, 3H), 1.98-1.85 (m, 1H), 1.82-1.70 (m, 4H) ppm. HRMS-ESI (m/z): Calcd for C₃₂H₃₆NO₈ [M+H]⁺ 562.2442 Found 562.2363.

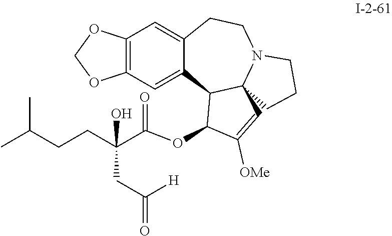

Compound I-2-61: column chromatography PE:EA=5:1, 0.5% TEA to give a yellow amorphous solid (68% yield). HRMS-ESI (m/z): Calcd for C₂₇H₃₆NO₇ [M+H]⁺ 486.2486 Found 486.2480.

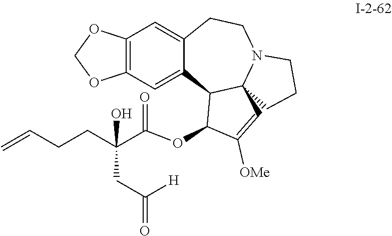

Compound I-2-62: column chromatography PE:EA=5:1, 0.5% TEA to give yellow amorphous solid (65% yield). HRMS-ESI (m/z): Calcd for C₂₆H₃₂NO₇ [M+H]⁺ 470.2173 Found 470.2170.

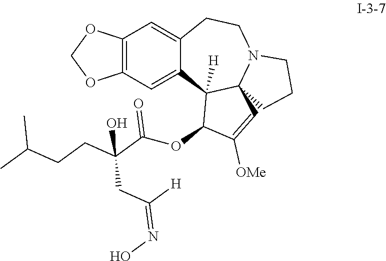

Compound I-3-7: column chromatography PE:EA=5:1, 0.5% TEA to give a white amorphous solid (42% yield). ¹H NMR (400 MHz, CDCl₃): 6.63-6.51 (m, 3H), 5.96 (d, J=10.6 Hz, 1H), 5.06 (s, 1H), 3.79 (d, J=9.7 Hz, 1H), 3.71 (s, 1H), 3.68 (s, 3H), 3.18-3.04 (m, 3H), 2.94 (td, J=11.6, 7.0 Hz, 1H), 2.59 (q, J=9.4, 8.8 Hz, 2H), 2.38 (dd, J=14.3, 6.9 Hz, 1H), 2.14-1.98 (m, 3H), 1.95-1.84 (m, 1H), 1.82-1.70 (m, 3H), 1.63-1.34 (m, 4H), 1.01-0.88 (m, 1H), 0.84 (dd, J=6.6, 2.2 Hz, 6H) ppm. HRMS-ESI (m/z): Calcd for C₂₇H₃₇N₂O₇ [M+H]⁺ 501.2602. Found 501.2523.

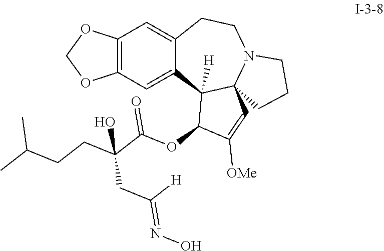

Compound I-3-8: Column chromatography PE:EA=5:1, 0.5% TEA to give white amorphous solid (32% yield). ¹H NMR (400 MHz, CDCl₃): δ 8.11 (s, 1H), 6.63 (s, 1H), 6.59 (s, 1H), 6.29 (dd, J=6.5, 3.6 Hz, 1H), 5.96 (d, J=9.7 Hz, 1H), 5.88 (q, J=1.7 Hz, 2H), 5.09 (s, 1H), 3.82 (d, J=9.7 Hz, 1H), 3.70 (s, 3H), 3.20-3.06 (m, 3H), 2.97 (td, J=11.5, 6.8 Hz, 1H), 2.66-2.57 (m, 2H), 2.55 (dd, J=16.7, 6.6 Hz, 1H), 2.41 (dd, J=14.1, 6.7 Hz, 1H), 2.06-2.02 (m, 1H), 1.92 (ddd, J=12.2, 8.0, 4.1 Hz, 1H), 1.78 (tq, J=8.5, 4.2, 3.7 Hz, 2H), 1.68 (dd, J=16.8, 3.6 Hz, 1H), 1.64-1.44 (m, 4H), 0.96 (td, J=12.4, 6.2 Hz, 1H), 0.86 (dd, J=6.6, 2.5 Hz, 6H) ppm. HRMS-ESI (m/z): Calcd for C₂₇H₃₇N₂O₇ [M+H]⁺ 501.2602. Found 501.2523.

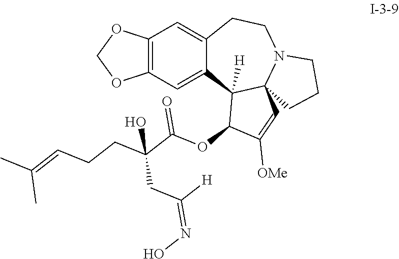

Compound I-3-9: column chromatography PE:EA=5:1, 0.5% TEA to give a white amorphous solid (22% yield). ¹H NMR (400 MHz, CDCl₃): δ 6.57 (dd, J=6.8, 3.2 Hz, 3H), 5.95 (d, J=9.7 Hz, 1H), 5.86 (dd, J=12.8, 1.7 Hz, 2H), 5.06 (s, 1H), 5.02 (t, J=7.0 Hz, 1H), 3.79 (d, J=9.7 Hz, 1H), 3.67 (s, 3H), 3.32 (s, 1H), 3.10 (ddd, J=21.7, 13.9, 7.9 Hz, 2H), 2.93 (td, J=11.6, 6.9 Hz, 1H), 2.63-2.53 (m, 2H), 2.37 (dd, J=14.1, 6.8 Hz, 1H), 2.11-1.92 (m, 2H), 1.89 (ddd, J=12.1, 7.9, 4.2 Hz, 1H), 1.82-1.67 (m, 2H), 1.65 (s, 3H), 1.57 (s, 3H), 1.54-1.39 (m, 1H), 0.92-0.80 (m, 4H) ppm. HRMS-ESI (m/z): Calcd for C₂₈H₃₇N₂O₇ [M+H]⁺ 513.2602. Found 513.2523.

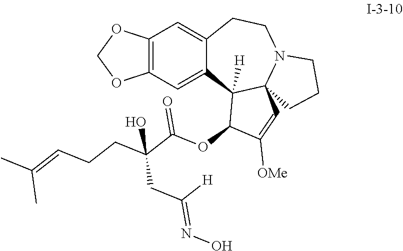

Compound I-3-10: column chromatography PE:EA=5:1, 0.5% TEA to give white amorphous solid (23% yield). ¹H NMR (400 MHz, CDCl₃): δ 6.60 (s, 2H), 6.56 (s, 1H), 6.27 (dd, J=6.5, 3.7 Hz, 1H), 5.93 (d, J=9.5 Hz, 1H), 5.87-5.80 (m, 2H), 5.07 (s, 1H), 5.02 (t, J=7.9 Hz, 1H), 3.79 (d, J=9.6 Hz, 1H), 3.68 (s, 3H), 3.16-3.03 (m, 2H), 2.94 (td, J=11.5, 6.8 Hz, 1H), 2.63-2.54 (m, 2H), 2.50 (dd, J=16.7, 6.6 Hz, 1H), 2.43-2.32 (m, 1H), 2.10-1.98 (m, 1H), 1.93-1.86 (m, 1H), 1.83-1.68 (m, 2H), 1.65 (s, 3H), 1.57 (s, 3H), 0.92-0.80 (m, 4H) ppm. HRMS-ESI (m/z): Calcd for C₂₈H₃₇N₂O, [M+H]⁺ 513.2602. Found 513.2523.

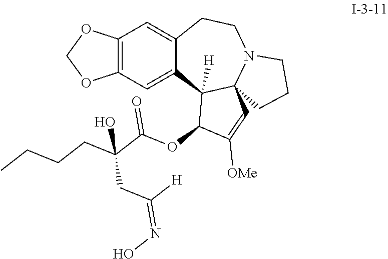

Compound I-3-11: Column chromatography PE:EA=5:1, 0.5% TEA to give white amorphous solid (28% yield). ¹H NMR (400 MHz, CDCl₃): δ 6.59 (s, 1H), 6.58 (s, 1H), 6.53 (dd, J=7.9, 4.2 Hz, 1H), 5.95 (d, J=9.5 Hz, 1H), 5.87 (dd, 2H), 5.05 (s, 1H), 3.79 (d, J=9.7 Hz, 1H), 3.67 (s, 3H), 3.29 (s, 1H), 3.18-3.03 (m, 2H), 2.94 (td, J=11.5, 6.9 Hz, 1H), 2.64-2.53 (m, 2H), 2.37 (dd, J=14.1, 6.8 Hz, 1H), 2.14-1.98 (m, 3H), 1.90 (ddd, J=12.1, 8.1, 4.1 Hz, 1H), 1.76 (ddt, J=14.3, 8.8, 4.4 Hz, 4H), 1.61-1.40 (m, 3H), 0.92-0.80 (m, 4H) ppm. HRMS-ESI (m/z): Calcd for C₂₆H₃₅N₂O₇ [M+H]⁺ 487.2445. Found 487.2366.

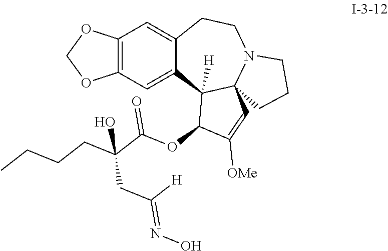

Compound I-3-12: column chromatography PE:EA=5:1, 0.5% TEA to give white amorphous solid (35% yield). ¹H NMR (400 MHz, CDCl₃): δ 6.61 (s, 1H), 6.56 (s, 1H), 6.26 (dd, J=6.5, 3.6 Hz, 1H), 5.93 (d, J=9.8 Hz, 1H), 5.86 (d, J=1.8 Hz, 2H), 5.06 (s, 1H), 3.79 (d, J=9.7 Hz, 1H), 3.68 (s, 3H), 3.13-3.05 (m, 2H), 2.99-2.90 (m, 1H), 2.64-2.48 (m, 3H), 2.39 (dd, J=14.1, 6.8 Hz, 1H), 2.11-1.98 (m, 1H), 1.95-1.84 (m, 1H), 1.83-1.60 (m, 4H), 1.61-1.44 (m, 2H), 1.09-0.99 (m, 1H), 0.91-0.80 (m, 5H) ppm. HRMS-ESI (m/z): Calcd for C₂₆H₃₅N₂O₇[M+H]⁺ 487.2445. Found 487.2366.

### Example 21: Preparation of Oxime Ester Compounds

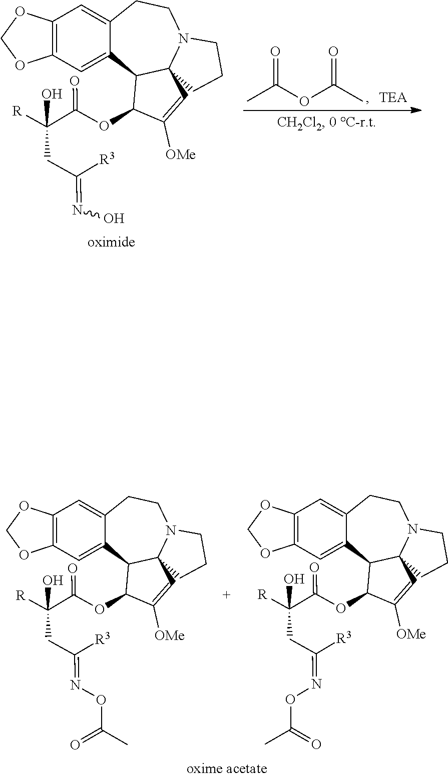

General operation: under argon protection at 0 °C, Et₃N (3 mmol) was added dropwise to a solution of CH₂Cl₂ (5 mL) in oxime (1 mmol), followed by acetic anhydride (1.2 mmol), the drop was completed and then moved to room temperature, the reaction was monitored on a TLC plate until complete. The reaction was quenched by addition of saturated NaHCO₃, extracted with CH₂Cl₂, the organic phases were combined, washed with saturated NaCl and dried over anhydrous Na₂SO₄. The solvent was removed by spin evaporation to give the crude product of oxime acetate.

With reference to the above method, the following compounds were obtained:

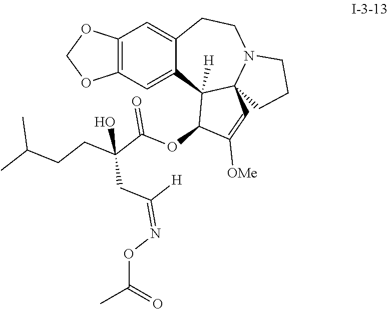

Compound I-3-13: column chromatography PE:EA=5:1, 0.5% TEA to give white amorphous solid (87% yield). HRMS-ESI (m/z): Calcd for C₂₉H₃₈N₂O₈ [M+H]⁺ 543.2707. Found 543.2628. ¹H NMR (400 MHz, CDCl₃): δ 6.82 (dd, J=8.8, 3.8 Hz, 1H), 6.53 (d, J=3.7 Hz, 2H), 5.85 (d, J=8.8 Hz, 1H), 5.81 (dd, J=18.3, 1.6 Hz, 2H), 5.01 (s, 1H), 3.74 (d, J=9.7 Hz, 1H), 3.64 (s, 1H), 3.61 (s, 3H), 3.05-2.97 (m, 4H), 2.87 (td, J=11.5, 6.8 Hz, 1H), 2.57-2.49 (m, 2H), 2.29 (ddd, J=18.0, 14.2, 7.8 Hz, 3H), 2.06 (s, 3H), 1.82 (ddd, J=14.9, 10.6, 4.4 Hz, 2H), 1.70 (dt, J=13.1, 7.4 Hz, 2H), 1.55 (td, J=13.0, 4.3 Hz, 1H), 1.40 (td, J=13.5, 13.1, 5.1 Hz, 1H), 0.88-0.82 (m, 1H), 0.77 (dd, J=6.6, 2.4 Hz, 6H) ppm. HRMS-ESI (m/z): Calcd for C₂₉H₃₉N₂O₈ [M+H]⁺ 543.2707. Found 543.2628.

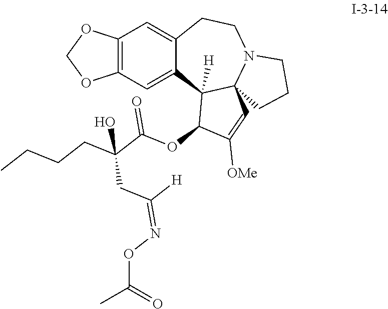

Compound I-3-14: column chromatography PE:EA=5:1, 0.5% TEA to give white amorphous solid (55% yield). ¹H NMR (400 MHz, CDCl₃): δ 6.89 (dd, J=8.8, 3.8 Hz, 1H), 6.60 (s, 1H), 6.59 (s, 1H), 5.92 (d, J=9.7 Hz, 1H), 5.87 (dd, J=17.3, 1.6 Hz, 2H), 5.07 (s, 1H), 3.80 (d, J=9.7 Hz, 1H), 3.68 (s, 3H), 3.13-3.06 (m, 2H), 3.04 (s, 1H), 2.98-2.89 (m, 1H), 2.60 (t, J=8.4 Hz, 2H), 2.43-2.29 (m, 2H), 2.13 (s, 3H), 2.10-1.97 (m, 2H), 1.95-1.85 (m, 2H), 1.80-1.70 (m, 3H), 1.66-1.55 (m, 3H), 1.47 (td, J=13.5, 12.7, 3.9 Hz, 1H), 0.86 (t, J=7.1 Hz, 3H) ppm. HRMS-ESI (m/z): Calcd for C₂₈H₃₇N₂O [M+H]⁺ 529.2551. Found 528.2472.

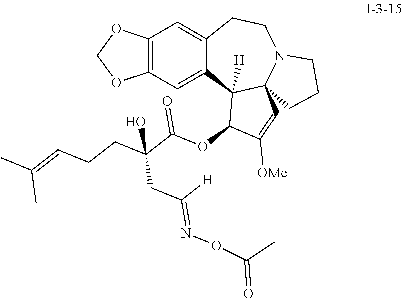

Compound I-3-15: column chromatography PE:EA=5:1, 0.5% TEA to give a white amorphous solid (49% yield). ¹H NMR (400 MHz, CDCl₃): δ 6.92 (dd, J=8.8, 3.9 Hz, 1H), 6.60 (s, 1H), 6.58 (s, 1H), 5.91 (d, J=9.7 Hz, 1H), 5.86 (dd, J=9.7, 1.6 Hz, 2H), 5.08 (s, 1H), 5.06-4.97 (m, 1H), 3.81 (d, J=9.7 Hz, 1H), 3.68 (s, 3H), 3.14-3.03 (m, 2H), 3.09 (s, 1H), 2.92 (td, J=11.5, 6.8 Hz, 1H), 2.62-2.54 (m, 2H), 2.42-2.26 (m, 2H), 2.13 (s, 3H), 2.10-1.97 (m, 2H), 1.97-1.79 (m, 3H), 1.81-1.71 (m, 3H), 1.65 (s, 3H), 1.56 (s, 3H), 1.55-1.43 (m, 1H) ppm. HRMS-ESI (m/z): Calcd for C₃₀H₃₉N₂O₈ [M+H]⁺ 555.2707. Found 555.2628.

### Example 22: Preparation of Oxime Esters

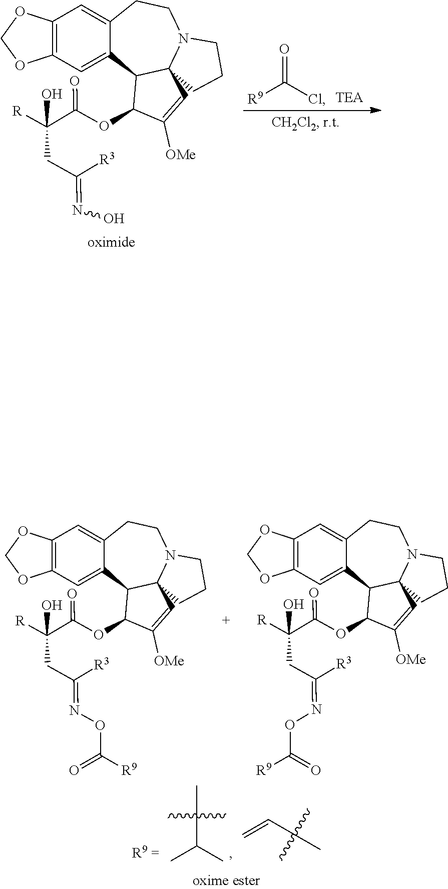

General operation: Et₃N (3 mmol) was added dropwise to a solution of oxime (1 mmol) in CH₂Cl₂ (5 mL) under Ar atmosphere, followed by a drop of chloroyl chloride (1.2 mmol), and the reaction was monitored on a TLC plate until complete. Quenched by addition of saturated NaHCO₃, extracted with CH₂Cl₂, the organic phases were combined, washed with saturated NaCl and dried over anhydrous Na₂SO₄. The solvent was removed by spin evaporation to give the oxime ester crude product.

Referring to the above method, the following compounds were obtained:

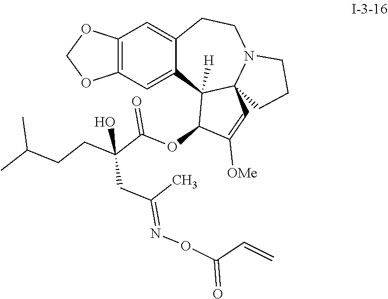

Compound I-3-16: column chromatography column chromatography PE:EA=5:1, 0.5% TEA to give light yellow amorphous solid (44% yield). ¹′H NMR (400 MHz, CDCl₃): δ 6.58 (d, J=17.9 Hz, 2H), 6.50 (d, J=17.5 Hz, 1H), 6.22 (ddd, J=17.4, 10.5, 1.9 Hz, 1H), 5.97-5.80 (m, 4H), 5.03 (s, 1H), 3.80 (d, J=8.8 Hz, 1H), 3.66 (s, 3H), 3.36 (s, 1H), 3.11 (dd, J=23.9, 8.5 Hz, 2H), 2.92 (q, J=10.5 Hz, 1H), 2.62-2.54 (m, 2H), 2.39 (d, J=14.8 Hz, 1H), 2.33 (d, J=16.9 Hz, 1H), 2.09-1.96 (m, 1H), 1.93-1.81 (m, 2H), 1.79 (d, J=1.8 Hz, 3H), 1.76-1.66 (m, 3H), 1.55-1.48 (m, 1H), 1.43 (dt, J=11.8, 5.1 Hz, 1H), 1.30-1.21 (m, 1H), 0.97-0.87 (m, 1H), 0.83 (d, J=6.7 Hz, 6H) ppm. HRMS-ESI (m/z): Calcd for C₃₁H₄₁N₂O₈ [M+H]⁺ 569.2864. Found 569.2785.

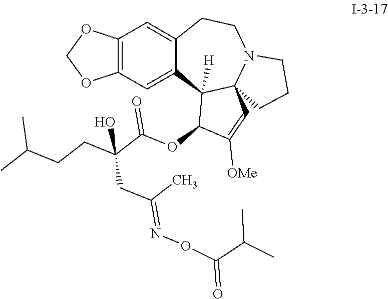

Compound I-3-17: Column chromatography PE:EA=5:1, 0.5% TEA to give light yellow amorphous solid (57% yield). ¹H NMR (400 MHz, CDCl₃): δ 6.61 (s, 1H), 6.56 (s, 1H), 5.93 (d, J=9.7 Hz, 1H), 5.85 (dd, J=13.7, 1.5 Hz, 2H), 5.03 (s, 1H), 3.79 (d, J=9.7 Hz, 1H), 3.66 (s, 3H), 3.35 (s, 1H), 3.21-3.04 (m, 2H), 2.93 (q, J=11.2 Hz, 1H), 2.67 (p, J=7.0 Hz, 1H), 2.62-2.54 (m, 2H), 2.38 (dd, J=14.2, 6.9 Hz, 1H), 2.30 (d, J=15.0 Hz, 1H), 2.10-1.96 (m, 1H), 1.93-1.86 (m, 1H), 1.82 (d, J=15.0 Hz, 1H), 1.75 (s, 3H), 1.57-1.36 (m, 4H), 1.23 (dd, J=7.0, 1.2 Hz, 6H), 1.18-1.06 (m, 1H), 0.95-0.87 (m, 2H), 0.83 (dd, J=6.6, 2.7 Hz, 6H) ppm. HRMS-ESI (m/z): Calcd for C₃₂H₄₅N₂O₈ [M+H]⁺ 585.3177. Found 584.3098.

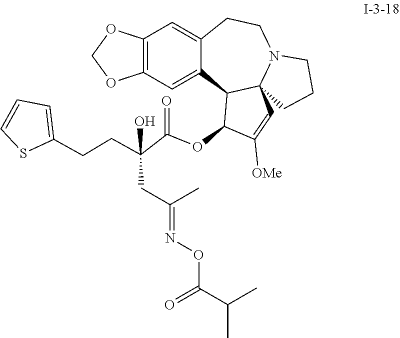

Compound I-3-18: Column chromatography PE:EA=2:1, 0.5% TEA to give light yellow amorphous solid (87% yield). ¹H NMR (400 MHz, CDCl₃): δ 7.09 (d, J=5.1 Hz, 1H), 6.90 (dd, J=5.1, 3.5 Hz, 1H), 6.73 (t, J=4.2 Hz, 1H), 6.60 (s, 1H), 6.56 (s, 1H), 6.01 (d, J=9.3 Hz, 1H), 5.79 (d, J=17.8 Hz, 2H), 5.06 (s, 1H), 3.81 (d, J=9.8 Hz, 1H), 3.67 (s, 3H), 3.53 (s, 1H), 3.18 (dt, J=13.0, 6.5 Hz, 1H), 3.15-3.04 (m, 1H), 3.00-2.79 (m, 2H), 2.73-2.48 (m, 4H), 2.40 (dd, J=14.2, 6.9 Hz, 1H), 2.33 (d, J=15.2 Hz, 1H), 2.02 (dd, J=20.7, 11.0 Hz, 2H), 1.96-1.81 (m, 3H), 1.63 (s, 1H), 1.27-1.21 (m, 6H) ppm. HRMS-ESI (m/z): Calcd for C₃₃H₄₁N₂O₈S [M+H]⁺ 625.2581. Found 625.2505.

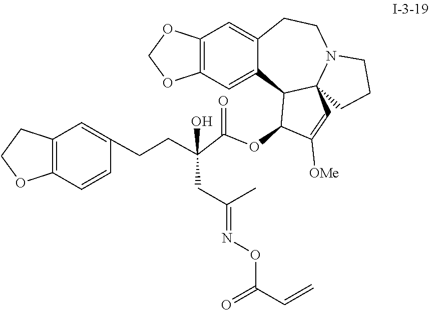

Compound I-3-19: column chromatography PE:EA=2:1, 0.5% TEA to give a white amorphous solid (89% yield). ¹H NMR (400 MHz, CDCl₃): δ 6.95 (s, 1H), 6.84 (d, J=8.1 Hz, 1H), 6.67 (d, J=8.1 Hz, 1H), 6.59 (s, 1H), 6.58 (s, 1H), 6.51 (dd, J=17.4, 1.4 Hz, 1H), 6.22 (dd, J=17.4, 10.5 Hz, 1H), 5.99 (d, J=9.7 Hz, 1H), 5.91 (dd, J=10.5, 1.5 Hz, 1H), 5.75 (dd, J=40.8, 1.6 Hz, 2H), 5.08 (s, 1H), 4.53 (t, J=8.6 Hz, 2H), 3.82 (d, J=9.7 Hz, 1H), 3.68 (s, 3H), 3.46 (s, 1H), 3.32-3.14 (m, 1H), 3.16 (t, J=8.8 Hz, 2H), 3.09 (td, J=8.5, 5.1 Hz, 1H), 2.93 (td, J=11.7, 7.0 Hz, 1H), 2.64-2.56 (m, 2H), 2.49 (td, J=12.9, 4.9 Hz, 1H), 2.40 (dd, J=14.4, 6.7 Hz, 1H), 2.36 (d, J=17.4 Hz, 1H), 2.19 (td, J=13.0, 4.7 Hz, 1H), 2.08-1.85 (m, 2H), 1.96 (d, J=17.4 Hz, 1H), 1.83 (s, 3H), 1.80-1.65 (m, 4H) ppm. HRMS-ESI (m/z): Calcd for C₃₆H₄₁N₂O₉ [M+H]⁺ 645.2810. Found 645.2734.

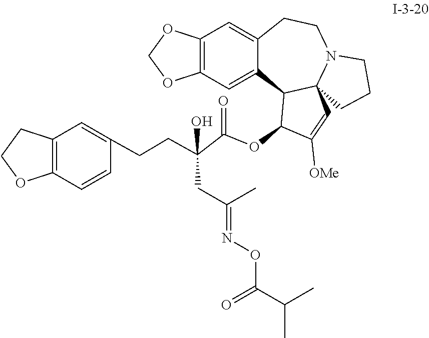

Compound I-3-20: column chromatography PE:EA=2:1, 0.5% TEA to give a pale yellow amorphous solid (38% yield). ¹H NMR (400 MHz, CDCl₃): δ 6.96 (s, 1H), 6.84 (d, J=8.1 Hz, 1H), 6.67 (d, J=8.1 Hz, 1H), 6.58 (d, J=6.3 Hz, 2H), 5.99 (d, J=9.7 Hz, 1H), 5.76 (dd, J=33.8, 1.6 Hz, 2H), 5.08 (s, 1H), 4.53 (t, J=8.6 Hz, 2H), 3.83 (d, J=9.7 Hz, 1H), 3.69 (s, 3H), 3.44 (s, 1H), 3.16 (t, J=8.8 Hz, 2H), 3.13-3.04 (m, 2H), 2.94 (td, J=11.5, 6.9 Hz, 1H), 2.72-2.54 (m, 3H), 2.56-2.44 (m, 1H), 2.40 (dd, J=13.7, 7.2 Hz, 1H), 2.33 (d, J=15.0 Hz, 1H), 2.22 (td, J=12.9, 4.5 Hz, 1H), 2.08-1.98 (m, 1H), 1.90 (dd, J=13.2, 5.1 Hz, 2H), 1.79 (s, 3H), 1.74 (dd, J=12.7, 5.4 Hz, 3H), 1.68-1.48 (m, 1H), 1.23 (d, J=7.0 Hz, 6H), 1.20-1.08 (m, 2H) ppm. HRMS-ESI (m/z): Calcd for C₃₇H₄₅N₂O₉[M+H]⁺ 661.3120. Found 661.3122.

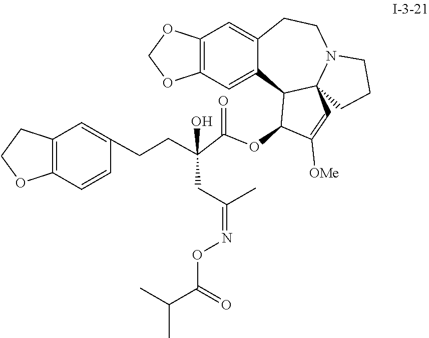

Compound I-3-21: Column chromatography PE:EA=2:1, 0.5% TEA to give light yellow amorphous solid (30% yield). ¹H NMR (400 MHz, CDCl₃): δ 6.92 (s, 1H), 6.82 (d, J=8.1 Hz, 1H), 6.66 (d, J=8.1 Hz, 1H), 6.59 (d, J=8.7 Hz, 2H), 5.99 (d, J=9.6 Hz, 1H), 5.74 (dd, J=54.6, 1.6 Hz, 2H), 5.12 (s, 1H), 4.53 (t, J=8.7 Hz, 2H), 3.85 (d, J=9.6 Hz, 1H), 3.71 (s, 3H), 3.15 (t, J=8.8 Hz, 2H), 3.21-3.06 (m, 2H), 3.05 (s, 1H), 2.95 (dt, J=11.5, 5.5 Hz, 1H), 2.74 (d, J=13.7 Hz, 1H), 2.67-2.52 (m, 3H), 2.45-2.41 (m, 1H), 2.38 (dd, J=13.7, 7.2 Hz, 1H), 2.15-1.98 (m, 2H), 1.99-1.91 (m, 1H), 1.90 (s, 3H), 1.86-1.61 (m, 5H), 1.16 (dd, J=15.8, 7.0 Hz, 6H) ppm. HRMS-ESI (m/z): Calcd for C₃₇H₄₅N₂O₉[M+H]⁺ 661.3120. Found, 661.3122.

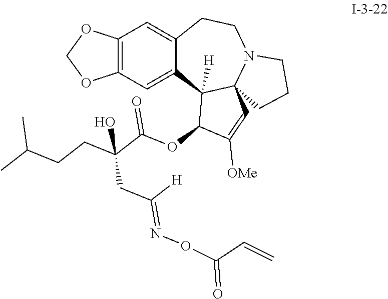

Compound I-3-22: Column chromatography PE:EA=5:1, 0.5% TEA to give yellow amorphous solid (89% yield). ¹H NMR (400 MHz, CDCl₃): δ 6.63-6.45 (m, 3H), 6.14 (dd, J=17.3, 10.5 Hz, 1H), 5.96-5.83 (m, 3H), 5.80 (d, J=11.5 Hz, 2H), 5.06 (s, 1H), 3.78 (d, J=9.6 Hz, 1H), 3.65 (s, 3H), 3.17-3.01 (m, 3H), 3.00-2.87 (m, 1H), 2.66-2.54 (m, 2H), 2.42-2.29 (m, 2H), 2.11-1.96 (m, 1H), 1.95-1.82 (m, 1H), 1.80-1.67 (m, 2H), 1.66-1.51 (m, 2H), 1.51-1.30 (m, 4H), 0.87-0.77 (m, 6H) ppm. HRMS-ESI (m/z): Calcd for C₃₀H₃₉N₂O₈ [M+H]⁺ 555.2707. Found 555.2628.

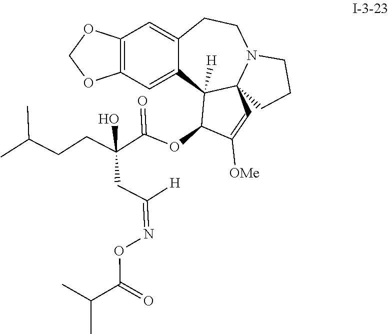

Compound I-3-23: column chromatography PE:EA=5:1, 0.5% TEA to give white amorphous solid (43% yield). ¹H NMR (400 MHz, CDCl₃): δ 7.03 (dd, J=8.9, 3.8 Hz, 1H), 6.58 (d, J=7.0 Hz, 2H), 5.91 (d, J=9.7 Hz, 1H), 5.84 (dd, J=9.8, 1.5 Hz, 2H), 5.06 (s, 1H), 3.79 (d, J=9.7 Hz, 1H), 3.66 (s, 3H), 3.12-3.03 (m, 3H), 2.92 (td, J=11.5, 6.8 Hz, 1H), 2.63-2.53 (m, 4H), 2.40-2.28 (m, 2H), 1.89 (ddd, J=12.2, 7.8, 4.4 Hz, 1H), 1.76 (ddd, J=17.9, 11.8, 5.5 Hz, 4H), 1.59 (td, J=13.1, 4.3 Hz, 1H), 1.49-1.39 (m, 2H), 1.20 (dd, J=7.0, 1.5 Hz, 6H), 0.89 (ddd, J=12.3, 7.5, 4.5 Hz, 1H), 0.82 (dd, J=6.6, 3.1 Hz, 6H) ppm. HRMS-ESI (m/z): Calcd for C₃₁H₄₃N₂O₈ [M+H]⁺ 571.3020. Found 571.2941.

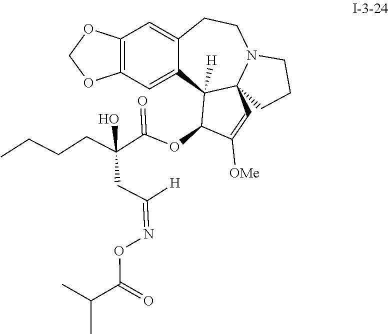

Compound I-3-24: column chromatography PE:EA=5:1, 0.5% TEA to give white amorphous solid (43% yield). ¹H NMR (400 MHz, CDCl₃): δ 7.03 (dd, J=8.9, 3.8 Hz, 1H), 6.58 (d, J=7.0 Hz, 2H), 5.91 (d, J=9.7 Hz, 1H), 5.84 (dd, J=9.8, 1.5 Hz, 2H), 5.06 (s, 1H), 3.79 (d, J=9.7 Hz, 1H), 3.66 (s, 3H), 3.12-3.03 (m, 3H), 2.92 (td, J=11.5, 6.8 Hz, 1H), 2.63-2.53 (m, 4H), 2.40-2.28 (m, 2H), 1.89 (ddd, J=12.2, 7.8, 4.4 Hz, 1H), 1.76 (ddd, J=17.9, 11.8, 5.5 Hz, 4H), 1.59 (td, J=13.1, 4.3 Hz, 1H), 1.49-1.39 (m, 2H), 1.20 (dd, J=7.0, 1.5 Hz, 6H), 0.89 (ddd, J=12.3, 7.5, 4.5 Hz, 1H), 0.82 (dd, J=6.6, 3.1 Hz, 6H) ppm. HRMS-ESI (m/z): Calcd for C₃₀H₄₁N₂O₈ [M+H]⁺ 557.2860. Found 557.2785.

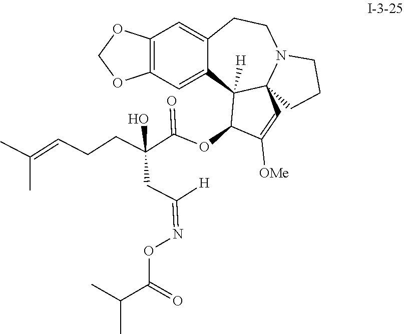

Compound I-3-25: column chromatography column chromatography PE:EA=5:1, 0.5% TEA to give white amorphous solid (87% yield). ¹H NMR (400 MHz, CDCl₃): δ 7.09 (dd, J=8.8, 3.9 Hz, 1H), 6.59 (s, 1H), 6.59 (s, 1H), 5.90 (d, J=5.1 Hz, 1H), 5.85 (dd, J=1.6 Hz, 2H), 5.08 (s, 1H), 5.02 (t, J=6.8 Hz, 1H), 3.81 (d, J=9.7 Hz, 1H), 3.68 (s, 3H), 3.08 (q, J=6.0 Hz, 3H), 2.93 (td, J=11.5, 7.0 Hz, 1H), 2.61 (dt, J=13.9, 7.0 Hz, 3H), 2.38 (dd, J=14.0, 6.7 Hz, 1H), 2.31 (dd, J=14.8, 8.8 Hz, 1H), 2.10-1.97 (m, 1H), 1.95-1.87 (m, 1H), 1.82-1.71 (m, 4H), 1.65 (s, 3H), 1.56 (s, 3H), 1.55-1.42 (m, 1H), 1.22 (dd, J=7.0, 1.3 Hz, 6H), 0.85 (d, J=6.6, 3.5 Hz, 2H) ppm. HRMS-ESI (m/z): Calcd for C₃₂H₄₃N₂O₈ [M+H]⁺ 583.3020. Found 583.2941.

### Example 23: Preparation of Oxime Esters

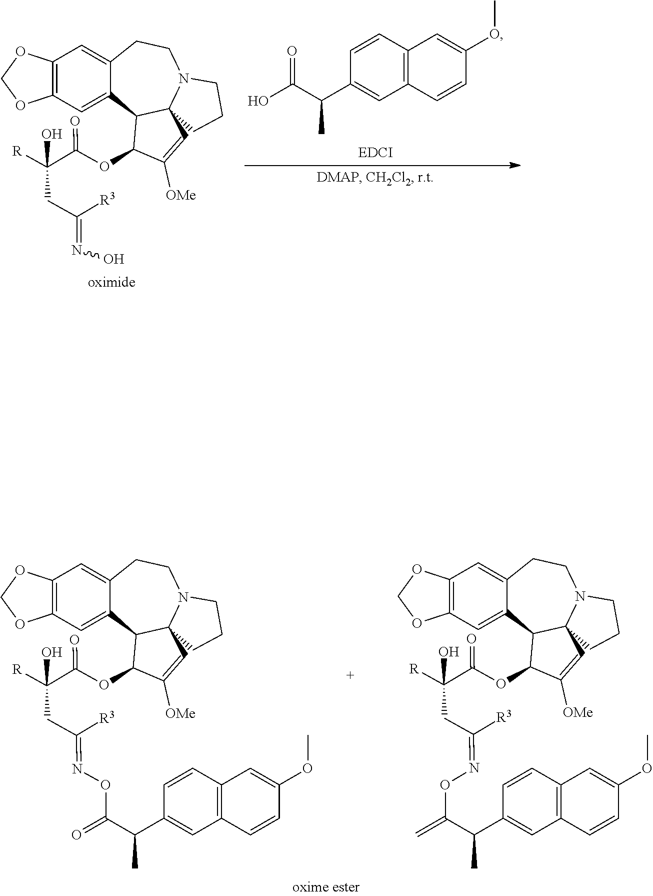

General operation: To a solution of CH₂Cl₂ (2 mL) of oxime (1 mmol) was added CH₂Cl₂ (2 mL) of naproxen (1 mmol), EDCI (1.1 mmol), and DMAP (0.2 mmol) dropwise in an General operation, and the reaction was monitored on a TLC plate until complete. The reaction was quenched by addition of saturated NaHCO₃, extracted with CH₂Cl₂, the organic phases were combined, washed with saturated NaCl and dried over anhydrous Na₂SO₄. The solvent was removed by spin evaporation to give the oxime ester crude product. Referring to the above method, the following compounds were obtained:

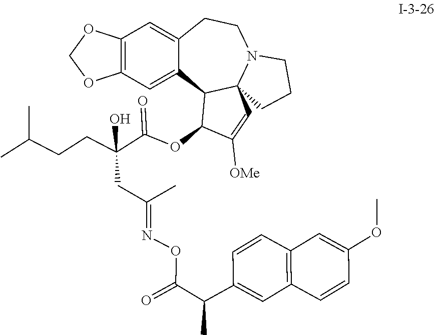

Compound (I-3-26): column chromatography PE:EA=4:1, 0.5% TEA to give light yellow amorphous solid (44% yield). ¹H NMR (400 MHz, CDCl₃): δ 7.70 (d, J=9.0 Hz, 3H), 7.42 (dd, J=8.4, 1.9 Hz, 1H), 7.16-7.09 (m, 2H), 6.56 (s, 1H), 6.47 (s, 1H), 5.90 (d, J=9.6 Hz, 1H), 5.69 (dd, J=23.7, 1.5 Hz, 2H), 5.01 (s, 1H), 3.91 (s, 3H), 3.72 (d, J=8.9 Hz, 1H), 3.64 (s, 3H), 3.28 (s, 1H), 3.15-3.03 (m, 2H), 2.95-2.85 (m, 1H), 2.61-2.53 (m, 2H), 2.34 (dd, J=14.1, 6.8 Hz, 1H), 2.25 (d, J=15.0 Hz, 1H), 2.04 (s, 3H), 2.02-1.93 (m, 2H), 1.92-1.81 (m, 2H), 1.79-1.71 (m, 3H), 1.63 (d, J=7.2 Hz, 3H), 1.54-1.36 (m, 4H), 0.82 (dd, J=6.6, 2.7 Hz, 6H) ppm. HRMS-ESI (m/z): Calcd for C₄₂H₅₁N₂O₉ [M+H]⁺ 727.3594. Found 727.3516.

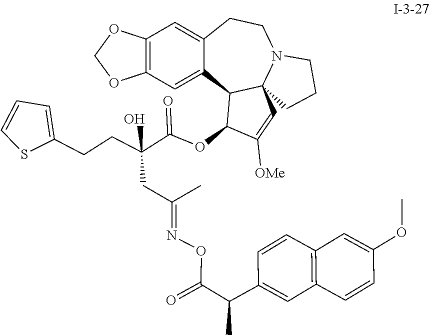

Compound I-3-27: column chromatography PE:EA=2:1, 0.5% TEA to give light yellow amorphous solid (45% yield). ¹H NMR (400 MHz, CDCl₃): δ 7.71 (d, J=9.1 Hz, 3H), 7.43 (d, J=8.4 Hz, 1H), 7.12 (ddd, J=15.9, 12.3, 3.8 Hz, 3H), 6.89 (dd, J=5.1, 3.4 Hz, 1H), 6.73 (d, J=3.3 Hz, 1H), 6.56 (s, 1H), 6.48 (s, 1H), 5.98 (d, J=9.7 Hz, 1H), 5.65 (dd, J=55.3, 1.5 Hz, 2H), 5.04 (s, 1H), 3.91 (s, 3H), 3.75 (s, 1H), 3.64 (s, 3H), 3.51 (s, 1H), 3.17-3.04 (m, 2H), 2.98-2.80 (m, 2H), 2.65-2.48 (m, 3H), 2.37 (dd, J=14.1, 6.7 Hz, 1H), 2.28 (d, J=15.3 Hz, 1H), 2.00 (dd, J=12.9, 8.9 Hz, 1H), 1.95-1.81 (m, 4H), 1.81-1.70 (m, 3H), 1.63 (d, J=7.2 Hz, 3H), 1.57 (s, 3H) ppm. HRMS-ESI(m/z): Calcd for C₄₃H₄₇N₂O₉S [M+H]⁺ 767.2999. Found 767.2964.

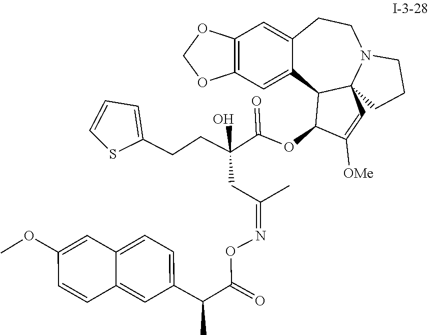

Compound I-3-28: Column chromatography PE:EA=2:1, 0.5% TEA to give light yellow amorphous solid (31% yield). ¹H NMR (400 MHz, CDCl₃): δ 7.68-7.62 (m, 3H), 7.38 (dd, J=8.4, 1.8 Hz, 1H), 7.14-7.03 (m, 3H), 6.86 (dd, J=5.2, 3.4 Hz, 1H), 6.60 (d, J=3.7 Hz, 1H), 6.50 (s, 1H), 6.49 (s, 1H), 5.97 (d, J=9.5 Hz, 1H), 5.73 (dd, J=53.8, 1.6 Hz, 2H), 5.08 (s, 1H), 3.89 (s, 3H), 3.76 (d, J=9.4 Hz, 1H), 3.66 (s, 3H), 3.53-3.43 (m, 1H), 3.14-3.02 (m, 2H), 2.92 (dd, J=11.2, 7.0 Hz, 1H), 2.87 (s, 1H), 2.65-2.53 (m, 3H), 2.52 (d, J=14.1 Hz, 1H), 2.36-2.22 (m, 2H), 2.02-1.98 (m, 1H), 1.91 (td, J=12.4, 11.9, 5.2 Hz, 2H), 1.78 (s, 3H), 1.77-1.73 (m, 2H), 1.70 (d, J=14.1 Hz, 2H), 1.63 (d, J=7.1 Hz, 3H) ppm. HRMS-ESI (m/z): Calcd for C₄₃H₄₇N₂OS [M+H]⁺ 767.2999. Found 767.2964.

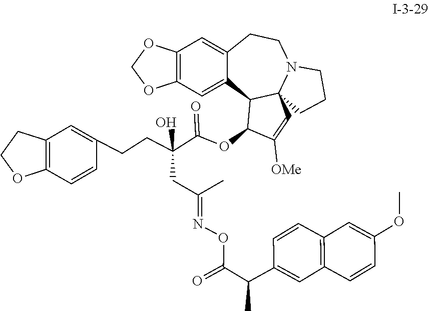

Compound I-3-29: Column chromatography PE:EA=2:1, 0.5% TEA to give light yellow amorphous solid (40% yield). ¹H NMR (400 MHz, CDCl₃): δ 7.70 (d, J=8.9 Hz, 2H), 7.42 (d, J=8.6 Hz, 1H), 7.17-7.08 (m, 2H), 6.94 (s, 1H), 6.83 (d, J=7.9 Hz, 1H), 6.67 (d, J=8.1 Hz, 1H), 6.52 (d, J=16.3 Hz, 2H), 5.96 (d, J=9.7 Hz, 1H), 5.71 (s, 1H), 5.54 (s, 1H), 5.06 (s, 1H), 4.53 (t, J=8.6 Hz, 2H), 3.91 (s, 3H), 3.76 (d, J=9.8 Hz, 1H), 3.66 (s, 3H), 3.40 (s, 1H), 3.15 (t, J=8.7 Hz, 2H), 3.12-3.02 (m, 2H), 2.91 (td, J=11.6, 7.0 Hz, 1H), 2.63-2.55 (m, 2H), 2.53-2.42 (m, 1H), 2.35 (dd, J=14.3, 6.9 Hz, 1H), 2.29 (d, J=15.0 Hz, 1H), 2.26-2.14 (m, 1H), 2.00 (s, 2H), 1.94-1.70 (m, 6H), 1.63 (d, J=7.1 Hz, 3H), 1.59 (s, 3H) ppm. HRMS-ESI (m/z): Calcd for C₄₇H₅₁FN₂O₁₀ [M+H]⁺ 803.3542. Found 803.3465.

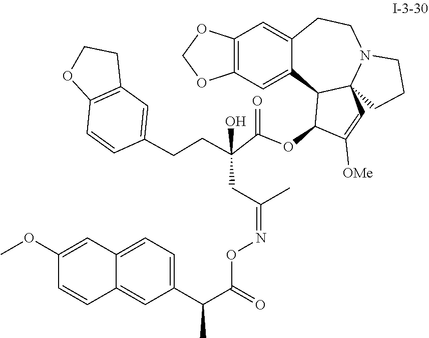

Compound I-3-30: Column chromatography PE:EA=2:1, 0.5% TEA to give light yellow amorphous solid (20% yield). ¹H NMR (400 MHz, CDCl₃): δ 7.64 (d, J=8.8 Hz, 2H), 7.58 (d, J=8.5 Hz, 1H), 7.34 (d, J=8.3 Hz, 1H), 7.09 (d, J=9.0 Hz, 1H), 7.04 (s, 1H), 6.81 (s, 1H), 6.73 (d, J=8.2 Hz, 1H), 6.66 (t, J=7.2 Hz, 1H), 6.50 (dd, J=12.3, 2.5 Hz, 2H), 5.98 (d, J=9.5 Hz, 2H), 5.79 (d, J=10.4 Hz, 1H), 5.58 (s, 1H), 5.10 (s, 1H), 4.53 (t, J=8.6 Hz, 2H), 3.89 (s, 3H), 3.78 (d, J=9.7 Hz, 1H), 3.69 (s, 3H), 3.21-3.05 (m, 4H), 2.95-2.87 (m, 1H), 2.85 (s, 1H), 2.65-2.49 (m, 3H), 2.32-2.20 (m, 2H), 2.07-1.97 (m, 2H), 1.93-1.88 (m, 1H), 1.82 (s, 3H), 1.79-1.72 (m, 3H), 1.72-1.64 (m, 2H), 1.61 (d, J=7.2 Hz, 3H) ppm. HRMS-ESI (m/z): Calcd for C₄₇H₅₁FN₂O₁₀ [M+H]⁺ 803.3542. Found 803.3465.

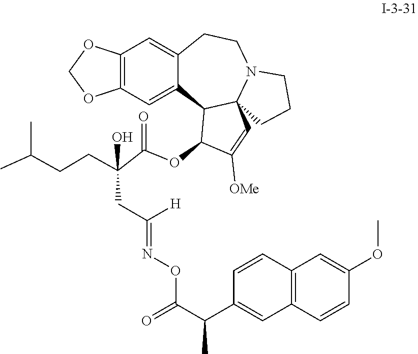

Compound I-3-31 column chromatography PE:EA=2:1, 0.5% TEA to give light yellow amorphous solid (20% yield). ¹H NMR (400 MHz, CDCl₃): δ 7.71 (d, J=8.2 Hz, 3H), 7.44 (d, J=8.5 Hz, 1H), 7.14 (dd, J=13.0, 4.1 Hz, 2H), 7.01 (dd, J=8.9, 3.8 Hz, 1H), 6.51 (d, J=10.7 Hz, 2H), 5.93-5.88 (m, 1H), 5.52 (d, J=22.8 Hz, 2H), 5.05 (s, 1H), 3.91 (s, 3H), 3.77-3.72 (m, 1H), 3.65 (s, 3H), 3.12-2.98 (m, 3H), 2.89 (td, J=11.4, 6.9 Hz, 1H), 2.57 (q, J=8.5 Hz, 2H), 2.30 (td, J=14.8, 7.8 Hz, 2H), 2.08-1.95 (m, 1H), 1.94-1.82 (m, 1H), 1.74 (ddd, J=18.9, 12.4, 5.4 Hz, 4H), 1.61 (d, J=7.1 Hz, 3H), 1.50 (ddd, J=49.4, 13.4, 6.0 Hz, 3H), 0.94-0.86 (m, 2H), 0.83 (dd, J=6.7, 3.1 Hz, 6H) ppm. HRMS-ESI (m/z): Calcd for C₄₁H₄₉N₂O₉ [M+H]⁺ 713.3439. Found 713.3360.

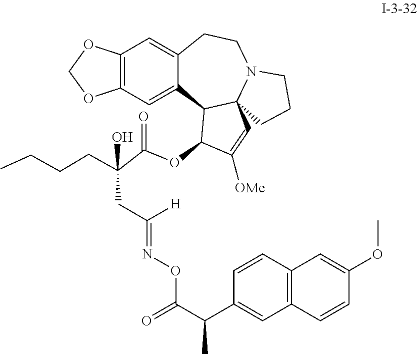

Compound I-3-32: Column chromatography PE:EA=4:1, 0.5% TEA to give light yellow amorphous solid (54% yield). ¹H NMR (400 MHz, CDCl₃): δ 7.71 (d, J=8.2 Hz, 3H), 7.44 (d, J=8.5 Hz, 1H), 7.14 (dd, J=13.0, 4.1 Hz, 2H), 7.01 (dd, J=8.9, 3.8 Hz, 1H), 6.51 (d, J=10.7 Hz, 2H), 5.93-5.88 (m, 1H), 5.52 (d, J=22.8 Hz, 2H), 5.05 (s, 1H), 3.91 (s, 3H), 3.77-3.72 (m, 1H), 3.65 (s, 3H), 3.12-2.98 (m, 3H), 2.89 (td, J=11.4, 6.9 Hz, 1H), 2.57 (q, J=8.5 Hz, 2H), 2.30 (td, J=14.8, 7.8 Hz, 2H), 2.08-1.95 (m, 1H), 1.94-1.82 (m, 1H), 1.74 (ddd, J=18.9, 12.4, 5.4 Hz, 4H), 1.61 (d, J=7.1 Hz, 3H), 1.50 (ddd, J=49.4, 13.4, 6.0 Hz, 3H), 0.94-0.86 (m, 2H), 0.83 (dd, J=6.7, 3.1 Hz, 6H) ppm. HRMS-ESI (m/z): Calcd for C₄₀H₄₇N₂O₉ [M+H]⁺ 699.3203. Found 699.3280.

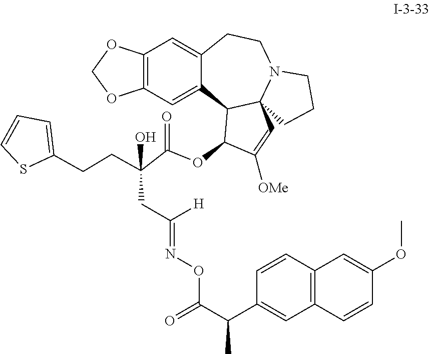

Compound I-3-33: Column chromatography PE:EA=2:1, 0.5% TEA to give light yellow amorphous solid (58% yield). ¹′H NMR (400 MHz, CDCl₃): δ 7.71 (d, J=8.6 Hz, 3H), 7.43 (dd, J=8.4, 1.8 Hz, 1H), 7.18-7.06 (m, 3H), 7.00 (dd, J=8.6, 4.0 Hz, 1H), 6.89 (dd, J=5.1, 3.4 Hz, 1H), 6.75-6.71 (m, 1H), 6.52 (d, J=4.7 Hz, 2H), 5.96 (d, J=9.6 Hz, 1H), 5.46 (dd, J=46.8, 1.5 Hz, 2H), 5.08 (s, 1H), 3.92 (s, 3H), 3.77 (d, J=9.6 Hz, 1H), 3.67 (s, 3H), 3.14-3.00 (m, 3H), 2.88 (ddt, J=17.7, 13.6, 5.8 Hz, 2H), 2.62-2.52 (m, 3H), 2.39-2.25 (m, 2H), 2.09-1.95 (m, 2H), 1.98-1.82 (m, 3H), 1.84-1.70 (m, 3H), 1.61 (d, J=7.1 Hz, 3H) ppm. HRMS-ESI (m/z): Calcd for C₄₂H₄₅N₂O₉S [M+H]⁺ 753.2845. Found 753.2768.

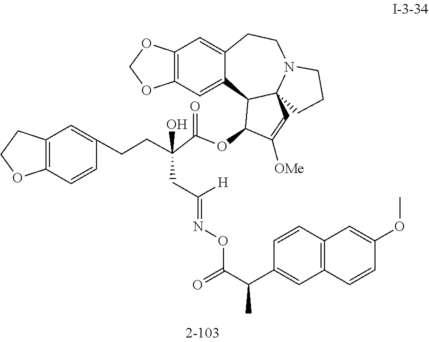

Compound I-3-34: column chromatography PE:EA=2:1, 0.5% TEA to give a pale yellow amorphous solid (83% yield). ¹′H NMR (400 MHz, CDCl₃): δ 7.71 (d, J=8.6 Hz, 3H), 7.43 (dd, J=8.3, 1.9 Hz, 1H), 7.16-7.10 (m, 2H), 7.04 (dd, J=8.7, 4.0 Hz, 1H), 6.95 (s, 1H), 6.84 (d, J=8.8 Hz, 1H), 6.67 (d, J=8.1 Hz, 1H), 6.53 (s, 1H), 6.51 (s, 1H), 5.95 (d, J=9.6 Hz, 1H), 5.48 (dd, J=48.8, 1.6 Hz, 2H), 5.09 (s, 1H), 4.53 (t, J=8.7 Hz, 2H), 3.92 (s, 3H), 3.79 (d, J=9.7 Hz, 1H), 3.69 (s, 3H), 3.16 (t, J=8.7 Hz, 2H), 3.11-3.02 (m, 2H), 3.08 (s, 1H), 2.90 (td, J=11.5, 6.8 Hz, 1H), 2.64-2.47 (m, 4H), 2.36-2.20 (m, 4H), 2.09-1.97 (m, 1H), 1.93-1.85 (m, 1H), 1.82-1.71 (m, 4H), 1.61 (d, J=7.1 Hz, 3H) ppm. HRMS-ESI (m/z): Calcd for C₄₆H₄₉N₂O₁₀ [M+H]⁺ 789.3385. Found 789.3309.

### Example 24: Preparation of Ketoxime Ether Analogs

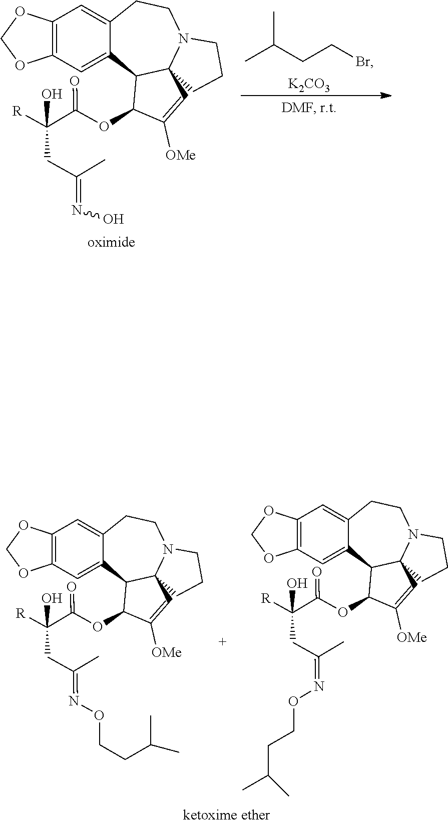

General operation: Isoamyl bromide (1.2 mmol) was added dropwise to a solution of DMF (2 mL) containing the oxime (1 mmol) of K₂CO₃ (2 mmol) under argon protection, and the reaction was monitored on a TLC plate until complete. Quenched by addition of saturated NaHCO₃, extracted with Et₂O, the organic phases were combined, washed sequentially with H₂O, saturated NaCl and dried over anhydrous Na₂SO₄. The solvent was removed by spin evaporation to give the ketoxime ether crude product.

Referring to the above method, the following compounds were obtained:

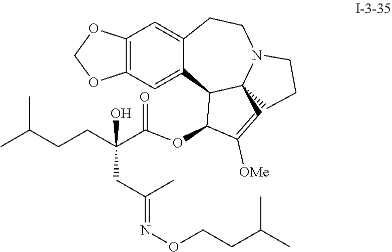

Compound I-3-35: Column chromatography PE:EA=7: 1, 0.5% TEA to give a pale yellow amorphous solid (63% yield). ¹H NMR (400 MHz, CDCl₃): δ 6.59 (s, 1H), 6.55 (s, 1H), 5.93 (d, J=9.0 Hz, 1H), 5.85 (dd, J=9.2, 1.6 Hz, 2H), 5.02 (s, 1H), 3.76 (d, J=9.8 Hz, 1H), 3.72 (s, 1H), 3.66 (s, 3H), 3.21-3.10 (m, 1H), 3.13-3.02 (m, 1H), 2.92 (td, J=11.6, 7.1 Hz, 1H), 2.63-2.52 (m, 2H), 2.37 (dd, J=14.1, 6.9 Hz, 1H), 2.11-1.96 (m, 3H), 1.94-1.82 (m, 2H), 1.74 (ddd, J=17.1, 8.7, 4.6 Hz, 3H), 1.61 (s, 3H), 1.54-1.35 (m, 3H), 1.34-1.22 (m, 5H), 1.13 (dt, J=13.1, 6.9 Hz, 1H), 0.90-0.86 (m, 3H), 0.83 (dt, J=6.6, 3.4 Hz, 8H) ppm. HRMS-ESI (m/z): Calcd for C₃₃H₄₉N₂O₇ [M+H]⁺ 585.3541. Found 584.3462.

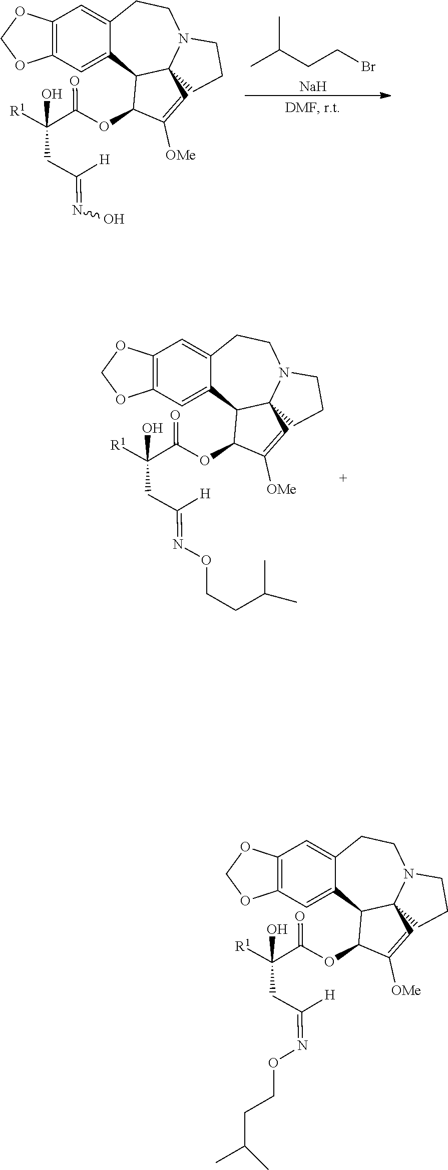

General operation: Isoamyl bromide (1.2 mmol) was added dropwise to a solution of DMF (2 mL) containing oxime (1 mmol) of NaH (1.2 mmol) under argon protection, and the reaction was monitored on a TLC plate until complete. The reaction was quenched by addition of half-saturated and NH₄Cl, extracted with Et₂O, and the organic phases were combined, washed sequentially with H₂O, saturated NaCl, and dried over anhydrous Na₂SO₄. The solvent was removed by spin evaporation to give the crude product of aldoxime ether.

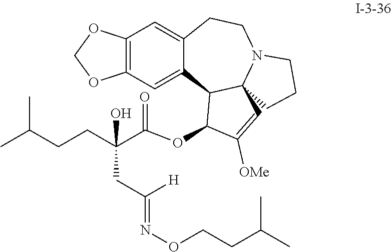

Compound I-3-36: column chromatography PE:EA=6:1, 0.5% TEA to give a light yellow amorphous solid (66% yield). ¹H NMR (400 MHz, CDCl₃): δ 6.57 (d, J=4.4 Hz, 2H), 6.23 (dd, J=6.7, 3.5 Hz, 1H), 5.92 (d, J=9.7 Hz, 1H), 5.87 (s, 2H), 5.06 (s, 1H), 4.07 (td, J=7.0, 2.2 Hz, 2H), 3.79 (d, J=9.7 Hz, 1H), 3.68 (s, 3H), 3.15-3.06 (m, 2H), 3.01 (s, 1H), 2.98-2.89 (m, 1H), 2.60 (t, J=8.3 Hz, 2H), 2.50 (dd, J=16.7, 6.7 Hz, 1H), 2.37 (dd, J=14.1, 6.7 Hz, 1H), 2.03 (t, J=10.4 Hz, 1H), 1.90 (ddd, J=12.2, 8.1, 4.1 Hz, 1H), 1.79-1.59 (m, 6H), 1.49 (dq, J=37.9, 6.8 Hz, 6H), 0.93 (dd, J=6.7, 2.2 Hz, 6H), 0.89 (d, J=6.7 Hz, 1H), 0.84 (dd, J=6.6, 2.2 Hz, 6H) ppm. HRMS-ESI (m/z): Calcd for C₃₂H₄₇N₂O₇ [M+H]⁺ 571.3384. Found 570.3305.

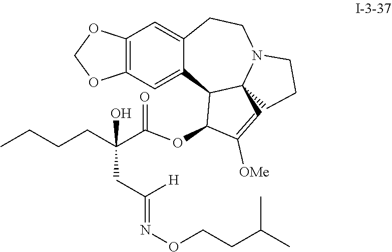

Compound I-3-37: column chromatography PE:EA=6:1, 0.5% TEA to give a pale yellow amorphous solid (35% yield). ¹H NMR (400 MHz, CDCl₃) δ 6.60 (s, 1H), 6.58 (s, 1H), 5.93 (d, J=9.8 Hz, 1H), 5.86 (d, J=13.8 Hz, 2H), 5.05 (s, 1H), 4.20-4.01 (m, 1H), 3.99 (t, J=7.0 Hz, 2H), 3.79 (d, J=9.6 Hz, 1H), 3.67 (s, 3H), 3.19-3.04 (m, 2H), 2.94 (dd, J=21.0, 9.1 Hz, 1H), 2.64-2.53 (m, 2H), 2.38 (dd, J=13.7, 6.7 Hz, 1H), 2.15-1.97 (m, 2H), 1.95-1.84 (m, 1H), 1.83-1.69 (m, 3H), 1.70-1.39 (m, 5H), 1.27 (h, J=8.6, 7.5 Hz, 6H), 1.16-0.98 (m, 1H), 0.90 (d, J=6.8 Hz, 6H), 0.89-0.80 (m, 3H) ppm. HRMS-ESI (m/z): Calcd for C₃₁H₄₅N₂O₇ [M+H]⁺ 557.3225. Found 557.3149.

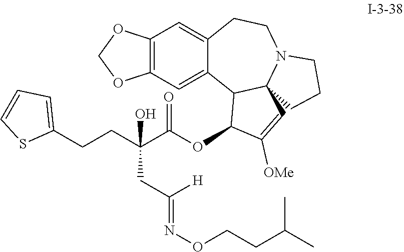

Compound I-3-38: column chromatography PE:EA=3:1, 0.5% TEA to give a pale yellow amorphous solid (40% yield). ¹H NMR (400 MHz, CDCl₃): δ 7.10 (dd, J=5.2, 1.2 Hz, 1H), 6.90 (dd, J=5.2, 3.4 Hz, 1H), 6.74 (d, J=2.2 Hz, 1H), 6.58 (s, 1H), 6.58 (s, 1H), 6.23 (dd, J=6.7, 3.6 Hz, 1H), 5.98 (d, J=9.6 Hz, 1H), 5.83 (dd, J=26.4, 1.7 Hz, 2H), 5.09 (s, 1H), 4.07 (td, J=7.0, 2.4 Hz, 2H), 3.82 (d, J=9.7 Hz, 1H), 3.70 (s, 3H), 3.19-3.05 (m, 3H), 3.01-2.80 (m, 2H), 2.66-2.49 (m, 4H), 2.38 (dd, J=14.2, 6.8 Hz, 1H), 2.12-1.99 (m, 1H), 1.97-1.85 (m, 3H), 1.81-1.72 (m, 3H), 1.73-1.61 (m, 3H), 0.91 (dd, J=6.6, 4.0 Hz, 6H) ppm. HRMS-ESI (m/z): Calcd for C₃₃H₄₃N₂O₇S [M+H]⁺ 611.2789. Found 611.2713.

### Example 25: Preparation of Carbamic Acid Oxime Ester Compounds

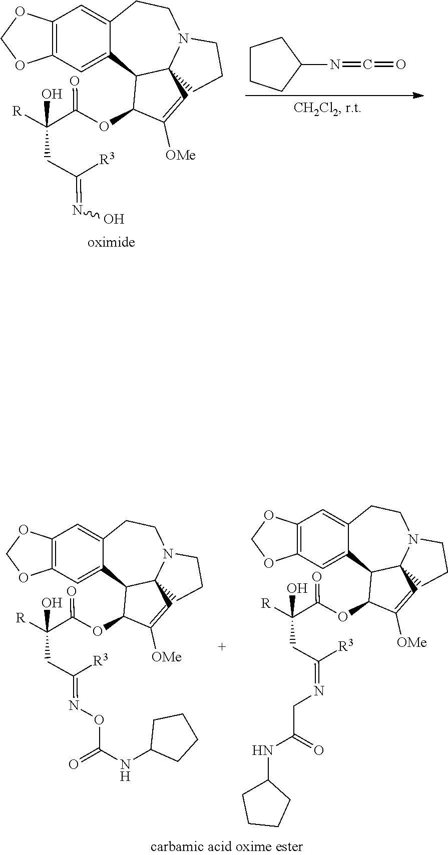

General operation: Cyclopentyl carbamate (1.2 mmol) was added dropwise to a solution of oxime (1 mmol) in CH₂Cl₂ (2 mL) under Ar atmosphere and reacted for 12 h. The reaction was quenched by the addition of saturated NaHCO₃, extracted by CH₂Cl₂, the organic phases were combined, washed with saturated NaCl and dried over anhydrous Na₂SO₄. The solvent was removed by spin evaporation to give the crude product of carbamic acid oxime ester.

Referring to the above method, the following compounds were obtained:

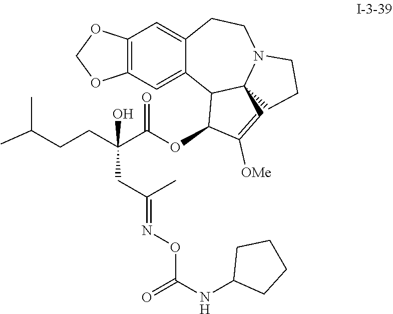

Compound I-3-39: Column chromatography PE:EA=6:1, 0.5% TEA to give a light yellow amorphous product (32% yield). ¹H NMR (400 MHz, CDCl₃): δ 6.49 (s, 1H), 6.42 (s, 1H), 6.07 (d, J=7.2 Hz, 1H), 5.96 (d, J=9.7 Hz, 1H), 5.88 (d, J=1.6 Hz, 2H), 5.05 (s, 1H), 4.09 (h, J=6.9 Hz, 1H), 3.71 (d, J=9.3 Hz, 1H), 3.67 (s, 3H), 3.30 (s, 1H), 3.18-3.00 (m, 2H), 2.90 (td, J=11.5, 7.0 Hz, 1H), 2.63-2.51 (m, 2H), 2.34 (dd, J=14.1, 7.0 Hz, 1H), 2.27 (d, J=17.5 Hz, 1H), 2.13 (d, J=17.5 Hz, 1H), 2.11-1.93 (m, 4H), 1.96-1.83 (m, 2H), 1.85-1.74 (m, 2H), 1.74 (s, 3H), 1.72-1.51 (m, 6H), 1.52-1.42 (m, 2H), 1.41-1.28 (m, 1H), 0.86 (d, J=6.6 Hz, 6H) ppm. HRMS-ESI (m/z): Calcd for C₃₄H₄₈N₃O₈ [M+H]⁺ 626.3438. Found 626.3363.

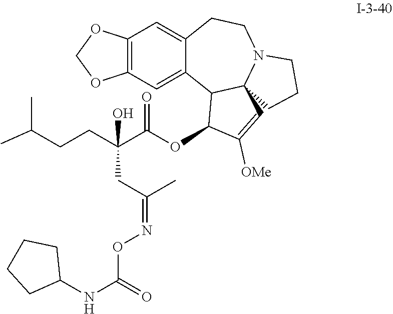

Compound I-3-40: column chromatography PE:EA=6:1, 0.5% TEA to give light yellow amorphous solid (41% yield). ¹H NMR (400 MHz, CDCl₃): δ 6.68 (s, 1H), 6.63 (s, 1H), 6.21 (d, J=7.4 Hz, 1H), 5.97 (d, J=9.6 Hz, 1H), 5.85 (s, 2H), 5.08 (s, 1H), 4.08 (h, J=6.8 Hz, 1H), 3.85 (d, J=9.7 Hz, 1H), 3.69 (s, 3H), 3.21-2.99 (m, 4H), 2.75-2.55 (m, 3H), 2.13-1.89 (m, 6H), 1.79 (s, 3H), 1.73-1.53 (m, 6H), 1.55-1.41 (m, 3H), 1.43-1.30 (m, 4H), 0.82 (d, J=6.6 Hz, 6H) ppm. HRMS-ESI (m/z): Calcd for C₃₄H₄₈N₃O₈ [M+H]⁺ 626.3438. Found 626.3363.

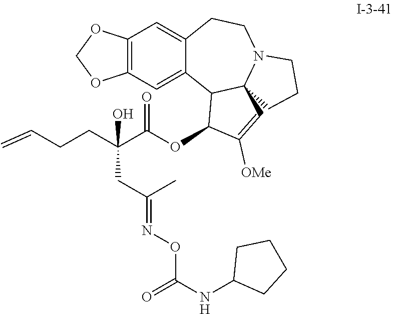

Compound (I-3-41): column chromatography PE:EA=6:1, 0.5% TEA to give light yellow amorphous solid (32% yield). ¹H NMR (400 MHz, CDCl₃): δ 6.48 (s, 1H), 6.42 (s, 1H), 6.03 (d, J=1.7 Hz, 1H), 5.97 (d, J=9.7 Hz, 1H), 5.86 (d, J=1.7 Hz, 1H), 5.86-5.68 (m, 1H), 5.06 (s, 1H), 5.05-4.90 (m, 2H), 4.17-4.04 (m, 1H), 3.69 (d, J=10.1 Hz, 1H), 3.67 (s, 3H), 3.31 (d, J=1.5 Hz, 1H), 3.18-3.01 (m, 3H), 2.97-2.85 (m, 1H), 2.63-2.52 (m, 2H), 2.34 (dd, J=13.6, 6.3 Hz, 1H), 2.30 (d, J=17.3 Hz, 1H), 2.24-2.15 (m, 1H), 2.18 (d, J=17.3 Hz, 1H), 2.13-1.87 (m, 6H), 1.85-1.76 (m, 2H), 1.76 (s, 3H), 1.75 (s, 1H), 1.72-1.52 (m, 6H) ppm. HRMS-ESI (m/z): Calcd for C₃₃H₄₄N₃O₈ [M+H]⁺ 610.3126. Found 610.3050.

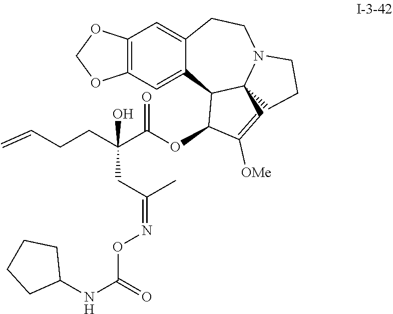

Compound I-3-42: column chromatography PE:EA=6:1, 0.5% TEA to give light yellow amorphous solid (42% yield). ¹H NMR (400 MHz, CDCl₃): δ 6.48 (s, 1H), 6.42 (s, 1H), 6.03 (s, 1H), 5.97 (d, J=9.7 Hz, 1H), 5.86 (s, 1H), 5.85-5.68 (m, 1H), 5.05 (s, 1H), 5.04-4.88 (m, 2H), 4.08 (dd, J=8.4, 4.9 Hz, 1H), 3.69 (d, J=10.1 Hz, 1H), 3.67 (s, 3H), 3.41-3.27 (m, 1H), 3.18-2.99 (m, 2H), 2.98-2.81 (m, 1H), 2.64-2.52 (m, 3H), 2.40-2.25 (m, 2H), 2.18 (d, J=17.3 Hz, 2H), 2.12-1.94 (m, 4H), 1.96-1.84 (m, 2H), 1.87-1.75 (m, 2H), 1.75 (s, 3H), 1.76-1.69 (m, 1H), 1.73-1.54 (m, 6H) ppm. HRMS-ESI (m/z): Calcd for C₃₃H₄₄N₃O₈ [M+H]⁺ 610.3126. Found 610.3050.

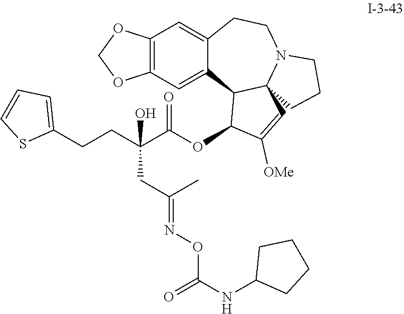

Compound I-3-43: column chromatography PE:EA=3:1, 0.5% TEA to give a pale yellow amorphous solid (21% yield). ¹H NMR (400 MHz, CDCl₃): δ 7.09 (dd, J=5.2, 1.2 Hz, 1H), 6.89 (dd, J=5.2, 3.4 Hz, 1H), 6.73 (d, J=3.4 Hz, 1H), 6.68 (s, 1H), 6.63 (s, 1H), 6.17 (d, J=7.4 Hz, 1H), 6.01 (d, J=9.6 Hz, 1H), 5.80 (dd, J=14.1, 1.5 Hz, 2H), 5.09 (s, 1H), 4.07 (h, J=7.3 Hz, 1H), 3.86 (d, J=9.6 Hz, 1H), 3.67 (s, 3H), 3.23 (s, 1H), 3.17-3.07 (m, 2H), 2.94 (td, J=11.6, 6.8 Hz, 1H), 2.89-2.76 (m, 1H), 2.67 (d, J=14.2 Hz, 1H), 2.66-2.53 (m, 3H), 2.06-1.86 (m, 5H), 1.81 (s, 3H), 1.74 (dd, J=13.1, 4.7 Hz, 2H), 1.71-1.55 (m, 6H), 1.53-1.40 (m, 2H), 1.43-1.31 (m, 1H) ppm. HRMS-ESI (m/z): Calcd for C₃₅H₄₄N₃O₈S [M+H]⁺ 666.2680. Found 666.2771.

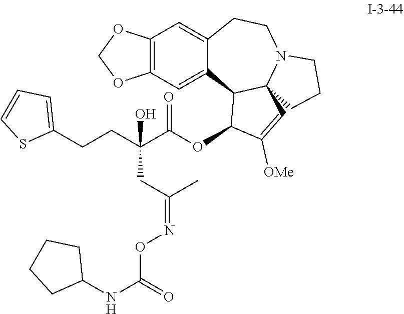

Compound I-3-44: Column chromatography PE:EA=3:1, 0.5% TEA to give light yellow amorphous solid (18% yield). ¹′H NMR (400 MHz, CDCl₃): δ 7.11 (dd, J=5.1, 1.2 Hz, 1H), 6.92 (dd, J=5.2, 3.4 Hz, 1H), 6.76 (d, J=2.2 Hz, 1H), 6.49 (s, 1H), 6.44 (s, 1H), 6.06 (d, J=7.2 Hz, 1H), 6.02 (d, J=1.7 Hz, 1H), 5.86 (d, J=1.7 Hz, 1H), 5.07 (s, 1H), 4.10 (h, J=6.8 Hz, 1H), 3.72 (d, J=9.7 Hz, 1H), 3.65 (s, 3H), 3.41 (d, J=1.6 Hz, 1H), 3.18-3.01 (m, 2H), 3.02-2.87 (m, 2H), 2.70 (td, J=13.6, 4.6 Hz, 1H), 2.62-2.55 (m, 2H), 2.35 (dd, J=14.4, 7.0 Hz, 1H), 2.22 (d, J=17.5 Hz, 1H), 2.20 (d, J=17.5 Hz, 1H), 2.11-1.95 (m, 4H), 1.98-1.85 (m, 3H), 1.86-1.78 (m, 2H), 1.77 (s, 3H), 1.73-1.55 (m 6H) ppm. HRMS-ESI (m/z): Calcd for C₃₅H₄₄N₃O₈S [M+H]⁺ 666.2680. Found 666.2771.

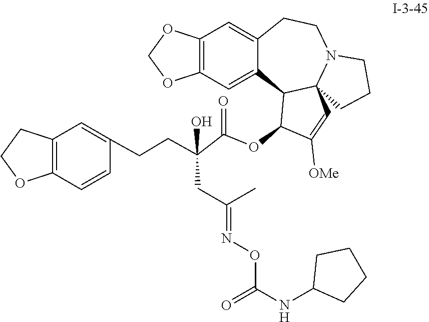

Compound I-3-45: Column chromatography PE:EA=3:1, 0.5% TEA to give light yellow amorphous solid (44% yield). ¹H NMR (400 MHz, CDCl₃): δ 6.94 (s, 1H), 6.83 (d, J=8.1 Hz, 1H), 6.66 (d, J=8.1 Hz, 1H), 6.66 (s, 1H), 6.62 (s, 1H), 6.17 (d, J=7.4 Hz, 1H), 6.00 (d, J=9.6 Hz, 1H), 5.78 (dd, J=22.7, 1.6 Hz, 2H), 5.11 (s, 1H), 4.53 (t, J=8.6 Hz, 2H), 4.06 (q, J=6.9 Hz, 1H), 3.86 (d, J=9.7 Hz, 1H), 3.70 (s, 3H), 3.20 (s, 1H), 3.16 (t, J=8.5 Hz, 2H), 3.15-3.04 (m, 2H), 2.94 (td, J=11.5, 6.9 Hz, 1H), 2.70 (d, J=14.1 Hz, 1H), 2.60 (td, J=14.5, 6.9 Hz, 3H), 2.49 (td, J=12.8, 4.7 Hz, 1H), 2.24 (td, J=13.7, 13.2, 3.9 Hz, 1H), 2.10-1.95 (m, 2H), 1.91 (dt, J=8.4, 5.2 Hz, 2H), 1.82 (s, 3H), 1.82-1.71 (m, 2H), 1.72-1.54 (m, 6H), 1.53-1.40 (m, 3H) ppm. HRMS-ESI (m/z): Calcd for C₃₉H₄₈N₃O₉ [M+H]⁺ 702.3388. Found 702.3312.

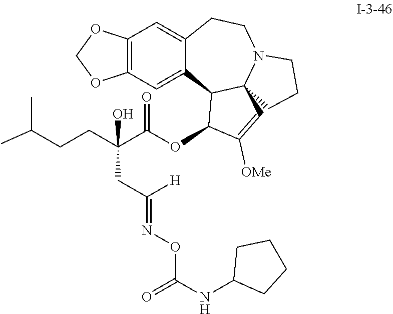

Compound I-3-46: column chromatography PE:EA=6:1, 0.5% TEA to give a pale yellow amorphous solid (38% yield). ¹H NMR (400 MHz, CDCl₃): δ 6.64 (s, 1H), 6.60 (s, 1H), 5.94 (d, J=9.7 Hz, 1H), 5.91-5.88 (m, 2H), 5.09 (s, 1H), 3.81 (d, J=9.7 Hz, 1H), 3.68 (s, 4H), 3.34 (s, 1H), 3.13-3.01 (m, 2H), 3.01-2.89 (m, 2H), 2.66-2.54 (m, 2H), 2.38 (dd, J=13.6, 6.3 Hz, 1H), 2.10-2.01 (m, 2H), 2.00-1.85 (m, 2H), 1.80-1.71 (m, 4H), 1.67-1.53 (m, 4H), 1.47 (dt, J=13.2, 6.5 Hz, 1H), 1.40-1.23 (m, 4H), 0.97-0.88 (m, 3H), 0.85 (dd, J=6.6, 4.5 Hz, 6H) ppm. HRMS-ESI (m/z): Calcd for C₃₃H₄₆N₃O₈ [M+H]⁺ 612.3286. Found 612.3207.

### Example 26: In Vitro Bioactivity Test

### Test Cells:

Human breast cancer cell MCF-7, hepatocellular carcinoma cell HepG2, lung adenocarcinoma cell A549 were purchased from Shanghai Cell Bank of Chinese Academy of Sciences. Human leukemia cells HL-60, human leukemia cells adriamycin-resistant strain HL-60/ADR, and human glioblastoma cells U-87MG were purchased from Shanghai Chuanqiu Biotechnology Co. Human cervical cancer HeLa was purchased from Shanghai Jikai Gene Chemistry Technology Co. Human colon cancer cells HCT-116, human brain microvascular endothelial cells hCMEC/D3 were preserved by Tianjin Suntech Pharmaceutical Technology Co. Human colon cancer cells Sw620 and human colon cancer cells LoVo were provided by Tianjin Medical University Hospital.

### Main Instruments and Consumables:

Carbon dioxide constant temperature cell culture incubator, Thermo Fisher, USA,

Inverted biomicroscope, Chongqing Aote Optical Instrument Co,

Enzyme labeling instrument, Thermo Fisher, USA,

Precision adjustable micropipette, Eppendorf (Germany),

96-well cell culture plate, Wuxi Ness Life Science and Technology Co,

Cell culture dish, Wuxi Ness Life Science and Technology Co.

### Main Reagents:

Fetal bovine serum, BI (Israel),

Trypsin, Gibco,

Double antibody, Gibco,

DMEM medium, BI, Israel,

DMSO, Tianjin Bohua Chemical Reagent Co,

MTT, Shanghai Yuanye Biotechnology Co.

The experimental steps were as follows:

### Cell Culture:

Human breast cancer cell MCF-7, hepatocellular carcinoma cell HepG2, lung adenocarcinoma cell A549, human leukemia cell HL-60, human leukemia cell adriamycin-resistant strain HL-60/ADR, human glioblastoma cell U-87MG, human cervical carcinoma cell HeLa, human colon carcinoma cell HCT-116, human colon carcinoma cell Sw620, human colon carcinoma cell LoVo, Human brain microvascular endothelial cells HCMEC/D3 were cultured using DMEM medium. The medium was supplemented with 10% fetal bovine serum and 1% double antibody (penicillin+streptomycin), and the culture environment was an incubator filled with 5% CO2 at a temperature of 37 °C, when the cells basically grew all over the bottom of the dish (about 80%-90%), passaging culture was performed.

### Preparation of Working Solution:

Preparation of MTT solution: Weigh 50 mg of MTT, dissolve it in 10 mL of phosphate buffer solution (PBS), dispense it, and store it at 4 °C away from light, it will be effective within two weeks.

### Configuration of Test Drug:

Take the series of compounds diluted with medium to form 1 mg/ml master batch, before use, precise aspiration of the appropriate amount of sample master batch diluted with medium to form working solutions with different concentration gradients, such as 100 μg/ml, 10 μg/ml, 1 μg/ml, 0.1 μg/ml, 0.01 μg/ml, 0.001 μg/ml and so on.

### Determination Method:

1. Take log phase cells, digest, centrifuge and resuspend. The cell density was adjusted to 1×105 cells/mL by adding culture medium, and 100 μl of prepared cell suspension per well was added and inoculated into 96-well cell culture plates, after which they were placed in a cell culture incubator for incubation.

2. After 24 h of culture, the test group was added 2 μl of the series of compounds with concentrations of 100 μg/ml, 10 μg/ml, 1 μg/ml, 0.1 μg/ml, 0.01 μg/ml, 0.001 μg/ml, and 4 replicate wells were set up for each concentration, as well as a positive control group (etoposide) and a blank control group.

3. The 96-well plates were incubated separately for 48 h and then 20 μl of MTT solution (concentration of 5 mg/ml) was added to each well and incubation was continued for 4 hours in the incubator.

4. Remove the 96-well plate from the incubator, carefully aspirate the medium in the wells with a micropipette, and add 150 μl of dimethyl sulfoxide to each well, shake to dissolve the crystals completely, and then measure the absorbance OD value using an enzyme counter at 492 nm.

5. The test was repeated three times consecutively and the cell inhibition rate was calculated according to the following formula: cell inhibition rate (%)=[1−OD value of the test group/OD of the blank control group)]×100%, and the IC50 value of the half inhibitory concentration of the samples for inhibiting cell growth was calculated.

The experimental results showed that below 0.1 v % DMSO solvent had negligible effect on the cells, and the experimental results of IC50 values are summarized in Table 1.

TABLE 1
Activity data of Example compounds (IC50 (nM))

| compound | | | | | | | | | |
|---|---|---|---|---|---|---|---|---|---|
| number | HL-60 | HCT116 | A549 | HCMEC/D3 | MCF-7 | HepG2 | HeLa | Sw620 | LoVo |
| HHT | 40 | 26 | 190 | 171 | 40 | 22 | 34 | 35 | 335 |
| I-1-1 | — | — | — | — | 4.3 | 25 | — | | |
| I-1-2 | 7.7 | — | 70 | — | 0.5 | 14 | — | 12 | 57 |
| I-1-9 | 8.9 | — | — | — | — | — | — | — | — |
| I-1-22 | 13.5 | — | — | — | — | — | — | — | — |
| I-1-73 | 9.3 | — | — | — | 22 | 28 | — | — | — |
| I-2-2 | — | — | — | — | 14 | 29 | — | — | — |
| I-2-5 | 3.1 | — | — | — | 11 | 40 | — | — | — |
| I-2-9 | 3.3 | — | — | — | 34 | 48 | — | — | — |
| I-2-29 | — | — | 61 | — | — | — | 25 | — | — |
| I-2-30 | — | — | 21 | — | — | — | 28 | — | — |
| I-2-35 | — | — | 60 | — | 110 | 23 | — | — | — |
| I-2-38 | — | — | 290 | — | 220 | 75 | — | — | — |
| I-2-41 | — | — | 150 | — | 60 | 10 | — | — | — |
| I-2-43 | — | — | — | — | — | 15 | — | — | — |
| I-2-44 | 8.7 | — | 20 | — | 65 | 95 | — | — | — |
| I-2-45 | — | — | 210 | — | 90 | 17 | — | — | — |
| I-2-52 | — | 46 | — | — | — | — | — | — | — |
| I-3-13 | 18 | 16 | 45 | — | — | — | — | — | — |
| I-3-14 | — | 69 | 44 | — | — | — | — | — | — |
| I-3-15 | — | — | 83 | — | — | — | — | — | — |
| I-3-22 | — | 48 | 39 | — | — | — | — | — | — |
| I-3-23 | 34 | 71 | 21 | — | — | — | — | — | — |
| I-3-24 | 67 | 24 | 36 | 245 | — | — | — | — | — |
| I-3-25 | — | — | 92 | — | — | — | — | — | — |
| I-3-31 | 20 | — | 21 | 24 | — | — | — | 3.4 | 45 |
| I-3-34 | — | 26 | 283 | — | — | — | — | — | — |
| I-3-46 | 42 | — | 53 | 60 | — | — | — | — | — |

The above illustrates exemplary embodiments of the present disclosure. However, the scope of protection of the present disclosure is not limited to the above embodiments. Any modifications, equivalent substitutions or improvements made by a person skilled in the art within the spirit and principles of the present disclosure shall be covered by the scope of protection of the present disclosure.

1. A compound as shown in formula (I) or racemate, stereoisomer, reciprocal isomer, isotopic marker, N-oxide, hydrate, solvate, polymorph, metabolite, pharmaceutically acceptable salt, pharmaceutically acceptable ester, or prodrug thereof: 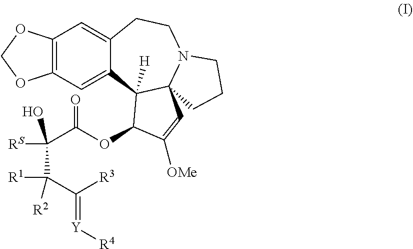
wherein R^{S} is optionally selected from (R^{N})ₘ-G-(CH₂)ₙ— and (R^{Q})ₘ-G-(CH₂)ₙ—.
R^{N} represents a substituent comprising a nitrogen atom, such as a nitro unsubstituted or optionally substituted with one, two or more of the following groups of R^{a}: —NR⁶R⁷, —NR⁶—C(═Y—R⁴)—NR⁶R⁷.
each of R⁶, R^{6′}, and R⁷ is the same or different and is independently selected from H, or the following groups which are unsubstituted or optionally substituted with one, two, or more R^{b}: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹⁰C(═O)—, R¹⁰C(═O)O—, R¹⁰C(═O)NH—, R¹⁰OC(═O)—, R¹⁰NHC(═O)—, R¹⁰S(O)₂—, R¹⁰S(O)₂O—, R¹⁰OS(O)₂—, R¹⁰S(O)—, tri(C₁₋₂₀ alkyloxy)silyl, C₁₋₂₀ alkyl, tri(C₁₋₂₀ alkyl)silyl.
R^{Q} represents H, halogen, —OH, —CN, unsubstituted or optionally substituted with one, two or more R^{e} groups: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹⁰C(═O)—, R¹⁰C(═O)O—, R¹⁰OC(═O)—, R¹⁰S(O)₂—, R¹⁰S(O)₂O—, R¹⁰OS(O)₂—, R¹⁰S(O)—, tri(C₁₋₂₀ alkyloxy)silyl C₁₋₂₀ alkyl, tri(C₁₋₂₀ alkyl)silyl.
G represents a chemical bond, C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, when G is not a chemical bond, the positions of substitution of different groups connected on G may be separated by from 1-10 atoms, e.g., spaced by 1, 2, 3, 4, 5, 6, 7, 8, 9, or 10 atoms, for example, when G is a phenyl, the different groups to which it is attached can be separated 1, 2, 3, 4, or 5 carbon atoms between the positions substituted on G, e.g. where the different groups are ortho-substituted, meta-substituted and/or para-substituted, preferably meta- and/or para-substitutents on G.
Each m is the same or different, independently of each other, selected from an integer above 1.
Each n is the same or different, independently of one another, selected from an integer above 1.
Each Y is the same or different, independently of each other, selected from O, S, or N; provided that R⁴ does not exist when Y is selected from O or S.
R¹ and R² are the same or different and are independently of each other selected from H, C₁₋₆ alkyl or halogen.
R³ is selected from R⁸, R⁸—O—, R⁸—S—.
R⁴ is absent or is selected from H, unsubstituted or optionally substituted by one, two or more R^{d} groups:
R⁸—O—, R⁸—S—, R⁹C(═O)O—, R¹⁰C(═O)O—, R¹⁰NHC(═O)—, R¹⁰NHC(═O)O—, R¹⁰C(═O)NH—,
R⁵ is selected from H or C₁₋₆ alkyl.
Each of R^{a}, R^{b}, R^{c} and R^{d} is the same or different, independently of each other, selected from halogen, —OH, —CN, —NO₂, oxo (═O), unsubstituted or optionally substituted with one, two or more of the following groups of R^{e}: a C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹¹C(═O)—, R¹¹C(═O)O—, R¹¹C(═O)NH—, R¹¹OC(═O)—, R¹¹NHC(═O)—, R¹¹S(O)₂—, R¹¹S(O)₂O—, R¹¹OS(O)₂—, R¹¹S(O)—, —NR¹²R¹³, —NH—C(═Y)—NR¹²R¹³, tris(C1-20 alkyloxy)silyl C₁₋₂₀ alkyl, tris(C₁₋₂₀ alkyl)silyl.
Each of R⁸, R⁹, R¹⁰ and R¹¹ is the same or different and is independently of each other selected from H, C₁₋₂₀ alkyl, C₂₋₂₀ alkenyl, C₃₋₂₀ cycloalkyl, C₃₋₂₀ cycloalkenyl, 3-20 metaheterocycloalkyl, C₆₋₂₀ aryl, 5-20 metaheteroaryl, R^{11′}C(═O)—, R^{11′}C(═O)O—, R^{11′}C(═O)NH—, R^{11′}OC(═O)—, R^{11′}NHC(═O)—, R^{11′}S(O)₂—, R^{11′}S(O)₂O—, R^{11′}OS(O)₂—, R^{11′}S(O)—, —NR¹²R¹³, —NH—C(═Y)—NR¹²R¹³, tris(C₁₋₂₀ alkyloxy)silyl C₁₋₂₀ alkyl, tris(C₁₋₂₀ alkyl)silyl.
Each of R^{11′}, R¹² and R³ is the same or different, independently of each other, from the following groups selected from H, unsubstituted or optionally substituted with one, two or more R^{f} groups: C₁₋₂₀ alkyl, C₂₋₂₀ alkenyl, C₃₋₂₀ cycloalkyl, C₃₋₂₀ cycloalkenyl, 3-20-membered heterocyclyl, C₆₋₂₀ aryl, 5-20-membered heteroaryl.
Each R^{e} and R^{f} are the same or different, independently of each other, selected from halogen, —OH, —CN, —NO₂, —NH₂, oxo (═O), unsubstituted or optionally substituted by one, two or more R⁸ groups: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₁₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio, R¹⁴C(═O)—, R¹⁴C(═O)O—, R¹⁴OC(═O)—, R¹⁴S(O)₂—, R¹⁴S(O)₂O—, R¹⁴OS(O)₂—, R¹⁴S(O)—.
Each R¹⁴ is the same or different, independently of each other, selected from C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl,
Each R^{g} is the same or different, independently selected from the halogen, —OH, —CN, —NO₂, —NH₂, oxygen (═O), C₁₋₂₀ alkyloxy, C₂₋₂₀ alkenyl alkyloxy, C₃₋₂₀ cycloalkyloxy, C₃₋₂₀ cycloalkenyloxy, 3-20 membered heteroaryl cycloyloxy, C₆₋₂₀ aryloxy, 5-20 membered heteroaryloxy, C₆₋₂₀ alkylthio, C₂₋₂₀ alkenylthio, C₃₋₂₀ cycloalkylthio, C₃₋₂₀ cycloalkenylthio, 3-20 membered heterocyclylthio, C₆₋₂₀ arylthio, 5-20 membered heteroarylthio.
Optionally, one, two or more carbon atoms of the C₃₋₂₀ cycloalkyl or 3-20 metaheterocycloalkyl group may optionally be substituted by oxygen to form a carbonyl group.

2. The compound or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein:
G represents a chemical bond, C₁₋₈ alkyl group, C₂₋₈ alkenyl group, C₃₋₁₅ cycloalkyl group, C₃₋₁₅ cycloalkenyl group, 3-15-membered heterocyclic group, C₆₋₁₅ aryl group, 5-15-membered heteroaryl group, such as a chemical bond, C₁₋₈ alkyl group, C₂₋₈ alkenyl group, phenyl group, naphthyl group.
m represents an integer from 1 to 20, preferably an integer from 1 to 10.
n represents an integer from 1 to 20, preferably an integer from 1 to 10.
Halogen represents F, Cl, Br or I.
Y is selected from O, N or S.
R³ is selected from H, C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, C₁₋₈ alkyl —O—, C₂₋₈ alkenyl —O—, C₃₋₁₅ cycloalkyl —O—, C₃₋₁₅ cycloalkenyl —O—, 3-15 membered heterocyclyl —O—, C₆₋₁₅ aryl —O—, 5-15 membered heteroaryl —O—, C₁₋₈ alkyl —S—, C₂₋₈ alkenyl —S—, C₃₋₁₅ cycloalkyl —S—, C₃₋₁₅ cycloalkenyl —S—, 3-15 membered heterocyclyl —S—, C₆₋₁₅ aryl —S—, 5-15 membered heteroaryl —S—.
R⁴ is absent or selected from H.
R^{N} represents a nitro, unsubstituted or optionally substituted by one, two or more of the following groups of R^{a}: —NR⁶R⁷, —NH—C(═Y—R⁴)—NR⁶R⁷.
Preferably, R^{N} is selected from —NR⁶R⁷, —NH—C(═O)—NR⁶R⁷, —NH—C(═S)—NR⁶R⁷ or —NH—C(═NH)—NR⁶R⁷.
Alternatively, R^{N} is selected from a nitro-group, an amino group; or a phenyl group substituted by a nitro-group or an amino group in the ortho-group, meta- and/or para-position. The amino group is not substituted or is substituted by one, two or more substituents selected from C₁₋₈ alkyl carbonyl group, C₁₋₈ alkyl oxy group, C₃₋₈ cycloalkyl carbonyl group, C₃₋₈ cycloalkyl oxy group, C₆₋₁₀ aryl carbonyl group, C₆₋₁₀ aryl oxy group, C₂₋₈ enyl group.

3. The compound or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein:
R⁶ is selected from H, unsubstituted or optionally substituted with one, two or more R^{b} groups: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, R¹⁰C(═O)—, R¹⁰C(═O)O—, R¹⁰OC(═O)—, R¹⁰S(O)₂—, R¹⁰S(O)₂O—, R¹⁰S(O)₂—, R¹⁰S(O)—, tris(C₁₋₂₀ alkyloxy)silyl C₁₋₂₀ alkyl, tris(C₁₋₂₀ alkyl)silyl.
R⁷ is selected from H, unsubstituted or optionally substituted with one, two or more R^{b} groups: C₁₋₂₀ alkyl, C₂₋₂₀ alkenyl, C₃₋₂₀ cycloalkyl, C₃₋₂₀ cycloalkenyl, 3-20 membered heterocyclyl, C₆₋₂₀ aryl, 5-20 membered heteroaryl, R¹⁰C(═O)—, R¹⁰C(═O)O—, R¹⁰OC(═O)—, R¹⁰S(O)₂—, R¹⁰S(O)₂O—, R¹⁰OS(O)₂—, R¹⁰S(O)—, tris(C₁₋₂₀ alkyloxy)silyl C₁₋₂₀ (alkyl, tris(C₁₋₂₀ alkyl)silyl.
Preferably, R⁶ is selected from H, unsubstituted or optionally substituted with one, two or more R^{b}: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, R¹⁰C(═O)—, R¹⁰C(═O)O—, R¹⁰OC(═O)—, R¹⁰S(O)₂—, R¹⁰S(O)₂O—, R¹⁰OS(O)₂—, R¹⁰S(O)—, tris(C₁₋₈ alkyloxy)silyl C₁₋₈ alkyl, tris(C₁₋₈ alkyl)silyl.
R⁷ is selected from H, unsubstituted or optionally substituted with one, two or more R^{b} groups: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, R¹⁰C(═O)—, R¹⁰C(═O)O—, R¹⁰OC(═O)—, R¹⁰S(O)₂—, R¹⁰S(O)₂O—, R¹⁰OS(O)₂—, R¹⁰S(O)—, tris(C₁₋₈ alkyloxy)silyl C₁₋₈ alkyl, tris(C₁₋₈ alkyl)silyl.
R¹⁰ is selected from C₁₋₈ alkyl, C₂₋₈ alkenyl, C₃₋₁₀ cycloalkyl, C₃₋₁₀ cycloalkenyl, 3-8-membered heterocyclic, C₆₋₁₀ aryl, 5-10-membered heteroaryl, NH₂.
Each R^{a} and R^{b} are the same or different, independently of each other, selected from halogen, —OH, —CN, —NO₂, unsubstituted or optionally substituted by one, two or more R^{d} groups: C₁₋₈ alkyl, C₂₋₈ alkenyl, C₃₋₁₅ cycloalkyl, C₃₋₁₅ cycloalkenyl, 3-15 membered heterocyclyl, C₃₋₁₅ aryl, 5-15 membered heteroaryl, C₁₋₈ alkyloxy, C₂₋₈ alkenyl alkyloxy, C₃₋₁₅ cycloalkyloxy, C₃₋₁₅ cycloalkenyloxy, 3-15 membered heteroaryl cycloyloxy, C₆₋₁₅ aryloxy, 5-15 membered heteroaryloxy, C₁₋₈ alkylthio, C₂₋₈ alkenylthio, C₃₋₁₅ cycloalkylthio, C₃₋₁₅ cycloalkenylthio, 3-15 membered heterocyclylthio, C₆₋₁₅ arylthio, 5-15 membered heteroarylthio, R¹¹C(═O)—, R¹¹C(═O)O—, R¹¹OC(═O)—, R¹¹S(O)₂—, R¹¹S(O)₂O—, R¹¹OS(O)₂—, R¹¹S(O)—, —NR¹²R¹³, —NH—C(═Y)—NR¹²R¹³, tris(C₁₋₈ alkyloxy)silyl C₁₋₂₀ alkyl.
Preferably, R⁷ is selected from, for example, H, unsubstituted or optionally substituted with one, two or more R^{e} groups: C₁₋₈ alkyl, C₃₋₈ cycloalkyl, C₂₋₈ alkenyl, tris(C₁₋₈ alkyloxy)silyl C₁₋₈ alkyl, phenyl, biphenyl, naphthyl.
For example, R⁷ is selected from H, unsubstituted or optionally substituted with one, two or more R^{e} groups: methyl, ethyl, n-propyl, isopropyl, n-butyl, isobutyl, tert-butyl, n-pentyl, n-hexyl, n-heptyl, cyclopropyl, cyclobutyl, cyclopentyl, cyclohexyl, cycloheptyl, allyl, phenyl,

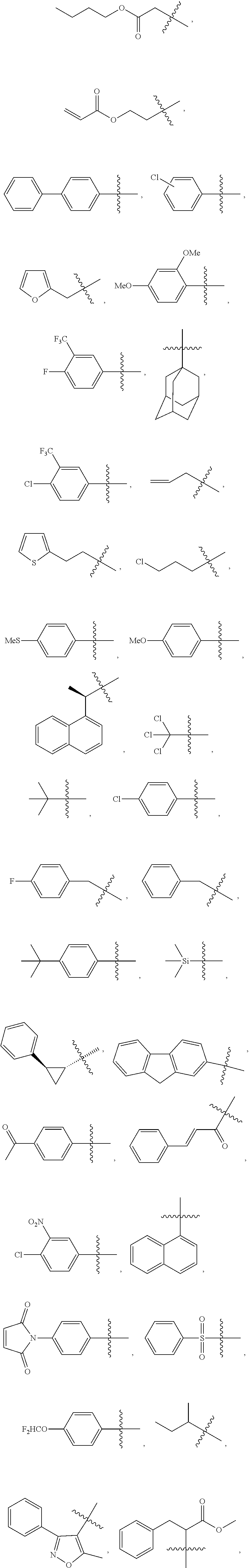

4. The compound or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein:
R^{Q} is selected from H, halogen, —OH, —CN, unsubstituted or optionally substituted with one, two or more R^{e} groups: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, C₁₋₈ alkyloxy, C₂₋₈ alkenyl alkyloxy, C₃₋₁₅ cycloalkyloxy, C₃₋₁₅ cycloalkenyloxy, 3-15 membered heteroaryl cycloyloxy, C₁₋₈ aryloxy, 5-15 membered heteroaryloxy, C₁₋₈ alkylthio, C₂₋₈ alkenylthio, C₃₋₁₅ cycloalkylthio, C₃₋₁₅ cycloalkenylthio, 3-15 membered heterocyclylthio, C₂₋₁₅ arylthio, 5-15 membered heteroarylthio.
Preferably, R^{Q} is selected from H, halogen, —OH, —CN, unsubstituted or optionally substituted by one, two, or more of the following groups: C₁₋₆ alkyl, C₂₋₆ enyl, C₃₋₁₀ cycloalkyl, C₃₋₁₀ cycloenyl, 3-10 heterocyclic, C₆₋₁₀ aryl, 5-10 heteraryl, such as the following groups that are not substituted or are optionally substituted by one, two or more R^{e} groups: methyl, ethyl, propyl, isopropyl, vinyl, allyl, allyl, trifluoromethyl-methyl, pentafluoroethyl, phenyl, naphthyl, thienyl, dihydrobenzo-furanyl;
R³ is selected from H, C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, C₁₋₈ alkyl —O—, C₂₋₈ alkenyl —O—, C₃₋₁₅ cycloalkyl —O—, C₃₋₁₅ cycloalkenyl —O—, 3-15 membered heterocyclyl —O—, C₆₋₁₅ aryl —O—, 5-15 membered heteroaryl —O—, C₁₋₈ alkyl —S—, C₂₋₈ alkenyl —S—, C₃₋₁₅ cycloalkyl —S—, C₃₋₁₅ cycloalkenyl —S—, 3-15 membered heterocyclyl —S—, C₆₋₁₅ aryl —S—, 5-15 membered heteroaryl —S—.
Preferably, R³ is selected from H, methyl, methoxy, methylthio.
Y is selected from N.

5. The compound or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein:
R^{Q} is selected from H, unsubstituted or optionally substituted with one, two or more R groups: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl.
Preferably, R^{Q} is selected from the following groups unsubstituted or optionally substituted by one, two or more R groups: C₁₋₆ alkyl, C₂₋₆ alkenyl, C₃₋₁₀ cycloalkyl, C₃₋₁₀ cycloalkenyl, 3-10 membered heterocyclyl, C₃₋₁₀ aryl, 5-10 membered heteroaryl, e.g. unsubstituted or optionally substituted by one, two or more R^{e} groups: methyl, ethyl, propyl, isopropyl, vinyl, propenyl, allyl, thienyl, dihydrobenzofuranyl.
R³ is selected from H, C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl,
R⁴ is selected from the following groups unsubstituted or optionally substituted with one, two or more R^{d}: R⁸—O—, R⁸—S—, R⁹C(═O)O—,
Each R⁸ and R⁹ are the same or different, independently of each other, selected from H, C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl, —NR¹²R¹³,
Each R¹² and R¹³ are the same or different, independently of each other, selected from H, unsubstituted or optionally substituted with one, two or more R^{f} of the following groups: C₁₋₈ alkyl, C₂₋₈ alkenyl, C₃₋₁₅ cycloalkyl, C₃₋₁₅ cycloalkenyl, 3-15 membered heterocyclyl, C₆₋₁₅ aryl, 5-15 membered heteroaryl.
Preferably, R⁸ is selected from H, —OH, acryloyl, isobutyryl, isoamyl, 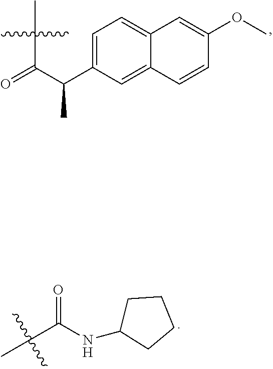 [SMILES: *C(=O)[C@H](C)c1ccc2cc(OC)ccc2c1 ; *C(=O)NC1CCCC1]
R⁹ is selected from C₁₋₈ alkyl, C₂₋₈ alkenyl, NH₂.

6. The compound or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein the compound shown in formula (I) may be selected from the compounds shown in the following formulas (I-1), (I-2), (I-3)), (I-4) and (I-5): 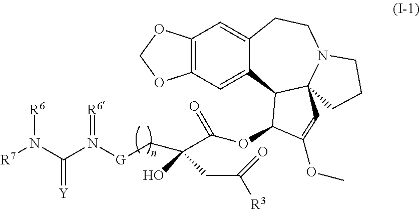
wherein R⁶, R^{6′}, R⁷, G, n, R³ are selected independently of each other from the definitions according to claim 1, and Y represents O, S, or NH. 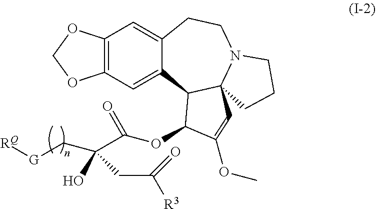
wherein R^{Q}, G, n, R³ are selected independently of each other from the definitions according to claim 1. 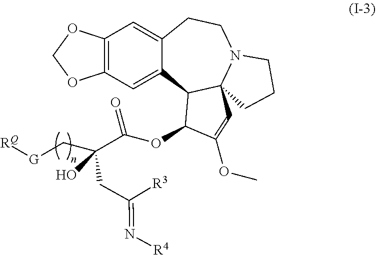
wherein R^{Q}, G, n, R³, R⁴ are selected independently of each other from the definitions according to claim 1. 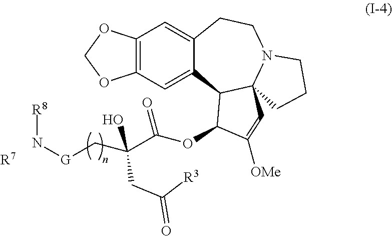
wherein R⁶, R^{6′}, G, n, R³ are independently of each other selected from the definitions according to claim 1. 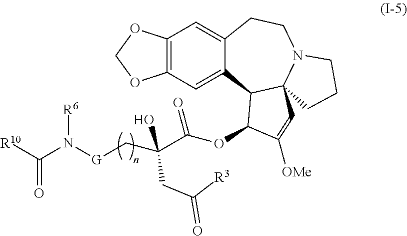
wherein R⁶, R¹⁰, G, n, R³ are independently of each other selected from the definitions according to claim 1.

7. The compound or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein the compound shown in formula (I) is selected from the following compounds: 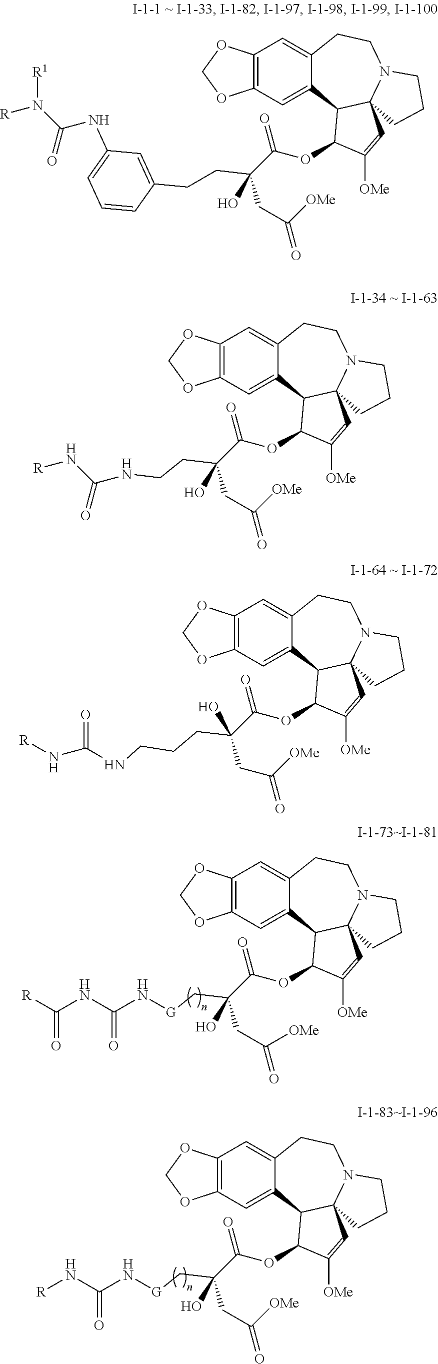
wherein the groups R, G, R⁴ and n have the definitions described in the following table:

| No. | Compds | R | R¹ | No. | Compds | R | No. | Compds | R | G | n | R⁴ |
|---|---|---|---|---|---|---|---|---|---|---|---|---|
| 1 | I-1-1 | 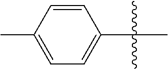 [SMILES: *c1ccc(C)cc1] | H | 39 | I-1-34 | H | 69 | I-1-64 | H | | | |
| 2 | I-1-2 | cyclopentyl | H | 40 | I-1-35 | n-propyl | 70 | I-1-65 | n-pentyl | | | |
| 3 | I-1-3 | H | H | 41 | I-1-36 | n-pentyl | 71 | I-1-66 | cyclopentyl | | | |
| 4 | I-1-4 | n-propyl | H | 42 | I-1-37 | n-hexyl | 72 | I-1-67 | 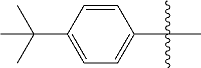 [SMILES: *c1ccc(C(C)(C)C)cc1] | | | |
| 5 | I-1-5 | n-pentyl | H | 43 | I-1-38 | cyclopropyl | 73 | I-1-68 | 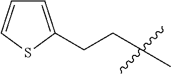 [SMILES: *CCc1cccs1] | | | |
| 6 | I-1-6 | n-hexyl | H | 44 | I-1-39 | cyclobutyl | 74 | I-1-69 | 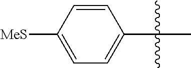 [SMILES: *c1ccc(SC)cc1] | | | |
| 7 | I-1-7 | cyclopropyl | H | 45 | I-1-40 | cyclohexyl | 75 | I-1-70 | 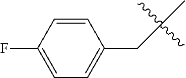 [SMILES: *Cc1ccc(F)cc1] | | | |
| 8 | I-1-8 | cyclobutyl | H | 46 | I-1-41 | cycloheptyl | 76 | I-1-71 | 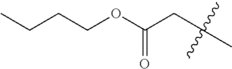 [SMILES: *CC(=O)OCCCC] | | | |
| 9 | I-1-9 | cyclohexyl | H | 47 | I-1-42 | allyl | 77 | I-1-72 | 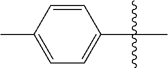 [SMILES: *c1ccc(C)cc1] | | | |
| 10 | I-1-10 | cycloheptyl | H | 48 | I-1-43 | t-butyl | 78 | I-1-73 | 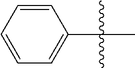 [SMILES: *c1ccccc1] | 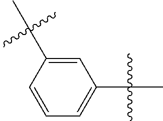 [SMILES: *c1cccc(*)c1] | 2 | |
| 11 | I-1-11 | allyl | H | 49 | I-1-44 | CCl₃ | 79 | I-1-74 | n-pentyl | 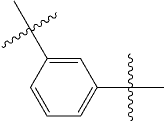 [SMILES: *c1cccc(*)c1] | 2 | |
| 12 | I-1-12 | t-butyl | H | 50 | I-1-45 | TMS | 80 | I-1-75 | cyclopentyl | 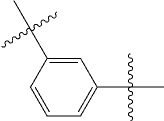 [SMILES: *c1cccc(*)c1] | 2 | |
| 13 | I-1-13 | CCl₃ | H | 51 | I-1-46 | 3-chloropropyl | 81 | 1-1-76 | 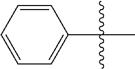 [SMILES: *c1ccccc1] | — | 2 | |
| 14 | I-1-14 | TMS | H | 52 | I-1-47 | 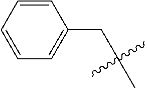 [SMILES: *Cc1ccccc1] | 82 | I-1-77 | n-pentyl | — | 2 | |
| 15 | I-1-15 | 3-chloropropyl | H | 53 | I-1-48 | 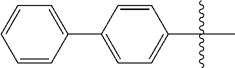 [SMILES: *c1ccc(-c2ccccc2)cc1] | 83 | I-1-78 | cyclopentyl | — | 2 | |
| 16 | I-1-16 | 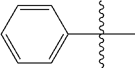 [SMILES: *c1ccccc1] | H | 54 | I-1-49 | 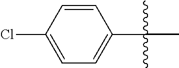 [SMILES: *c1ccc(Cl)cc1] | 84 | I-1-79 | 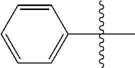 [SMILES: *c1ccccc1] | — | 3 | |
| 17 | I-1-17 | 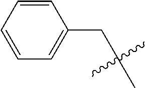 [SMILES: *Cc1ccccc1] | H | 55 | I-1-50 | 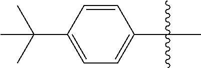 [SMILES: *c1ccc(C(C)(C)C)cc1] | 85 | I-1-80 | n-pentyl | — | 3 | |
| 18 | I-1-18 | 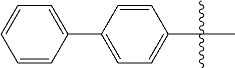 [SMILES: *c1ccc(-c2ccccc2)cc1] | H | 56 | I-1-51 | 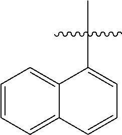 [SMILES: *c1cccc2ccccc12] | 86 | I-1-81 | cyclopentyl | | 3 | |
| 19 | I-1-19 | 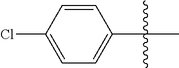 [SMILES: *c1ccc(Cl)cc1] | H | 57 | I-1-52 | 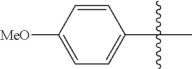 [SMILES: *c1ccc(OC)cc1] | 87 | I-1-83 | n-pentyl | 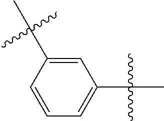 [SMILES: *c1cccc(*)c1] | 2 | H |
| 20 | I-1-20 | 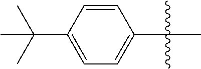 [SMILES: *c1ccc(C(C)(C)C)cc1] | H | 58 | I-1-53 | 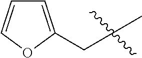 [SMILES: *Cc1ccco1] | 88 | I-1-84 | cyclopentyl | 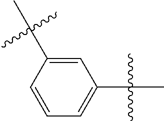 [SMILES: *c1cccc(*)c1] | 2 | H |
| 21 | I-1-21 | 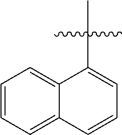 [SMILES: *c1cccc2ccccc12] | H | 59 | I-1-54 | 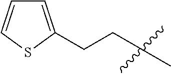 [SMILES: *CCc1cccs1] | 89 | I-1-85 | 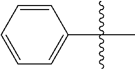 [SMILES: *c1ccccc1] | 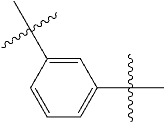 [SMILES: *c1cccc(*)c1] | 2 | H |
| 22 | I-1-22 | 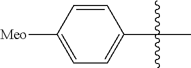 | H | 60 | I-1-55 | 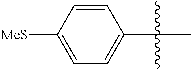 [SMILES: *c1ccc(SC)cc1] | 90 | I-1-86 | i-Pr | 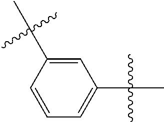 [SMILES: *c1cccc(*)c1] | 2 | i-Pr |
| 23 | I-1-23 | 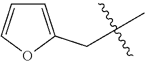 [SMILES: *Cc1ccco1] | H | 61 | I-1-56 | 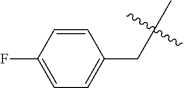 [SMILES: *Cc1ccc(F)cc1] | 91 | I-1-87 | i-Pr | — | 2 | i-Pr |
| 24 | I-1-24 | 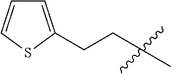 [SMILES: *CCc1cccs1] | H | 62 | I-1-57 | 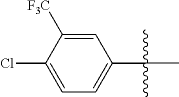 [SMILES: *c1ccc(Cl)c(C(F)(F)F)c1] | 92 | I-1-88 | 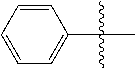 [SMILES: *c1ccccc1] | — | 2 | H |
| 25 | I-1-25 | 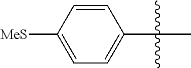 [SMILES: *c1ccc(SC)cc1] | H | 63 | I-1-58 | 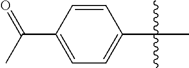 [SMILES: *c1ccc(C(C)=O)cc1] | 93 | I-1-89 | n-pentyl | — | 2 | H |
| 26 | I-1-26 | 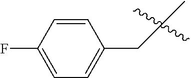 [SMILES: *Cc1ccc(F)cc1] | H | 64 | I-1-59 | 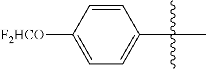 [SMILES: *c1ccc(OC(F)F)cc1] | 94 | I-1-90 | cyclopentyl | — | 2 | H |
| 27 | I-1-27 | 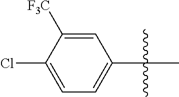 [SMILES: *c1ccc(Cl)c(C(F)(F)F)c1] | H | 65 | I-1-60 | 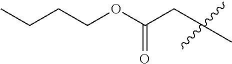 [SMILES: *CC(=O)OCCCC] | 95 | I-1-91 | 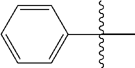 [SMILES: *c1ccccc1] | — | 3 | H |
| 28 | I-1-28 | 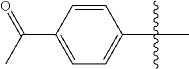 [SMILES: *c1ccc(C(C)=O)cc1] | H | 66 | I-1-61 | 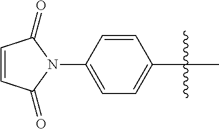 [SMILES: *c1ccc(N2C(=O)C=CC2=O)cc1] | 96 | I-1-92 | n-pentyl | — | 3 | H |
| 29 | I-1-29 | 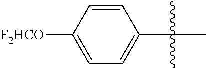 [SMILES: *c1ccc(OC(F)F)cc1] | H | 67 | I-1-62 | 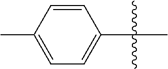 [SMILES: *c1ccc(C)cc1] | 97 | I-1-93 | cyclopentyl | | 3 | H |
| 30 | I-1-30 | 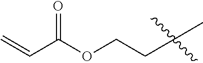 [SMILES: *CCOC(=O)C=C] | H | 68 | I-1-63 | cyclopentyl | 98 | I-1-94 | n-pentanoyl | 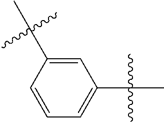 [SMILES: *c1cccc(*)c1] | 2 | H |
| 31 | I-1-31 | 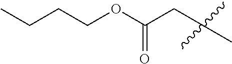 [SMILES: *CC(=O)OCCCC] | H | | | | 99 | I-1-95 | cyclopentyloxy | 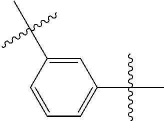 [SMILES: *c1cccc(*)c1] | 2 | H |
| 32 | I-1-32 | 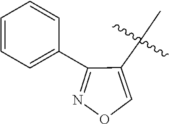 [SMILES: *c1conc1-c1ccccc1] | H | | | | 100 | I-1-96 | benzoyl | 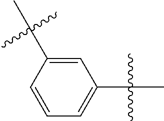 [SMILES: *c1cccc(*)c1] | 2 | H |
| 33 | I-1-33 | 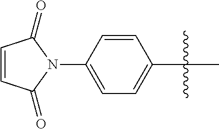 [SMILES: *c1ccc(N2C(=O)C=CC2=O)cc1] | H | | | | | | | | | |
| 34 | I-1-82 | 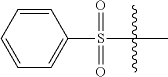 [SMILES: *S(=O)(=O)c1ccccc1] | H | | | | | | | | | |
| 35 | I-1-97 | 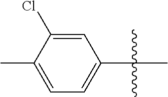 [SMILES: *c1ccc(C)c(Cl)c1] | H | | | | | | | | | |
| 36 | I-1-98 | 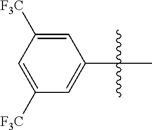 [SMILES: *c1cc(C(F)(F)F)cc(C(F)(F)F)c1] | H | | | | | | | | | |
| 37 | I-1-99 | 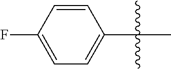 [SMILES: *c1ccc(F)cc1] | H | | | | | | | | | |
| 38 | I-1-100 | CH₃ | CH₃ | | | | | | | | | |

Alternatively, the compound as shown in formula (I) is selected from the following compounds: 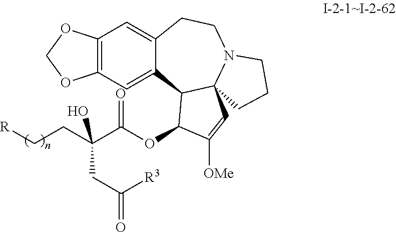
wherein the groups R, R³ and n have the definitions described in the following table:

| number | compound | R | R³ | n |
|---|---|---|---|---|
| 101 | I-2-1 | 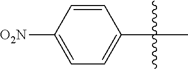 [SMILES: *c1ccc([N+](=O)[O-])cc1] | OMe | 1 |
| 102 | I-2-2 | 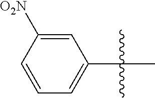 [SMILES: *c1cccc([N+](=O)[O-])c1] | OMe | 1 |
| 103 | I-2-3 | 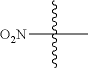 [SMILES: *[N+](=O)[O-]] | OMe | 1 |
| 104 | I-2-4 | 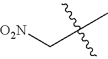 [SMILES: *C[N+](=O)[O-]] | OMe | 1 |
| 105 | I-2-5 | 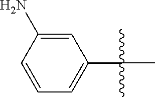 [SMILES: *c1cccc(N)c1] | OMe | 1 |
| 106 | I-2-6 | 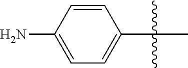 [SMILES: *c1ccc(N)cc1] | OMe | 1 |
| 107 | I-2-7 | 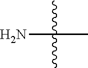 [SMILES: *N] | OMe | 1 |
| 108 | I-2-8 | 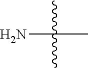 [SMILES: *N] | OMe | 2 |
| 109 | I-2-9 | 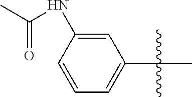 [SMILES: *c1cccc(NC(C)=O)c1] | OMe | 1 |
| 110 | I-2-10 | 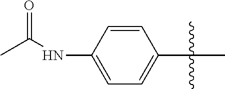 [SMILES: *c1ccc(NC(C)=O)cc1] | OMe | 1 |
| 111 | I-2-11 | 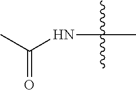 [SMILES: *NC(C)=O] | OMe | 1 |
| 112 | I-2-12 | 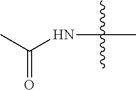 [SMILES: *NC(C)=O] | OMe | 2 |
| 113 | I-2-13 | 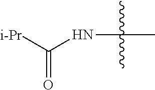 [SMILES: *NC(=O)C(C)C] | OMe | 1 |
| 114 | I-2-14 | 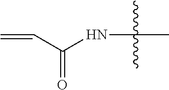 [SMILES: *NC(=O)C=C] | OMe | 1 |
| 115 | I-2-15 | 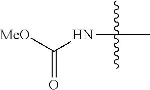 [SMILES: *NC(=O)OC] | OMe | 1 |
| 116 | I-2-16 | 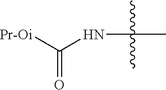 | OMe | 1 |
| 117 | I-2-17 | 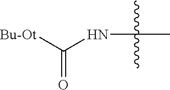 | OMe | 1 |
| 118 | I-2-18 | 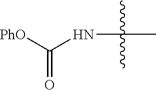 [SMILES: *NC(=O)Oc1ccccc1] | OMe | 1 |
| 119 | I-2-19 | 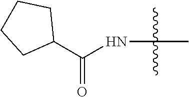 [SMILES: *NC(=O)C1CCCC1] | OMe | 2 |
| 120 | I-2-20 | 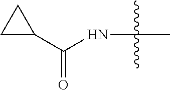 [SMILES: *NC(=O)C1CC1] | OMe | 2 |
| 121 | I-2-21 | 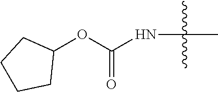 [SMILES: *NC(=O)OC1CCCC1] | OMe | 2 |
| 122 | I-2-22 | 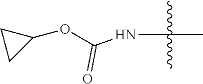 [SMILES: *NC(=O)OC1CC1] | OMe | 2 |
| 123 | I-2-23 | 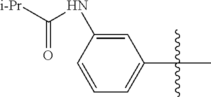 [SMILES: *c1cccc(NC(=O)C(C)C)c1] | OMe | 1 |
| 124 | I-2-24 | 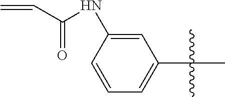 [SMILES: *c1cccc(NC(=O)C=C)c1] | OMe | 1 |
| 125 | I-2-25 | 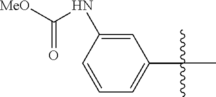 [SMILES: *c1cccc(NC(=O)OC)c1] | OMe | 1 |
| 126 | I-2-26 | 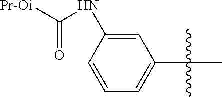 | OMe | 1 |
| 127 | I-2-27 | 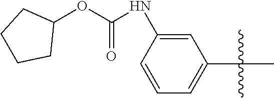 [SMILES: *c1cccc(NC(=O)OC2CCCC2)c1] | OMe | 1 |
| 128 | I-2-28 | 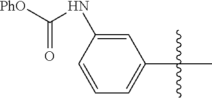 [SMILES: *c1cccc(NC(=O)Oc2ccccc2)c1] | OMe | 1 |
| 129 | I-2-29 | 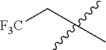 [SMILES: *CC(F)(F)F] | OMe | 1 |
| 130 | I-2-30 | 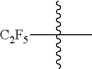 | OMe | 1 |
| 131 | I-2-31 | 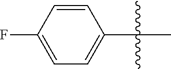 [SMILES: *c1ccc(F)cc1] | OMe | 1 |
| 132 | I-2-32 | 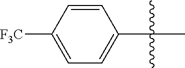 [SMILES: *c1ccc(C(F)(F)F)cc1] | OMe | 1 |
| 133 | I-2-33 | 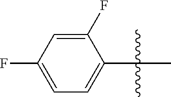 [SMILES: *c1ccc(F)cc1F] | OMe | 1 |
| 134 | I-2-34 | 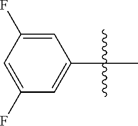 [SMILES: *c1cc(F)cc(F)c1] | OMe | 1 |
| 135 | I-2-35 | 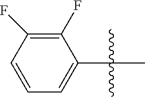 [SMILES: *c1cccc(F)c1F] | OMe | 1 |
| 136 | I-2-36 | 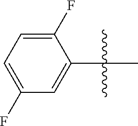 [SMILES: *c1cc(F)ccc1F] | OMe | 1 |
| 137 | I-2-37 | 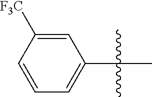 [SMILES: *c1cccc(C(F)(F)F)c1] | OMe | 1 |
| 138 | I-2-38 | 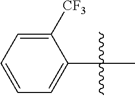 [SMILES: *c1ccccc1C(F)(F)F] | OMe | 1 |
| 139 | I-2-39 | 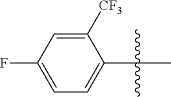 [SMILES: *c1ccc(F)cc1C(F)(F)F] | OMe | 1 |
| 140 | I-2-40 | 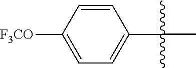 [SMILES: *c1ccc(OC(F)(F)F)cc1] | OMe | 1 |
| 141 | I-2-41 | 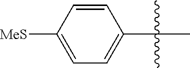 [SMILES: *c1ccc(SC)cc1] | OMe | 1 |
| 142 | I-2-42 | 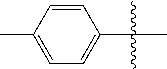 [SMILES: *c1ccc(C)cc1] | OMe | 1 |
| 143 | I-2-43 | 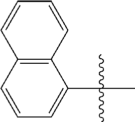 [SMILES: *c1cccc2ccccc12] | OMe | 1 |
| 144 | I-2-44 | 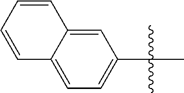 [SMILES: *c1ccc2ccccc2c1] | OMe | 1 |
| 145 | I-2-45 | 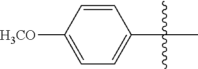 [SMILES: *c1ccc(OC)cc1] | OMe | 1 |
| 146 | I-2-46 | 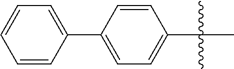 [SMILES: *c1ccc(-c2ccccc2)cc1] | OMe | 1 |
| 147 | I-2-47 | 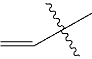 [SMILES: *C=C] | Me | 1 |
| 148 | I-2-48 | 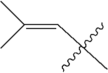 [SMILES: *C=C(C)C] | Me | 1 |
| 149 | I-2-49 | 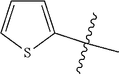 [SMILES: *c1cccs1] | Me | 1 |
| 150 | I-2-50 | 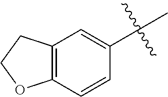 [SMILES: *c1ccc2c(c1)CCO2] | Me | 1 |
| 151 | I-2-51 | i-Pr | Me | 1 |
| 152 | I-2-52 | i-Pr | SMe | 1 |
| 153 | I-2-53 | 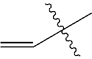 [SMILES: *C=C] | SMe | 1 |
| 154 | I-2-54 | 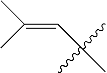 [SMILES: *C=C(C)C] | SMe | 1 |
| 155 | I-2-55 | 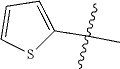 [SMILES: *c1cccs1] | SMe | 1 |
| 156 | I-2-56 | 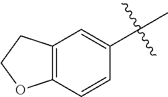 [SMILES: *c1ccc2c(c1)CCO2] | SMe | 1 |
| 157 | I-2-57 | C₂H₅ | H | 1 |
| 158 | I-2-58 | 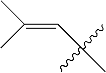 [SMILES: *C=C(C)C] | H | 1 |
| 159 | I-2-59 | 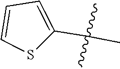 [SMILES: *c1cccs1] | H | 1 |
| 160 | I-2-60 | 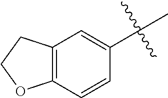 [SMILES: *c1ccc2c(c1)CCO2] | H | 1 |
| 161 | I-2-61 | i-Pr | H | 1 |
| 162 | I-2-62 | 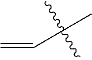 [SMILES: *C=C] | H | 1 |

Alternatively, the compound as shown in formula (I) is selected from the following compounds: 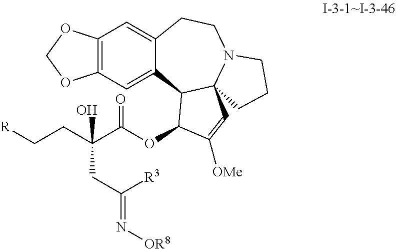
wherein the groups R, R³ and R⁸ have the definitions described in the following table:

| number | compound | R | R³ | R⁸ | Z/E |
|---|---|---|---|---|---|
| 163 | I-3-1 | 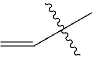 [SMILES: *C=C] | Me | H | E |
| 164 | I-3-2 | 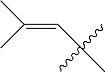 [SMILES: *C=C(C)C] | Me | H | E |
| 165 | I-3-3 | 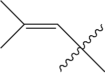 [SMILES: *C=C(C)C] | Me | H | Z |
| 166 | I-3-4 | 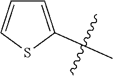 [SMILES: *c1cccs1] | Me | H | E |
| 167 | I-3-5 | 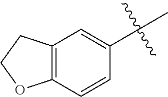 [SMILES: *c1ccc2c(c1)CCO2] | Me | H | Z |
| 168 | I-3-6 | 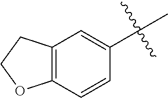 [SMILES: *c1ccc2c(c1)CCO2] | Me | H | E |
| 169 | I-3-7 | i-Pr | H | H | Z |
| 170 | I-3-8 | i-Pr | H | H | E |
| 171 | I-3-9 | 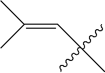 [SMILES: *C=C(C)C] | H | H | Z |
| 172 | I-3-10 | 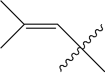 [SMILES: *C=C(C)C] | H | H | E |
| 173 | I-3-11 | Et | H | H | Z |
| 174 | I-3-12 | Et | H | H | E |
| 175 | I-3-13 | i-Pr | H | Ac | Z |
| 176 | I-3-14 | 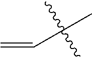 [SMILES: *C=C] | H | Ac | Z |
| 177 | I-3-15 | 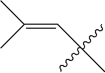 [SMILES: *C=C(C)C] | H | Ac | E |
| 178 | I-3-16 | i-Pr | Me | acryloyl | E |
| 179 | I-3-17 | i-Pr | Me | isobutyryl | E |
| 180 | I-3-18 | 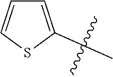 [SMILES: *c1cccs1] | Me | isobutyryl | E |
| 181 | I-3-19 | 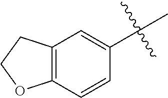 [SMILES: *c1ccc2c(c1)CCO2] | Me | acryloyl | E |
| 182 | I-3-20 | 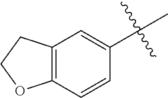 [SMILES: *c1ccc2c(c1)CCO2] | Me | isobutyryl | E |
| 183 | I-3-21 | 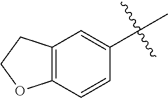 [SMILES: *c1ccc2c(c1)CCO2] | Me | isobutyryl | Z |
| 184 | I-3-22 | i-Pr | H | acryloyl | E |
| 185 | I-3-23 | i-Pr | H | isobutyryl | E |
| 186 | I-3-24 | Et | H | isobutyryl | Z |
| 187 | I-3-25 | 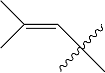 [SMILES: *C=C(C)C] | H | isobutyryl | Z |
| 188 | I-3-26 | i-Pr | Me | 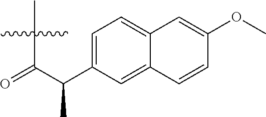 [SMILES: *C(=O)[C@H](C)c1ccc2cc(OC)ccc2c1] | E |
| 189 | I-3-27 | 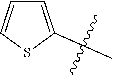 [SMILES: *c1cccs1] | Me | 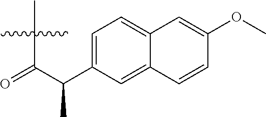 [SMILES: *C(=O)[C@H](C)c1ccc2cc(OC)ccc2c1] | E |
| 190 | I-3-28 | 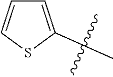 [SMILES: *c1cccs1] | Me | 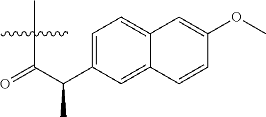 [SMILES: *C(=O)[C@H](C)c1ccc2cc(OC)ccc2c1] | Z |
| 191 | I-3-29 | 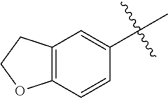 [SMILES: *c1ccc2c(c1)CCO2] | Me | 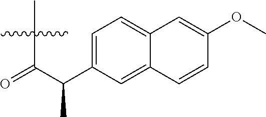 [SMILES: *C(=O)[C@H](C)c1ccc2cc(OC)ccc2c1] | E |
| 192 | I-3-30 | 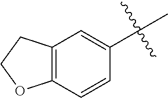 [SMILES: *c1ccc2c(c1)CCO2] | Me | 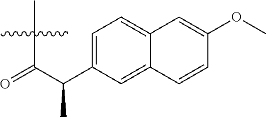 [SMILES: *C(=O)[C@H](C)c1ccc2cc(OC)ccc2c1] | Z |
| 193 | I-3-31 | i-Pr | H | 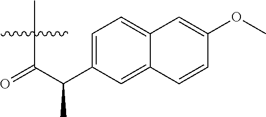 [SMILES: *C(=O)[C@H](C)c1ccc2cc(OC)ccc2c1] | E |
| 194 | I-3-32 | Et | H | 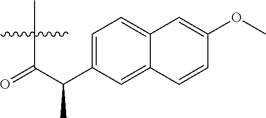 [SMILES: *C(=O)[C@H](C)c1ccc2cc(OC)ccc2c1] | E |
| 195 | I-3-33 | 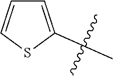 [SMILES: *c1cccs1] | R | 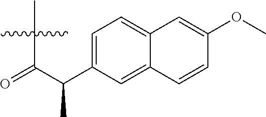 [SMILES: *C(=O)[C@H](C)c1ccc2cc(OC)ccc2c1] | E |
| 196 | I-3-34 | 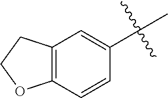 [SMILES: *c1ccc2c(c1)CCO2] | H | 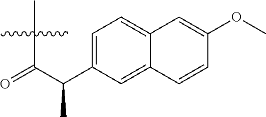 [SMILES: *C(=O)[C@H](C)c1ccc2cc(OC)ccc2c1] | E |
| 197 | I-3-35 | i-Pr | Me | isopentyl | E |
| 198 | I-3-36 | i-Pr | H | isopentyl | E |
| 199 | I-3-37 | Et | H | isopentyl | E |
| 200 | I-3-38 | 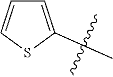 [SMILES: *c1cccs1] | H | isopentyl | E |
| 201 | I-3-39 | i-Pr | Me | 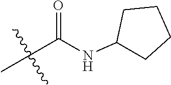 [SMILES: *C(=O)NC1CCCC1] | E |
| 202 | I-3-40 | i-Pr | Me | 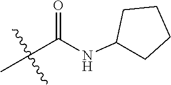 [SMILES: *C(=O)NC1CCCC1] | Z |
| 203 | I-3-41 | 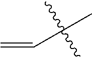 [SMILES: *C=C] | Me | 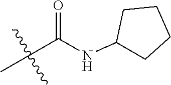 [SMILES: *C(=O)NC1CCCC1] | E |
| 204 | I-3-42 | 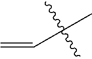 [SMILES: *C=C] | Me | 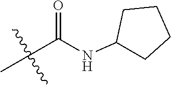 [SMILES: *C(=O)NC1CCCC1] | Z |
| 205 | I-3-43 | 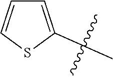 [SMILES: *c1cccs1] | Me | 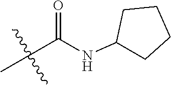 [SMILES: *C(=O)NC1CCCC1] | E |
| 206 | I-3-44 | 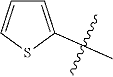 [SMILES: *c1cccs1] | Me | 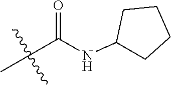 [SMILES: *C(=O)NC1CCCC1] | Z |
| 207 | I-3-45 | 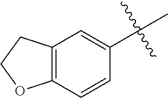 [SMILES: *c1ccc2c(c1)CCO2] | Me | 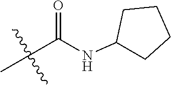 [SMILES: *C(=O)NC1CCCC1] | E |
| 208 | I-3-46 | i-Pr | H | 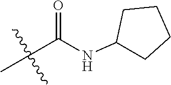 [SMILES: *C(=O)NC1CCCC1] | E |

8. Method for the preparation of the compound as shown in formula (I) or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1, wherein the method of preparation may comprise a method selected from the following methods:
Method (1) 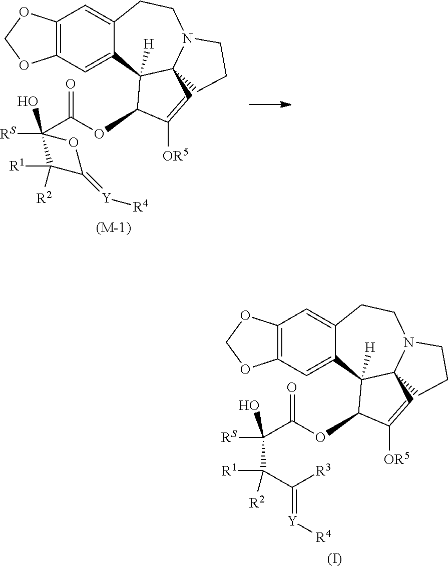
wherein R¹, R², R⁴, R⁵, Y have the definitions described above,
R³ is selected from R⁸—O—,
the step (1) is carried out in the presence of a base and R⁸—OH to prepare a compound of formula (I),
Method (2)
when the substituent R^{S} of the compound of formula (I) contains an amino group, the compound of formula (I) is prepared by reduction using the corresponding nitro compound as substrate,
preferably, the reduction is carried out in the presence of Zn and AcOH,
Method (3)
when the substituent R^{S} of a compound of formula (I) contains an acylamino group, the compound of formula (I) is prepared by acylation reaction using the corresponding amino compound as substrate,
Method (4)
when the substituent group R^{S} of the compound of formula (I) contains a urea group, the compound of formula (I) is prepared by reacting with an isocyanate compound using the corresponding amino compound as substrate,
Method (5)
when the substituent group R^{S} of the compound of formula (I) contains a guanidine group, the compound of formula (I) is prepared by reacting with nitrile amines, acyl nitrile amines, or differently substituted carbodiimide analogs using the corresponding amino compound as substrate,
Method (6) 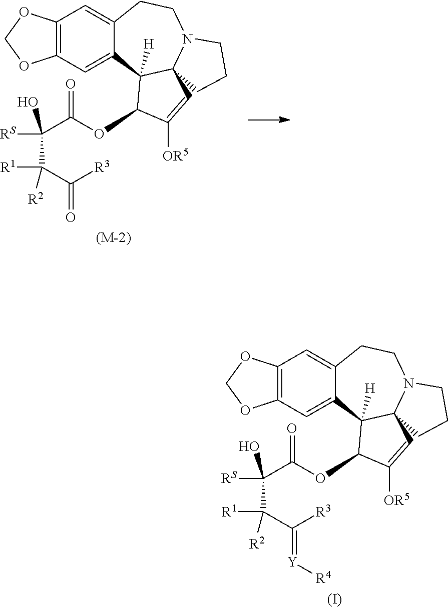
wherein R¹, R², R³, R⁵ have the definitions described above,
R⁴ is selected from hydroxyl,
Y is selected from N,
the step (1) is carried out in the presence of base and hydroxylamine hydrochloride to prepare the compound of formula (I),
Method (7) 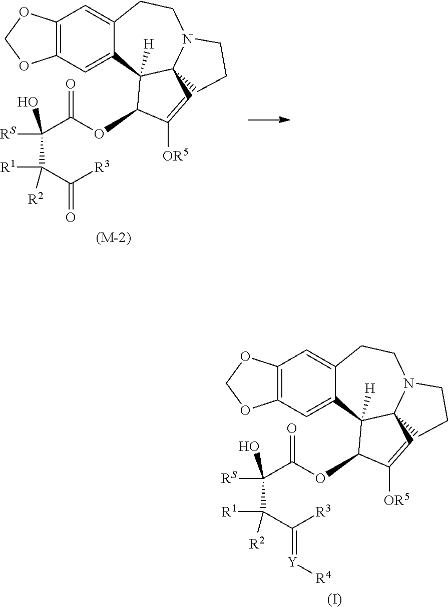
wherein R¹, R², R³, R⁵ have the definitions described above,
R⁴ is selected from R⁹C(═O)O—,
Y is selected from N,
the step (1) is carried out in the presence of a base and C₁₋₂₀ alkyl-C(O)—O—C(O)—C₁₋₂₀ alkyl to prepare the compound of formula (I),
Method (8) 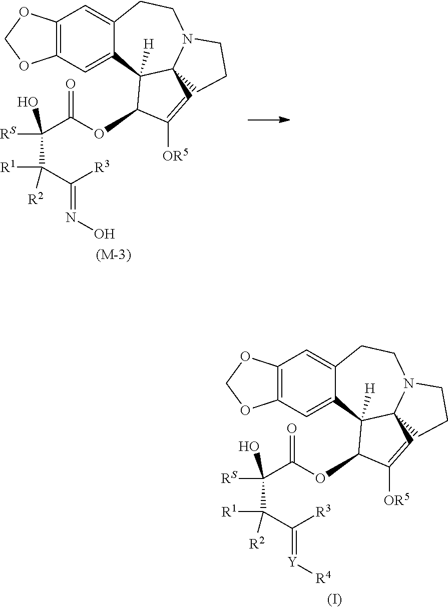
wherein R¹, R², R³, R⁵ have the definitions described above,
R⁴ is selected from R⁹C(═O)O—,
Y is selected from N,
the step (1) is reacted in the presence of a base and R⁹C(═O)Cl or R⁹C(═O)OH to prepare a compound of formula (I),
Method (9) 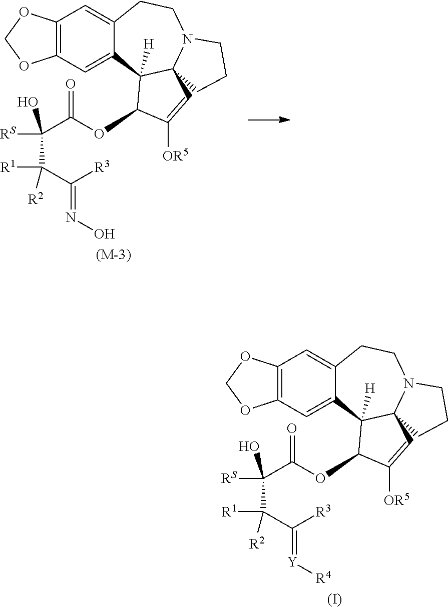
wherein R¹, R², R³, R⁵ have the definitions described above,
R⁴ is selected from R⁸—O—.
R⁸ is selected from C₁₋₂₀ alkyl, C₂₋₂₀ alkenyl, C₃₋₂₀ cycloalkyl, C₃₋₂₀ cycloalkenyl, 3-20 meta heterocyclic, C₆₋₂₀ aryl, 5-20 metaheteroaryl,
Y is selected from N,
the step (1) is performed in the presence of a base and R⁸-L to prepare a compound of formula (I).
L is a leaving group, e.g. halogen, preferably Br or I,
Method (10) 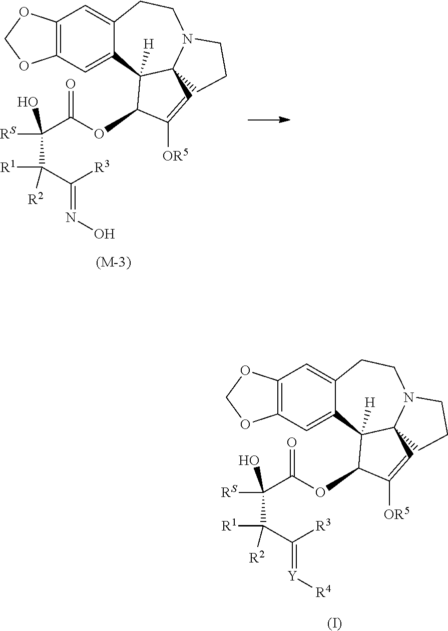
wherein R¹, R², R³, R⁵ have the definitions described above,
R⁴ is selected from R¹⁰NHC(═O)O—.
Y is selected from N.
the step (1) is performed in the presence of R¹⁰—N═C═O to prepare the compound of formula (I), Optionally, the method of preparation further comprises the step of reacting the prepared compound of formula (I) with a pharmaceutically acceptable acid or base to form a salt.

9. A pharmaceutical composition comprising at least one selected from compound as shown in formula (I) or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1.

10. The use of at least one selected from compound as shown in formula (I) or racemate, stereoisomer, reciprocal isomer, isotopic marker, N-oxide, hydrate, solvate, polymorph, metabolite, pharmaceutically acceptable salt, pharmaceutically acceptable ester, or prodrug thereof according to claim 1 in manufacturing a drug, wherein the drug is used for the prevention and/or treatment of diseases caused by uncontrolled cell growth, proliferation and/or survival, inappropriate cellular immune response or inappropriate cellular inflammatory response, or diseases associated with uncontrolled cell growth, proliferation and/or survival, inappropriate cellular immune response or inappropriate cellular inflammatory response, in particular. The diseases are, for example, hematologic tumors, solid tumors and/or their metastases, Such as leukemia and myelodysplastic syndrome, malignant lymphoma, head and neck tumors, including brain tumors and brain metastases, thoracic tumors, including non-small cell lung tumors and small cell lung tumors, gastrointestinal tumors, endocrine tumors, breast tumors and other gynecological tumors, urinary system tumors, including kidney tumors, bladder tumors and prostate tumors, skin tumors and sarcomas, and/or their transfer;
alternatively, the drug is used to inhibit tumor cells, wherein the tumor cells are selected from tumor cells of the blood and/or solid tumors, such as breast cancer cells, liver cancer cells, lung adenocarcinoma cells, leukemia cells, adriamycin resistant strains of leukemia cells, glioblastoma cells, cervical cancer cells, colon cancer cells and/or cerebral microvascular endothelial cells; For example, The tumor cells were selected from human breast cancer cell MCF-7, liver cancer cell HepG2, lung adenocarcinoma cell A549, human leukemia cell HL-60, human leukemia cell adriamycin resistant strain HL-60/ADR, human glioblastoma cell U-87MG, human cervical cancer cell HeLa, human colon cancer cell HCT-116, and human colon cancer cell line. At least one of cell Sw620, human colon cancer cell LoVo, and human microvascular endothelial cell hCMEC/D3;
alternatively, the drug is used to treat hyperproliferative disorders in mammals or to inhibit, block, reduce, reduce, etc., cell proliferation and/or cell division and/or cause apoptosis; Preferably, hyperproliferative conditions include, but are not limited to, psoriasis, keloids and other hyperplasia affecting the skin, benign prostatic hyperplasia (BpH), solid tumors such as breast, respiratory, lung, brain, genital, digestive, urinary, eye, liver, skin, head and neck, thyroid, parathyroid, and their distal metastases thereof;
alternatively, the conditions also include lymphoma, sarcoma and leukemia.
